(19) United States
(12) Patent Application Publication
(10) Pub. No.: US 2018/0354909 A1
(43) Pub. Date: Dec. 13, 2018

(54) SUBSTITUTED BENZIMIDAZOLIUM, PYRIDO-IMIDAZOLIUM, OR PYRAZINO-IMIDAZOLIUM COMPOUNDS AS CHEMOTHERAPEUTICS

(71) Applicant: The Board of Regents of the University of Texas System, Austin, TX (US)

(72) Inventors: Jef DE BRABANDER, Flower Mound, TX (US); Luis F. PARADA, Dallas, TX (US); S. Kyun LIM, Coppell, TX (US); Qiren LIANG, Plano, TX (US); Hua-yu WANG, San Antonio, TX (US); Yufeng SHI, Dallas, TX (US)

(73) Assignee: The Board of Regents of the University of Texas System, Austin, TX (US)

(21) Appl. No.: 16/060,913

(22) PCT Filed: Dec. 9, 2016
(86) PCT No.: PCT/US2016/065751

§ 371 Date: Jun. 8, 2018

Related U.S. Application Data
(60) Provisional application No. 62/266,427, filed on Dec. 11, 2015.

(51) Int. Cl. C07D 235/18; C07D 235/16; C07D 405/04; C07D 401/04; C07D 487/04; C07D 471/04; A61P 35/00

(52) U.S. Cl. CPC C07D 235/18; C07D 235/16; C07D 405/04; A61P 35/00; C07D 487/04; C07D 471/04; C07D 401/04

(57) ABSTRACT

Provided herein are compounds of the formula:(I) wherein: R1, R2, R3, R4, R5, X, A1, A2, A3, and A4 are as defined herein. In some aspects, these compounds may be used to treat cancer and other hyperproliferative disease. In some aspects, compositions, methods of treatment, and methods of synthesis are also provided herein.

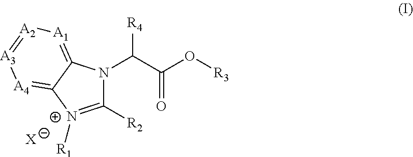

This application claims benefit of priority to U.S. Provisional Application Ser. No. 62/266,427, filed Dec. 11, 2015, the entire contents of which are hereby incorporated by reference.

### BACKGROUND

### 1. Field

The present disclosure relates generally to the field of medicinal chemistry and chemotherapeutic agents. More particularly, it concerns compounds which inhibit replication of cancerous cells.

### 2. Description of Related Art

One of the most common ways of treating cancer is by using compounds which result in cell death particular for rapidly dividing cells. Many of these agents are not selective for the type of cells but rather target all cells which are dividing rapidly and thus lead to significant and, in some cases, life threatening complications. Therefore, there exists a need to develop compounds which exhibit specificity for cancer targets while showing reduced toxicity to non-cancerous cells.

### SUMMARY

In some aspects, the present disclosure provides benzimidazolium, pyridoimidazolium, and pyrazinoimidazole compounds which may be used in the treatment of cancer.

In some aspects, the present disclosure provides compounds of the formula:

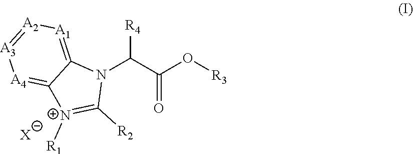

wherein:
   R₁ is alkyl_{(C≤12)}, cycloalkyl_{(C≤12)}, alkenyl_{(C≤12)}, alkynyl_{(C≤12)}, aralkyl_{(C≤12)}, heteroaralkyl_{(C≤12)}, -alkanediyl_{(C≤6)}-cycloalkyl_{(C≤12)}, or a substituted version of any of these groups;
   R₂ is alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, aralkenyl_{(C≤12)}, heteroaryl_{(C≤12)}, heteroaralkyl_{(C≤12)}, heteroaralkenyl_{(C≤12)}, heterocycloalkyl_{(C≤12)}, or a substituted version of any of these groups; or a group of the formula:

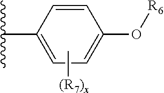

wherein:
   R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
      R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula:

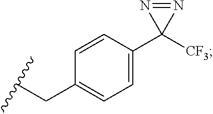 [SMILES: *Cc1ccc(C2(C(F)(F)F)N=N2)cc1]

R₇ is amino, azido, carboxy, cyano, halo, hydroxy, nitro, hydroxysulfonyl, sulfonamide, or
   alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
   —S(O)₂N(Rₐ)R_{b}, —NR_{c}C(O)Rₐ, —C(O)NRₐ(R_{b}), or —NRₐ(R_{b}), or a substituted version of any of these groups;
   wherein:
      Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
      R_{c} is hydrogen, alkyl_{(C≤8)}, or substituted alkyl_{(C≤8)};
x is 0, 1, 2, or 3; or
a group of the formula:

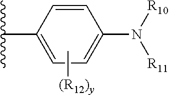

wherein:
   R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
   R₁₂ is amino, azido, carboxy, cyano, halo, hydroxy, nitro, hydroxysulfonyl, sulfonamide, or
      alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
      —S(O)₂N(Rₐ)R_{b}, —NR_{c}C(O)Rₐ, —C(O)NRₐ(R_{b}), or —NRₐ(R_{b}), or a substituted version of any of these groups;
      wherein:
         Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
         R_{c} is hydrogen, alkyl_{(C≤8)}, or substituted alkyl_{(C≤8)};
   y is 0, 1, 2, or 3;
   R₃ is alkyl_{(C≤12)}, cycloalkyl_{(C≤12)}, bicycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, or a substituted version of any of these groups;
   R₄ is hydrogen or alkyl_{(C≤12)}, cycloalkyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)},
      -alkanediyl_{(C≤6)}-heterocycloalkyl_{(C≤12)} or a substituted version of any of these groups;
   A₁, A₂, A₃, and A₄ are independently selected from the group CH, N, or CR₅, wherein:
      R₅ is amino, azido, carboxy, cyano, halo, hydroxy, nitro, hydroxysulfonyl, sulfonamide, or
      alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
      —S(O)₂N(Rₐ)R_{b}, —NR_{c}C(O)Rₐ, —C(O)NRₐ(R_{b}), or —NRₐ(R_{b}), or a substituted version of any of these groups;
         wherein:
            Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
            R_{c} is hydrogen, alkyl_{(C≤8)}, or substituted alkyl_{(C≤8)}; and
   X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, nitrate, or another pharmaceutically acceptable salt;
   provided that when R₁ is methyl, R₂ is ethyl, A₁, A₂, A₃, and A₄ are CH, and R₄ is hydrogen then R₃ is not menthol or methyl;
or a stereoisomer thereof. In some embodiments, the compounds are further defined as:

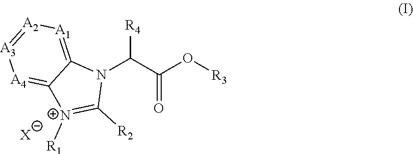

wherein:
   R₁ is alkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, -alkanediyl_{(C≤4)}-cycloalkyl_{(C≤6)}, or a substituted version of any of these groups;
   R₂ is alkyl_{(C≤12)}, cycloalkyl_{(C≤12)}, alkynyl_{(C≤12)}, aryl_{(C≤12)}, aralkyl_{(C≤12)}, heteroaryl_{(C≤12)}, heteroararyl_{(C≤12)}, heterocycloalkyl_{(C≤12)}, or a substituted version of any of these groups; or a group of the formula:

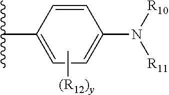

wherein:
   R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
   R₁₂ is amino, azido, carboxy, cyano, halo, hydroxy, nitro, hydroxysulfonyl, sulfonamide, or
   alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤8)}, alkenylthio_{(C≤8)}, alkynylthio_{(C≤8)}, aryl_{(C≤8)}, heteroaryl_{(C≤8)}, heterocycloalkyl_{(C≤8)}, alkylsulfonyl_{(C≤8)}, or a substituted version of any of these groups;
   —S(O)₂N(Rₐ)R_{b}, —NR_{c}C(O)Rₐ, —C(O)NRₐ(R_{b}), or —NRₐ(R_{b}), or a substituted version of any of these groups;
      wherein:
         Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
         R_{c} is hydrogen, alkyl_{(C≤8)}, or substituted alkyl_{(C≤8)}; and
   y is 0, 1, 2, or 3;
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aryl_{(C≤12)}, aralkyl_{(C≤12)}, aryl_{(C≤12)}, aralkyl_{(C≤12)}, heteroaryl_{(C≤12)}, heterocycloalkyl_{(C≤12)}, -alkanediyl_{(C≤6)}-heterocycloalkyl_{(C≤12)}, or a substituted version of any of these groups;
A₁, A₂, A₃, and A₄ are independently selected from the group CH, N, or CR₅, wherein:
   R₅ is amino, azido, carboxy, cyano, halo, hydroxy, nitro, hydroxysulfonyl, sulfonamide, or
   alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
   —S(O)₂N(Rₐ)R_{b}, —NR_{c}C(O)Rₐ, —C(O)NRₐ(R_{b}), or —NRₐ(R_{b}), or a substituted version of any of these groups;
      wherein:
         Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
         R_{c} is hydrogen, alkyl_{(C≤8)}, or substituted alkyl_{(C≤8)}; and
X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, or nitrate;
provided that when R₁ is methyl, R₂ is ethyl, A₁, A₂, A₃, and A₄ are CH, and R₄ is hydrogen then R₃ is not menthol or methyl; or
a stereoisomer thereof. In some embodiments, the compounds are further defined as:

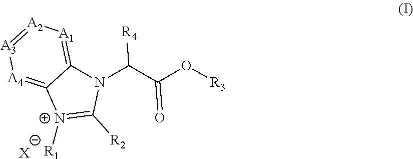

wherein:
   R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
   R₂ is alkyl_{(C≤12)}, cycloalkyl_{(C≤12)}, alkynyl_{(C≤12)}, aryl_{(C≤12)}, aralkyl_{(C≤12)}, heteroaryl_{(C≤12)}, heteroararyl_{(C≤12)}, heterocycloalkyl_{(C≤12)}, or a substituted version of any of these groups; or a group of the formula:

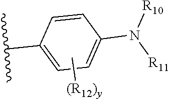

wherein:
   R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
   R₁₂ is azido, carboxy, cyano, halo, nitro, or
   alkyl_{(C≤8)}, acyl_{(C≤8)}, alkoxy_{(C≤8)}, alkylthio_{(C≤12)}, or a substituted version of any of these groups; or
   y is 0, 1, 2, or 3;
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aralkyl_{(C≤12)}, heteroaralkyl_{(C≤12)}, or a substituted version of any of these groups;
A₁, A₂, A₃, and A₄ are independently selected from the group CH, N, or CR₅, wherein:
   R₅ is azido, cyano, halo, nitro, or
   alkyl_{(C≤8)}, alkoxy_{(C≤8)}, alkylthio_{(C≤12)}, or a substituted version of any of these groups;
X is halide, hydroxide, bicarbonate, biphosphate, acetate, formate, citrate, tosylate, mesylate, camphorsulfonate, benzenesulfonate, picrate, nitrate, or a pharmaceutically acceptable salt;
or a stereoisomer thereof. In some embodiments, the compounds are further defined as:

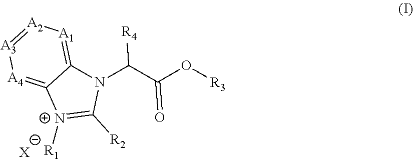

wherein:
   R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
   R₂ is aryl_{(C≤12)}, heteroaryl_{(C≤12)}, or a substituted version of either of these groups wherein the substitution is:
      amino, azido, carboxy, cyano, halo, hydroxy, nitro, hydroxysulfonyl, sulfonamide, or
      alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
      —S(O)₂N(Rₐ)R_{b}, —NR_{c}C(O)Rₐ, —C(O)NRₐ(R_{b}), or —NRₐ(R_{b}), or a substituted version of any of these groups;
         wherein:
            Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
            R_{c} is hydrogen, alkyl_{(C≤8)}, or substituted alkyl_{(C≤8)};
   R₃ is cycloalkyl_{(C≤12)}, fused cycloalkyl_{(C≤12)}, or a substituted version of any of either of these groups;
   R₄ is hydrogen, alkyl_{(C≤12)}, cycloalkyl_{(C≤12)}, aralkyl_{(C≤12)}, heteroaralkyl_{(C≤12)}, or a substituted version of any of these groups;
   A₁, A₂, A₃, and A₄ are independently selected from the group CH, N, or CR₅, wherein:
      R₅ is azido, cyano, halo, nitro, or
      alkyl_{(C≤8)}, alkoxy_{(C≤8)}, alkylthio_{(C≤12)}, or a substituted version of any of these groups;
   X is halide, hydroxide, bicarbonate, biphosphate, acetate, formate, citrate, tosylate, mesylate, camphorsulfonate, benzenesulfonate, picrate, nitrate, or a pharmaceutically acceptable salt; or
a stereoisomer thereof. In some embodiments, the compounds are further defined as:

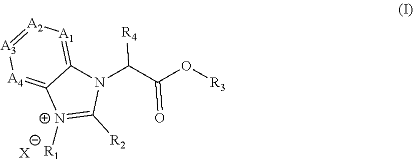

wherein:
   R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
   R₂ is aryl_{(C≤12)}, heteroaryl_{(C≤12)}, or a substituted version of either of these groups wherein the substitution is azido, cyano, or halo; or alkyl_{(C≤8)}, cycloalkyl_{(C≤8)}, heterocycloalkyl_{(C≤8)}, alkoxy_{(C≤8)}, alkenyloxy_{(C≤8)}, alkynyloxy_{(C≤8)}, alkylthio_{(C≤8)}, alkenylthio_{(C≤8)}, alkynylthio_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
   R₃ is cycloalkyl_{(C≤12)}, fused cycloalkyl_{(C≤12)}, or a substituted version of any of either of these groups;
   R₄ is hydrogen, alkyl_{(C≤12)}, cycloalkyl_{(C≤12)}, aralkyl_{(C≤12)}, heteroaralkyl_{(C≤12)}, or a substituted version of any of these groups;
   A₁, A₂, A₃, and A₄ are independently selected from the group CH, N, or CR₅, wherein:
      R₅ is azido, cyano, halo, nitro, or
      alkyl_{(C≤8)}, alkoxy_{(C≤8)}, alkylthio_{(C≤12)}, or a substituted version of any of these groups;
   X is halide, hydroxide, bicarbonate, biphosphate, acetate, formate, citrate, tosylate, mesylate, camphorsulfonate, benzenesulfonate, picrate, nitrate, or a pharmaceutically acceptable salt; or
a stereoisomer thereof.

In some embodiments, R₁ is alkyl_{(C≤12)} such as methyl or ethyl. In other embodiments, R₁ is substituted alkyl_{(C≤12)}. In some embodiments, R₁ is haloalkyl_{(C≤12)} such as fluoromethyl, difluoromethyl, trifluoromethyl, 2,2,2-trifluoroethyl, or pentafluoroethyl. In other embodiments, R₁ is -alkanediyl_{(C≤4)}-cycloalkyl_{(C≤6)} or substituted -alkanediyl_{(C≤4)}-cycloalkyl_{(C≤6)}. In some embodiments, the alkanediyl_{(C≤4)} is —CH₂—. In some embodiments, the cycloalkyl_{(C≤6)} is cyclopropyl. In other embodiments, R₁ is alkenyl_{(C≤12)} such as allyl. In other embodiments, R₁ is alkynyl_{(C≤12)} such as propargyl. In other embodiments, R₁ is aralkyl_{(C≤12)} such as benzyl.

In some embodiments, R₂ is alkyl_{(C≤12)} such as ethyl. In other embodiments, R₂ is alkenyl_{(C≤12)} such as 1-propenyl. In other embodiments, R₂ is aryl_{(C≤12)} or substituted aryl_{(C≤12)} such as phenyl, 2-methylphenyl, 2-nitrophenyl, 3-azidophenyl, 3-bromophenyl, 3-chlorophenyl, 3-cyanophenyl, 3-fluorophenyl, 3-nitrophenyl, 3-trifluoromethylphenyl, 4-azidophenyl, 4-dimethylaminophenyl, 4-dibutylaminophenyl, 4-dicyclopropylaminophenyl, 4-hydroxyphenyl, 4-methylphenyl, 4-tertbutylphenyl, 4-methoxyphenyl, 4-methylthiophenyl, 4-nitrophenyl, 4-trifluoromethylphenyl, 4-chlorophenyl, 4-fluorophenyl, 4-bromophenyl, 3,4-dichlorophenyl, 4-dimethylamino-3-fluorophenyl, 4-dimethylamino-3-methylphenyl, 3-azido-4-propargyloxyphenyl, 4-chloro-3-trifluoromethylphenyl, or 3,5-dichlorophenyl. In other embodiments, R₂ is aralkenyl_{(C≤12)} such as —CH═CHC₆H₅. In other embodiments, R₂ is heteroaryl_{(C≤12)} such as 2-pyrimidyl or 2-furanyl. In other embodiments, R₂ is:

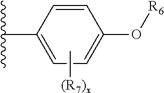

wherein:
   R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
      R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula:

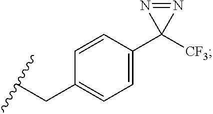 [SMILES: *Cc1ccc(C2(C(F)(F)F)N=N2)cc1]

R₇ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups; or
—S(O)₂N(Rₐ)R_{b}, —NR_{c}C(O)Rₐ, —C(O)NRₐ(R_{b}), or —NRₐ(R_{b}), or a substituted version of any of these groups;
   wherein:
      Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
      R_{c} is hydrogen, alkyl_{(C≤8)}, or substituted alkyl_{(C≤8)};
x is 0, 1, 2, or 3.

In some embodiments, R₆ is alkyl_{(C≤8)} such as methyl or tert-butyl. In other embodiments, R₆ is alkynyl_{(C≤8)}. In some embodiments, x is 0 or 1.

In other embodiments, R₂ is:

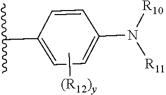

wherein:
   R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
   R₁₂ is azido, carboxy, cyano, halo, nitro, or
   acyl_{(C≤8)}, alkoxy_{(C≤8)}, alkylthio_{(C≤12)}, or a substituted version of any of these groups;
   y is 0, 1, 2, or 3.

In some embodiments, R₁₀ is alkyl_{(C≤6)} such as methyl, ethyl, or butyl. In other embodiments, R₁₀ is cycloalkyl_{(C≤6)} such as cyclopropyl. In some embodiments, R₁₁ is alkyl_{(C≤6)} such as methyl, ethyl, or butyl. In other embodiments, R₁₁ is cycloalkyl_{(C≤6)} such as cyclopropyl. In some embodiments, R₁₀ and R₁₁ are the same. In some embodiments, R₁₀ and R₁₁ are different.

In some embodiments, R₁₂ is alkyl_{(C≤12)} or substituted alkyl_{(C≤12)}. In some embodiments, R₁₂ is alkyl_{(C≤12)} such as methyl. In other embodiments, R₁₂ is substituted alkyl_{(C≤12)} such as trifluoromethyl. In some embodiments, y is 0 or 1.

In some embodiments, R₃ is cycloalkyl_{(C≤12)} or substituted cycloalkyl_{(C≤12)}. In some embodiments, R₃ is cycloalkyl_{(C≤12)}. In some embodiments, R₃ is a monoalkyl substituted cycloalkyl_{(C≤12)} or stereoisomer thereof. In some embodiments, R₃ is a monomethyl cycloalkyl_{(C≤12)} or stereoisomer thereof such as 3-methylcyclohexyl, 4-methylcyclohexyl, or a stereoisomer thereof. In other embodiments, R₃ is a dialkyl substituted cycloalkyl_{(C≤12)} or stereoisomer thereof such as 2-isopropyl-5-methylcyclohexyl or a stereoisomer thereof. In other embodiments, R₃ is adamantanyl. In other embodiments, R₃ is aryl_{(C≤12)} or substituted aryl_{(C≤12)} such as phenyl. In other embodiments, R₃ is aralkyl_{(C≤12)} or substituted aralkyl_{(C≤12)} such as benzyl.

In some embodiments, R₄ is hydrogen. In other embodiments, R₄ is alkyl_{(C≤12)} or substituted alkyl_{(C≤12)} such as methyl, isopropyl, or t-butyl. In other embodiments, R₄ is cycloalkyl_{(C≤12)} or substituted cycloalkyl_{(C≤12)} such as cyclopropyl or cyclopentyl. In other embodiments, R₄ is aralkyl_{(C≤12)} or substituted aralkyl_{(C≤12)} such as benzyl, 4-methylbenzyl, or 4-hydroxybenzyl.

In some embodiments, A₁, A₂, A₃, and A₄ are CH. In other embodiments, one of A₁, A₂, A₃, and A₄ are CR₅ and the remaining three A₁, A₂, A₃, and A₄ are CH. In other embodiments, two of A₁, A₂, A₃, and A₄ are CR₅ and the remaining two A₁, A₂, A₃, and A₄ are CH. In other embodiments, one of A₁, A₂, A₃, and A₄ are N and the remaining three A₁, A₂, A₃, and A₄ are CH or CR₅. In other embodiments, two of A₁, A₂, A₃, and A₄ are N and the remaining two A₁, A₂, A₃, and A₄ are CH or CR₅. In some embodiments, A₁ is N. In some embodiments, A₂ is N. In other embodiments, A₃ is N. In other embodiments, A₄ is N. In other embodiments, A₁ and A₃ are N.

In some embodiments, R₅ is azido, cyano, halo, nitro, or alkyl_{(C≤6)}, alkoxy_{(C≤6)}, alkylthio_{(C≤6)}, or a substituted version of any of these groups. In some embodiments, R₅ is cyano, halo, nitro, or alkyl_{(C≤6)}, alkoxy_{(C≤6)}, or a substituted version of either of these groups. In some embodiments, R₅ is cyano, nitro, fluoro, or chloro. In other embodiments, R₅ is alkyl_{(C≤6)} or substituted alkyl_{(C≤6)} such as methyl or trifluoromethyl. In other embodiments, R₅ is alkoxy_{(C≤6)} such as methoxy.

In some embodiments, X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, nitrate, or another pharmaceutically acceptable counter-ion. In some embodiments, X is halide, hydroxide, bicarbonate, biphosphate, formate, acetate, citrate, mesylate, tosylate, camphorsulfonate, benzenesulfonate, picrate, or nitrate. In some embodiments, X is halide such as chloride or iodide.

In some embodiments, the compounds are further defined as:

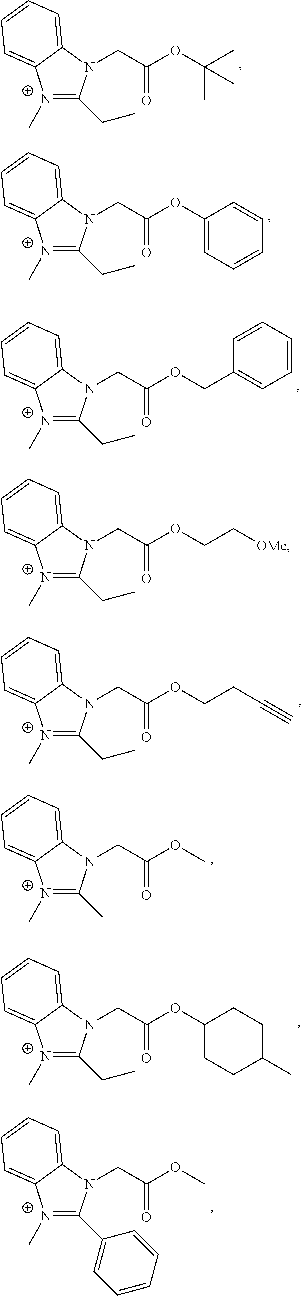

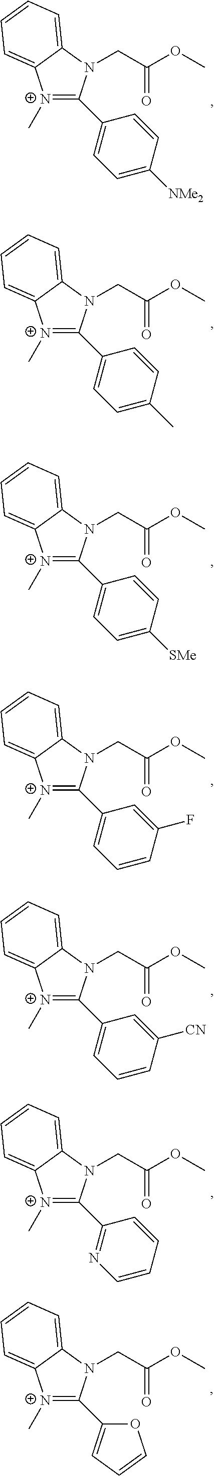

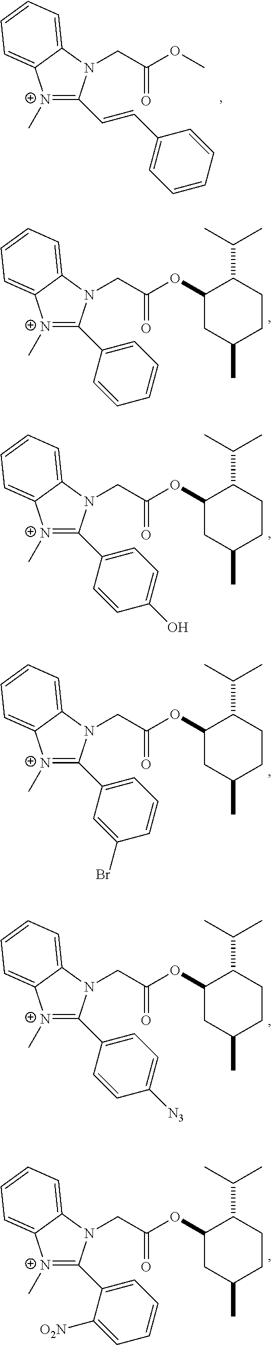

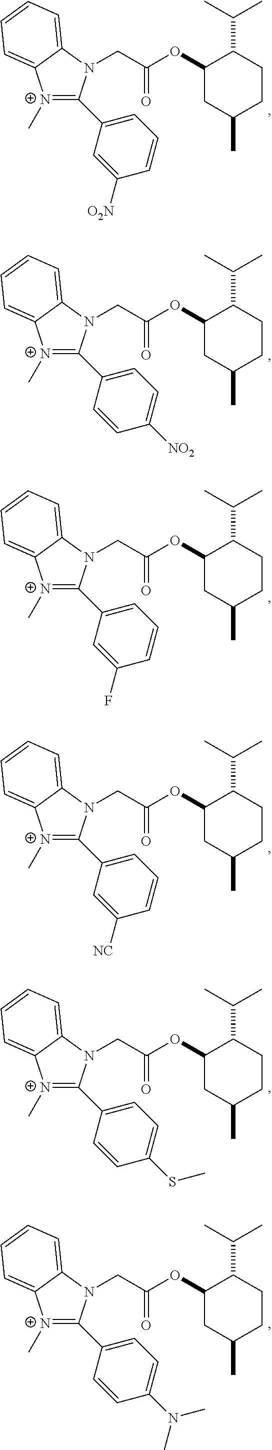

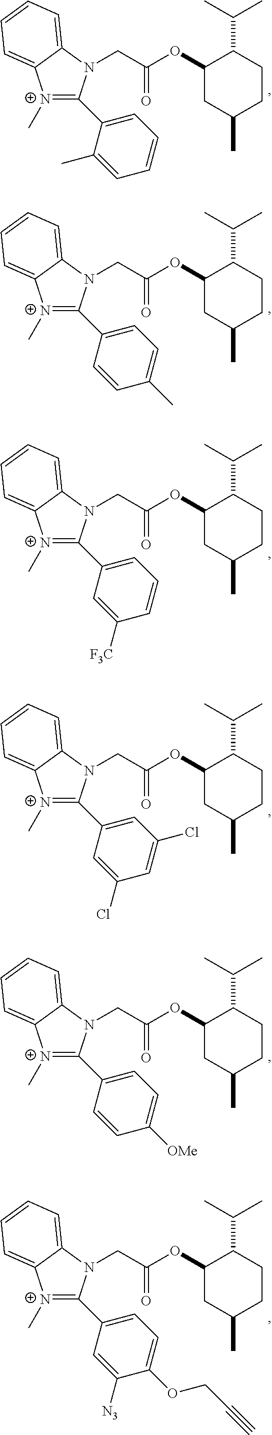

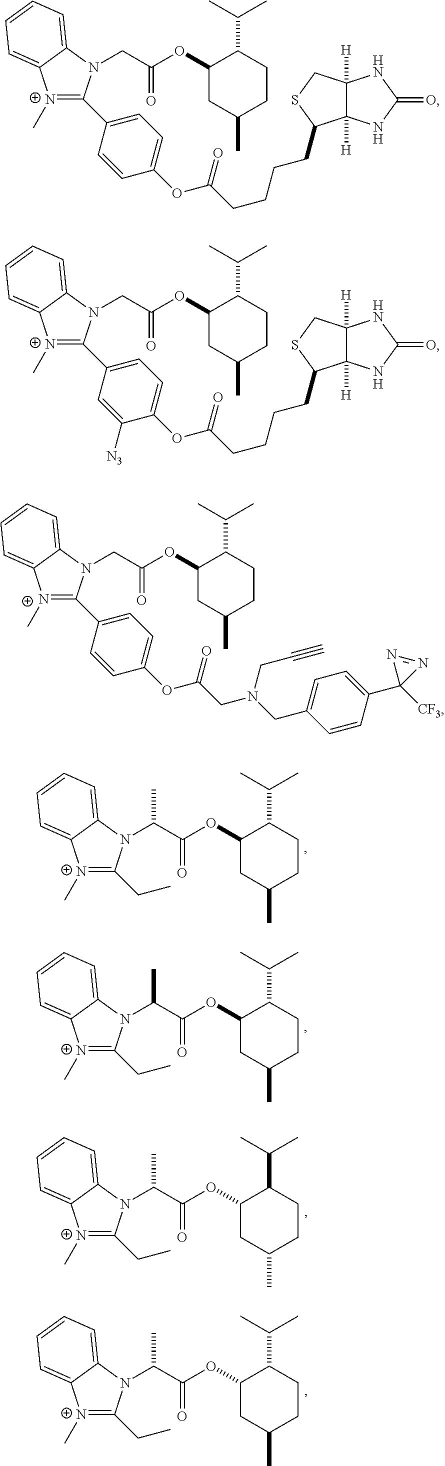

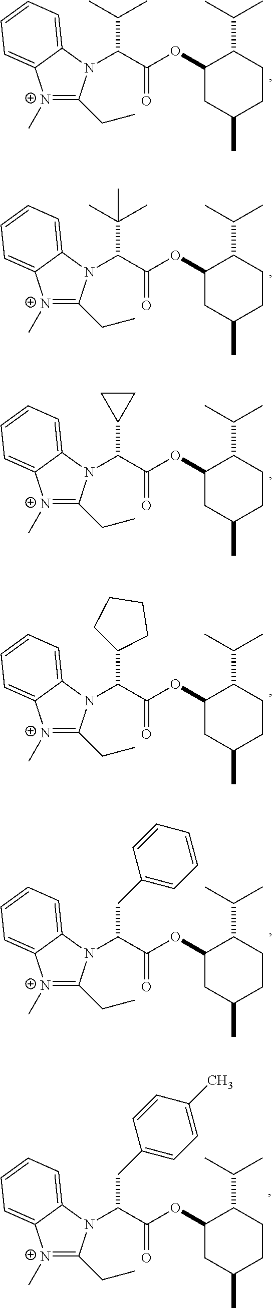

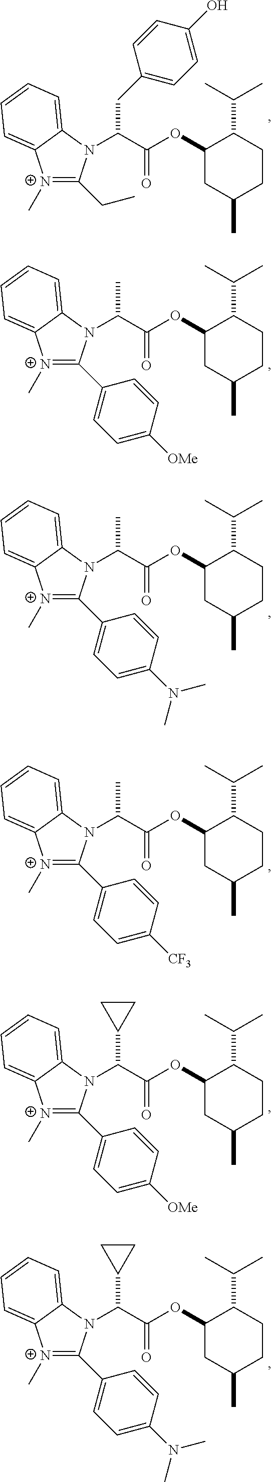

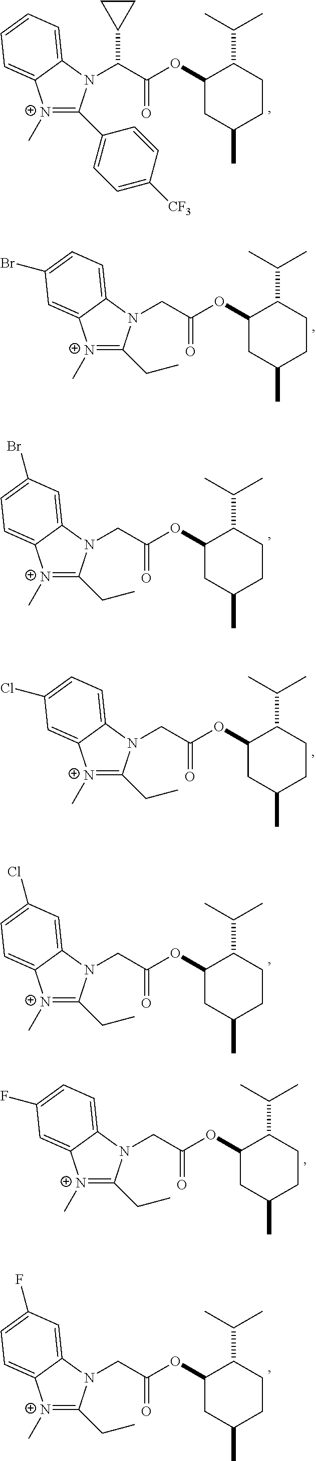

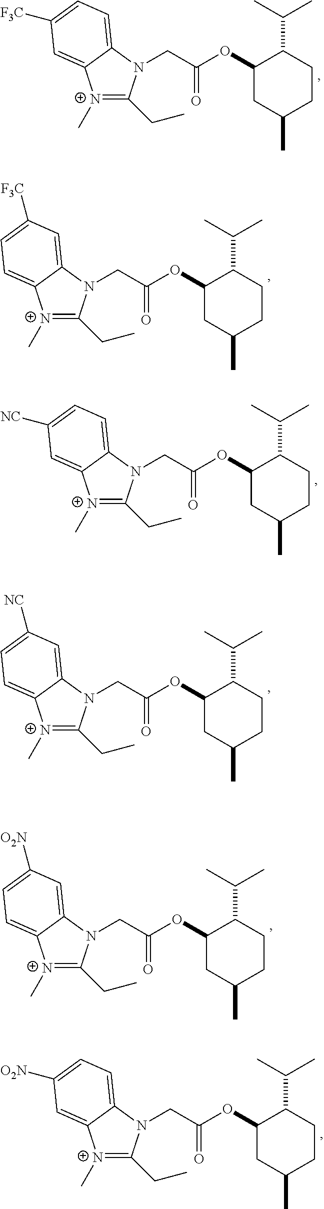

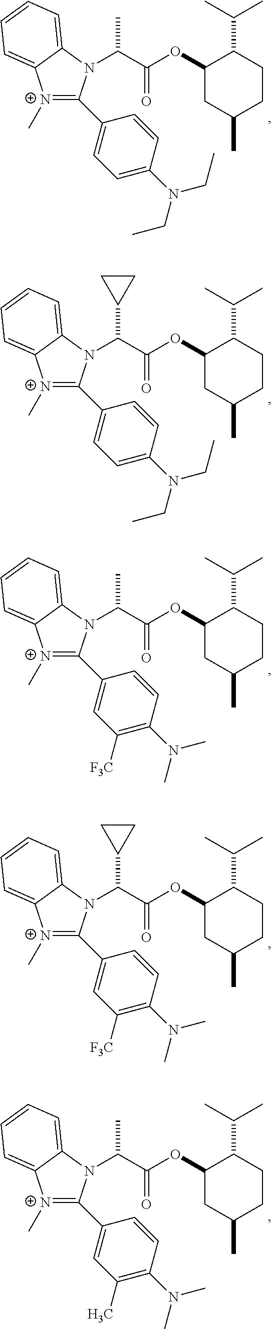

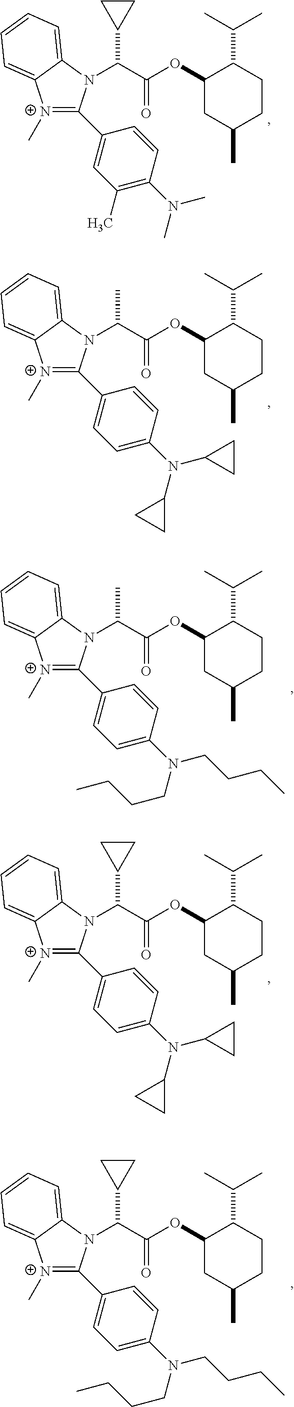

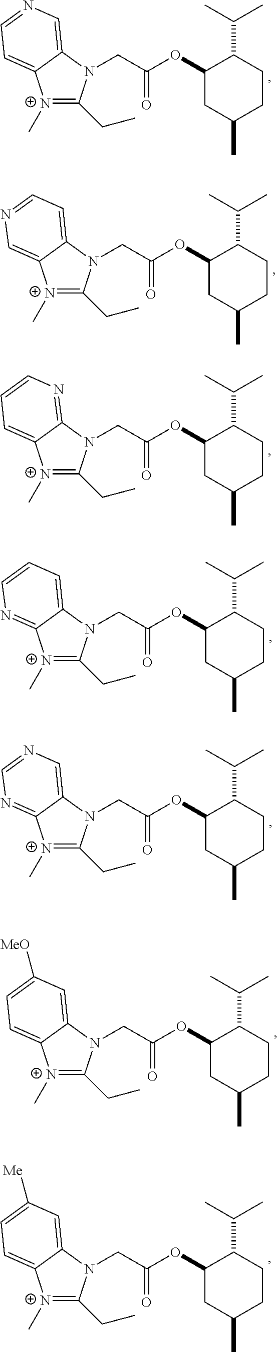

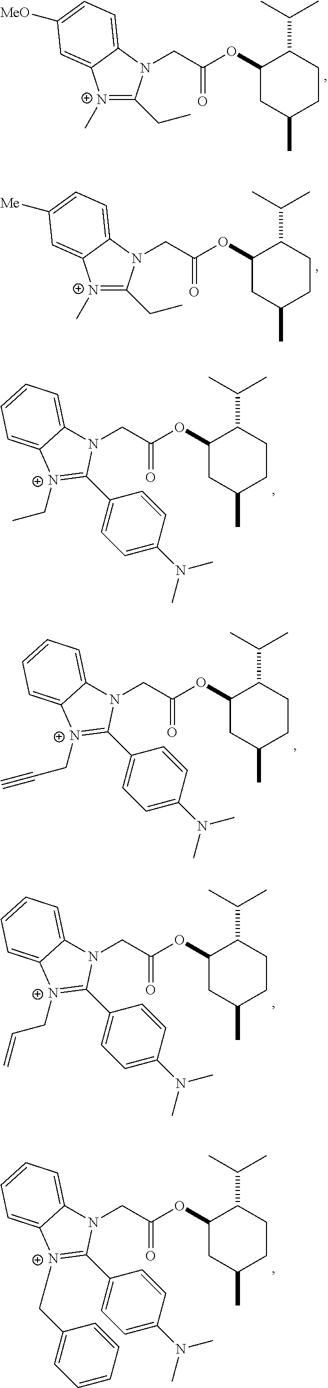

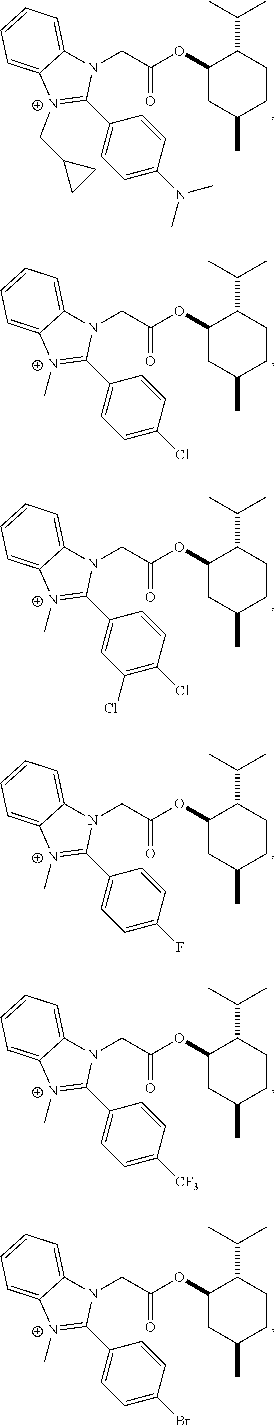

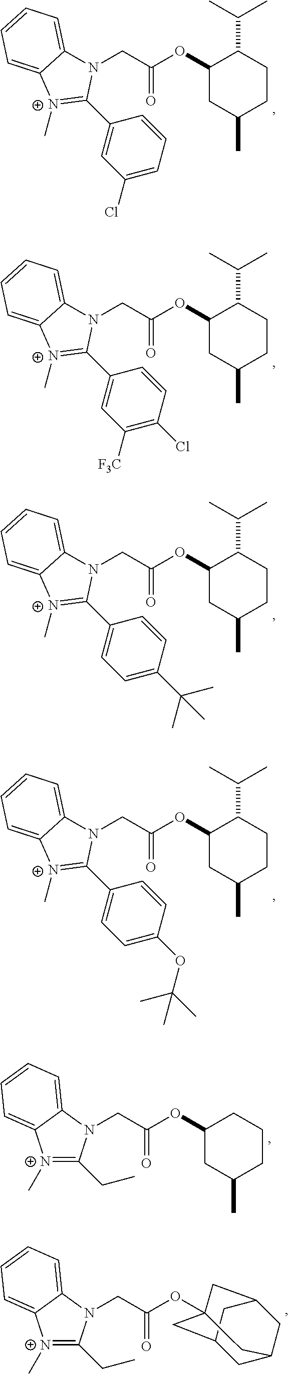

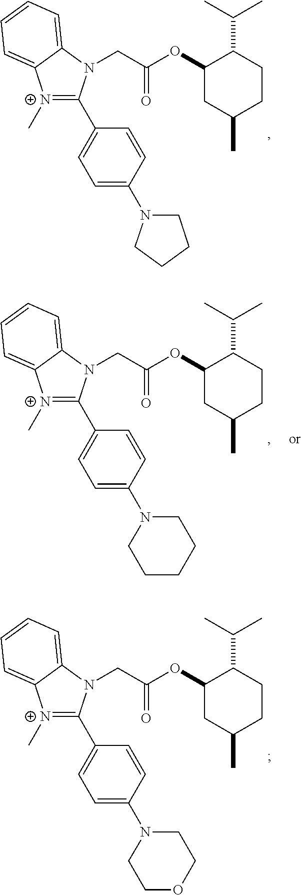

wherein the compound further comprises a pharmaceutically acceptable anion. In some embodiments, the compounds are further defined as:

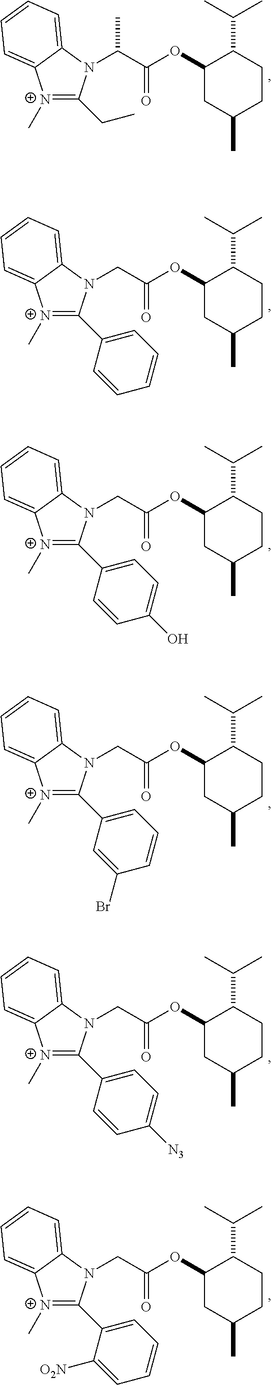

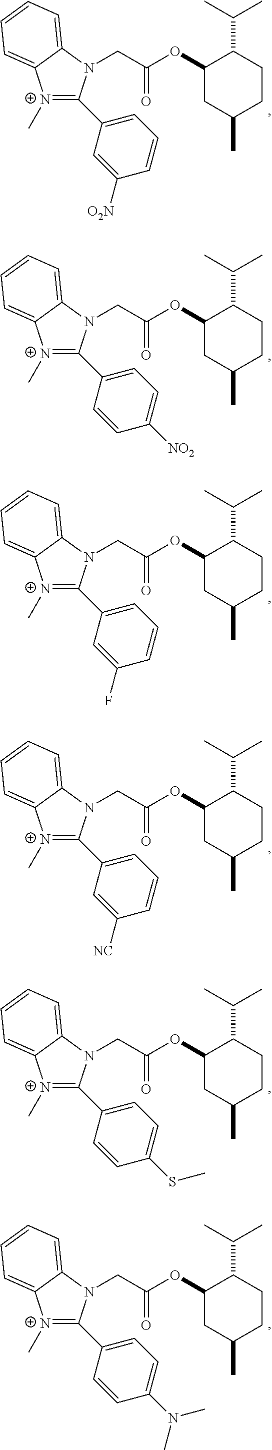

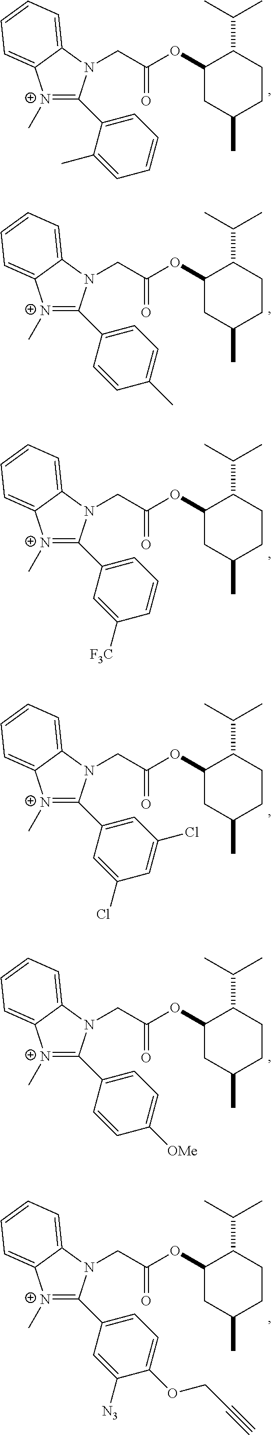

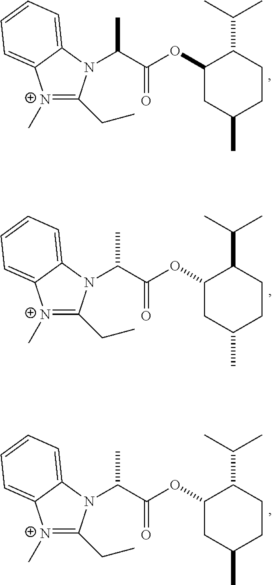

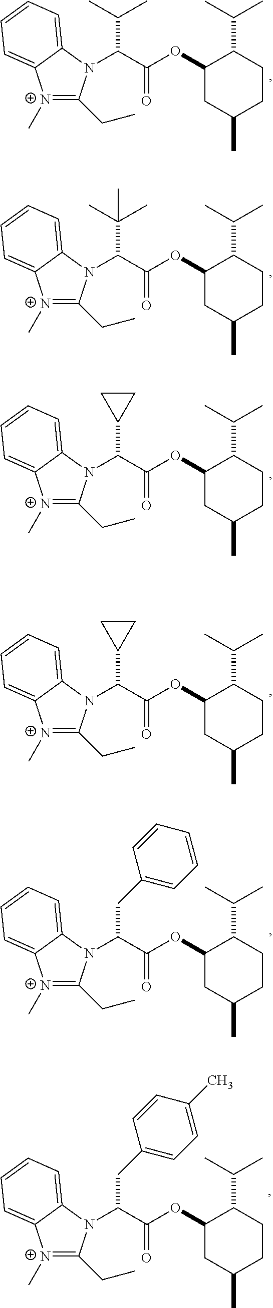

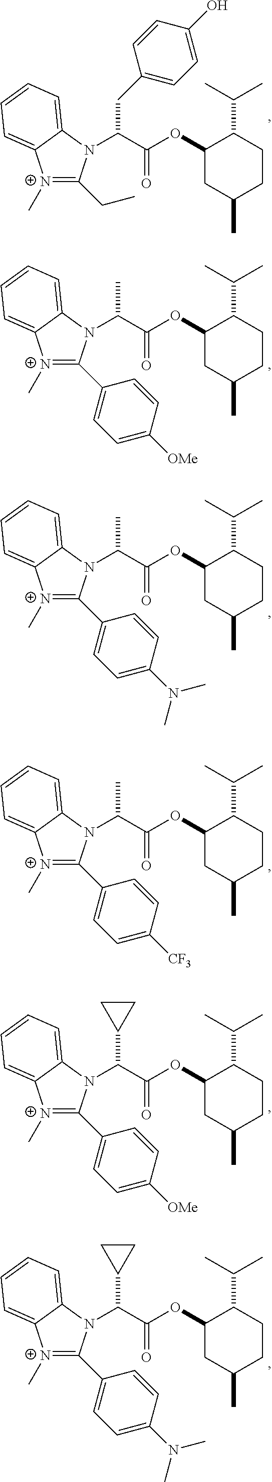

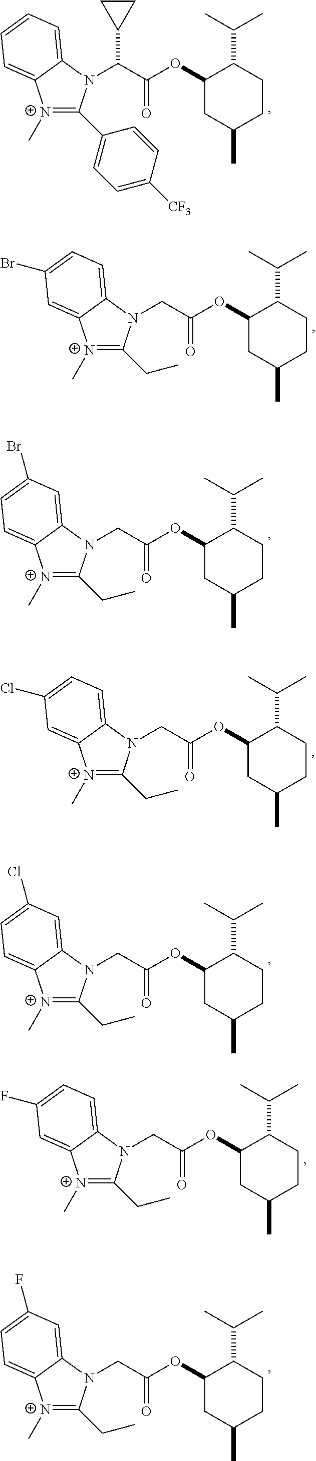

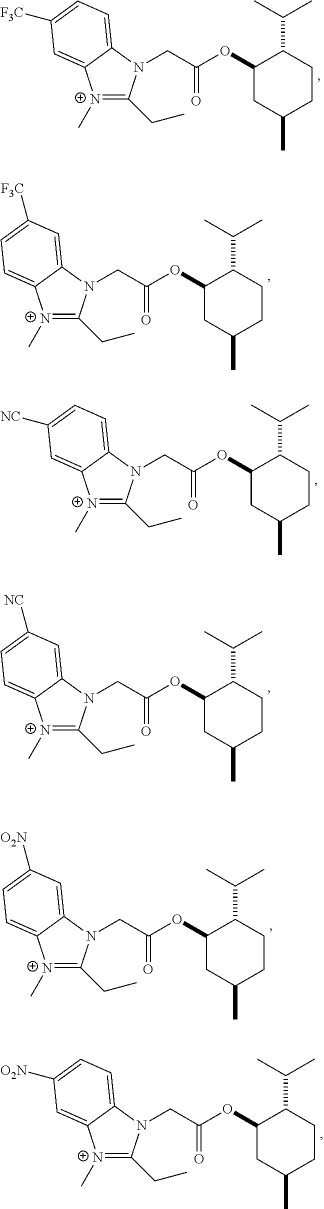

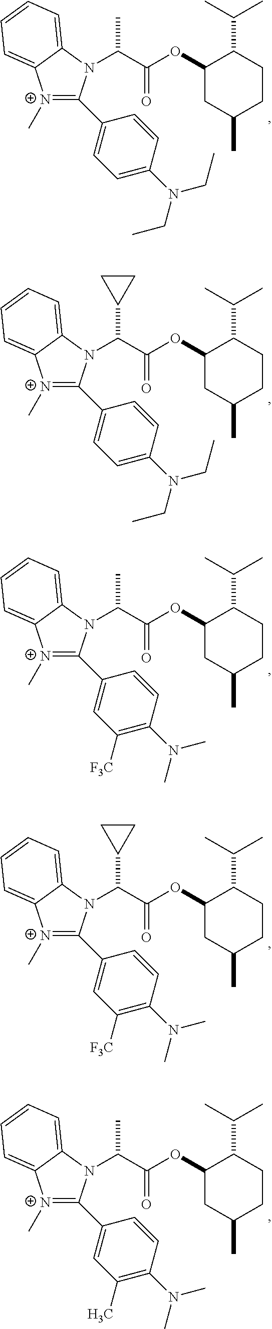

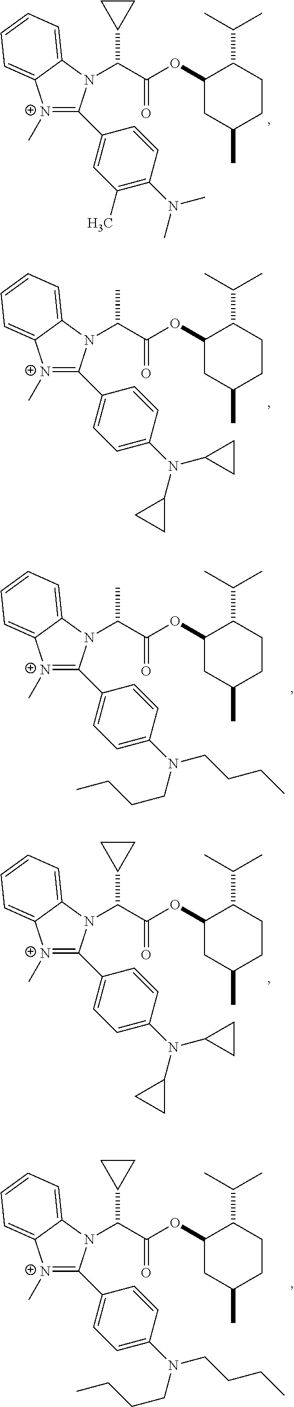

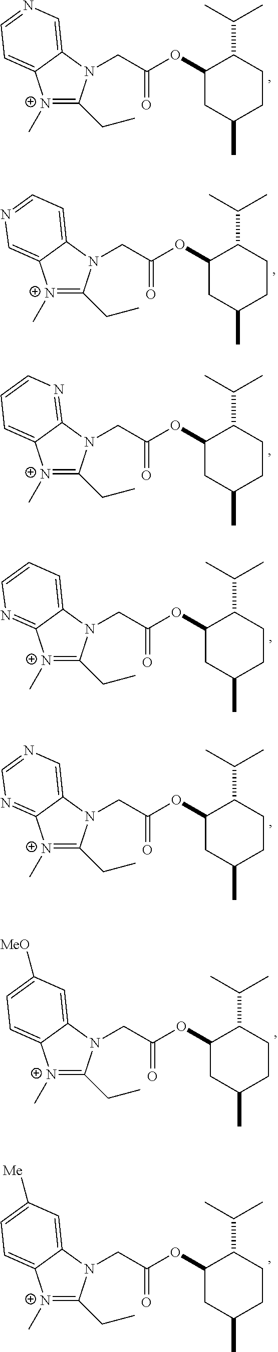

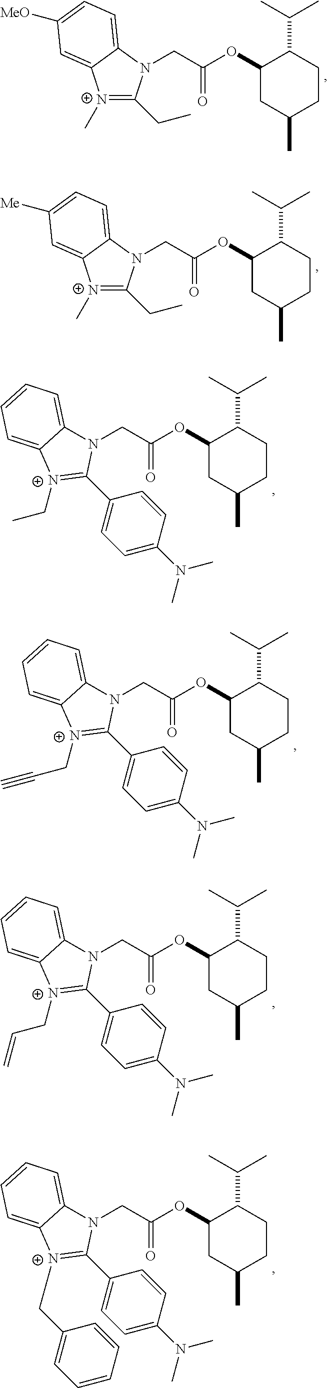

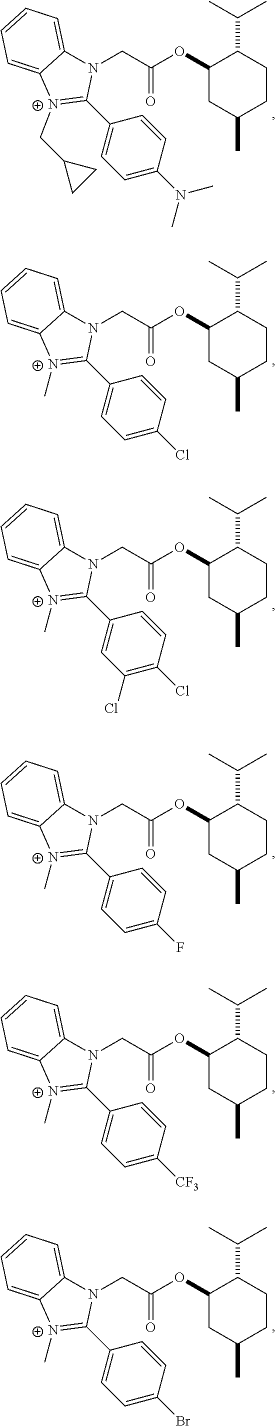

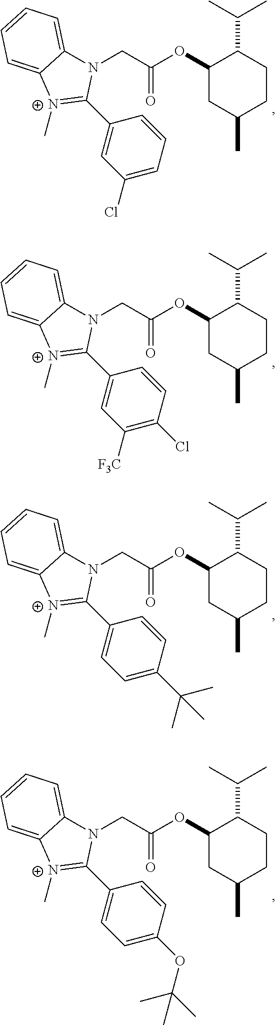

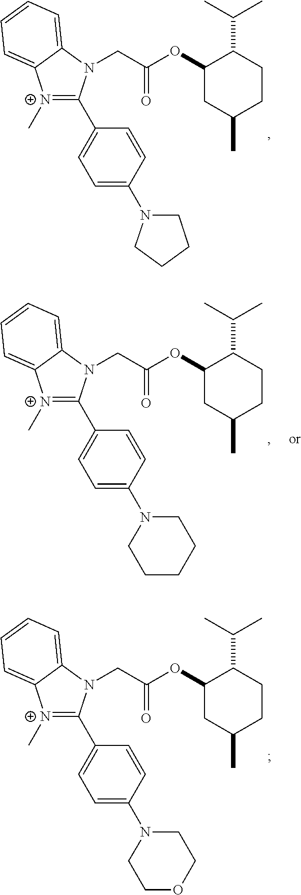

or a pharmaceutically acceptable salt thereof.

In still yet another aspect, the present disclosure provides compounds of the formula:

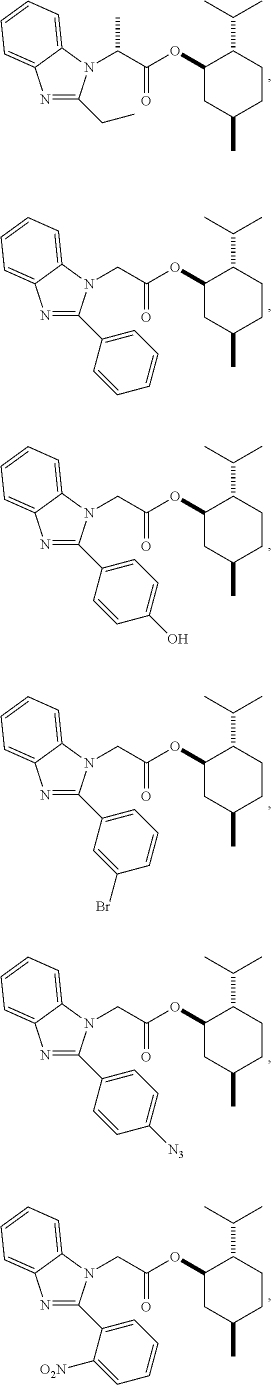

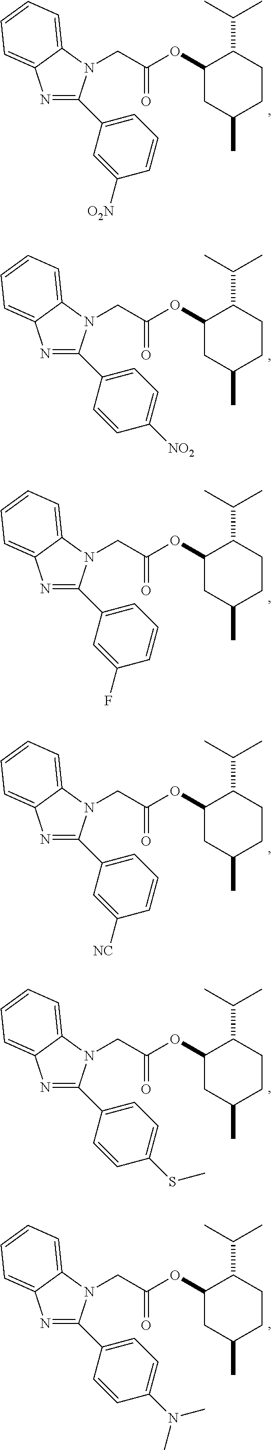

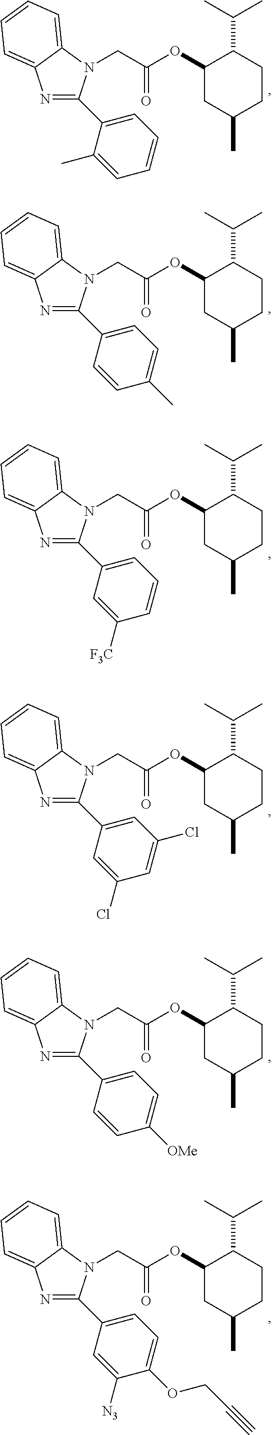

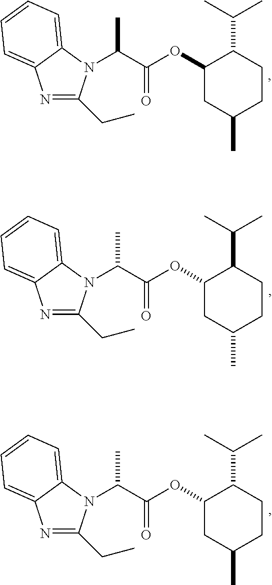

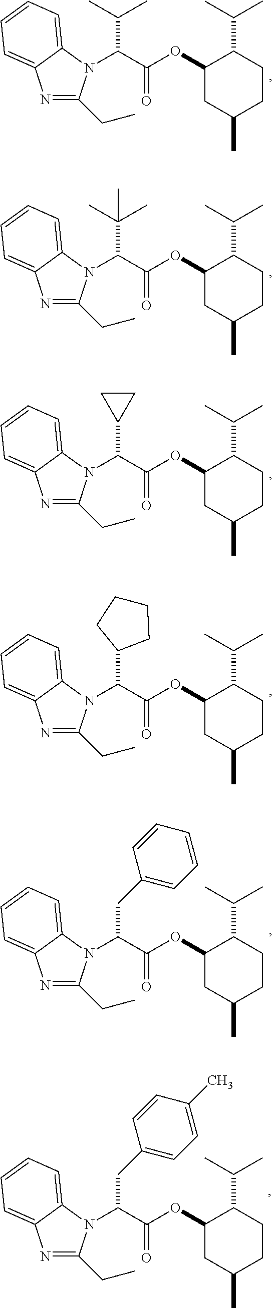

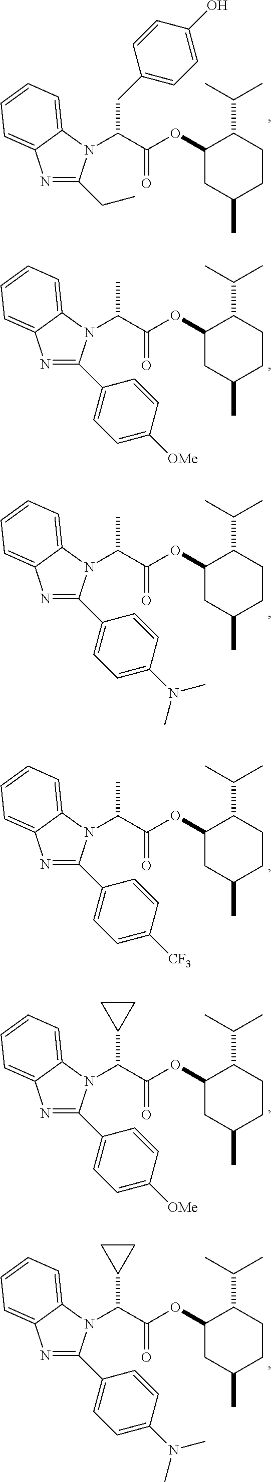

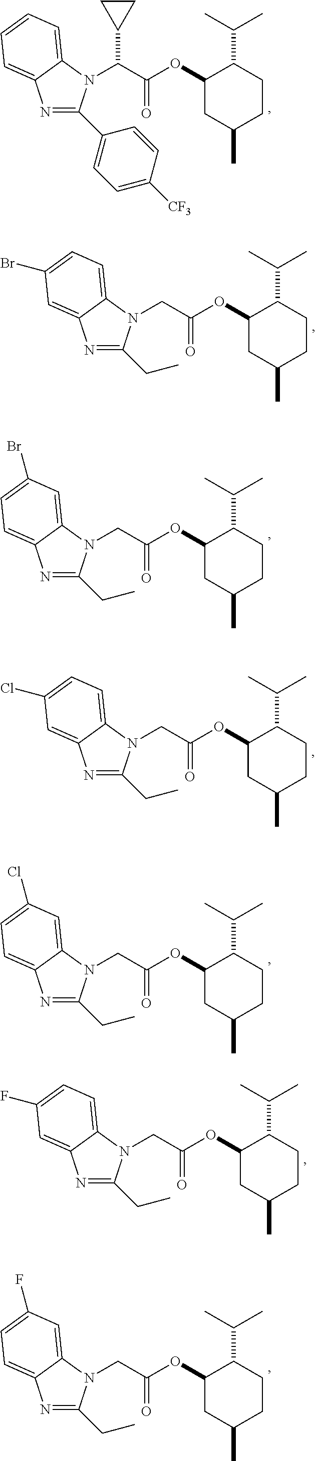

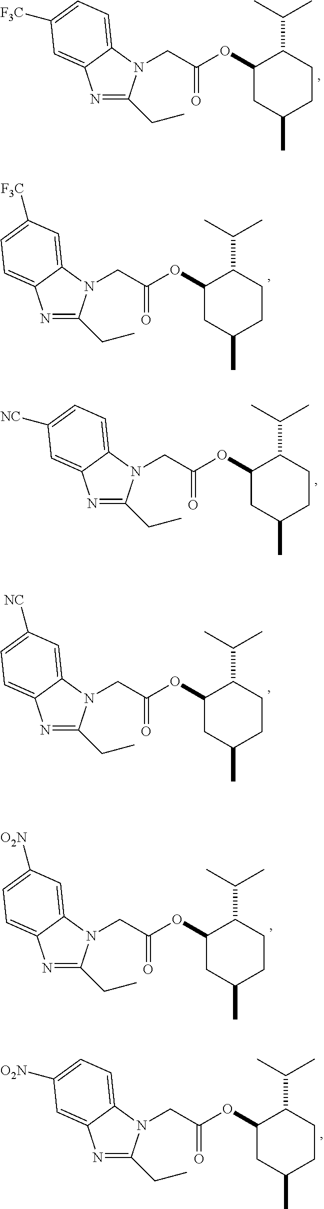

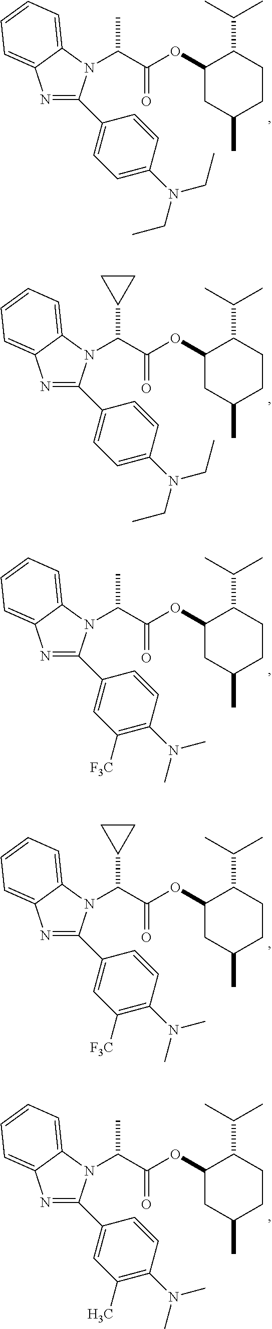

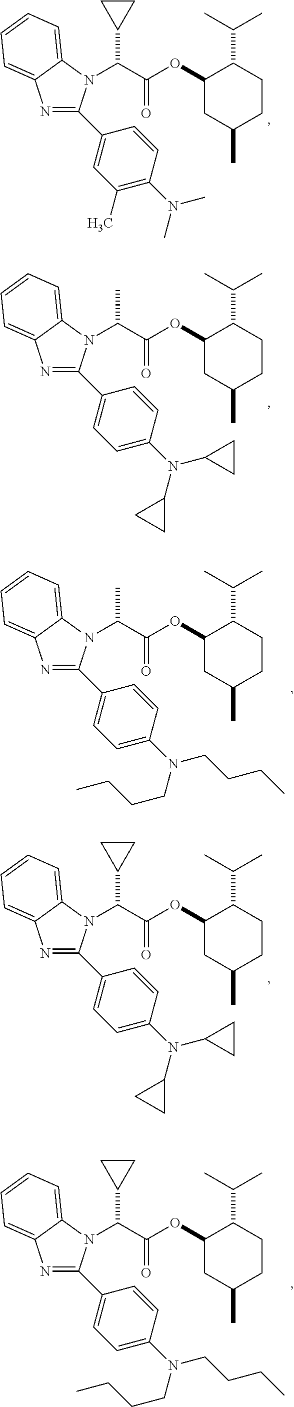

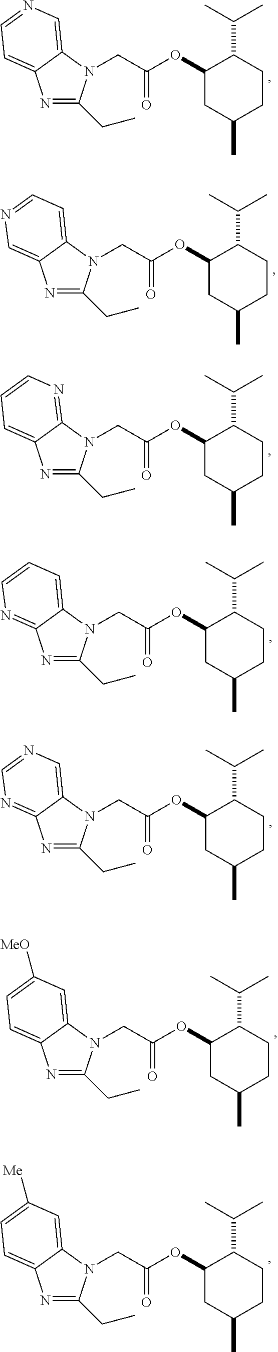

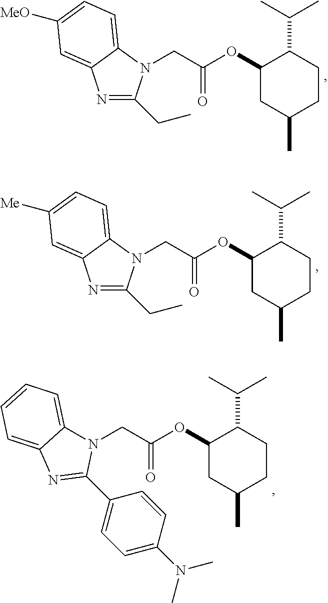

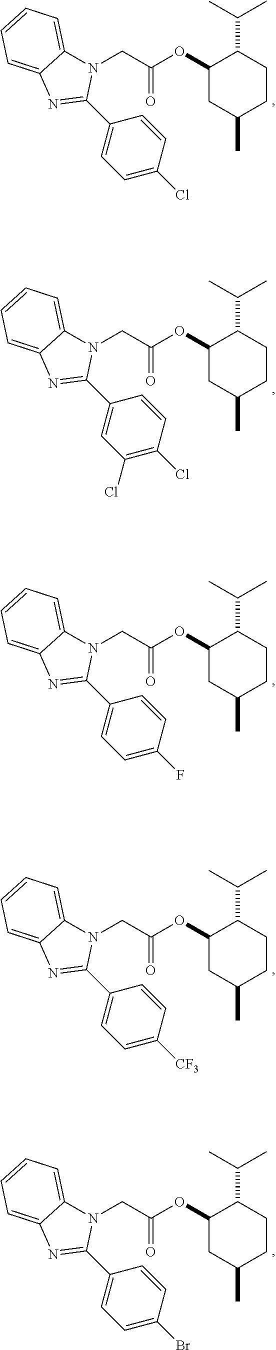

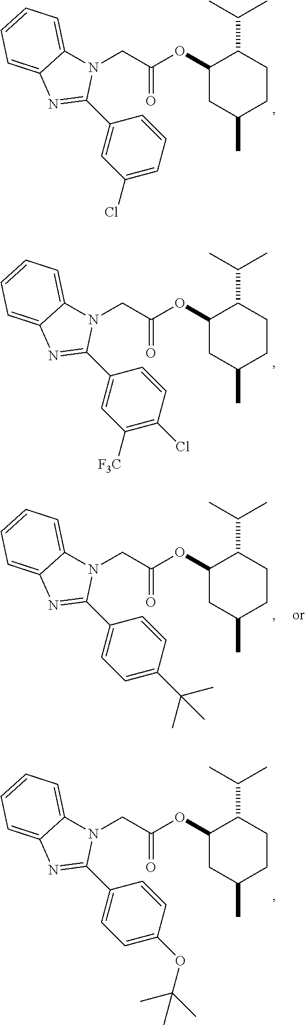

or a pharmaceutically acceptable salt thereof.

In yet another aspect, the present disclosure provides pharmaceutical compositions comprising:

(a) a compound described herein; and

(b) a pharmaceutically acceptable carrier.

In some aspects, the compound is a compound of formula I. In other embodiments, the compound is a compound of the formula:

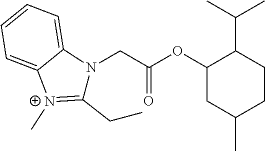 [SMILES: CCc1n(CC(=O)OC2CC(C)CCC2C(C)C)c2ccccc2[n+]1C]

wherein the compound further comprises a pharmaceutically acceptable anion or a pharmaceutically acceptable salt or a stereoisomer thereof.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion. In some embodiments, the pharmaceutical composition is formulated as a unit dose.

In still yet another aspect, the present disclosure provides methods of treating a disease or disorder in a patient comprising administering to the patient in need thereof a pharmaceutically effective amount of a compound or composition described herein. In some embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is a primary brain cancer or a secondary brain cancer. In some embodiments, the cancer has an alter usage of either the glycolysis pathway or the citric acid cycle.

In some embodiments, the methods further comprise administering a second therapeutic agent or modality. In some embodiments, the second therapeutic agent or modality is a second chemotherapeutic agent, surgery, radiotherapy, or immunotherapy. In some embodiments, the methods comprise administering the compound to the patient once. In other embodiments, the methods comprise administering the compound to the patient two or more times.

In yet another aspect, the present disclosure provides methods of inhibiting the oxidative phosphorylation pathway in a cell comprising administering to the cell a therapeutically effective amount of a compound or composition described herein. In some embodiments, the compound inhibits the oxidative phosphorylation pathway in a cancer cell but not in a non-cancerous cell. In some embodiments, the compound inhibits one or more protein(s) which supports the activity of the oxidative phosphorylation pathway. In some embodiments, the cell is contacted in vivo. In other embodiments, the cell is contacted in vitro. In other embodiments, the cell is contacted ex vivo.

In still yet another aspect, the present disclosure provides methods of preparing a compound of formula I comprising reacting a compound with a compound of the formula:

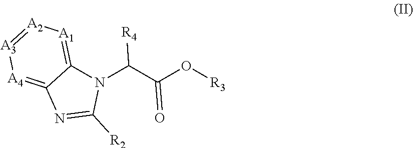

wherein:
   R₂ is alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, aralkenyl_{(C≤12)}, heteroaryl_{(C≤12)}, heteroaralkyl_{(C≤12)}, heteroaralkenyl_{(C≤12)}, heterocycloalkyl_{(C≤12)}, or a substituted version of any of these groups; or a group of the formula:

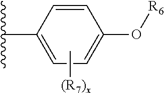

wherein:
   R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
      R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula:

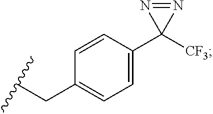 [SMILES: *Cc1ccc(C2(C(F)(F)F)N=N2)cc1]

R₇ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
   wherein:
      Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
      R_{c} is hydrogen, alkyl_{(C≤8)}, or substituted alkyl_{(C≤8)};
x is 0, 1, 2, or 3; or
a group of the formula:

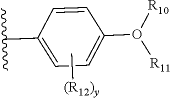

wherein:
   R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
   R₁₂ is amino, azido, carboxy, cyano, halo, hydroxy, nitro, hydroxysulfonyl, sulfonamide, or
   alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
   —S(O)₂N(Rₐ)R_{b}, —NR_{c}C(O)Rₐ, —C(O)NRₐ(R_{b}), or —NRₐ(R_{b}), or a substituted version of any of these groups;
      wherein:
         Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
      R_{c} is hydrogen, alkyl_{(C≤8)}, or substituted alkyl_{(C≤8)};
   y is 0, 1, 2, or 3;
   R₃ is alkyl_{(C≤12)}, cycloalkyl_{(C≤12)}, bicycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, or a substituted version of any of these groups;
   R₄ is hydrogen or alkyl_{(C≤12)}, cycloalkyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, -alkanediyl_{(C≤6)}-heterocycloalkyl_{(C≤12)} or a substituted version of any of these groups; abd
   A₁, A₂, A₃, and A₄ are each independently CH, N, or CR₅, wherein:
      R₅ is amino, azido, carboxy, cyano, halo, hydroxy, nitro, hydroxysulfonyl, sulfonamide, or
      alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
      —S(O)₂N(Rₐ)R_{b}, —NR_{c}C(O)Rₐ, —C(O)NRₐ(R_{b}), or —NRₐ(R_{b}), or a substituted version of any of these groups;
         wherein:
            Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
            R_{c} is hydrogen, alkyl_{(C≤8)}, or substituted alkyl_{(C≤8)};
with a compound of the formula:

R₁—X (III)
wherein:
   R₁ is alkyl_{(C≤12)}, cycloalkyl_{(C≤12)}, alkenyl_{(C≤12)}, alkynyl_{(C≤12)}, aralkyl_{(C≤12)}, heteroaralkyl_{(C≤12)}, -alkanediyl_{(C≤6)}-cycloalkyl_{(C≤12)}, or a substituted version of any of these groups; and
   X is an activating group.

In some embodiments, X is a halo, mesyl, tosyl, or triflyl. In some embodiments, X is chloro or iodo. In some embodiments, the compound of formula II is dissolved in the compound of formula III. In some embodiments, the methods further comprise heating the compounds of formulas II and III to a temperature from about 25° C. to about 100° C. such as a temperature of about 65° C. In some embodiments, the methods further comprise reacting for a time period from about 30 minutes to about 24 hours. In some embodiments, the time period is from about 3 hours to about 12 hours. In some embodiments, the time period is about 6 hours.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the relative ATP activity. 12M11 has specific nanomolar activity against Mut6 cells with no to minimal effect on normal astrocytes and mouse embyonic fibroblasts (MEF). ATP-activity measured with CellTiter-Glo® assay from Promega.

FIGS. 2A-2C show the activity of 12M11 in several cancer cell lines. 12M11 selectively kills some other cancer cell lines (CellTiter-Glo® assay from Promega). FIG. 2A shows the activity of various cancer cell lines of various tumor origin. FIG. 2B shows the activity of 12M11 in primary human prostate cancer cell lines. FIG. 2B shows the activity of 12M11 in primary human GBM cell lines.

FIGS. 3A & 3B show the microarray profiling at 6 hrs revealed a distinctive gene expression profile for Mut6 cells exposed to 12M11 (FIG. 3A) compared to MEFs (FIG. 3B) or astrocytes whose expression profiles were not affected, including several cell cycle arrest, pro-apoptotic and stress response genes.

FIG. 4 shows the quantitative reverse transcription polymerase chain reaction (qRT-PCR) demonstrates deregulated mRNA levels in Mut6 cells but not in MEFs.

FIGS. 5A & 5B show the western blot analysis of Mut6 cells (FIG. 5A) and MEFs (FIG. 5B) after treatment with 12M11 at 3 and 48 hr time points. These results indicate that 12M11 activates the ATF4 pathway and represses the mTOR pathway.

FIG. 6 shows that 12M11 disrupts mitochondrial membrane potential. The proton uncoupler, FCCP, impairs oxidative phosphorylation in Mut6 cells as assayed by TMRE staining within 30 min. Similarly, 12M11 impairs oxidative phosphorylation within 30 min and is sustained over 48 hours.

FIG. 7 shows increased uptake of glucose and increased lactate secretion. Mut6 cells were incubated with 12M11 for 7 hours, and the collected culture media was measured for glucose, lactate, glutamine, and glutamate.

FIG. 8 shows the mitochondrial respiration (oxygen consumption rate, OCR) is measured. Rotenone and oligomycin are compounds that block complex I and V, respectively. FCCP is a proton uncoupler. The diagram at bottom schematically represents oxidative phosphorylation complexes I-V.

FIGS. 9A-9D show cells were treated with 12M11 at 0 (dark gray), 1 (black), or 2 mM (light gray). Mut6 and MEF both decrease OCR (FIG. 9A and FIG. 9B); 12M11-treated Mut6 dramatically increases ECAR (FIG. 9C), in contrast to MEF (FIG. 9D). Oligomycin, FCCP, and rotenone serve as controls for general block of oxidative phosphorylation intermediates.

FIGS. 10A-10C show glucose deprivation mimics effects of 12M11. Glucose deprivation induces apoptosis in Mut6 (FIG. 10A); induces 12M11-like stress response transcrip-tional program (FIG. 10B); and induces ATF4 and decreases P-S6 protein in Mut6 but not MEF. Glucose starvation in Mut6 tumor cells, not in MEFs, induces the expression changes of 12M11 effector genes (FIG. 10C).

FIGS. 11A-11C show Mut6 cells use full activity of Oxyphos in basal state, while total ATP levels are low. FIG. 11A shows treatment of Mut6 and MEF with FCCP demonstrate that Mut6 use full activity of Oxyphos, while MEF have additional capacity (higher OCR) after FCCP treatment. FIG. 11B shows Mut 6 have lower basel levels of ATP compared to MEF (CellTiter-Glo® assay from Promega). FIG. 11C shows TMRE-staining shows that Mut 6 have higher oxyphos activity than MEF (higher mitochondrial membrane potential).

FIGS. 12A & 12B show (FIG. 12A) Mut6 cells are more sensitive than MEF cells to OxyPhos inhibitors (CellTiter-Glo® assay from Promega). (FIG. 12B) The ATP levels (% change from basal) of Mut6 cells decrease significantly upon treatment with OxyPhos inhibitors oligomycin, rotenone, or 12M11, whereas corresponding ATP levels increase for MEF cells upon similar OxyPhos-inhibitor treatment.

FIGS. 13A-13C show (FIG. 13A) several cancer cell lines are more sensitive than DAOY and MEF cells to OxyPhos inhibitor 12M11 (CellTiter-Glo® cell viability assay from Promega). (FIG. 13B) The relative ATP levels DAOY and MEF cells are significantly higher than for sensitive cancer cell lines. (FIG. 13C) DAOY cells have lower OxyPhos activity than cancer cells as determined by TMRE

FIGS. 14A-14C show (FIG. 14A) specificity of siRNA knock-down for OxyPhos complex proteins in Mut6 cells. (FIG. 14B) siRNA knock-down of OxyPhos complex proteins in Mut6 cells induces ATF4 and represses phospho-S6 protein levels. (FIG. 14C) The OxyPhos inhibitors rotenone and oligomycin also induce ATF4 and suppress phospho-S6 in Mut6 cells.

FIGS. 15A & 15B show cell viability of Mut6 cells (FIG. 15A) and astrocytes (FIG. 15B) upon treatment with protein synthesis inhibitor cycloheximide (CHX), general OxyPhos inhibitors (antimicyn, rotenone, oligomycin), and compound 12M11 (CellTiter-Glo® cell viability assay from Promega).

FIGS. 16A-16C show (FIG. 16A) the cell viability of Mut6 cells upon treatment with 12M11 or biotin-12M11 (CellTiter-Glo® cell viability assay from Promega). (FIG. 16B) biotin-12M11, just as the parent 12M11 induces ATF4 and represses phospho-S6 protein levels. (FIG. 16C) Structure of biotin-12M11.

FIG. 17 shows general schematic protocol for Avidin Agarose pull-down of Biotin-12M11 interacting proteins.

FIGS. 18A & 18B show (FIG. 18A) SDS gel and silver stain of avidin pull-down bands that are competed by 12M11 (the arrow heads). (FIG. 18B) Mass-spec of specific pull-down bands (the arrow heads in silver stain) identifies primarily mitochondrial proteins (members of OxyPhos complex in the boxes). Acaca and Pcca are known to non-specifically interact with biotin and were discounted.

FIGS. 19A-19C show 12M11 interacts with OxyPhos complexes. (FIG. 19A) Diagram of the electron transport complexes I-V. (FIG. 19B) Mut6 cells exposed to 12M11, Biotin-12M11, or 12M11 followed by Biotin-12M11 (see FIG. 18 for protocol) were pulled down with agarose avidin beads. Biotin-12M11 pull-down is reduced when pre-incubated with 12M11 (lane 3). (FIG. 19C) 12M11 excludes Biot-12M11 from associating with OxyPhos proteins in a concentration-dependent manner.

FIGS. 20A & 20B show the native gels from mitochondrial extracts from 12M11 pretreated astrocytes (FIG. 20A) or Mut6 cells (FIG. 20B) probed with antibodies against proteins in complexes I-IV reveals different complex size and composition between astrocytes and Mut6 cells for complexes containing complex I and III proteins (NDUFV2 and UQCRC2), and a transient destabilization of complexes II and IV in astrocytes (FIG. 20A) at the 1-hour time-point. In Mut6 cells (FIG. 20B), the same complexes II and IV remain stable at the 1 hour time-point and accumulate at the 24 hour time-point. (SC is Super-Complex)

FIGS. 21A & 21B show the denaturing SDS gels from 12M11 pretreated astrocytes (FIG. 21A) or Mut6 cells (FIG. 21B) probed with antibodies against proteins in complexes I-IV indicate that the availability of total mitochondrial protein for each of the antibodies used remains equivalent and stable over time for both cell types.

FIGS. 22A-22F show 12M11 (FIGS. 22A-22C) and L129 (FIGS. 22D-22E) Murine S9 Half-Life (FIG. 22A & FIG. 22D), in vivo plasma PK for 10 mg/kg IP dosing (FIG. 22B & FIG. 22E), and in vitro plasma Half-Life (FIG. 22C & FIG. 22F).

FIGS. 23A-23C shows (FIG. 23A) the structure of analog L129. (FIG. 23B) Western blot indicating that L129 induces ATF4 and represses phospho-S6 in a manner similar to 12M11 (see FIG. 5A). (FIG. 23C) qRT-PCR demonstrating that L129 deregulates mRNA levels in Mut6 cells in a manner similar to 12M11 (see FIG. 4).

FIGS. 24A-24D shows (FIG. 23A) (FIG. 24A) Tumor PK of L129. (FIG. 24B) Tumor weight of vehicle (red dots) and L129-treated (purple dots) animals after 4 weeks of daily ip injection (10 mg/kg). (FIG. 24C) Body weight of control and treated animals. (FIG. 24D) Tumor Histopathology of control and treated animals.

### DESCRIPTION OF ILLUSTRATIVE EMBODIMENTS

In certain aspects, the present disclosure provides compounds containing a cationic imidazolium group which may be used as chemotherapeutics. In some embodiments, the compounds may inhibit one or more proteins which is altered in cancerous cells. These compounds may be used in the treatment of hyperproliferative diseases such as cancer. In some embodiments, these compounds show selective growth inhibition in cancerous cell lines relative to non-cancerous cell lines.

### I. COMPOUNDS OF THE PRESENT DISCLOSURE

The benzimidazolium, pyridoimidazolium, and pyrazinoimidiazolium compounds provided by the present disclosure are shown, for example, above in the Summary section and in the claims below. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

Benzimidazolium, pyridoimidazolium, and pyrazinoimidiazolium compounds of the disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent the compounds of the disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

Compounds of the disclosure may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the benzimidazolium, pyridoimidazolium, and pyrazinoimidiazolium compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### II. CANCER AND OTHER HYPERPROLIFERATIVE DISEASES

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, the benzimidazolium, pyridoimidazolium, and pyrazinoimidiazolium compounds described herein may be used to lead to decreased cell counts and as such can potentially be used to treat a variety of types of cancer types.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

1. Gliomas

Gliomas are a diverse group of brain tumors that arise from the normal “glial” cells of the brain. The most important determinant of survival for gliomas is the “grade” of the glioma. The low-grade gliomas have a protracted natural history, while the high grade gliomas (anaplastic astrocytoma and glioblastoma multiforme) are much more difficult to successfully treat. The gliomas have specific signs and symptoms that are primarily related to the location of the glioma.

The temporal lobe gliomas, for example, may cause epilepsy, difficulty with speech or loss of memory. The frontal lobe gliomas may cause behavioral changes, weakness of the arms or legs or difficulty with speech. The occipital gliomas may cause loss of vision. The parietal gliomas may cause loss of spatial orientation, diminished sensation on the opposite side of the body, or inability to recognize once familiar objects or persons.

Grading according to degree of malignancy was first proposed in 1949. In this classification, astrocytomas and glioblastomas represent different grades of malignancy of the same tumor. Grade I tumors, typically slow growing, are characterized by most cells having normal characteristics, and few mitotic features. Endothelial proliferation is absent. Grade II tumors, previously designated “astroblastomas,” are characterized by an increased number of cells with polymorphic nuclei in mitoses. There is no clear line of demarcation from normal tissue. Grade III tumors represent anaplastic astrocytomas and Grade IV tumors represent the typical glioblastoma multiforme, characterized by cellular pleomorphism, vascular proliferation, mitoses, and multinucleated giant cells.

Surgery. The role of surgical resection in the treatment of malignant gliomas remains controversial even after 75 years of experience with primary malignant gliomas. Surgery permits a pathologic diagnosis to be established while the patient is still alive. However, many physicians argue that current radiologic imaging methods, including computed tomography (CT) and magnetic resonance imaging (MRI), permit a malignant brain tumor to be diagnosed without the necessity for attempted tumor resection and, thus, avoid the risks of surgery.

There is evidence that surgical reduction of tumor to very small residual amounts can prolong survival and permit patients to return to active lives. However, retrospective studies are subject to the criticism that the extent of attempted resection depends on the condition of the patient at the time of surgery (age, tumor location, clinical state), and that favorable conditions usually lead the surgeon to attempt a greater resection. Therefore, in such studies, it is not clear that the extent of surgery is as important to survival as are the more favorable prognostic variables. Nevertheless, these results support the surgical removal of the largest possible tumor volume that can be done safely. Patients are frequently able to return to a full, active life without the need for large doses of corticosteroids to ameliorate incapacitating symptoms.

Radiation. The proper portals and doses of radiation for brain tumors have changed with the advent of better imaging techniques. It has been reported in controlled studies that postoperative whole-brain radiation therapy increases patient survival over surgery alone. Other data showed that patients receiving 5,500 to 6,000 cGy of radiation live significantly longer than those receiving 5,000 cGy.

Prolonged survival has been reported in patients with recurrent malignant gliomas who were treated with temporarily implanted I¹²⁵ sources. A phase III trial randomized newly diagnosed patients to receive either (a) postoperative temporary I¹²⁵ seed implantation in the residual tumor bed, followed by standard external-beam radiotherapy plus IV carmustine; or (b) external radiotherapy plus carmustine, without seed implantation. Preliminary review of the results demonstrated that patients who received I¹²⁵ seeds lived longer than those who did not receive seeds, although the difference did not quite reach statistical significance. The study suggests but does not prove that brachytherapy extends survival beyond that achievable with external radiotherapy alone.

Radiosurgery. Radiosurgery, either by gamma knife or linear accelerator, has been shown to be effective in the treatment of arteriovenous malformations, small primary and metastatic brain tumors, and benign brain tumors, such as meningiomas and acoustic neuromas. Its investigational use in the treatment of gliomas has been addressed in several reports. In one trial, 37 patients received radiosurgery (1,000 to 2,000 cGy) to residual contrast-enhancing tumor after treatment with conventional external-beam radiation therapy. Local recurrence still occurred, but overall survival time may have been prolonged. Of the 37 patients, 7 (19%) required reoperation at a median time of 5 months after radiosurgery to remove necrotic tumor.

A major problem with radiosurgery (as with brachytherapy) is bias in the selection of patients for treatment. However, radiosurgery may be of benefit in a small group of good-prognosis patients with small tumors.

Chemotherapy. In 1983, it was reported that surgery plus radiation therapy and carmustine chemotherapy significantly adds to the survival of patients with malignant glioma, as compared with surgery plus radiation therapy without chemotherapy. High-dose methylprednisolone does not prolong survival. Both procarbazine and streptozotocin have demonstrated effectiveness similar to that of carmustine. Carmustine alone is as effective as carmustine followed by procarbazine, or carmustine plus hydroxyurea followed by procarbazine plus teniposide. Methotrexate also has been reported to be effective in treating gliomas.

Intra-arterial carmustine is no more effective than intravenous carmustine and substantially more toxic. Serious toxicity induced by intra-arterial carmustine included irreversible encephalopathy and/or visual loss ipsilateral to the infused carotid artery. In the same study, fluorouracil did not influence survival. Neuropathologically, intra-arterial carmustine produced white matter necrosis. Intra-arterial cisplatinum is safer than carmustine administered by the same route but is no more effective than another nitrosourea, PCNU.

Over the past several years, there has been increasing interest in the use of targeted interstitial drug delivery using biodegradable microspheres and wafers. In a multicenter controlled trial, 222 patients with recurrent malignant gliomas who required reoperation were randomly assigned to receive surgically implanted biodegradable polymer discs containing 3.85% of carmustine or discs containing placebo. Median survival of the 110 patients who received carmustine polymers was significantly longer than that of the 112 patients who received placebo polymers (31 versus 23 weeks).

In addition to these controlled survival-based clinical trials, a large number of agents have also been tested in response-based studies in glioma patients. To date, however, no drug has been found to be more effective than the nitrosoureas. The combination of procarbazine, lomustine, and vincristine (PCV) has become a popular chemotherapeutic regimen for malignant glioma, and may be more effective than carmustine alone.

A. Glioblastoma Multiforme

Glioma-glioblastoma multiforme (GBM), referred to a Grade IV glioma, is the most malignant of the neuroepithelial neoplasms, characterized by cellular pleomorphism, numerous mitotic figures, and often multinucleated giant cell. Proliferation of the vascular endothelium is seen as well as areas of necrosis with circumjacent pseudopalisading of the neoplastic cells. It can appear as either a well-circumscribed globular mass or a more diffuse mass lesion. The cut surface reveals necrosis, fatty degeneration, and hemorrhage. Hemorrhages have been found in 40%, with necrosis in up to 52% of the cases. The tumor is usually solid, although cysts may be present. Rarely the tumor consists of a solitary cyst and mural nodule.

Glioblastoma multiforme constitutes approximately 7% of childhood intracranial neoplasms. The overall male to female ratio in children is 3:2. In adults, glioblastomas are noted most frequently in the frontal lobe with the temporal lobe second in frequency. Childhood glioblastomas of the cerebral hemispheres are also located most often in the frontal lobe; with the second most frequent site being the parietal lobe. Primary glioblastoma of the spinal cord in childhood is rare.

Glioblastoma multiforme in children appears to have two characteristic courses, each of which is related to the location of the tumor. Glioblastomas of the brainstem, a more primitive part of the central nervous system, occur at a younger age and have a shorter mean survival relative to those of the cerebral hemispheres. Glioblastoma multiforme of the cerebral hemisphere, a more highly developed part of the central nervous system, is characterized by onset in older children (13 years) and by a longer mean survival.

Headache is the most common complaint and papilledema the most common physical finding in children with hemispheric glioblastoma. Seizures are noted in up to one third of the children. Survival rates in patients with glioblastoma multiforme are uniformly poor. In studies of children treated with surgery and intracranial radiation, only one third of the children are alive one year after diagnosis. Survival of children with glioblastoma multiforme of either of the cerebral hemispheres or the brainstem has significantly increased since the advent of dexamethasone therapy. Presently therapy consists of surgery plus combination chemotherapy.

In summary it can be said that glioblastoma multiforme behaves similarly in both children and adults. The course of intracranial glioblastomas in children is more rapidly fatal than that of other similarly situated gliomas in childhood. While the overall survival rate is very poor in patients with a glioblastoma multiforme, intensive chemotherapy with surgical resection does offer some hope in increasing survival time among children.

B. Astrocytoma

Astrocytomas are tumors that arise from brain cells called astrocytes. Gliomas originate from glial cells, most often astrocytes. Sometimes the terms “astrocytoma” and “glioma” are used interchangeably. Astrocytomas are of two main types—high-grade and low-grade. High-grade tumors grow rapidly and can easily spread through the brain. Low-grade astrocytomas are usually localized and grow slowly over a long period of time. High-grade tumors are much more aggressive and require very intense therapy. The majority of astrocytic tumors in children are low-grade, whereas the majority in adults are high-grade. These tumors can occur anywhere in the brain and spinal cord. Common sites in children are the cerebellum (the area just above the back of the neck), cerebral hemispheres (the top part of the brain), and the thalamus or hypothalamus (located in the center of the brain).

Astrocytomas account for the majority of pediatric brain tumors. About 700 children are diagnosed with low-grade astrocytomas each year. In children, about 90 percent of astrocytomas are low-grade; only about 10 percent are high-grade.

Clinical features and symptoms depend on the location of the tumor and the child's age. The most common location is the cerebellum. Patients with cerebellar tumors have symptoms that include headache, vomiting and unsteadiness in walking. Tumors in the cerebral hemispheres commonly present with seizures: occasionally there is weakness of the arms and legs. Tumors in the hypothalamus often present with visual problems, while thalamic tumors cause headaches and arm or leg weakness.

Complete surgical removal of the tumor (resection) is the best option for tumors in areas where this can be done without damaging the normal, surrounding brain. For low-grade astrocytomas that are completely removed, further therapy is usually not needed. If the surgeon cannot completely remove the tumor, chemotherapy or radiation therapy may be given. The choice of treatments depends on the age of the patient, tumor location; some patients may even be followed without treatment. Radiation therapy is used for older children and those whose tumors keep growing despite chemotherapy. About 90 percent of children with low-grade astrocytomas are alive five years from diagnosis.

High-grade astrocytomas can rarely be removed totally because they often affect large areas of the brain by the time symptoms are obvious. All patients with high-grade astrocytomas usually receive chemotherapy regardless of age. Most, except the very youngest, also receive radiation therapy. Currently, the prognosis is poor in the group of patients. The subset of patients who have high-grade tumors that can be removed may have survival rates of 35 to 40 percent after postsurgical irradiation with chemotherapy. The survival of other patients is very poor.

Research efforts for the low-grade astrocytomas focus on developing chemotherapy regimens that control tumor growth with fewer side effects on other organs of the body. Because these tumors grow slowly, the strategy is to give less intensive chemotherapy over longer periods of time. For older children and those whose tumors progress despite chemotherapy, new radiation techniques are under study to deliver more localized therapy with minimal effects on the normal brain.

For high-grade tumors, new approaches include use of new chemotherapy drugs, and the potential option of high doses of chemotherapy. Investigational new approaches, including new chemotherapy drugs and gene therapy to help protect the bone marrow from the side effects so that more intensive chemotherapy can be given are in various stages of development.

C. Oligodendroglioma and Anaplastic Oliogodendroglioma

Oligodendrogliomas are believed to be tumors of cells called oligodendrocytes that have a role in the structure and function of the brain. However, the origin of these tumor cells has been questioned. Oligodendrogliomas are classified as low grade oligodendroglioma (less aggressive) and anaplastic oligodendroglioma (more aggressive). More common that pure oligodendrogliomas are low grade and anaplastic tumors that are a mixture of astrocytoma and oligodendroglioma (“oligoastrocytomas”).

The initial treatment of low grade oligodendroglioma and oligoastrocytoma consists of maximal surgery. The role of radiation therapy has been disputed, but younger people with minimal residual disease after surgery may have radiation therapy deferred as long as there is adequate monitoring of the tumor by MRI or CT scanning.

Anaplastic oligodendrogliomas and mixed oligoastrocytomas are more sensitive to chemotherapy than astrocytomas. A high rate of response to the use of PCV (procarbazine, lomustine, vincristine) chemotherapy has made the use of chemotherapy prior to radiation therapy the standard of care for these tumors. The actual effectiveness of this treatment regimen is currently being investigated in a large multinational trial.

Additionally, low grade oligodendrogliomas are also sensitive to chemotherapy, and PCV can be used when low grade tumors begin to grow despite prior radiation therapy.

### III. THERAPIES

1. Pharmaceutical Formulations and Routes of Administration

Where clinical applications are contemplated, it will be necessary to prepare pharmaceutical compositions in a form appropriate for the intended application. In some embodiments, such formulation with the compounds of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the compounds, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the compounds of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

In some embodiments of the present disclosure, the compounds are included a pharmaceutical formulation. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., the compounds analogs described herein) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the benzimidazolium, pyridoimidazolium, and pyrazinoimidiazolium compounds described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The benzimidazolium, pyridoimidazolium, and pyrazinoimidiazolium compounds of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 mL of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

2. Methods of Treatment

In particular, the benzimidazolium, pyridoimidazolium, and pyrazinoimidiazolium compounds that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (complete blood count—CBC), or cancer cell growth or proliferation. In some embodiments, amounts of the benzimidazolium, pyridoimidazolium, and pyrazinoimidiazolium compounds used to inhibit bacterial growth or induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the benzimidazolium, pyridoimidazolium, and pyrazinoimidiazolium compounds may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

3. Combination Therapies

It is envisioned that the benzimidazolium, pyridoimidazolium, and pyrazinoimidiazolium compounds described herein may be used in combination therapies with one or more cancer therapies or a compound which mitigates one or more of the side effects experienced by the patient. It is common in the field of cancer therapy to combine therapeutic modalities. The following is a general discussion of therapies that may be used in conjunction with the therapies of the present disclosure.

To treat cancers using the methods and compositions of the present disclosure, one would generally contact a tumor cell or subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the benzimidazolium, pyridoimidazolium, and pyrazinoimidiazolium compounds described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated. The following is a general discussion of cancer therapies that may be used combination with the compounds of the present disclosure.

A. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γ1 and calicheamicin ω1; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

B. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

C. Immunotherapy

In the context of cancer treatment, immunotherapeutics, generally, rely on the use of immune effector cells and molecules to target and destroy cancer cells. Temozolomide and bevacizumab are two non-limiting examples. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

D. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurance of the tumor. Additionally, the compounds of the present disclosure can also be used in a neoadjuvant setting.

E. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents may be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

### IV. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof in a divalent context “phosphate” means —OP(O)(OH)O— or a deprotonated form thereof; “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, for example, the formula

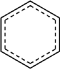

includes

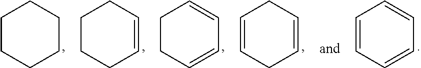

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

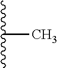

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

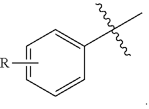

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

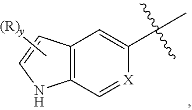

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱTr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). A “monoalkyl substituted” cycloalkyl group refers to a cycloalkyl radical which has been substituted with one “alkyl” group as that term is defined above. Similarly, a “dialkyl substituted” cycloalkyl group refers to a cycloalkyl radical which has been substituted with two “alkyl” groups as that term is defined above. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

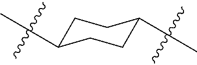 [SMILES: *C1CCC(*)CC1]

is a non-limiting example of cycloalkanediyl group. A “cycloalkane” refers to the class of compounds having the formula H—R, wherein R is cycloalkyl as this term is defined above. The term “bicycloalkyl” refers to a monovalent saturated aliphatic group with a carbon atom as the point of attachment, said carbon atom forming part of two or more non-aromatic ring structures, wherein two or more of the rings share two or more bridgehead carbons, no carbon-carbon double or triple bonds, and no atoms other than carbon and hydrogen. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

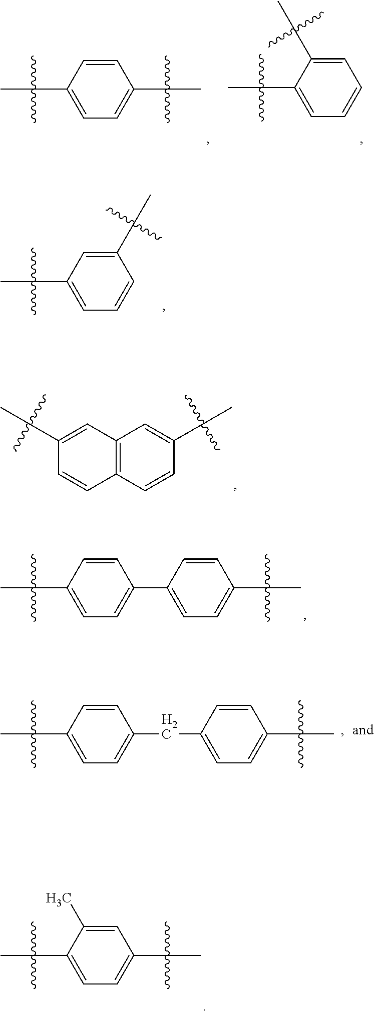

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “aralkenyl” when used without the “substituted” modifier refers to the monovalent group -alkenediyl-aryl, in which the terms alkenediyl and aryl are each used in a manner consistent with the definitions provided above. Non-limiting examples are: 4-phenyl-3-butene and 2-phenylethenyl. When the term aralkyl is used with the “substituted” modifier one or more hydrogen atom from the alkenediyl and/or the aryl group has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples are: pyramidylmethyl and 2-quinolyl-ethyl. When the term heteroaralkyl is used with the “substituted” modifier one or more hydrogen atom from the alkanediyl and/or the heteroaryl group has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heteroaralkenyl” when used without the “substituted” modifier refers to the monovalent group -alkenediyl-heteroaryl, in which the terms alkenediyl and heteroaryl are each used in a manner consistent with the definitions provided above. Non-limiting examples are: phenylethenyl and 4-phenyl-2-butenyl. When the term heteroaralkenyl is used with the “substituted” modifier one or more hydrogen atom from the alkenediyl and/or the heteroaryl group has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. When these terms are used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The terms “alkylsulfonyl” and “alkylsulfinyl” when used without the “substituted” modifier refers to the groups —S(O)₂R and —S(O)R, respectively, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, and “heterocycloalkylsulfonyl” are defined in an analogous manner. When any of these terms is used with the “substituted” modifier one or more hydrogen atom has been independently replaced by —OH, —F, —Cl, —Br, —I, —NH₂, —NO₂, —N₃, —CO₂H, —CO₂CH₃, —CN, —SH, —OCH₃, —OCH₂CH₃, —SCH₃, —SCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “activating group” in the context of this application is a reagent which enhances the reactivity of the compound. In some embodiments, the activating group is a leaving group. A “leaving group” in the context of this application is a group which has the ability to be displaced from the molecule through nucleophilic attack. This group may also convert a hydroxyl group into a better leaving group by stabilizing the charge on the oxygen when the atom bears a negative charge thus making the hydroxyl group more susceptible to a nucleophilic attack and displacement. Additionally, the leaving group could be a halogen atom especially a bromide or iodide.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxy ethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Synthesis and Characterization of the Compounds

A. General Experimental

All reactions were carried out under nitrogen atmosphere with dry solvents under anhydrous conditions, unless otherwise noted. Anhydrous solvents were obtained by passing them through commercially available alumina columns (Innovative technology, Inc., MA). All reagents were commercial compounds of the highest purity available. Analytical thin layer chromatography (TLC) was performed on aluminium plates with Merck Kieselgel 60F254 and visualized by UV irradiation (254 nm) or by staining with a solution of potassium permanganate. Flash column chromatography was carried out using Merck Kieselgel 60 (230-400 mesh) under pressure. Infrared spectra were obtained on a Perkin-Elmer I1000 FTIR series, from a thin film deposited onto a NaCl glass. Optical rotations were measured on a Rudolph Research Analytical Autopol® IV polarimeter at 20° C. 1H NMR spectra were recorded in CDCl₃, CD₃OD, DMSOd₆ and (CD₃)₂CO at ambient temperature on a Varian Inova-400 spectrometer at 400 MHz with residual protic solvent as the internal reference (CDCl₃, d_{H}=7.26 ppm; (CD₃)₂CO, d_{H}=2.05 ppm; CD₃OD, d_{H}=3.31 ppm; DMSO-d₆, d_{H}=2.50 ppm); chemical shifts (d) are given in parts per million (ppm), and coupling constants (J) are given in Hertz (Hz). The proton spectra are reported as follows: d (multiplicity, coupling constant J, number of protons). The following abbreviations were used to explain the multiplicities: app=apparent, b=broad, d=doublet, dd=doublet of doublets, ddd=doublet of doublet of doublets, dddd=doublet of doublet of doublet of doublets, m=multiplet, s=singlet, t=triplet. ¹³C NMR spectra were recorded in CDCl₃, CD₃OD, DMSO-d₆ and (CD₃)₂CO at ambient temperature on the same spectrometer at 100 MHz with the central peak of CDCl₃ (d_{C}=77.0 ppm), CD₃OD (d_{C}=49.0 ppm), DMSO-d6 (d_{C}=39.4 ppm) or (CD3)2C0 (dc =30.8 ppm) as the internal reference. Electrospray ionization mass spectra (ESI-MS) were recorded on a Shimadzu 2010-LCMS. HRMS were performed on a Shimadzu IT-TOF. Microwave reactions were carried out on a Biotage® Initiator Classic. DMAP=4-(dimethylamino)pyridine, DMF=N,N-dimethylformamide, DIPEA=N,N-diisopropylethylamine, DHP=tetrahydro-2H-pyran, EDC=N-ethylcarbodiimide hydrochloride, HATU=1-[bis(dimethylamino)methylene]-1H-1,2,3-triazolo [4,5-b]pyridinium 3-oxid hexafluorophosphate, MW=microwave, NMP=1-methyl-2-pyrrolidinone, py=pyridine, TBDMS=tert-butyldimethylsilyl, THF=tetrahydrofuran, THP=3,4-dihydro-2H-pyran. Unless otherwise noted, commercially available materials were used without further purification. All solvents were of HPLC or ACS grade. Solvents used for moisture sensitive operations were distilled from drying reagents under a nitrogen atmosphere: Et₂O and THF from sodium benzophenone ketyl; benzene and toluene from sodium; CH₂Cl₂ from CaH₂, pyridine over solid KOH, anhydrous N,N-dimethylformamide, and CH₃CN were purchased from commercial sources. Reactions were performed under an atmosphere of argon with magnetic stirring unless noted otherwise. Flash chromatography (FC) was performed using E Merck silica gel 60 (240-400 mesh) according to the protocol of Still, Kahn, and Mitral.

B. Experimental Procedure

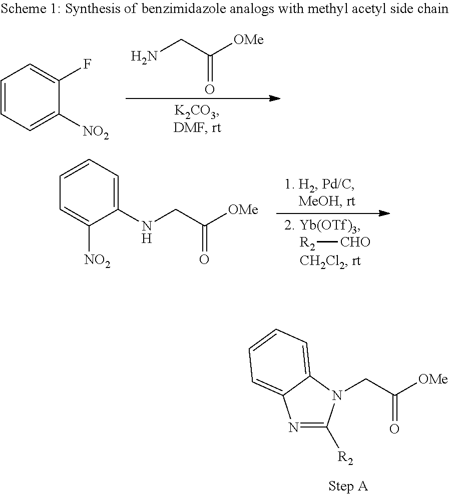

Step A: General Procedure for the Preparation of Benzimidazole from Nitrobenzene

A mixture of nitrobenzene (1.0 equiv.) and Pd on activated carbon (5% activated, 0.05 equiv.) was dissolved in MeOH (3 mL). The mixture was degassed under vacuum and re-purged with H₂, and this process was repeated 3 times. The reaction was completed in an hour based on TLC analysis. The solvent was removed under reduced pressure and the residue was filtered through a small column of silica gel to give the resulting aniline. The corresponding aniline (1.0 equiv.) was dissolved in anhydrous CH₂Cl₂ (0.3 M) and cooled to 0° C. The corresponding aldehyde (1.2 equiv.) and Yb(OTf)₃ (0.1 equiv.) were added sequentially to the reaction. The mixture was raised to rt and stirred for overnight. The solvent was removed under reduced pressure and purified by flash chromatography on silica gel as indicated to give the desired benzimidazole.

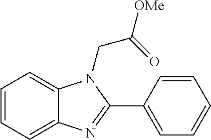 [SMILES: COC(=O)Cn1c(-c2ccccc2)nc2ccccc21]

The title compound was obtained following the general procedure (Step A) described above. Purification of the residue by flash chromatography on silica gel (gradient elution, 40% EtOAc in hexanes) afforded Lqr-4-138 as a yellow gel (28%). IR (cm⁻¹) 2950, 1850, 1450, 1225, 972, 820, 745; ¹H NMR (400 MHz, CDCl₃) δ 7.87-7.79 (m, 1H), 7.72-7.64 (m, 2H), 7.55-7.47 (m, 3H), 7.35-7.25 (m, 3H), 4.90 (s, 2H), 3.79 (s, 3H); ¹³C NMR (100 MHz, CDCl₃) δ 168.4, 154.1, 143.0, 136.1, 130.3, 129.8 (2C), 129.5 (2C), 129.1, 123.5, 123.1, 120.3, 109.6, 53.1, 46.5; ES-API MS: m/z calcd for C₁₆H₁₄N₂O₂, found 267.1 [M+H].

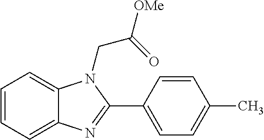 [SMILES: COC(=O)Cn1c(-c2ccc(C)cc2)nc2ccccc21]

The title compound was obtained following the general procedure (Step A) described above. Purification of the residue by flash chromatography on silica gel (gradient elution, 40% EtOAc in hexanes) afforded Lqr-4-134 as a yellow gel (35%). IR (cm⁻¹) 2954, 1750, 1458, 1215, 982, 821, 745; ¹H NMR (400 MHz, CDCl₃) δ 7.87-7.78 (m, 1H), 7.65-7.52 (m, 2H), 7.37-7.22 (m, 5H), 4.89 (d, J=1.6 Hz, 2H), 3.78 (d, J=2.0 Hz, 3H), 2.40 (s, 3H); ¹³C NMR (100 MHz, CDCl₃) δ 168.5, 154.3, 143.1, 140.5, 129.8 (2C), 129.4 (2C), 126.9, 126.8, 123.4, 123.0, 120.2, 109.5, 53.1, 46.5, 21.6; ES-API MS: m/z calcd for C₁₇H₁₆N₂O₂, found 281.1 [M+H].

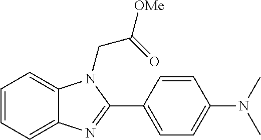 [SMILES: COC(=O)Cn1c(-c2ccc(N(C)C)cc2)nc2ccccc21]

The title compound was obtained following the general procedure (Step A) described above. Purification of the residue by flash chromatography on silica gel (gradient elution, 40% EtOAc in hexanes) afforded Lqr-4-139 as a yellow gel (28%). IR (cm⁻¹) 1749, 1612, 1489, 1364, 1196, 825; ¹H NMR (400 MHz, CDCl₃) δ 7.83-7.76 (m, 1H), 7.58 (d, J=8.8 Hz, 2H), 7.34-7.19 (m, 3H), 6.78 (d, J=8.8 Hz, 2H), 4.91 (s, 2H), 3.80 (s, 3H), 3.03 (s, 6H); ¹³C NMR (100 MHz, CDCl₃) δ 168.8, 155.1, 151.5, 143.2, 141.1, 136.3, 130.5 (2C), 122.8 (2C), 119.8, 116.7, 112.1, 109.3, 53.0, 46.7, 40.4; ES-API MS: m/z calcd for C₁₈H₁₉N₃O₂, found 310.2 [M+H].

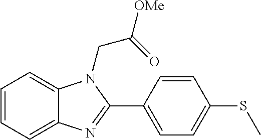 [SMILES: COC(=O)Cn1c(-c2ccc(SC)cc2)nc2ccccc21]

The title compound was obtained following the general procedure (Step A) described above. Purification of the residue by flash chromatography on silica gel (gradient elution, 40% EtOAc in hexanes) afforded Lqr-4-140 as a yellow gel (32%). IR (cm⁻¹) 1749, 1723, 1457, 1181, 748; ¹H NMR (400 MHz, CDCl₃) δ 7.85-7.77 (m, 1H), 7.61 (d, J=8.4 Hz, 2H), 7.39-7.25 (m, 5H), 4.89 (s, 2H), 3.79 (s, 3H), 2.52 (s, 3H); ¹³C NMR (100 MHz, CDCl₃) δ 168.2, 153.5, 142.9, 141.6, 135.9, 129.7, 129.5 (2C), 126.0 (2C), 123.2, 122.9, 120.0, 109.3, 46.3, 29.6, 15.1; ES-API MS: m/z calcd for C₁₇H₁₆N₂O₂S, found 313.1 [M+H].

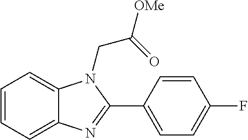 [SMILES: COC(=O)Cn1c(-c2ccc(F)cc2)nc2ccccc21]

The title compound was obtained following the general procedure (Step A) described above. Purification of the residue by flash chromatography on silica gel (gradient elution, 40% EtOAc in hexanes) afforded Lqr-4-141 as a yellow gel (36%). IR (cm⁻¹) 1747, 1484, 1221, 748; ¹H NMR (400 MHz, CDCl₃) δ 7.88-7.79 (m, 1H), 7.76-7.65 (m, 2H), 7.39-7.26 (m, 3H), 7.21 (t, J=8.4 Hz, 2H), 4.89 (s, 2H), 3.81 (s, 3H); ¹³C NMR (100 MHz, CDCl₃) δ 168.4, 165.3, 162.8, 153.2, 143.0, 136.0, 131.6 (d, J=8.6 Hz, 2C), 126.0 (d, J=3.3 Hz, 1C), 123.7, 120.3, 116.3 (d, J=21.8 Hz, 2C), 109.6, 53.1, 46.4; ES-API MS: m/z calcd for C₁₆H₁₃FN₂O₂, found 285.1 [M+H].

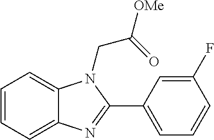 [SMILES: COC(=O)Cn1c(-c2cccc(F)c2)nc2ccccc21]

The title compound was obtained following the general procedure (Step A) described above. Purification of the residue by flash chromatography on silica gel (gradient elution, 40% EtOAc in hexanes) afforded Lqr-4-142 as a yellow gel (36%). IR (cm⁻¹) 1749, 1588, 1458, 1218, 890, 745; ¹H NMR (400 MHz, CDCl₃) δ 7.91-7.82 (m, 1H), 7.54-7.42 (m, 3H), 7.37-7.25 (m, 3H), 7.25-7.17 (m, 1H), 4.90 (s, 2H), 3.80 (s, 3H); ¹³C NMR (100 MHz, CDCl₃) δ 168.2, 164.1, 161.6, 152.7, 142.8, 136.0, 130.9 (d, J=8.4 Hz, 1C), 125.2, 123.9, 123.4, 120.4, 117.5 (d, J=21.0 Hz, 1C), 116.9 (d, J=23.0 Hz, 1C), 109.72, 53.23, 46.48; ES-API MS: m/z calcd for C₁₆H₁₃FN₂O₂, found 285.1 [M+H].

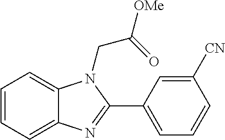 [SMILES: COC(=O)Cn1c(-c2cccc(C#N)c2)nc2ccccc21]

The title compound was obtained following the general procedure (Step A) described above. Purification of the residue by flash chromatography on silica gel (gradient elution, 40% EtOAc in hexanes) afforded Lqr-4-143 as a yellow gel (33%). IR (cm⁻¹) 1748, 1456, 1219, 745, 890, 745; ¹H NMR (400 MHz, CDCl₃) δ 8.09-8.01 (m, 1H), 8.01-7.94 (m, 1H), 7.91-7.76 (m, 2H), 7.65 (t, J=8.0 Hz, 1H), 7.43-7.28 (m, 3H), 4.89 (s, 2H), 3.82 (s, 3H); ¹³C NMR (100 MHz, CDCl₃) δ 168.0, 161.1, 136.0, 133.7, 133.0, 133.0, 130.1, 129.5, 124.4, 123.8, 120.5, 118.1, 113.6, 109.8, 53.4, 46.4; ES-API MS: m/z calcd for C₁₇H₁₃N₃O₂, found 292.1 [M+H].

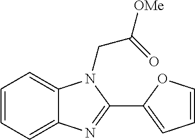 [SMILES: COC(=O)Cn1c(-c2ccco2)nc2ccccc21]

The title compound was obtained following the general procedure (Step A) described above. Purification of the residue by flash chromatography on silica gel (gradient elution, 40% EtOAc in hexanes) afforded Lqr-4-145 as a yellow gel (36%). IR (cm⁻¹) 1751, 1458, 1387, 1216, 1009, 743; ¹H NMR (400 MHz, CDCl₃) δ 7.88-7.70 (m, 1H), 7.57 (d, J=1.2 Hz, 1H), 7.39-7.17 (m, 4H), 6.58 (dd, J=3.2, 1.6 Hz, 1H), 5.22 (s, 2H), 3.73 (s, 3H); ¹³C NMR (100 MHz, CDCl₃) δ 168.4, 145.7, 144.4, 144.2, 143.1, 135.7, 123.6, 123.3, 120.1, 113.0, 112.3, 109.0, 53.0, 46.3; ES-API MS: m/z calcd for C₁₄H₁₂N₂O₃, found 257.1 [M+H].

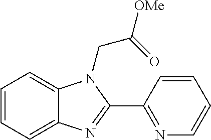 [SMILES: COC(=O)Cn1c(-c2ccccn2)nc2ccccc21]

The title compound was obtained following the general procedure (Step A) described above. Purification of the residue by flash chromatography on silica gel (gradient elution, 30% EtOAc in hexanes) afforded Lqr-4-146 as a yellow gel (38%). IR (cm⁻¹) 1750, 1447, 1214, 997, 743; ¹H NMR (400 MHz, CDCl₃) δ 8.69-8.54 (m, 1H), 8.50-8.46 (m, 1H), 7.96-7.72 (m, 2H), 7.43-7.17 (m, 4H), 5.61 (s, 2H), 3.69 (s, 3H); ¹³C NMR (100 MHz, CDCl₃) δ 169.2, 150.3, 149.7, 148.4, 142.7, 137.1, 137.0, 124.3, 124.0, 123.2, 120.5, 109.4, 104.9, 52.6, 47.6; ES-API MS: m/z calcd for C₁₅H₁₃N₃O₃, found 268.1 [M+H].

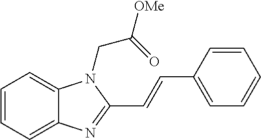 [SMILES: COC(=O)Cn1c(/C=C/c2ccccc2)nc2ccccc21]

The title compound was obtained following the general procedure (Step A) described above. Purification of the residue by flash chromatography on silica gel (gradient elution, 30% EtOAc in hexanes) afforded Lqr-4-147 as a yellow gel (40%). IR (cm⁻¹) 1748, 1455, 1219, 966, 742; ¹H NMR (400 MHz, CDCl₃) δ 7.97 (d, J=15.6 Hz, 1H), 7.80-7.75 (m, 1H), 7.62-7.54 (m, 2H), 7.48-7.20 (m, 6H), 6.96 (d, J=15.6 Hz, 1H), 4.95 (s, 2H), 3.75 (s, 3H); ¹³C NMR (100 MHz, CDCl₃) δ 167.9, 151.2, 143.2, 138.4, 136.0, 135.6, 129.4, 129.0 (2C), 127.5 (2C), 123.3, 123.2, 119.8, 112.5, 109.1, 53.1, 44.9; ES-API MS: m/z calcd for C₁₈H₁₆N₂O₂, found 293.1 [M+H].

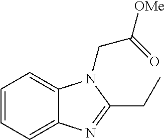 [SMILES: CCc1nc2ccccc2n1CC(=O)OC]

The title compound was obtained following the general procedure (Step A) described above. Purification of the residue by flash chromatography on silica gel (gradient elution, 40% EtOAc in hexanes) afforded Lqr-4-149 as a yellow gel (18%). IR (cm⁻¹) 2226, 1745, 1355, 1219, 845, 742; ¹H NMR (400 MHz, CDCl₃) δ 7.77-7.70 (m, 1H), 7.28-7.14 (m, 3H), 4.81 (s, 2H), 3.74 (s, 3H), 2.83 (q, J=7.6 Hz, 2H), 1.45 (t, J=7.6 Hz, 3H); ¹³C NMR (100 MHz, CDCl₃) δ 168.0, 156.3, 142.7, 135.4, 122.7, 122.4, 119.6, 108.7, 53.0, 44.8, 20.8, 11.6; ES-API MS: m/z calcd for C₁₂H₁₄N₂O₂, found 219.1 [M+H].

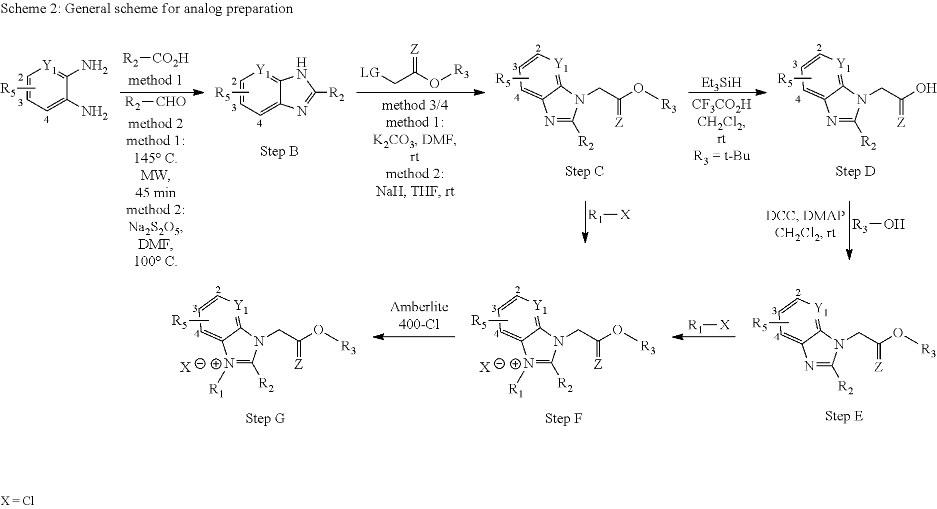

Step B: General Procedure for the Preparation of Benzimidazole from Phenylenediamine

Method 1. O-phenylenediamine (10.0 mmol) was dissolved in propionic acid (5 mL), and the resulting solution was sealed in the microwave reactor tube and irradiated via microwave to 145° C. for 45 min. The mixture was carefully quenched with cold saturated NaHCO₃ solution and extracted with EtOAc (×3). The organic extract was washed with brine and dried over anhydrous MgSO₄, and filtered. The solvent was removed under reduced pressure and the residue was purified by flash chromatography on silica gel as indicated to give the desired benzimidazole.

Method 2. Propionic aldehyde (10.0 mmol) was added to the solution of o-phenylenediamine (10.0 mmol) in anhydrous DMF (10 mL) at rt. After 10 min stirring, Na₂S₂O₅ (10.0 mmol) was added to the reaction and the resulting mixture was heated to 100° C. for 4 h. Reaction was checked by TLC for completion. The solvent was removed under reduced pressure and the residue was purified by flash chromatography on silica gel as indicated to give the desired benzimidazole.

### Step C: General Procedure of Benzimidazole Alkylation

Method 1. To a mixture of 1H-benzimidazole (2.0 mmol, 1.0 equiv.) and K₂CO₃ (4.0 mmol, 2.0 equiv.) in anhydrous DMF (10 mL) was added tert-butyl-2-bromoacetate (4.0 mmol, 2.0 equiv.) at rt. The resulting mixture was stirred at 90° C. for 30 min, and cooled to rt. The reaction was diluted with H₂O, and extracted with Et₂O (3×) and dried over anhydrous MgSO₄, and filtered. The solvent was removed under reduced pressure and the residue was purified by flash chromatography on silica gel as indicated to give the alkylated benzimidazole.

Method 2. NaH (60% NaH in mineral oil) (0.82 mmol, 1.2 equiv.) was added at 0° C. to a reaction flask that contains a solution of the corresponding benzimidazole (0.68 mmol, 1.0 equiv.) in anhydrous THF (1.0 mL). The reaction mixture was stirred at 0° C. for 30 min until frothing discharged and the mixture became homogeneous, and then the corresponding coupling partner (i.e., bromide or mesylate) (1.37 mmol, 2.0 equiv.) was added to the reaction mixture. TLC monitored the reaction and the reaction was quenched with water. The mixture was extracted with EtOAc (×3), and the combined organic extracts was washed with brine and dried over anhydrous MgSO₄ and filtrated. The solvent was removed under reduced pressure and the residue was purified by flash chromatography on silica gel as indicated to give the alkylated benzimidazole.

### Step D: Procedure for Removal of Tert-Butyl Ester Using Et₃SiH

To a solution of tert-butyl 2-(2-ethyl-1H-benzimidazol-1-yl)acetate (70 mg, 0.27 mmol) in CH₂Cl₂ (3 mL) was added Et₃SiH (313 mg, 2.7 mmol) and TFA (3 mL) at rt. The solution was stirred at rt for overnight. Adjusted the pH of the reaction mixture to pH9 using saturated NaHCO₃ solution, washed with EtOAc (3×20 mL). The water solution was adjusted to pH6 using 3N HCl solution, and extracted with EtOAc (3×20 mL). The combined extract was washed with brine and dried over anhydrous MgSO₄, and filtered. The solvent was removed under reduced pressure and the residue was purified by flash chromatography on silica gel as indicated to give the alkylated benzimidazole.

### Step E: General Procedure for Esterification

To a mixture of 2-(2-ethyl-1H-benzimidazol-1-yl) acetic acid (1 equiv.), DCC (1.1 equiv.) and DMAP (0.1 equiv.) in CH₂Cl₂ (0.3 M) was added the corresponding alcohol (R₃OH) (1.1 equiv.) at rt. The solution was stirred at rt for overnight. The reaction mixture was diluted with EtOAc, washed with brine and dried over anhydrous MgSO₄, and filtered. The solvent was removed under reduced pressure and the residue was purified by flash chromatography on silica gel as indicated to give the esterified benzimidazole.

Step F: General Procedure for the Preparation of Alkyl Iodide Salt from Benzimidazole

The corresponding benzimidazole (0.10 mmol, 1 equiv.) was dissolved in the corresponding alkyl iodide (1.0 mL). The resulting mixture was stirred at 65° C. from 6 h to overnight depending upon TLC analysis. The excess alkyl iodide was removed under reduced pressure and the resulting residue was purified by either prep-TLC or flash chromatography on silica gel as indicated to give the salt.

Step G: General Procedure for the Preparation of Alkyl Chloride Salt from the Iodide Salt

The corresponding alkly iodide salt of benzimidazole (0.10 mmol, 1 equiv.) was dissolved in H₂O. This solution was added to Amberlite® IRA-400 Cl-exchange resin, and eluted with H₂O. Water was finally removed under reduced pressure and afforded the product as indicated.

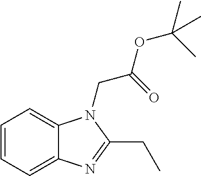 [SMILES: CCc1nc2ccccc2n1CC(=O)OC(C)(C)C]

The title compound was obtained following the general procedure (Step C, method 1) described above. Purification of the residue by flash chromatography on silica gel (gradient elution, 065% EtOAc in hexanes) afforded Lqr-5-040 as a yellow gel (18%). IR (cm⁻¹) 2929, 1732, 1544, 1465, 1360, 1238, 1156, 745; ¹H NMR (400 MHz, CDCl₃) δ 7.75-7.65 (m, 1H), 7.24-7.12 (m, 3H), 4.63 (s, 2H), 2.79 (q, J=7.6 Hz, 2H), 1.41 (t, J=7.6 Hz, 3H), 1.38 (s, 9H); ¹³C NMR (100 MHz, CDCl₃) δ 166.4, 156.1, 142.3, 135.2, 122.3, 122.0, 119.2, 108.6, 83.1, 45.5, 27.8 (3C), 20.6, 11.5; ES-API MS: m/z calcd for C₁₅H₂₀N₂O₂, found 261.1 [M+H].

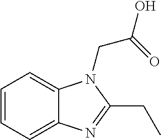

The title compound was obtained following the general procedure (Step D) described above. Purification of the residue by flash chromatography on silica gel (gradient elution, 010% MeOH in CH₂Cl₂) afforded Lqr-5-042 (50 mg, 0.24 mmol, 90%) as a yellow gel. IR (cm⁻¹) 2929, 1782, 1644, 1485, 1320, 1218, 1256, 745; ¹H NMR (400 MHz, CDCl₃) δ 7.61-7.55 (m, 1H), 7.46-7.36 (m, 3H), 4.90 (s, 2H), 3.04 (q, J=7.6 Hz, 2H), 1.45 (t, J=7.6 Hz, 3H); ¹³C NMR (100 MHz, CDCl₃) δ 170.6, 155.9, 134.9, 133.7, 124.0, 124.0, 115.0, 110.8, 46.6, 19.2, 9.7; ES-API MS: m/z calcd for C₁₁H₁₂N₂O₂, found 205.1 [M+H].

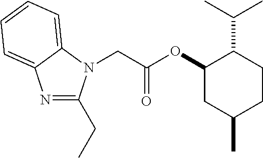

The title compound was obtained following the general procedure (Step E) described above. Purification of the residue by flash chromatography on silica gel (gradient elution, 15% MeOH in CH₂Cl₂) afforded Lqr-5-043 (23%) as a yellow gel. IR (cm⁻¹) 2926, 1739, 1734, 1545, 1468, 1360, 1220, 745; [α]_{D}²⁰−29.827 (c 0.4, CHCl₃); ¹H NMR (400 MHz, CDCl₃) δ 7.80-7.69 (m, 1H), 7.31-7.15 (m, 3H), 4.80 (s, 2H), 4.71 (td, J=10.8, 4.4 Hz, 1H), 2.86 (q, J=7.6 Hz, 2H), 1.96 (d, J=12.0 Hz, 1H), 1.75-1.57 (m, 3H), 1.57-1.37 (m, 1H), 1.47 (t, J=7.6 Hz, 3H), 1.29-1.18 (m, 2H), 1.07-0.78 (m, 2H), 0.88 (d, J=6.4 Hz, 3H), 0.76 (d, J=6.8 Hz, 3H), 0.64 (d, J=6.8 Hz, 3H); ¹³C NMR (100 MHz, CDCl₃) δ 167.0, 156.1, 142.4, 135.2, 122.4, 122.1, 119.3, 108.5, 76.4, 46.7, 45.1, 40.5, 33.9, 31.3, 26.1, 23.1, 21.9, 20.6, 20.5, 16.0, 11.5; ES-API MS: m/z calcd for C₂₁H₃₀N₂O₂, found 343.2 [M+H].

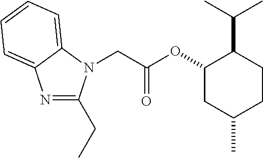

The title compound was obtained following the general procedure (Step E) described above. Purification of the residue by flash chromatography on silica gel (gradient elution, 15% MeOH in CH₂Cl₂) afforded Lqr-5-044 (46%) as a yellow gel. IR (cm⁻¹) 2926, 1739, 1734, 1545, 1468, 1360, 1220, 745; [α]_{D}²⁰ +30.026 (c 0.5, CHCl₃); ¹H NMR (400 MHz, CDCl₃) δ 7.77-7.71 (m, 1H), 7.31-7.22 (m, 2H), 7.2-7.17 (m, 1H), 4.80 (s, 2H), 4.71 (td, J=10.8, 4.4 Hz, 1H), 2.86 (q, J=7.2 Hz, 2H), 2.00-1.92 (m, 1H), 1.72-1.58 (m, 3H), 1.47 (t, J=7.2 Hz, 3H), 1.57-1.38 (m, 1H), 1.30-1.19 (m, 1H), 1.06-0.77 (m, 3H), 0.88 (d, J=6.4 Hz, 3H), 0.76 (d, J=6.8 Hz, 3H), 0.64 (d, J=6.8 Hz, 3H); ¹³C NMR (100 MHz, CDCl₃) δ 167.0, 156.0, 142.4, 135.3, 122.4, 122.1, 119.3, 108.5, 76.4, 46.7, 45.1, 40.5, 33.9, 31.3, 26.1, 23.1, 21.9, 20.6, 20.5, 16.0, 11.5; ES-API MS: m/z calcd for C₂₁H₃₀N₂O₂, found 343.2 [M+H].

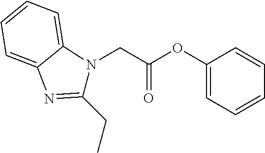 [SMILES: CCc1nc2ccccc2n1CC(=O)Oc1ccccc1]

The title compound was obtained following the general procedure (Step E) described above. Purification of the residue by flash chromatography on silica gel (gradient elution, 15% MeOH in CH₂Cl₂) afforded Lqr-5-053 (71%) as a yellow gel. ¹H NMR (400 MHz, CDCl₃) δ 7.81-7.70 (m, 1H), 7.42-7.18 (m, 6H), 7.04 (d, J=8.4 Hz, 2H), 5.06 (s, 2H), 2.95 (q, J=7.6 Hz, 2H), 1.52 (t, J=7.6 Hz, 3H); ¹³C NMR (100 MHz, CDCl₃) δ 165.9, 156.0, 149.97, 142.4, 135.1, 129.5 (2C), 126.4, 122.7, 122.4, 120.9 (2C), 119.5, 108.4, 44.9, 20.7, 11.5; ES-API MS: m/z calcd for C₁₇H₁₆N₂O₂, found 281.2 [M+H].

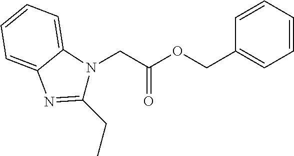 [SMILES: CCc1nc2ccccc2n1CC(=O)OCc1ccccc1]

The title compound was obtained following the general procedure (Step E) described above. Purification of the residue by flash chromatography on silica gel (gradient elution, 15% MeOH in CH₂Cl₂) afforded Lqr-5-054 (50%) as a yellow gel. ¹H NMR (400 MHz, CDCl₃) δ 7.78-7.68 (m, 1H), 7.41-7.30 (m, 3H), 7.30-7.16 (m, 5H), 5.19 (s, 2H), 4.86 (s, 2H), 2.83 (q, J=7.6 Hz, 2H), 1.43 (t, J=7.6 Hz, 3H); ¹³C NMR (100 MHz, CDCl₃) δ 167.2, 156.0, 142.4, 135.2, 134.7, 128.7, 128.6 (2C), 128.3 (2C), 122.4, 122.2, 119.4, 108.5, 67.6, 44.8, 20.6, 11.4; ES-API MS: m/z calcd for C₁₈H₁₈N₂O₂, found 295.2 [M+H].

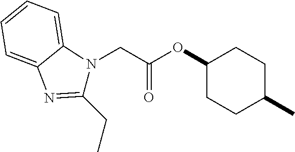 [SMILES: CCc1nc2ccccc2n1CC(=O)O[C@H]1CC[C@@H](C)CC1]

The title compound was obtained following the general procedure (Step E) described above. Purification of the residue by flash chromatography on silica gel (gradient elution, 10% MeOH in CH₂Cl₂) afforded Lqr-5-055 (66%) as a yellow gel. IR (cm⁻¹) 2926, 1739, 1734, 1545, 1468, 1360, 1220, 745; ¹H NMR (400 MHz, CDCl₃) δ 7.78-7.68 (m, 1H), 7.25-7.22 (m, 3H), 5.09-5.4.95 (m, 1H), 4.83 (s, 2H), 2.88 (q, J=7.6 Hz, 2H), 1.79-1.56 (m, 4H), 1.52-1.20 (m, 4H), 1.47 (t, J=7.6 Hz, 3H), 0.89-0.66 (m, 1H), 0.70 (d, J=6.0 Hz, 3H); ¹³C NMR (100 MHz, CDCl₃) δ 166.7, 156.1, 142.5, 135.3, 122.4, 122.1, 119.4, 108.6, 72.0, 45.3, 31.0, 29.3 (2C), 28.8 (2C), 21.8, 20.7, 11.5; ES-API MS: m/z calcd for C₁₈H₂₄N₂O₂, found 301.1 [M+H].

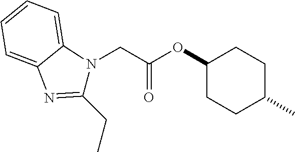 [SMILES: CCc1nc2ccccc2n1CC(=O)O[C@H]1CC[C@H](C)CC1]

The title compound was obtained following the general procedure (Step E) described above. Purification of the residue by flash chromatography on silica gel (gradient elution, 010% MeOH in CH₂Cl₂) afforded Lqr-5-056 (66%) as a yellow gel. IR (cm⁻¹) 2936, 1739, 1777, 1548, 1470, 1360, 1211, 745; ¹H NMR (400 MHz, CDCl₃) δ 7.78-7.70 (m, 1H), 7.25-7.18 (m, 3H), 4.79 (s, 2H), 4.80-4.68 (m, 1H), 2.86 (q, J=7.6 Hz, 2H), 1.98-1.87 (m, 2H), 1.76-1.54 (m, 4H), 1.46 (t, J=7.6 Hz, 3H), 1.39-1.26 (m, 2H), 1.06-0.95 (m, 1H), 0.87 (d, J=6.4 Hz, 3H); ¹³C NMR (100 MHz, CDCl₃) δ 166.7, 156.1, 142.5, 135.3, 122.4, 122.1, 119.4, 108.6, 72.0, 45.3, 31.0, 29.4 (2C), 28.8 (2C), 21.8, 20.7, 11.5; ES-API MS: m/z calcd for C₁₈H₂₄N₂O₂, found 301.1 [M+H].

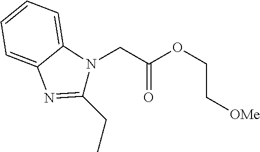 [SMILES: CCc1nc2ccccc2n1CC(=O)OCCOC]

The title compound was obtained following the general procedure (Step E) described above. Purification of the residue by flash chromatography on silica gel (gradient elution, 010% MeOH in CH₂Cl₂) afforded Lqr-5-062 (46%) as a yellow gel. ¹H NMR (400 MHz, CDCl₃) δ 7.76-7.69 (m, 1H), 7.25-7.19 (m, 3H), 4.86 (s, 2H), 4.31 (t, J=4.4 Hz, 2H), 3.55 (t, J=4.4 Hz, 2H), 3.34 (s, 3H), 2.85 (q, J=7.6 Hz, 2H), 1.47 (t, J=7.6 Hz, 3H); ¹³C NMR (100 MHz, CDCl₃) δ 167.3, 156.1, 142.3, 135.2, 122.4, 122.2, 119.4, 108.5, 69.9, 64.8, 58.9, 44.6, 20.6, 11.4; ES-API MS: m/z calcd for C₁₄H₁₈N₂O₃, found 263.1 [M+H].

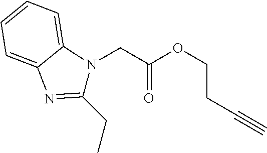 [SMILES: C#CCCOC(=O)Cn1c(CC)nc2ccccc21]

The title compound was obtained following the general procedure (Step E) described above. Purification of the residue by flash chromatography on silica gel (gradient elution, 010% MeOH in CH₂Cl₂) afforded Lqr-5-063 (28%) as a yellow gel. ¹H NMR (400 MHz, CDCl₃) δ 7.78-7.68 (m, 1H), 7.29-7.18 (m, 3H), 4.86 (s, 2H), 4.28 (t, J=6.4 Hz, 2H), 2.87 (q, J=7.6 Hz, 2H), 2.52 (td, J=6.4, 2.4 Hz, 2H), 1.96 (t, J=2.4 Hz, 1H), 1.47 (t, J=7.6 Hz, 3H); ¹³C NMR (100 MHz, CDCl₃) δ 167.1, 156.0, 142.4, 135.1, 122.5, 122.2, 119.4, 108.5, 79.3, 70.3, 63.3, 44.6, 20.6, 18.9, 11.4; ES-API MS: m/z calcd for C₁₅H₁₆N₂O₂, found 257.1 [M+H].

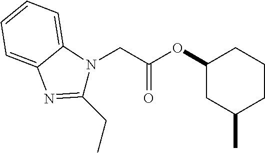 [SMILES: CCc1nc2ccccc2n1CC(=O)O[C@H]1CCC[C@@H](C)C1]

The title compound was obtained following the general procedure (Step C, method 2) described above. Purification of the residue by flash chromatography on silica gel (gradient elution, 070% EtOAc/hexanes) afforded LW-V-262 as a white solid (99%). IR (cm⁻¹) 2938, 2864, 1742, 1463, 1198, 742; [α]_{D}²⁰+2.264 (c 0.53, CHCl₃); ¹H NMR (400 MHz, CDCl₃) δ 7.68 (m, 1H), 7.19-7.11 (m, 3H), 4.69 (m, 1H), 4.66 (s, 2H), 2.76 (q, J=7.6 Hz, 2H), 1.85 (m, 2H), 1.68 (m, 1H), 1.54 (m, 1H), 1.39 (s, J=7.6 Hz, 3H), 1.39 (m, 1H), 1.24 (m, 1H), 1.11 (m, 1H), 0.88 (m, 1H), 0.84 (d, J=6.8 Hz, 3H), 0.72 (m, 1H); ¹³C NMR (100 MHz, CDCl₃) δ 166.9, 156.2, 142.5, 135.3, 122.4, 122.1, 119.3, 108.6, 75.3, 45.0, 40.1, 33.7, 31.3, 31.2, 23.7, 22.2, 20.6, 11.5; ES-API MS: m/z calcd for C₁₈H₂₄N₂O₂, found 301.1 [M+H].

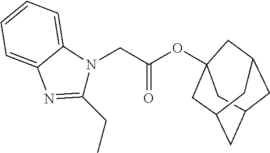 [SMILES: CCc1nc2ccccc2n1CC(=O)OC12CC3CC(CC(C3)C1)C2]

The title compound was obtained following the general procedure (Step C, method 2) described above. Purification of the residue by flash chromatography on silica gel (gradient elution, 065% EtOAc/hexanes) afforded LW-V-267 as a colorless gel (89%). IR (cm⁻¹) 2913, 2854, 1738, 1463, 1200, 1052, 741; ¹H NMR (400 MHz, CDCl₃) δ 7.70 (m, 1H), 7.21-7.14 (m, 3H), 4.62 (s, 2H), 2.79 (q, J=7.6 Hz, 2H), 2.10 (br, 3H), 2.01 (m, 6H), 1.58 (m, 6H), 1.42 (t, J=7.6 Hz, 3H); ¹³C NMR (100 MHz, CDCl₃) δ 166.1, 156.2, 142.5, 135.4, 122.3, 122.0, 119.3, 108.7, 83.3, 45.7, 41.2 (3C), 36.0 (3C), 30.9 (3C), 20.7, 11.6; ES-API MS: m/z calcd for C₂₁H₂₆N₂O₂, found 339.2 [M+H].

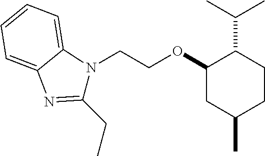 [SMILES: CCc1nc2ccccc2n1CCO[C@@H]1C[C@H](C)CC[C@H]1C(C)C]

The title compound was obtained following the general procedure (Step C, method 2) described above. Purification of the residue by flash chromatography on silica gel (gradient elution, 055% EtOAc/hexanes) afforded LW-V-265 as a yellow gel (89%). IR (cm⁻¹) 2954, 2922, 2869, 1463, 1456, 1111, 742; [α]_{D}²⁰−40.92 (c 0.86, CHCl₃); ¹H NMR (400 MHz, CDCl₃) δ 7.70 (m, 1H), 7.24 (m, 1H), 7.17 (m, 2H), 4.22 (m, 2H), 3.87 (ddd, J=9.6, 5.2, 5.2 Hz, 1H), 3.54 (ddd, J=9.6, 6.4, 5.2 Hz, 1H), 2.90 (q, J=7.6 Hz, 2H), 2.87 (m, 1H), 1.85 (m, 1H), 1.79 (m, 1H), 1.58-1.48 (m, 2H), 1.43 (t, J=7.6 Hz, 3H), 1.22 (m, 1H), 1.08 (m, 1H), 0.82 (m, 1H), 0.81 (d, J=6.4 Hz, 3H), 0.78 (m, 1H), 0.74 (d, J=7.2 Hz, 3H), 0.69 (m, 1H), 0.43 (d, J=6.8 Hz, 3H); ¹³C NMR (100 MHz, CDCl₃) δ 156.8, 142.9, 135.1, 121.8, 121.7, 119.3, 109.2, 80.2, 66.4, 48.1, 44.0, 40.2, 34.5, 31.5, 25.5, 23.2, 22.3, 20.93, 20.89, 15.9, 11.8; ES-API MS: m/z calcd for C₂₁H₃₂N₂O, found 329.2 [M+H].

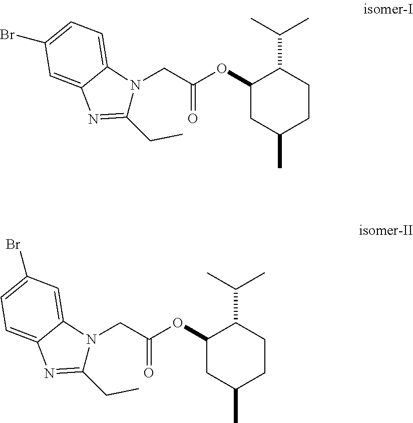 [SMILES: CCc1nc2cc(Br)ccc2n1CC(=O)O[C@@H]1C[C@H](C)CC[C@H]1C(C)C ; CCc1nc2ccc(Br)cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 030% EtOAc in hexanes) afforded LW-IV-31 isomer-I (48%) and isomer-II (48%) as yellow gel.

Isomer-I: IR (cm⁻¹) 3390, 2956, 2872, 1748, 1732, 1516, 1470, 1220, 981, 961, 793, 754; [α]_{D}²⁰−22.59 (c 1.62, CHCl₃); ¹H NMR (400 MHz, CDCl₃) δ 7.86 (d, J=1.6 Hz, 1H), 7.33 (dd, J=8.8, 1.6 Hz, 1H), 7.05 (d, J=8.8 Hz, 1H), 4.76 (s, 2H), 4.70 (ddd, J=10.8, 10.8, 4.4 Hz, 1H), 2.83 (q, J=7.6 Hz, 2H), 1.94 (m, 1H), 1.64-1.59 (m, 2H), 1.50 (m, 1H), 1.44 (t, J=7.6 Hz, 3H), 1.42 (m, 1H), 1.24 (m, 1H), 1.00 (m, 1H), 0.92 (m, 1H), 0.88 (d, J=6.8 Hz, 3H), 0.83 (m, 1H), 0.77 (d, J=6.8 Hz, 3H), 0.64 (d, J=6.8 Hz, 3H); ¹³C NMR (100 MHz, CDCl₃) δ 166.8, 157.5, 143.8, 134.5, 125.6, 122.4, 115.4, 110.0, 76.9, 46.9, 45.4, 40.8, 34.1, 31.5, 26.4, 23.3, 22.1, 20.9, 20.8, 16.2, 11.7; ES-API MS: m/z calcd for C₂₁H₂₉BrN₂O₂, found 421.2 [M+H].

Isomer-II: IR (cm⁻¹) 3389, 2957, 2872, 1739, 1732, 1614, 1269, 1202, 903, 810, 753; [α]_{D}²⁰−12.22 (c 1.80, CHCl₃); ¹H NMR (400 MHz, CDCl₃) δ 7.58 (d, J=8.8 Hz, 1H), 7.34 (d, J=2.0 Hz, 1H), 7.33 (dd, J=8.8, 2.0 Hz, 1H), 4.74 (s, 2H), 4.72 (ddd, J=11.2, 10.8, 4.4 Hz, 1H), 2.82 (q, J=7.6 Hz, 2H), 1.96 (m, 1H), 1.67-1.59 (m, 2H), 1.52 (m, 1H), 1.45 (t, J=7.6 Hz, 3H), 1.43 (m, 1H), 1.25 (m, 1H), 0.99 (m, 1H), 0.93 (m, 1H), 0.88 (d, J=6.8 Hz, 3H), 0.83 (m, 1H), 0.78 (d, J=6.8 Hz, 3H), 0.67 (d, J=6.8 Hz, 3H); ¹³C NMR (100 MHz, CDCl₃) δ 166.8, 157.1, 141.5, 136.6, 125.7, 120.8, 115.9, 112.1, 77.0, 47.0, 45.4, 40.8, 34.1, 31.6, 26.4, 23.3, 22.1, 20.9, 16.2, 11.6; ES-API MS: m/z calcd for C₂₁H₂₉BrN₂O₂, found 421.2 [M+H].

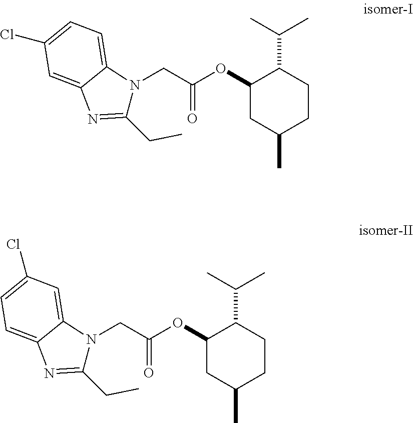 [SMILES: CCc1nc2cc(Cl)ccc2n1CC(=O)O[C@@H]1C[C@H](C)CC[C@H]1C(C)C ; CCc1nc2ccc(Cl)cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 035% EtOAc in hexanes) afforded LW-IV-34 isomer-I (50%) and isomer-II (47%) as yellow gel.

Isomer-I: IR (cm⁻¹) 3390, 2957, 2872, 1740, 1516, 1462, 1202, 1072, 918, 795; [α]_{D}²⁰−29.02 (c 1.13, CHCl₃); ¹H NMR (400 MHz, CDCl₃) δ 7.71 (d, J=1.6 Hz, 1H), 7.20 (dd, J=8.4, 2.0 Hz, 1H), 7.10 (d, J=8.4 Hz, 1H), 4.77 (s, 2H), 4.70 (ddd, J=10.8, 11.2, 4.4 Hz, 1H), 2.84 (q, J=7.6 Hz, 2H), 1.94 (m, 1H), 1.67-1.59 (m, 2H), 1.50 (m, 1H), 1.45 (t, J=7.6 Hz, 3H), 1.42 (m, 1H), 1.25 (m, 1H), 1.00 (m, 1H), 0.92 (m, 1H), 0.88 (d, J=6.4 Hz, 3H), 0.83 (m, 1H), 0.77 (d, J=6.8 Hz, 3H), 0.64 (d, J=6.8 Hz, 3H); ¹³C NMR (100 MHz, CDCl₃) δ 166.8, 157.7, 143.3, 134.1, 128.1, 123.0, 119.4, 109.6, 76.9, 47.0, 45.5, 40.8, 34.1, 31.5, 26.4, 23.3, 22.1, 20.9, 20.8, 16.3, 11.7; ES-API MS: m/z calcd for C₂₁H₂₉ClN₂O₂, found 377.2 [M+H].

Isomer-II: IR (cm⁻¹) 3390, 2957, 2871, 1740, 1464, 1269, 1203, 812; [α]_{D}²⁰−17.81 (c 1.10, CHCl₃); ¹H NMR (400 MHz, CDCl₃) δ 7.62 (d, J=8.4 Hz, 1H), 7.19 (dd, J=8.4, 2.0 Hz, 1H), 7.17 (d, J=1.2 Hz, 1H), 4.73 (s, 2H), 4.71 (ddd, J=10.8, 10.8, 4.4 Hz, 1H), 2.82 (q, J=7.6 Hz, 2H), 1.95 (m, 1H), 1.66-1.59 (m, 2H), 1.52 (m, 1H), 1.44 (t, J=7.6 Hz, 3H), 1.42 (m, 1H), 1.24 (m, 1H), 0.98 (m, 1H), 0.93 (m, 1H), 0.88 (d, J=6.4 Hz, 3H), 0.82 (m, 1H), 0.78 (d, J=7.2 Hz, 3H), 0.66 (d, J=7.2 Hz, 3H); ¹³C NMR (100 MHz, CDCl₃) δ 166.8, 157.2, 141.2, 136.1, 128.4, 123.0, 120.3, 109.1, 76.9, 47.0, 45.4, 40.8, 34.1, 31.5, 26.4, 23.3, 22.1, 20.9, 20.8, 16.1, 11.6; ES-API MS: m/z calcd for C₂₁H₂₉ClN₂O₂, found 377.2 [M+H].

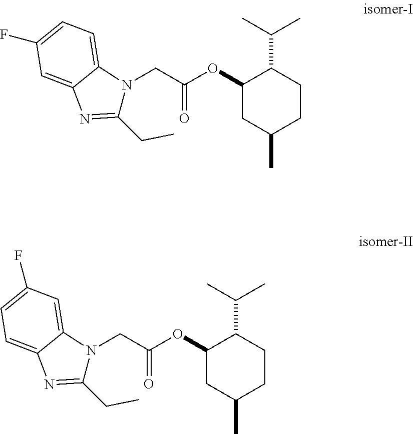 [SMILES: CCc1nc2cc(F)ccc2n1CC(=O)O[C@@H]1C[C@H](C)CC[C@H]1C(C)C ; CCc1nc2ccc(F)cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 030% EtOAc in hexanes) afforded LW-IV-35 isomer-I (45%) and isomer-II (40%) as yellow gel.

Isomer-I: IR (cm⁻¹) 3390, 2957, 2872, 1741, 1516, 1487, 1450, 1203, 1136, 958, 795; [α]D²⁰−31.35 (c 1.18, CHCl₃); ¹H NMR (400 MHz, CDCl₃) δ 7.40 (dd, J=9.2, 2.4 Hz, 1H), 7.09 (dd, J=8.8, 4.4 Hz, 1H), 6.97 (ddd, J=9.2, 8.8, 2.4 Hz, 1H), 4.77 (s, 2H), 4.70 (ddd, J=10.8, 10.8, 4.4 Hz, 1H), 2.83 (q, J=7.2 Hz, 2H), 1.94 (m, 1H), 1.66-1.59 (m, 2H), 1.49 (m, 1H), 1.44 (t, J=7.6 Hz, 3H), 1.42 (m, 1H), 1.24 (m, 1H), 0.96 (m, 1H), 0.92 (m, 1H), 0.87 (d, J=6.4 Hz, 3H), 0.82 (m, 1H), 0.77 (d, J=7.2 Hz, 3H), 0.63 (d, J=7.2 Hz, 3H); ¹³C NMR (100 MHz, CDCl₃) δ 166.9, 159.6 (d, J=235.7 Hz, 1C), 157.9, 143.0 (d, J=12.7 Hz, 1C), 131.9, 110.7 (d, J=26.0 Hz, 1C), 109.0 (d, J=10.2 Hz, 1C), 105.5 (d, J=24.2 Hz, 1C), 76.8, 47.0, 45.5, 40.8, 34.1, 31.5, 26.4, 23.3, 22.1, 21.0, 20.8, 16.2, 11.7; ES-API MS: m/z calcd for C₂₁H₂₉FN₂O₂, found 361.2 [M+H].

Isomer-II: IR (cm⁻¹) 3388, 2957, 2872, 1741, 1626, 1484, 1464, 1204, 1180, 828; [α]_{D}²⁰−31.81 (c 1.10, CHCl₃); ¹H NMR (400 MHz, CDCl₃) δ 7.64 (dd, J=8.8, 4.8 Hz, 1H), 6.97 (ddd, J=10.0, 9.6, 2.8 Hz, 1H), 6.87 (dd, J=8.4, 2.4 Hz, 1H), 4.73 (s, 2H), 4.71 (ddd, J=10.8, 10.8, 4.4 Hz, 1H), 2.82 (q, J=7.6 Hz, 2H), 1.95 (m, 1H), 1.67-1.58 (m, 2H), 1.52 (m, 1H), 1.44 (m, 1H), 1.44 (t, J=7.6 Hz, 3H), 1.25 (m, 1H), 0.99 (m, 1H), 0.93 (m, 1H), 0.88 (d, J=6.8 Hz, 3H), 0.83 (m, 1H), 0.77 (d, J=6.8 Hz, 3H), 0.65 (d, J=7.2 Hz, 3H); ¹³C NMR (100 MHz, CDCl₃) δ 166.9, 158.8 (d, J=238.3 Hz, 1C), 156.9 (d, J=3.0 Hz, 1C), 138.8, 135.6 (d, J=12.9 Hz, 1C), 120.2 (d, J=9.9 Hz, 1C), 110.5 (d, J=24.6 Hz, 1C), 95.9 (d, J=27.7 Hz, 1C), 76.9, 47.0, 45.4, 40.8, 34.1, 31.5, 26.4, 23.3, 22.1, 20.9, 20.8, 16.2, 11.6; ES-API MS: m/z calcd for C₂₁H₂₉FN₂O₂, found 361.2 [M+H].

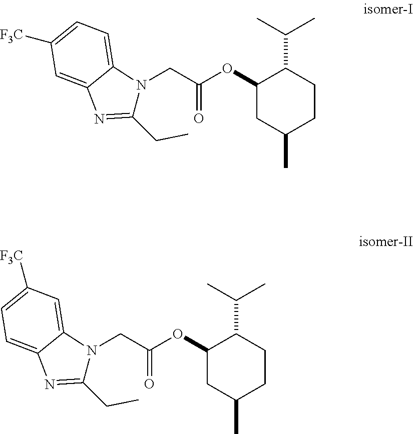 [SMILES: CCc1nc2cc(C(F)(F)F)ccc2n1CC(=O)O[C@@H]1C[C@H](C)CC[C@H]1C(C)C ; CCc1nc2ccc(C(F)(F)F)cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 035% EtOAc in hexanes) afforded LW-IV-36 isomer-I (41%) and isomer-II (49%) as yellow gel.

Isomer-I: IR (cm⁻¹) 3469, 2958, 2873, 1744, 1628, 1449, 1329, 1221, 1119, 809; [α]_{D}²⁰−28.56 (c 1.12, CHCl₃); ¹H NMR (400 MHz, CDCl₃) δ 8.00 (s, 1H), 7.48 (d, J=8.4 Hz, 1H), 7.26 (d, J=8.4 Hz, 1H), 4.81 (s, 2H), 4.70 (ddd, J=10.8, 10.8, 4.4 Hz, 1H), 2.86 (q, J=7.2 Hz, 2H), 1.95 (m, 1H), 1.66-1.58 (m, 2H), 1.51-1.38 (m, 2H), 1.46 (t, J=7.6 Hz, 3H), 1.24 (m, 1H), 0.98 (m, 1H), 0.92 (m, 1H), 0.87 (d, J=6.8 Hz, 3H), 0.82 (m, 1H), 0.75 (d, J=7.2 Hz, 3H), 0.63 (d, J=6.8 Hz, 3H); ¹³C NMR (100 MHz, CDCl₃) δ 166.7, 158.4, 142.1, 137.5, 125.0 (q, J=270.3 Hz, 1C), 125.0 (q, J=32.1 Hz, 1C), 119.6 (q, J=3.6 Hz, 1C), 117.2 (q, J=4.1 Hz, 1C), 109.2, 77.0, 46.9, 45.4, 40.8, 34.1, 31.5, 26.4, 23.3, 22.1, 20.9, 20.7, 16.2, 11.6; ES-API MS: m/z calcd for C₂₂H₂₉F₃N₂O₂, found 411.2 [M+H].

Isomer-II: IR (cm⁻¹) 3406, 2959, 2873, 1741, 1520, 1464, 1349, 1203, 1120, 824; [α]_{D}²⁰−18.23 (c 1.81, CHCl₃); ¹H NMR (400 MHz, CDCl₃) δ 7.79 (d, J=8.4 Hz, 1H), 7.48 (d, J=8.4 Hz, 1H), 7.46 (s, 1H), 4.82 (s, 2H), 4.71 (ddd, J=11.2, 10.8, 4.4 Hz, 1H), 2.86 (q, J=7.6 Hz, 2H), 1.94 (m, 1H), 1.65-1.58 (m, 2H), 1.92-1.38 (m, 2H), 1.46 (t, J=7.6 Hz, 3H), 1.23 (m, 1H), 0.97 (m, 1H), 0.92 (m, 1H), 0.87 (d, J=6.8 Hz, 3H), 0.81 (m, 1H), 0.74 (d, J=7.2 Hz, 3H), 0.63 (d, J=6.8 Hz, 3H); ¹³C NMR (100 MHz, CDCl₃) δ 166.7, 159.0, 144.9, 135.0, 124.9 (q, J=270.4 Hz, 1C), 124.9 (q, J=32.1 Hz, 1C), 119.9, 119.5 (q, J=3.5 Hz, 1C), 106.6 (q, J=4.3 Hz, 1C), 77.0, 47.0, 45.4, 40.8, 34.1, 31.5, 26.4, 23.3, 22.0, 21.0, 20.7, 16.0, 11.6; ES-API MS: m/z calcd for C₂₂H₂₉F₃N₂O₂, found 411.2 [M+H].

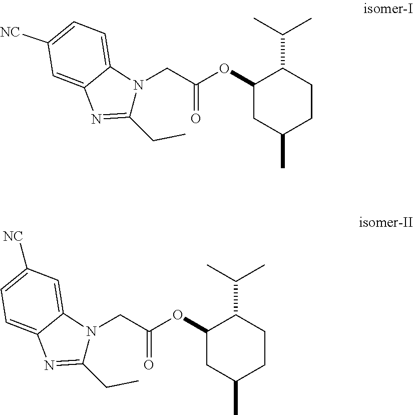 [SMILES: CCc1nc2cc(C#N)ccc2n1CC(=O)O[C@@H]1C[C@H](C)CC[C@H]1C(C)C ; CCc1nc2ccc(C#N)cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 038% EtOAc in hexanes) afforded LW-IV-37 isomer-I (45%) and isomer-II (23%) as yellow gel.

Isomer-I: IR (cm⁻¹) 3389, 2957, 2872, 2225, 1743, 1220, 961, 756; [α]_{D}²⁰−25.42 (c 1.07, CHCl₃); ¹H NMR (400 MHz, CDCl₃) δ 8.05 (s, 1H), 7.51 (dd, J=8.4, 1.2 Hz, 1H), 7.27 (d, J=8.0 Hz, 1H), 4.83 (s, 2H), 4.72 (ddd, J=10.8, 10.8, 4.4 Hz, 1H), 2.88 (q, J=7.6 Hz, 2H), 1.95 (m, 1H), 1.68-1.60 (m, 2H), 1.54-1.39 (m, 2H), 1.48 (t, J=7.6 Hz, 3H), 1.26 (m, 1H), 0.99 (m, 1H), 0.93 (m, 1H), 0.89 (d, J=6.8 Hz, 3H), 0.84 (m, 1H), 0.78 (d, J=7.2 Hz, 3H), 0.65 (d, J=6.8 Hz, 3H); ¹³C NMR (100 MHz, CDCl₃) δ 166.4, 159.1, 142.1, 138.3, 126.4, 124.4, 120.0, 110.0, 105.8, 77.2, 47.0, 45.5, 40.8, 34.1, 31.5, 26.5, 23.4, 22.1, 21.0, 20.8, 16.3, 11.5; ES-API MS: m/z calcd for C₂₂H₂₉N₃O₂, found 368.2 [M+H].

Isomer-II: IR (cm⁻¹) 3411, 2957, 2871, 2224, 1742, 1464, 1222, 822; [α]_{D}²⁰−23.35 (c 1.25, CHCl₃); ¹H NMR (400 MHz, CDCl₃) δ 7.78 (d, J=8.0 Hz, 1H), 7.53 (s, 1H), 7.51 (dd, J=8.4, 1.2 Hz, 1H), 4.82 (s, 2H), 4.73 (ddd, J=11.2, 10.8, 4.4 Hz, 1H), 2.88 (q, J=7.2 Hz, 2H), 1.96 (m, 1H), 1.67-1.61 (m, 2H), 1.53 (m, 1H), 1.47 (t, J=7.6 Hz, 3H), 1.44 (m, 1H), 1.27 (m, 1H), 1.00 (m, 1H), 0.95 (m, 1H), 0.89 (d, J=6.8 Hz, 3H), 0.84 (m, 1H), 0.80 (d, J=7.2 Hz, 3H), 0.67 (d, J=6.8 Hz, 3H); ¹³C NMR (100 MHz, CDCl₃) δ 166.4, 160.0, 145.6, 135.2, 126.3, 120.5, 119.9, 113.7, 105.6, 77.2, 47.0, 45.4, 40.8, 34.1, 31.5, 26.5, 23.3, 22.1, 21.0, 20.8, 16.2, 11.5; ES-API MS: m/z calcd for C₂₂H₂₉N₃O₂, found 368.2 [M+H].

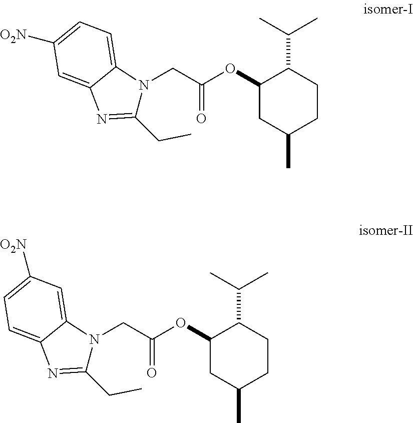 [SMILES: CCc1nc2cc([N+](=O)[O-])ccc2n1CC(=O)O[C@@H]1C[C@H](C)CC[C@H]1C(C)C ; CCc1nc2ccc([N+](=O)[O-])cc2n1CC(=O)O[C@@H]1C[C@H](C)CC[C@H]1C(C)C]

The title compounds were obtained following the general procedure (Step C, method 2) described above. Purification of the residue by flash chromatography on silica gel (gradient elution, 040% EtOAc in hexanes) afforded LW-IV-38 isomer-I (66%) and isomer-II (16%) as yellow gel.

Isomer-I: IR (cm⁻¹) 2957, 2872, 1742, 1620, 1523, 1340, 1221, 741; [α]_{D}²⁰−26.73 (c 1.04, CHCl₃); ¹H NMR (400 MHz, CDCl₃) δ 8.63 (d, J=2.0 Hz, 1H), 8.19 (dd, J=8.8, 2.0 Hz, 1H), 7.25 (d, J=9.2 Hz, 1H), 4.85 (s, 2H), 4.73 (ddd, J=11.2, 10.8, 4.4 Hz, 1H), 2.88 (q, J=7.6 Hz, 2H), 1.95 (m, 1H), 1.67-1.60 (m, 2H), 1.54 (m, 1H), 1.48 (t, J=7.6 Hz, 3H), 1.44 (m, 1H), 1.27 (m, 1H), 1.00 (m, 1H), 0.94 (m, 1H), 0.88 (d, J=6.8 Hz, 3H), 0.84 (m, 1H), 0.79 (d, J=6.8 Hz, 3H), 0.66 (d, J=7.2 Hz, 3H); ¹³C NMR (100 MHz, CDCl₃) δ 166.3, 160.1, 144.0, 142.0, 139.7, 118.7, 116.2, 108.8, 77.3, 47.0, 45.6, 40.8, 34.1, 31.5, 26.5, 23.4, 22.1, 21.1, 20.8, 16.3, 11.4; ES-API MS: m/z calcd for C₂₁H₂₉N₃O₄, found 388.2 [M+H].

Isomer-II: IR (cm⁻¹) 2957, 2872, 1741, 1524, 1463, 1342, 1223, 737; [α]_{D}²⁰−19.51 (c 1.23, CHCl₃); ¹H NMR (400 MHz, CDCl₃) δ 8.17 (dd, J=9.2, 2.0 Hz, 1H), 8.17 (d, J=2.0 Hz, 1H), 7.76 (d, J=9.2 Hz, 1H), 4.87 (s, 2H), 4.75 (ddd, J=10.8, 10.8, 4.4 Hz, 1H), 2.89 (q, J=7.6 Hz, 2H), 1.97 (m, 1H), 1.67-1.56 (m, 3H), 1.48 (t, J=7.6 Hz, 3H), 1.44 (m, 1H), 1.28 (m, 1H), 1.05-0.92 (m, 2H), 0.88 (d, J=6.4 Hz, 3H), 0.84 (m, 1H), 0.80 (d, J=7.2 Hz, 3H), 0.67 (d, J=6.8 Hz, 3H); ¹³C NMR (100 MHz, CDCl₃) δ 166.3, 161.6, 147.2, 143.6, 134.9, 119.5, 118.5, 105.9, 77.3, 47.0, 45.5, 40.8, 34.1, 31.5, 26.6, 23.3, 22.1, 21.2, 20.8, 16.2, 11.4; ES-API MS: m/z calcd for C₂₁H₂₉N₃O₄, found 388.2 [M+H].

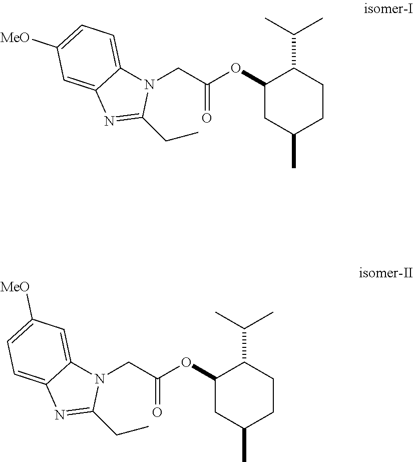

The title compounds were obtained following the general procedure (Step C, method 2) described above. Purification of the residue by flash chromatography on silica gel (gradient elution, 060% EtOAc in hexanes) afforded AP-I-25 isomer-I (33%) and isomer-II (30%) as yellow gel.

Isomer-I: IR (cm⁻¹) 2956, 2871, 1741, 1626, 1594, 1528, 1488, 1460, 1411, 1263, 1217; [α]_{D}²⁰−20.90 (c 1.014 CHCl₃); ¹H NMR (400 MHz, CDCl₃) δ 7.60 (d, J=8.7 Hz, 1H), 6.85 (dd, J=8.7, 2.4 Hz, 1H), 6.65 (d, J=2.3 Hz, 1H), 4.73 (s, 2H), 4.71 (ddd, J=11.2, 10.8, 4.4 Hz, 1H), 3.82 (s, 3H), 2.81 (q, J=7.5 Hz, 2H), 1.96 (m, 1H), 1.67-1.58 (m, 2H), 1.54 (m, 1H), 1.44 (t, J=7.5 Hz, 3H), 1.24 (m, 2H), 0.99 (m, 1H), 0.92 (m, 1H), 0.87 (d, J=6.5 Hz, 3H), 0.82 (m, 1H), 0.75 (d, J=7.0 Hz, 3H), 0.64 (d, J=7.0 Hz, 3H); ¹³C NMR (100 MHz, CDCl₃) δ 167.3, 156.6, 155.4, 137.1, 136.1, 120.0, 111.0, 93.1, 76.6, 56.0, 46.9, 45.4, 40.8, 34.1, 31.5, 26.3, 23.3, 22.1, 20.9, 20.8, 16.2, 11.8; ES-API MS: m/z calcd for C₂₂H₃₂N₂O₃, found 373.2 [M+H].

Isomer-II: IR (cm⁻¹) 2956, 2871, 1741, 1624, 1595, 1516, 1490, 1449, 1276, 1198, 1154; [α]_{D}²⁰−29.19 (c 1.000 CHCl₃); ¹H NMR (400 MHz, CDCl₃) δ 7.24 (d, J=2.4 Hz, 1H), 7.06 (d, J=8.7 Hz, 1H), 6.86 (dd, J=8.7, 2.4 Hz, 1H), 4.74 (s, 2H), 4.70 (ddd, J=11.2, 10.8, 4.4 Hz, 1H), 3.83 (s, 3H), 2.81 (q, J=7.5 Hz, 2H), 1.94 (m, 1H), 1.67-1.59 (m, 2H), 1.52 (m, 1H), 1.43 (d, J=7.5 Hz, 3H), 1.24 (m, 2H), 0.98 (m, 1H), 0.91 (m, 1H), 0.87 (d, J=6.5 Hz, 3H), 0.82 (m, 1H), 0.76 (d, J=7.0 Hz, 3H), 0.64 (d, J=7.0 Hz, 3H); ¹³C NMR (100 MHz, CDCl₃) δ 167.2, 156.6, 156.3, 143.4, 130.1, 112.2, 109.1, 102.3, 76.6, 56.0, 47.0, 45.4, 40.8, 34.2, 31.5, 26.3, 23.4, 22.1, 20.9, 20.8, 16.3, 11.8; ES-API MS: m/z calcd for C₂₂H₃₂N₂O₃, found 373.2 [M+H].

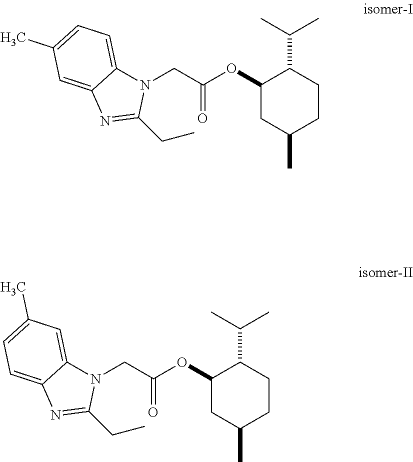

The title compounds were obtained following the general procedure (Step C, method 2) described above. Purification of the residue by flash chromatography on silica gel (gradient elution, 070% EtOAc in hexanes) afforded AP-I-25 isomer-I and isomer-II as an inseparable mixture (75%).

Isomer-I/II: IR (cm⁻¹) 2951, 2199, 1748, 1732, 1622, 1520, 1487, 1470, 1455; [α]_{D}²⁰−20.90 (c 1.014 CHCl₃); ¹H NMR (400 MHz, CDCl₃) δ 7.48 (m, 1H), 7.39 (s, 1H), 6.92 (m, 3H), 6.83 (s, 1H), 4.63-4.52 (m, 6H), 2.66 (q, J=7.2 Hz, 4H), 2.33 (s, 6H), 1.85 (m, 2H), 1.56-1.42 (m, 6H), 1.31 (t, J=7.2 Hz, 6H), 1.31 (m, 2H), 1.14 (m, 2H), 0.89 (m, 2H), 0.83 (m, 2H), 0.77 (d, J=6.7 Hz, 6H), 0.72 (m, 2H), 0.67 (d, J=6.8 Hz, 6H), 0.54 (d, J=6.8 Hz, 6H); ¹³C NMR (100 MHz, CDCl₃) δ 167.0, 166.9, 155.9, 155.4, 142.6, 140.4, 135.4, 133.3, 132.0, 131.4, 123.5, 123.4, 119.0, 118.6, 108.5, 108.0, 76.1 (2C), 46.6 (2C), 44.85, 44.79, 40.4 (2C), 33.8 (2C), 31.2 (2C), 26.0 (2C), 23.02, 22.97, 21.8 (2C), 21.6, 21.4, 20.5, 20.43 (2C), 20.39, 15.90, 15.85, 11.46, 11.39; ES-API MS: m/z calcd for C₂₂H₃₂N₂O₂, found 357.3 [M+H].

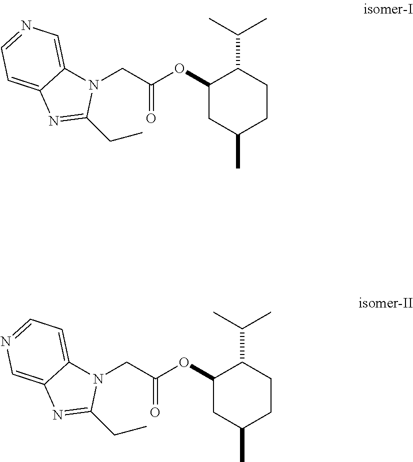

The title compounds were obtained following the general procedure (Step C, method 2) described above. Purification of the residue by flash chromatography on silica gel (gradient elution, 05% MeOH in CH₂Cl₂) afforded AP-I-13 isomer-I (37%) and isomer-II (33%) as white solids.

Isomer-I: IR (cm⁻¹) 2956, 2871, 2361, 1741, 1608, 1582, 1506, 1468; [α]_{D}²⁰−28.32 (c 0.96, CHCl₃); ¹H NMR (400 MHz, CDCl₃) δ 8.61 (m, 1H), 8.40 (m, 1H), 7.61 (m, 1H), 4.84 (m, 2H), 4.71 (m, 1H), 2.85 (m, 2H), 1.94 (m, 1H), 1.62 (m, 2H), 1.52 (m, 1H), 1.47 (m, 3H), 1.40 (m, 1H), 1.25 (m, 1H), 1.02-0.90 (m, 2H), 0.86 (m, 3H), 0.82 (m, 1H), 0.76 (m, 3H), 0.64 (m, 3H); ¹³C NMR (100 MHz, CDCl₃) δ 166.5, 159.6, 147.9, 142.5, 133.3, 132.1, 114.3, 77.1, 46.9, 45.4, 40.7, 34.1, 31.5, 26.5, 23.3, 22.1, 20.8, 20.7, 16.2, 11.5; ES-API MS: m/z calcd for C₂₀H₂₉N₃O₂, found 344.2 [M+H].

Isomer-II: IR (cm⁻¹) 2956, 2871, 1742, 1611, 1473, 1219, 962; [α]_{D}²⁰−35.50 (c 0.93, CHCl₃); ¹H NMR (400 MHz, CDCl₃) δ 8.99 (s, 1H), 8.35 (d, J=5.6 Hz, 1H), 7.11 (dd, J=5.6, 0.8 Hz, 1H), 4.76 (s, 2H), 4.68 (ddd, J=10.8, 10.8, 4.4 Hz, 1H), 2.82 (q, J=7.6 Hz, 2H), 1.91 (m, 1H), 1.65-1.56 (m, 2H), 1.50 (m, 1H), 1.44 (t, J=7.6 Hz, 3H), 1.40 (m, 1H), 1.22 (m, 1H), 0.95 (m, 1H), 0.90 (m, 1H), 0.84 (d, J=6.4 Hz, 3H), 0.80 (m, 1H), 0.75 (d, J=6.8 Hz, 3H), 0.62 (d, J=6.8 Hz, 3H); ¹³C NMR (100 MHz, CDCl₃) δ 166.5, 157.8, 142.2, 142.1, 140.5, 139.8, 104.4, 77.0, 46.9, 45.2, 40.7, 34.0, 31.5, 26.4, 23.3, 22.0, 20.8, 20.7, 16.2, 11.3; ES-API MS: m/z calcd for C₂₀H₂₉N₃O₂, found 344.2 [M+H].

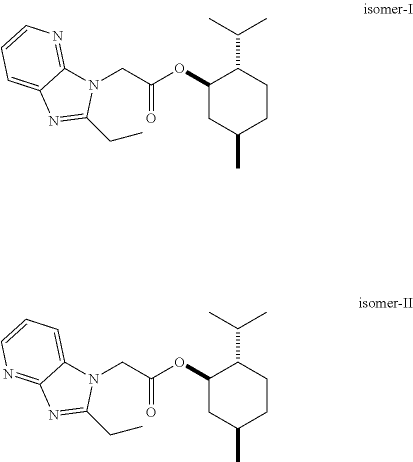

The title compounds were obtained following the general procedure (Step C, method 2) described above. Purification of the residue by flash chromatography on silica gel (gradient elution, 080% EtOAc in hexanes) afforded AP-I-15 isomer-I (37%) and isomer-II (33%) as orange gels.

Isomer-I: IR (cm⁻¹) 2956, 2870, 1744, 1604, 1514, 1450, 1218; [α]D²⁰−32.85 (c 0.980 CHCl₃); ¹H NMR (400 MHz, CDCl₃) δ 8.26 (dd, J=4.9, 1.4 Hz, 1H), 7.96 (dd, J=7.9, 1.4 Hz, 1H), 7.17 (dd, J=7.9, 4.8 Hz, 1H), 5.01 (d, J=18.0 Hz, 1H), 4.93 (d, J=18.0 Hz, 1H), 4.71 (ddd, J=10.8, 10.8, 4.4 Hz, 1H), 2.82 (q, J=7.6 Hz, 2H), 1.97 (m, 1H), 1.67-1.59 (m, 3H), 1.46 (t, J=7.6 Hz, 3H), 1.42 (m, 1H), 1.25 (m, 1H), 0.98 (m, 1H), 0.93 (m, 1H), 0.86 (d, J=6.8 Hz, 3H), 0.82 (m, 1H), 0.78 (d, J=6.8 Hz, 3H), 0.67 (d, J=6.8 Hz, 3H); ¹³C NMR (100 MHz, CDCl₃) δ 167.3, 157.6, 148.3, 143.3, 134.8, 126.7, 118.5, 76.5, 47.0, 43.4, 40.8, 34.2, 31.5, 26.3, 23.4, 22.1, 21.3, 20.8, 16.3, 11.1; ES-API MS: m/z calcd for C₂₀H₂₉N₃O₂, found 344.2 [M+H].

Isomer-II: IR (cm⁻¹) 2956, 2870, 1744, 1604, 1514, 1450, 1218; [α]D²⁰−32.00 (c 0.95, CHCl₃); ¹H NMR (400 MHz, CDCl₃) δ 8.35 (dd, J=4.8, 1.2 Hz, 1H), 7.39 (dd, J=8.0, 1.6 Hz, 1H), 7.01 (dd, J=8.0, 4.8 Hz, 1H), 4.72 (s, 2H), 4.60 (ddd, J=10.8, 10.8, 4.4 Hz, 1H), 2.75 (q, J=7.6 Hz, 2H), 1.82 (m, 1H), 1.57-1.48 (m, 2H), 1.45 (m, 1H), 1.35 (t, J=7.6 Hz, 3H), 1.31 (m, 1H), 1.15 (m, 1H), 0.88 (m, 1H), 0.82 (m, 1H), 0.77 (d, J=6.8 Hz, 3H), 0.72 (m, 1H), 0.78 (d, J=6.8 Hz, 3H), 0.55 (d, J=6.8 Hz, 3H); ¹³C NMR (100 MHz, CDCl₃) δ 166.6, 158.8, 155.6, 144.3, 127.5, 117.4, 116.5, 76.5, 46.7, 45.0, 40.5, 33.8, 31.3, 26.1, 23.1, 21.9, 20.8, 20.5, 16.0, 11.4; ES-API MS: m/z calcd for C₂₀H₂₉N₃O₂, found 344.2 [M+H].

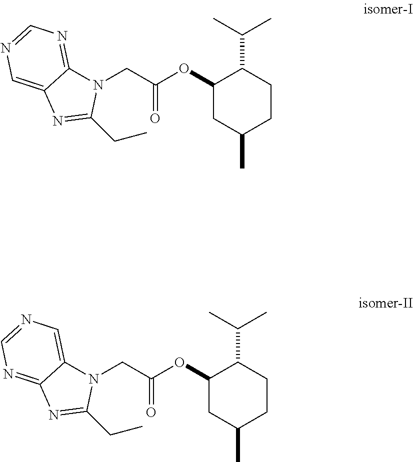

The title compounds were obtained following the general procedure (Step C, method 2) described above. Purification of the residue by flash chromatography on silica gel (gradient elution, 05% MeOH in CH₂Cl₂) afforded AP-I-26 isomer-I (36%) and isomer-II (18%) as orange and red gels, respectively.

Isomer-I: IR (cm⁻¹) 2950, 2353, 2218, 1748, 1732, 1715, 1634, 1600, 1516, 1506, 1456; [α]_{D}²⁰−29.56 (c 0.981 CHCl₃); ¹H NMR (400 MHz, CDCl₃) δ 8.90 (s, 1H), 8.76 (s, 1H), 4.89 (d, J=17.6 Hz, 1H), 4.83 (d, J=18.0 Hz, 1H), 4.62 (ddd, J=10.8, 10.8, 4.4 Hz, 1H), 2.74 (q, J=7.5 Hz, 2H), 1.86 (m, 1H), 1.60-1.50 (m, 3H), 1.36 (t, J=7.5 Hz, 3H), 1.32 (m, 1H), 1.18 (m, 1H), 0.87 (m, 2H), 0.76 (d, J=6.4 Hz, 3H), 0.73 (m, 1H), 0.71 (d, J=6.8 Hz, 3H), 0.58 (d, J=7.2 Hz, 3H); ¹³C NMR (100 MHz, CDCl₃) δ 166.4, 159.0, 152.8, 152.0, 146.6, 133.3, 76.7, 46.8, 43.1, 40.6, 33.9, 31.3, 26.2, 23.2, 21.9, 21.0, 20.6, 16.1, 10.6; ES-API MS: m/z calcd for C₁₉H₂₈N₄O₂, found 345.2 [M+H].

Isomer-II: IR (cm⁻¹) 2951, 2872, 2356, 1732, 1682, 1652, 1614, 1568, 1558, 1505, 1455; [α]_{D}²⁰−26.47(c 1.005 CHCl₃); ¹H NMR (400 MHz, CDCl₃) δ 9.05 (s, 1H), 8.68 (s, 1H), 4.88 (s, 2H), 4.73 (ddd, J=10.8, 10.8, 4.4 Hz, 1H), 2.88 (q, J=7.5 Hz, 2H), 1.92 (m, 1H), 1.66-1.59 (m, 2H), 1.55 (m, 1H), 1.46 (t, J=7.5 Hz, 3H), 1.42 (m, 1H), 1.26 (m, 1H), 1.03-0.88 (m, 2H), 0.86 (d, J=6.8 Hz, 3H), 0.82 (m, 1H), 0.78 (d, J=7.2 Hz, 3H), 0.65 (d, J=6.8 Hz, 3H); ¹³C NMR (100 MHz, CDCl₃) δ 166.0, 162.6, 160.4, 153.5, 138.1, 126.9, 77.4, 46.9, 45.5, 40.7, 34.0, 31.5, 26.6, 23.3, 22.0, 21.1, 20.7, 16.2, 11.3; ES-API MS: m/z calcd for C₁₉H₂₈N₄O₂, found 345.2 [M+H].

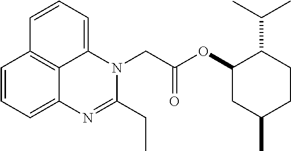

The title compounds were obtained following the general procedure (Step C, method 2) described above. Purification of the residue by flash chromatography on silica gel (gradient elution, 05% MeOH in CH₂Cl₂) afforded AP-I-24 (68%) as a green solid. IR (cm⁻¹) 3055, 2953, 1756, 1748, 1732, 1634, 1372, 1206, 911; [α]_{D}²⁰+9.07 (c 1.08 CHCl₃); ¹H NMR (400 MHz, CDCl₃) δ 7.26 (dd, J=8.0, 7.6 Hz, 1H), 7.16 (dd, J=8.4, 0.8 Hz, 1H), 7.12 (dd, J=8.0, 1.2 Hz, 1H), 7.08 (dd, J=7.2, 8.4 Hz, 1H), 6.87 (dd, J=7.2, 1.2 Hz, 1H), 4.72 (ddd, J=11.2, 10.8, 4.4 Hz, 1H), 4.30 (br, 2H), 2.49 (q, J=7.4 Hz, 2H), 1.97 (m, 1H), 1.65-1.59 (m, 3H), 1.44 (m, 1H), 1.32 (m, 1H), 1.29 (t, J=7.4 Hz, 3H), 1.03-0.90 (m, 2H), 0.87 (d, J=6.5 Hz, 3H), 0.81 (m, 2H), 0.73 (br, 3H), 0.63 (d, J=6.8 Hz, 3H); ¹³C NMR (100 MHz, CDCl₃) δ 168.2, 157.5, 142.6, 139.3, 135.2, 128.9, 127.3, 121.9, 120.4, 119.6, 115.4, 100.8, 76.6, 48.1, 46.9, 40.7, 34.1, 31.5, 28.4, 26.2, 23.2, 22.1, 20.8, 16.1, 11.4; ES-API MS: m/z calcd for C₂₅H₃₂N₂O₂, found 393.3 [M+H].

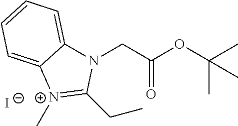 [SMILES: CCc1n(CC(=O)OC(C)(C)C)c2ccccc2[n+]1C ; [I-]]

The title compounds were obtained following the general procedure (Step F) described above. Purification of the residue by prep-TLC (8% MeOH in CH₂Cl₂) afforded the entitled salt Lqr-5-41 as a yellow gel (85%). IR (cm⁻¹) 2979, 1737, 1524, 1475, 1370, 1248, 1156, 758; ¹H NMR (400 MHz, CDCl₃) δ 7.74-7.67 (m, 1H), 7.64-7.51 (m, 3H), 5.34 (s, 2H), 4.13 (s, 3H), 3.55 (q, J=8.0 Hz, 2H), 1.49 (s, 9H), 1.39 (t, J=8.0 Hz, 3H); ¹³C NMR (100 MHz, CDCl₃) δ 164.9, 155.9, 131.5, 131.3, 127.2, 127.0, 112.6, 112.3, 85.1, 48.5, 33.1, 27.9 (3C), 20.4, 11.3; ES-API MS: m/z calcd for C₁₆H₂₃IN₂O₂, found 275.2 [M−I].

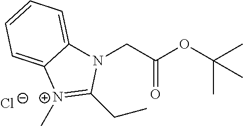 [SMILES: CCc1n(CC(=O)OC(C)(C)C)c2ccccc2[n+]1C ; [Cl-]]

The title compounds were obtained following the general procedure (Step G) described above. Purification of the residue by prep-TLC (8% MeOH in CH₂Cl₂) afforded the entitled salt Lqr-5-48 as a yellow gel (90%). IR (cm⁻¹) 2979, 1739, 1527, 1485, 1390, 1258, 1156, 760; ¹H NMR (400 MHz, CD₃OD) δ 7.99-7.89 (m, 1H), 7.85-7.77 (m, 1H), 7.77-7.60 (m, 2H), 5.43 (s, 2H), 4.16 (s, 3H), 3.31-3.27 (m, 2H), 1.50 (s, 9H), 1.39 (t, J=7.6 Hz, 3H); ¹³C NMR (100 MHz, CD₃OD) δ 165.5, 155.5, 131.7, 131.3, 126.7, 126.6, 112.5, 112.1, 84.1, 46.2, 30.9, 26.6 (3C), 16.9, 9.6; ES-API MS: m/z calcd for C₁₆H₂₃ClN₂O₂, found 275.2 [M−I].

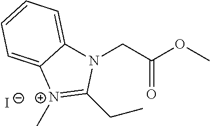 [SMILES: CCc1n(CC(=O)OC)c2ccccc2[n+]1C ; [I-]]

The title compounds were obtained following the general procedure (Step F) described above. Purification of the residue by prep-TLC (8% MeOH in CH₂Cl₂) afforded the entitled salt Lqr-4-167 as a yellow gel (90%). IR (cm⁻¹) 2889, 1746, 1522, 1455, 1223, 768; ¹H NMR (400 MHz, DMSO-d6) δ 8.04-8.00 (m, 1H), 7.98-7.94 (m, 1H), 7.70-7.60 (m, 2H), 5.65 (s, 2H), 4.08 (s, 3H), 3.76 (s, 3H), 3.38-3.25 (m, 2H), 1.22 (t, J=7.6 Hz, 3H); ¹³C NMR (100 MHz, DMSO-d6) δ 167.7, 156.2, 131.7, 131.4, 126.9, 126.8, 113.6, 113.4, 53.5, 46.2, 32.3, 17.2, 11.0; ES-API MS: m/z calcd for C₁₃H₁₇IN₂O₂, found 233.1 [M−I].

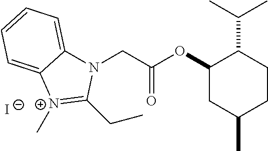

The title compounds were obtained following the general procedure (Step F) described above. Purification of the residue by prep-TLC (8% MeOH in CH₂Cl₂) afforded the entitled salt Lqr-5-045 as a yellow gel (85%). IR (cm⁻¹) 2926, 1739, 1634, 1539, 1472, 1370, 1220; [α]_{D}²⁰−27.997 (c 0.20, CHCl₃); ¹H NMR (400 MHz, CDCl₃) δ 7.73-7.67 (m, 1H), 7.68-7.56 (m, 2H), 7.56-7.47 (m, 1H), 5.40 (s, 2H), 4.79 (td, J=10.8, 4.4 Hz, 1H), 4.13 (s, 3H), 3.62-3.49 (m, 2H), 2.02-1.85 (m, 1H), 1.83-1.53 (m, 3H), 1.39 (t, J=7.6 Hz, 3H), 1.52-1.21 (m, 2H), 1.15-0.95 (m, 3H), 0.91 (d, J=6.8 Hz, 3H), 0.90 (d, J=7.2 Hz, 3H), 0.72 (d, J=6.8 Hz, 3H); ¹³C NMR (100 MHz, CDCl₃) δ 165.7, 156.2, 148.1, 131.8, 127.5, 127.3, 112.8, 112.4, 78.1, 48.3, 40.8, 34.0, 34.0, 33.2, 31.6, 30.5, 26.6, 23.3, 22.1, 20.9, 16.3, 11.5; ES-API MS: m/z calcd for C₂₂H₃₃IN₂O₂, found 357.2 [M−I].

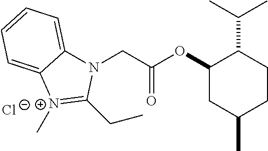

The title compounds were obtained following the general procedure (Step G) described above. Purification of the residue by prep-TLC (8% MeOH in CH₂Cl₂) afforded the entitled salt Lqr-5-049 as a yellow gel (90%). IR (cm⁻¹) 2925, 1729, 1634, 1539, 1472, 1370, 1220, 846, 745; [α]_{D}²⁰−25.997 (c 0.20, CHCl₃); ¹H NMR (400 MHz, CDCl₃) δ 7.71-7.65 (m, 1H), 7.65-7.57 (m, 2H), 7.55-7.49 (m, 1H), 5.62 (d, J=18.8 Hz, 1H), 5.53 (d, J=18.4 Hz, 1H), 4.79 (dt, J=11.2, 4.4 Hz, 1H), 4.14 (s, 3H), 3.77-3.55 (m, 2H), 2.03-1.87 (m, 1H), 1.86-1.52 (m, 3H), 1.52-1.22 (m, 2H), 1.39 (t, J=7.6 Hz, 3H), 1.18-0.97 (m, 3H), 0.91 (d, J=6.8 Hz, 3H), 0.90 (d, J=7.2 Hz, 3H), 0.72 (d, J=6.8 Hz, 3H); ¹³C NMR (100 MHz, CDCl₃) δ 166.0, 156.5, 131.5, 131.3, 127.1, 126.9, 112.3, 112.1, 77.7, 47.4, 46.7, 40.5, 33.8, 32.3, 31.4, 26.3, 23.1, 21.8, 20.7, 19.3, 16.0, 11.0; ES-API MS: m/z calcd for C₂₂H₃₃ClN₂O₂, found 357.2 [M−I].

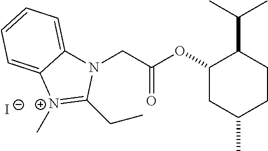

The title compounds were obtained following the general procedure (Step F) described above. Purification of the residue by prep-TLC (8% MeOH in CH₂Cl₂) afforded the entitled salt Lqr-5-046 as a yellow gel (89%). IR (cm⁻¹) 2926, 1739, 1634, 1539, 1472, 1370, 1220; [α]_{D}²⁰+36.659 (c 0.25, CHCl₃); ¹H NMR (400 MHz, CDCl₃) δ 7.75-7.68 (m, 1H), 7.66-7.57 (m, 2H), 7.55-7.49 (m, 1H), 5.41 (s, 2H), 4.81 (td, J=11.2, 4.4 Hz, 1H), 4.14 (s, 3H), 3.65-3.49 (m, 2H), 2.04-1.85 (m, 1H), 1.85-1.53 (m, 3H), 1.53-1.36 (m, 1H), 1.40 (t, J=8.0 Hz, 3H), 1.36-1.19 (m, 1H), 1.20-0.97 (m, 3H), 0.92 (d, J=4.2 Hz, 3H), 0.89 (d, J=6.8 Hz, 3H), 0.72 (d, J=6.8 Hz, 3H); ¹³C NMR (100 MHz, CDCl₃) δ 165.5, 156.0, 131.5, 131.3, 127.2, 127.1, 112.6, 112.1, 77.8, 46.7, 40.5, 33.9, 33.8, 33.1, 31.4, 30.2, 26.4, 23.1, 21.8, 20.7, 16.0, 11.3; ES-API MS: m/z calcd for C₂₂H₃₃IN₂O₂, found 357.2 [M−I].

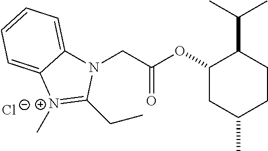

The title compounds were obtained following the general procedure (Step G) described above. Purification of the residue by prep-TLC (8% MeOH in CH₂Cl₂) afforded the entitled salt Lqr-5-050 as a yellow gel (95%). IR (cm⁻¹) 2926, 1739, 1634, 1539, 1472, 1370, 1220; [α]_{D}²⁰+27.996 (c 0.20, CHCl₃); ¹H NMR (400 MHz, CDCl₃) δ 7.72-7.65 (m, 1H), 7.64-7.56 (m, 2H), 7.54-7.47 (m, 1H), 5.63 (d, J=18.8 Hz, 1H), 5.53 (d, J=18.4 Hz, 1H), 4.79 (td, J=10.8, 4.4 Hz, 1H), 4.14 (s, 3H), 3.81-3.58 (m, 2H), 2.02-1.86 (m, 1H), 1.85-1.63 (m, 3H), 1.51-1.21 (m, 2H), 1.38 (t, J=7.6 Hz, 3H), 1.18-0.82 (m, 3H), 0.92 (d, J=6.8 Hz, 3H), 0.90 (d, J=6.8 Hz, 3H), 0.72 (d, J=6.8 Hz, 3H); ¹³C NMR (100 MHz, CDCl₃) δ 166.0, 156.6, 131.5, 131.3, 127.0, 126.9, 112.4, 112.1, 77.6, 46.7, 40.5, 33.9, 33.8, 32.4, 31.4, 26.3, 23.1, 21.8, 20.7, 19.3, 16.0, 11.0; ES-API MS: m/z calcd for C₂₂H₃₃ClN₂O₂, found 357.2 [M−I].

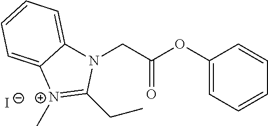 [SMILES: CCc1n(CC(=O)Oc2ccccc2)c2ccccc2[n+]1C ; [I-]]

The title compounds were obtained following the general procedure (Step F) described above. Purification of the residue by prep-TLC (8% MeOH in CH₂Cl₂) afforded the entitled salt Lqr-5-057 as a yellow gel (67%). IR (cm⁻¹) 2927, 2361, 1765, 1625, 1484, 1191, 752; ¹H NMR (400 MHz, CDCl₃) δ 7.79-7.71 (m, 1H), 7.71-7.64 (m, 3H), 7.44-7.37 (m, 2H), 7.31-7.27 (m, 1H), 7.25-7.16 (m, 2H), 5.89 (s, 2H), 4.13 (s, 3H), 3.69 (q, J=7.6 Hz, 2H), 1.45 (t, J=7.6 Hz, 3H); ¹³C NMR (100 MHz, CDCl₃) δ 164.8, 151.3, 145.8, 135.7, 131.5, 129.7 (2C), 127.5, 127.2, 126.8, 125.5 (2C), 121.0, 112.4, 48.3, 34.2, 30.3, 11.5; ES-API MS: m/z calcd for C₁₈H₁₉IN₂O₂, found 295.2 [M−I].

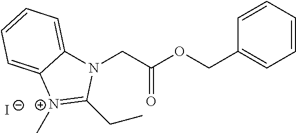 [SMILES: CCc1n(CC(=O)OCc2ccccc2)c2ccccc2[n+]1C ; [I-]]

The title compounds were obtained following the general procedure (Step F) described above. Purification of the residue by prep-TLC (8% MeOH in CH₂Cl₂) afforded the entitled salt Lqr-5-058 as a yellow gel (70%). IR (cm⁻¹) 2918, 1747, 1538, 1454, 1214, 751; ¹H NMR (400 MHz, CDCl₃) δ 7.69-7.56 (m, 3H), 7.55-7.49 (m, 1H), 7.43-7.29 (m, 5H), 5.49 (s, 2H), 5.28 (s, 2H), 4.09 (s, 3H), 3.54 (q, J=8.0 Hz, 2H), 1.36 (t, J=8.0 Hz, 3H); ES-API MS: m/z calcd for C₁₉H₂₁IN₂O₂, found 309.1 [M−I].

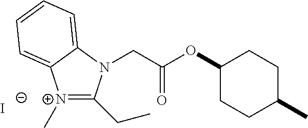

The title compounds were obtained following the general procedure (Step F) described above. Purification of the residue by prep-TLC (8% MeOH in CH₂Cl₂) afforded the entitled salt Lqr-5-059 as a yellow gel (75%). IR (cm⁻¹) 2926, 1740, 1538, 1446, 1360, 1219, 756; ¹H NMR (400 MHz, CDCl₃) δ 7.72-7.67 (m, 1H), 7.67-7.60 (m, 2H), 7.59-7.54 (m, 1H), 5.44 (s, 2H), 5.17-5.05 (m, 1H), 4.14 (s, 3H), 3.58 (q, J=8.0 Hz, 2H), 1.99-1.82 (m, 4H), 1.75-1.49 (m, 4H), 1.42 (t, J=8.0 Hz, 3H), 1.16-1.01 (m, 1H), 0.92 (d, J=6.0 Hz, 3H); ES-API MS: m/z calcd for C₁₉H₂₇IN₂O₂, found 315.2 [M−I].

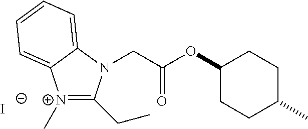

The title compounds were obtained following the general procedure (Step F) described above. Purification of the residue by prep-TLC (8% MeOH in CH₂Cl₂) afforded the entitled salt Lqr-5-060 as a yellow gel (75%). IR (cm⁻¹) 2927, 1742, 1538, 1383, 1214, 730; ¹H NMR (400 MHz, CDCl₃) δ 7.73-7.67 (m, 1H), 7.67-7.58 (m, 2H), 7.58-7.53 (m, 1H), 5.42 (s, 2H), 4.86-4.71 (m, 1H), 4.14 (s, 3H), 3.58 (q, J=7.6 Hz, 2H), 2.04-1.95 (m, 2H), 1.88-1.82 (m, 2H), 1.81-1.72 (m, 2H), 1.50-1.34 (m, 5H), 1.09-0.95 (m, 1H), 0.89 (d, J=6.4 Hz, 3H); ¹³C NMR (100 MHz, CDCl₃) δ 165.4, 156.0, 135.7, 131.2, 127.3, 127.14, 125.5, 112.5, 67.9, 48.1, 32.9, 32.7 (2C), 31.4, 30.3 (2C), 25.6, 21.6, 11.3; ES-API MS: m/z calcd for C₁₉H₂₇IN₂O₂, found 315.2 [M−I].

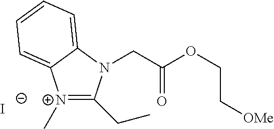

The title compounds were obtained following the general procedure (Step F) described above. Purification of the residue by prep-TLC (8% MeOH in CH₂Cl₂) afforded the entitled salt Lqr-5-064 as a yellow gel (78%). IR (cm⁻¹) 2937, 2361, 1800, 1522, 1500, 1278, 1127, 759; ¹H NMR (400 MHz, CDCl₃) δ 7.74-7.67 (m, 1H), 7.66-7.57 (m, 3H), 5.54 (s, 2H), 4.45-4.36 (m, 2H), 4.14 (s, 3H), 3.70-3.64 (m, 2H), 3.60 (q, J=7.6 Hz, 2H), 3.38 (s, 3H), 1.42 (t, J=7.6 Hz, 3H); ¹³C NMR (100 MHz, CDCl₃) δ 166.0, 156.2, 131.5, 131.2, 127.3, 127.1, 112.5, 112.4, 69.8, 65.6, 59.0, 47.9, 33.0, 20.5, 11.4; ES-API MS: m/z calcd for C₁₅H₂₁IN₂O₃, found 277.1 [M−I].

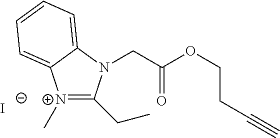

The title compounds were obtained following the general procedure (Step F) described above. Purification of the residue by prep-TLC (8% MeOH in CH₂Cl₂) afforded the entitled salt Lqr-5-065 as a yellow gel (80%). IR (cm⁻¹) 2938, 1746, 1524, 1474, 1207, 763; ¹H NMR (400 MHz, CDCl₃) δ 7.73-7.68 (m, 1H), 7.68-7.57 (m, 3H), 5.57 (s, 2H), 4.37 (t, J=6.8 Hz, 2H), 4.14 (s, 3H), 3.61 (q, J=7.6 Hz, 2H), 2.64 (td, J=6.8, 2.8 Hz, 2H), 2.02 (t, J=2.8 Hz, 1H), 1.42 (t, J=7.6 Hz, 3H); ¹³C NMR (100 MHz, CDCl₃) δ 165.8, 156.2, 131.5, 131.2, 127.3, 127.1, 112.5, 112.5, 79.1, 70.6, 64.2, 47.9, 33.0, 20.5, 18.9, 11.5; ES-API MS: m/z calcd for C₁₆H₁₉IN₂O₂, found 271.1 [M−I].

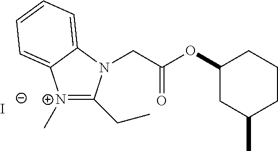

The title compounds were obtained following the general procedure (Step F) described above. Purification of the residue by flash chromatography on silica gel (gradient elution, 010% MeOH in CH₂Cl₂) afforded the entitled salt LW-V-263 as a white solid (58%). IR (cm⁻¹) 3456, 2931, 2865, 1741, 1475, 1224, 1211, 756; [α]_{D}²⁰+5.41 (c 0.48, CHCl₃); ¹H NMR (400 MHz, CDCl₃) δ 7.70 (m, 1H), 7.52-7.43 (m, 3H), 5.29 (s, 2H), 4.63 (m, 1H), 4.01 (s, 3H), 3.31 (q, J=7.6 Hz, 2H), 1.81 (m, 2H), 1.63 (m, 1H), 1.46 (m, 1H), 1.31 (m, 1H), 1.25 (t, J=7.6 Hz, 3H), 1.13 (m, 2H), 0.89 (m, 1H), 0.77 (d, J=6.8 Hz, 3H), 0.67 (m, 1H); ¹³C NMR (100 MHz, CDCl₃) δ 165.1, 155.2, 131.2, 130.9, 127.0, 126.9, 112.9, 112.2, 76.5, 49.7, 47.6, 39.7, 33.31, 33.25, 31.0, 23.5, 21.9, 19.1, 11.2; ES-API MS: m/z calcd for C₁₉H₂₇IN₂O₂, found 315.2 [M−I].

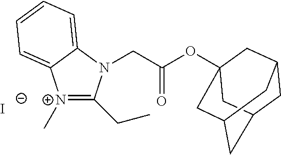

The title compounds were obtained following the general procedure (Step F) described above. Purification of the residue by flash chromatography on silica gel (gradient elution, 05% MeOH in CH₂Cl₂) afforded the entitled salt LW-V-268 as a white solid (92%). IR (cm⁻¹) 3459, 3036, 2915, 2853, 1738, 1472, 1224, 1051, 913, 729; ¹H NMR (400 MHz, CDCl₃) δ 7.72 (m, 1H), 7.56-7.47 (m, 3H), 5.23 (s, 2H), 4.05 (s, 3H), 3.36 (q, J=7.6 Hz, 2H), 2.05 (m, 3H), 1.99 (m, 6H), 1.52 (m, 6H), 1.29 (t, J=7.6 Hz, 3H); ¹³C NMR (100 MHz, CDCl₃) δ 164.3, 155.3, 131.3, 131.0, 127.1, 126.9, 112.9, 112.3, 85.0, 48.2, 41.0 (3C), 35.6 (3C), 33.4, 30.7 (3C), 19.4, 11.4; ES-API MS: m/z calcd for C₂₂H₂₉IN₂O₂, found 353.2 [M−I].

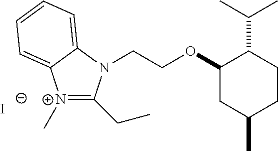

The title compounds were obtained following the general procedure (Step F) described above. Purification of the residue by flash chromatography on silica gel (gradient elution, 05% MeOH in CH₂Cl₂) afforded the entitled salt LW-V-266 as a yellow solid (93%). IR (cm⁻¹) 3445, 2954, 2930, 2871, 1526, 1470, 751; [α]_{D}²⁰−42.72 (c 0.44, CHCl₃); ¹H NMR (400 MHz, CDCl₃) δ 7.92 (m, 1H), 7.74 (m, 1H), 7.50 (m, 2H), 4.81 (ddd, J=15.2, 5.2, 3.2 Hz, 1H), 4.70 (ddd, J=14.8, 7.6, 3.2 Hz, 1H), 4.11 (s, 3H), 4.01 (ddd, J=8.8, 5.2, 3.6 Hz, 1H), 3.66 (ddd, J=10.8, 7.6, 3.2 Hz, 1H), 3.40 (q, J=7.6 Hz, 2H), 2.81 (ddd, J=10.4, 10.4, 4.4 Hz, 1H), 1.77 (m, 1H), 1.45 (m, 1H), 1.37 (t, J=7.6 Hz, 3H), 1.37 (m, 1H), 1.26 (m, 1H), 1.13 (m, 1H), 0.90 (m, 1H), 0.71 (d, J=6.4 Hz, 3H), 0.71 (m, 1H), 0.63 (m, 1H), 0.55 (d, J=7.2 Hz, 3H), 0.61 (m, 1H), 0.20 (d, J=7.2 Hz, 3H); ¹³C NMR (100 MHz, CDCl₃) δ 154.9, 131.4, 130.8, 126.9, 126.7, 113.9, 112.5, 79.8, 65.3, 47.6, 47.5, 39.8, 34.0, 35.6, 31.1, 25.4, 22.7, 22.0, 20.7, 19.0, 15.5, 11.9; ES-API MS: m/z calcd for C₂₂H₃₅IN₂O, found 343.2 [M−I].

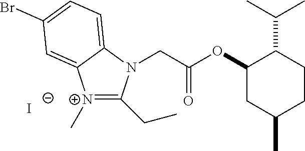

The title compounds were obtained following the general procedure (Step C) described above. Purification of the residue by prep-TLC (8% MeOH in CH₂Cl₂) afforded the entitled salt LW-IV-40 as a yellow gel (45%). IR (cm⁻¹) 3019, 2956, 2872, 1743, 1471, 1223, 752; [α]_{D}²⁰−20.61 (c 0.98, CHCl₃); ¹H NMR (400 MHz, CDCl₃) δ 7.90 (d, J=0.8 Hz, 1H), 7.68 (dd, J=8.4, 1.6 Hz, 1H), 7.49 (d, J=8.8 Hz, 1H), 5.45 (d, J=18.4 Hz, 1H), 5.39 (d, J=18.4 Hz, 1H), 4.79 (ddd, J=10.8, 10.8, 4.4 Hz, 1H), 4.11 (s, 3H), 3.52 (m, 2H), 1.97 (m, 1H), 1.79 (m, 1H), 1.71-1.64 (m, 3H), 1.44 (m, 1H), 1.37 (t, J=8.0 Hz, 3H), 1.10 (m, 1H), 1.02 (m, 1H), 0.91 (d, J=6.8 Hz, 3H), 0.90 (d, J=7.2 Hz, 3H), 0.86 (m, 1H), 0.72 (d, J=7.2 Hz, 3H); ¹³C NMR (100 MHz, CDCl₃) δ 165.6, 157.2, 132.8, 130.74, 130.65, 120.7, 116.1, 114.1, 78.2, 48.6, 46.9, 40.8, 34.0, 33.7, 31.6, 26.6, 23.3, 22.1, 21.0, 20.9, 16.3, 11.4; ES-API MS: m/z calcd for C₂₂H₃₂BrIN₂O₂, found 435.2 [M−I].

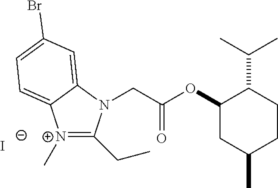

The title compounds were obtained following the general procedure (Step C) described above. Purification of the residue by prep-TLC (8% MeOH in CH₂Cl₂) afforded the entitled salt LW-IV-41 as a yellow gel (36%). IR (cm⁻¹) 3254, 2956, 2871, 1741, 1473, 1453, 1221, 737; [α]_{D}²⁰−17.61 (c 0.92, CHCl₃); ¹H NMR (400 MHz, CDCl₃) δ 7.73 (d, J=8.8 Hz, 1H), 7.67 (s, 1H), 7.66 (d, J=10.8 Hz, 1H), 5.32 (m, 2H), 4.83 (ddd, J=10.8, 10.8, 3.6 Hz, 1H), 4.14 (s, 3H), 3.52 (m, 2H), 2.00 (m, 1H), 1.80 (m, 1H), 1.72-1.62 (m, 3H), 1.46 (m, 1H), 1.40 (t, J=7.2 Hz, 3H), 1.17-1.00 (m, 2H), 0.93 (d, J=6.8 Hz, 6H), 0.89 (m, 1H), 0.75 (d, J=7.2 Hz, 3H); ¹³C NMR (100 MHz, CDCl₃) δ 165.5, 157.2, 132.5, 130.9, 130.7, 121.0, 115.6, 114.5, 78.3, 48.5, 47.0, 40.8, 34.0, 33.7, 31.7, 26.7, 23.3, 22.1, 21.2, 21.0, 16.2, 11.4; ES-API MS: m/z calcd for C₂₂H₃₂BrIN₂O₂, found 435.2 [M−I].

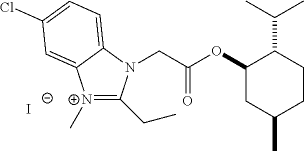

The title compounds were obtained following the general procedure (Step C) described above. Purification of the residue by prep-TLC (8% MeOH in CH₂Cl₂) afforded the entitled salt LW-IV-42 as a yellow gel (16%). IR (cm⁻¹) 3462, 2956, 2928, 2870, 1740, 1520, 1472, 1222; [α]_{D}²⁰−22.89 (c 0.76, CHCl₃); ¹H NMR (400 MHz, CDCl₃) δ 7.72 (d, J=1.2 Hz, 1H), 7.56 (dd, J=9.2, 1.6 Hz, 1H), 7.52 (d, J=8.4 Hz, 1H), 5.44 (d, J=18.4 Hz, 1H), 5.38 (d, J=18.4 Hz, 1H), 4.80 (ddd, J=11.2, 11.2, 4.4 Hz, 1H), 4.10 (s, 3H), 3.53 (m, 2H), 1.98 (m, 1H), 1.80 (m, 1H), 1.74-1.63 (m, 3H), 1.46 (m, 1H), 1.39 (t, J=7.6 Hz, 3H), 1.11 (m, 1H), 1.03 (m, 1H), 0.92 (d, J=6.4 Hz, 3H), 0.92 (d, J=7.2 Hz, 3H), 0.87 (m, 1H), 0.73 (d, J=6.8 Hz, 3H); ¹³C NMR (100 MHz, CDCl₃) δ 165.6, 157.5, 133.6, 132.5, 130.3, 128.1, 113.8, 113.1, 78.2, 48.6, 47.0, 40.8, 34.0, 33.6, 31.7, 26.6, 23.3, 22.1, 21.2, 20.9, 16.3, 11.4; ES-API MS: m/z calcd for C₂₂H₃₂ClIN₂O₂, found 391.2 [M−I].

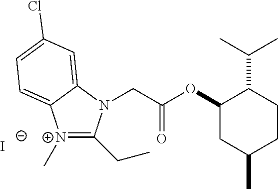

The title compounds were obtained following the general procedure (Step C) described above. Purification of the residue by prep-TLC (8% MeOH in CH₂Cl₂) afforded the entitled salt LW-IV-43 as a yellow gel (8%). IR (cm⁻¹) 3024, 2956, 2924, 1741, 1455, 1219, 1072, 755; [α]_{D}²⁰−17.27 (c 0.22, CHCl₃); ¹H NMR (400 MHz, CDCl₃) δ 7.68 (d, J=8.8 Hz, 1H), 7.59 (dd, J=8.8, 1.2 Hz, 1H), 7.51 (d, J=1.2 Hz, 1H), 5.32 (s, 2H), 4.83 (ddd, J=10.8, 10.8, 4.4 Hz, 1H), 4.13 (s, 3H), 3.53 (m, 2H), 2.00 (m, 1H), 1.80 (m, 1H), 1.74-1.62 (m, 3H), 1.47 (m, 1H), 1.40 (t, J=8.0 Hz, 3H), 1.13 (m, 1H), 1.05 (m, 1H), 0.94 (d, J=6.8 Hz, 6H), 0.86 (m, 1H), 0.75 (d, J=6.8 Hz, 3H); ¹³C NMR (100 MHz, CDCl₃) δ 165.5, 157.4, 133.8, 132.2, 130.5, 128.0, 114.2, 112.7, 78.3, 48.5, 47.0, 40.8, 34.0, 33.7, 31.7, 26.7, 23.3, 22.1, 21.3, 20.9, 16.2, 11.4; ES-API MS: m/z calcd for C₂₂H₃₂ClIN₂O₂, found 391.2 [M−I].

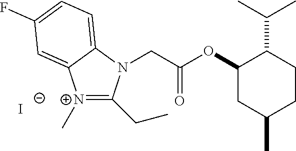

The title compounds were obtained following the general procedure (Step C) described above. Purification of the residue by prep-TLC (8% MeOH in CH₂Cl₂) afforded the entitled salt LW-IV-44 as a yellow gel (93%). IR (cm⁻¹) 3463, 2930, 2957, 2872, 1741, 1526, 1497, 1225, 1180, 755; [α]_{D}²⁰−27.04 (c 1.05, CHCl₃); ¹H NMR (400 MHz, CDCl₃) δ 7.61 (dd, J=9.2, 4.0 Hz, 1H), 7.54 (dd, J=7.6, 2.4 Hz, 1H), 7.28 (ddd, J=9.2, 8.8, 2.4 Hz, 1H), 5.46 (d, J=18.4 Hz, 1H), 5.39 (d, J=18.4 Hz, 1H), 4.74 (ddd, J=10.8, 10.8, 4.4 Hz, 1H), 4.11 (s, 3H), 3.47 (m, 2H), 1.93 (m, 1H), 1.73 (m, 1H), 1.67-1.62 (m, 2H), 1.46-1.36 (m, 2H), 1.34 (t, J=7.6 Hz, 3H), 1.05 (m, 1H), 0.97 (m, 1H), 0.86 (d, J=6.8 Hz, 3H), 0.84 (d, J=7.2 Hz, 3H), 0.82 (m, 1H), 0.66 (d, J=7.2 Hz, 3H); ¹³C NMR (100 MHz, CDCl₃) δ 165.4, 161.3 (d, J=247.1 Hz, 1C), 157.1 (d, J=1.7 Hz, 1C), 132.3 (d, J=12.7 Hz, 1C), 127.8 (d, J=1.1 Hz, 1C), 115.9 (d, J=25.9 Hz, 1C), 114.2 (d, J=9.9 Hz, 1C), 100.4 (d, J=28.3 Hz, 1C), 77.9, 48.6, 46.8, 40.6, 34.2, 33.9, 31.5, 26.4, 23.1, 21.9, 20.8, 20.5, 16.1, 11.5; ES-API MS: m/z calcd for C₂₂H₃₂FIN₂O₂, found 375.2 [M−I].

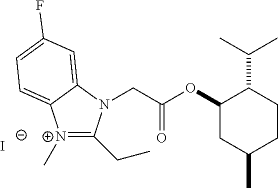

The title compounds were obtained following the general procedure (Step C) described above. Purification of the residue by prep-TLC (8% MeOH in CH₂Cl₂) afforded the entitled salt LW-IV-45 as a yellow gel (90%). IR (cm⁻¹) 3029, 2958, 2927, 2874, 1742, 1494, 1217, 1154, 758; [α]_{D}²⁰−28.37 (c 1.17, CHCl₃); ¹H NMR (400 MHz, CDCl₃) δ 7.87 (dd, J=9.2, 4.0 Hz, 1H), 7.32 (ddd, J=8.8, 8.8, 2.0 Hz, 1H), 7.27 (dd, J=7.6, 2.0 Hz, 1H), 5.39 (d, J=18.4 Hz, 1H), 5.33 (d, J=18.4 Hz, 1H), 4.76 (ddd, J=10.8, 10.8, 4.0 Hz, 1H), 4.16 (s, 3H), 3.48 (m, 2H), 1.96 (m, 1H), 1.75 (m, 1H), 1.69-1.65 (m, 2H), 1.47-1.37 (m, 2H), 1.35 (t, J=7.6 Hz, 3H), 1.07 (m, 1H), 1.00 (m, 1H), 0.89 (d, J=6.4 Hz, 3H), 0.87 (d, J=6.8 Hz, 3H), 0.84 (m, 1H), 0.69 (d, J=7.2 Hz, 3H); ¹³C NMR (100 MHz, CDCl₃) δ 165.4, 161.5 (d, J=247.6 Hz, 1C), 157.0 (d, J=1.9 Hz, 1C), 132.0 (d, J=12.7 Hz, 1C), 128.2 (d, J=0.7 Hz, 1C), 115.9 (d, J=25.7 Hz, 1C), 115.1 (d, J=10.0 Hz, 1C), 99.7 (d, J=28.5 Hz, 1C), 78.1, 48.6, 46.8, 40.7, 34.2, 33.9, 31.6, 26.5, 23.2, 22.0, 20.8, 20.6, 16.1, 11.5; ES-API MS: m/z calcd for C₂₂H₃₂FIN₂O₂, found 375.2 [M−I].

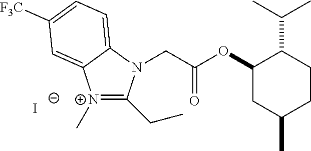

The title compounds were obtained following the general procedure (Step C) described above. Purification of the residue by prep-TLC (8% MeOH in CH₂Cl₂) afforded the entitled salt LW-IV-46 as a yellow gel (99%). IR (cm⁻¹) 3456, 3027, 2958, 2930, 2872, 1741, 1465, 1470, 1333, 1132, 756; [α]_{D}²⁰−21.68 (c 1.07, CHCl₃); ¹H NMR (400 MHz, CDCl₃) δ 8.02 (s, 1H), 7.81 (s, 2H), 5.53 (d, J=18.4 Hz, 1H), 5.46 (d, J=18.4 Hz, 1H), 4.76 (ddd, J=11.2, 10.8, 4.4 Hz, 1H), 4.19 (s, 3H), 3.54 (m, 2H), 1.96 (m, 1H), 1.76 (m, 1H), 1.71-1.61 (m, 2H), 1.47-1.39 (m, 2H), 1.36 (t, J=7.6 Hz, 3H), 1.08 (m, 1H), 0.99 (m, 1H), 0.89 (d, J=6.4 Hz, 3H), 0.87 (d, J=6.8 Hz, 3H), 0.84 (m, 1H), 0.69 (d, J=6.8 Hz, 3H); ¹³C NMR (100 MHz, CDCl₃) δ 165.4, 158.9, 133.5, 131.4, 129.3 (q, J=33.6 Hz, 1C), 124.2 (q, J=3.3 Hz, 1C), 123.4 (q, J=271.5 Hz, 1C), 114.0, 111.0 (d, J=4.1 Hz, 1C), 78.1, 48.8, 46.8, 40.7, 34.2, 33.9, 31.5, 26.5, 23.2, 22.0, 21.0, 20.8, 16.2, 11.4; ES-API MS: m/z calcd for C₂₃H₃₂F₃IN₂O₂, found 425.2 [M−I].

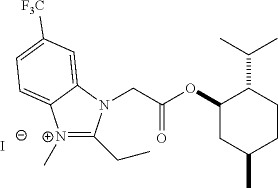

The title compounds were obtained following the general procedure (Step C) described above. Purification of the residue by prep-TLC (8% MeOH in CH₂Cl₂) afforded the entitled salt LW-IV-47 as a yellow gel (88%). IR (cm⁻¹) 3042, 2956, 1739, 1520, 1462, 1304, 1220, 1133, 1061, 736; [α]_{D}²⁰−22.47 (c 1.05, CHCl₃); ¹H NMR (400 MHz, CDCl₃) δ 8.05 (d, J=8.8 Hz, 1H), 7.85 (d, J=8.4 Hz, 1H), 7.78 (s, 1H), 5.46 (d, J=18.4 Hz, 1H), 5.37 (d, J=18.4 Hz, 1H), 4.81 (ddd, J=11.2, 10.8, 4.4 Hz, 1H), 4.22 (s, 3H), 3.56 (m, 2H), 1.98 (m, 1H), 1.76 (m, 1H), 1.71-1.64 (m, 2H), 1.49-1.43 (m, 2H), 1.38 (t, J=8.0 Hz, 3H), 1.09 (m, 1H), 1.01 (m, 1H), 0.90 (d, J=6.8 Hz, 3H), 0.89 (d, J=6.8 Hz, 3H), 0.86 (m, 1H), 0.70 (d, J=6.8 Hz, 3H); ¹³C NMR (100 MHz, CDCl₃) δ 165.3, 160.0, 133.8, 131.2, 129.7 (q, J=33.5 Hz, 1C), 124.2 (q, J=3.3 Hz, 1C), 123.4 (q, J=271.6 Hz, 1C), 114.7, 111.3 (d, J=4.3 Hz, 1C), 78.3, 48.7, 46.9, 40.7, 34.4, 34.0, 31.6, 26.7, 23.2, 22.0, 21.2, 20.8, 16.1, 11.5; ES-API MS: m/z calcd for C₂₃H₃₂F₃IN₂O₂, found 425.2 [M−I].

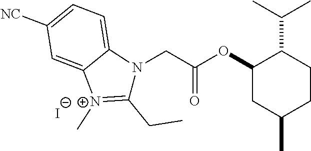 [SMILES: CCc1n(CC(=O)O[C@@H]2C[C@H](C)CC[C@H]2C(C)C)c2ccc(C#N)cc2[n+]1C ; [I-]]

The title compounds were obtained following the general procedure (Step C) described above. Purification of the residue by prep-TLC (8% MeOH in CH₂Cl₂) afforded the entitled salt LW-IV-48 as a yellow gel (99%). IR (cm⁻¹) 3455, 3019, 2957, 2929, 2871, 1741, 1224, 756; [α]_{D}²⁰−19.03 (c 2.08, CHCl₃); ¹H NMR (400 MHz, CDCl₃) δ 8.44 (s, 1H), 7.83 (d, J=8.4 Hz, 1H), 7.76 (dd, J=8.8, 0.8 Hz, 1H), 5.53 (d, J=18.4 Hz, 1H), 5.46 (d, J=18.4 Hz, 1H), 4.75 (ddd, J=10.8, 10.8, 4.4 Hz, 1H), 4.23 (s, 3H), 3.49 (m, 2H), 1.94 (m, 1H), 1.76 (m, 1H), 1.68-1.59 (m, 2H), 1.45-1.38 (m, 2H), 1.35 (t, J=8.0 Hz, 3H), 1.06 (m, 1H), 0.97 (m, 1H), 0.86 (d, J=6.0 Hz, 3H), 0.85 (d, J=6.8 Hz, 3H), 0.82 (m, 1H), 0.67 (d, J=7.2 Hz, 3H); ¹³C NMR (100 MHz, CDCl₃) δ 165.2, 159.1, 133.8, 131.6, 130.3, 118.8, 117.5, 114.4, 110.3, 78.1, 48.7, 46.7, 40.6, 34.8, 33.8, 31.5, 26.4, 23.1, 21.9, 20.8, 20.6, 16.1, 11.3; ES-API MS: m/z calcd for C₂₃H₃₂IN₃O₂, found 382.3 [M−I].

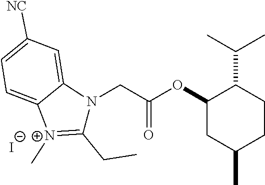 [SMILES: CCc1n(CC(=O)O[C@@H]2C[C@H](C)CC[C@H]2C(C)C)c2cc(C#N)ccc2[n+]1C ; [I-]]

The title compounds were obtained following the general procedure (Step C) described above. Purification of the residue by prep-TLC (8% MeOH in CH₂Cl₂) afforded the entitled salt LW-IV-49 as a yellow gel (81%). IR (cm⁻¹) 3023, 2955, 2870, 1739, 1454, 1223; [α]_{D}²⁰−28.17 (c 0.88, CHCl₃); ¹H NMR (400 MHz, CDCl₃) δ 7.98 (s, 1H), 7.91 (d, J=8.4 Hz, 1H), 7.83 (dd, J=8.4, 1.2 Hz, 1H), 5.37 (s, 2H), 4.76 (ddd, J=10.8, 10.8, 4.4 Hz, 1H), 4.10 (s, 3H), 3.37 (m, 2H), 1.93 (m, 1H), 1.75 (m, 1H), 1.69-1.60 (m, 2H), 1.46-1.36 (m, 2H), 1.33 (t, J=7.6 Hz, 3H), 1.06 (m, 1H), 0.98 (m, 1H), 0.87 (d, J=6.8 Hz, 6H), 0.83 (m, 1H), 0.68 (d, J=6.8 Hz, 3H); ¹³C NMR (100 MHz, CDCl₃) δ 165.5, 159.1, 134.2, 131.4, 130.3, 117.7, 117.4, 114.6, 110.8, 110.1, 78.4, 47.7, 46.9, 40.6, 33.9, 33.4, 31.5, 26.5, 23.1, 21.9, 20.7, 20.1, 16.0, 10.9; ES-API MS: m/z calcd for C₂₃H₃₂IN₃O₂, found 383.2 [M−I+H].

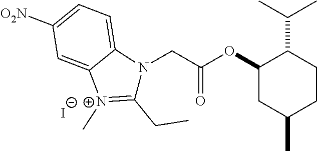 [SMILES: CCc1n(CC(=O)O[C@@H]2C[C@H](C)CC[C@H]2C(C)C)c2ccc([N+](=O)[O-])cc2[n+]1C ; [I-]]

The title compounds were obtained following the general procedure (Step C) described above. Purification of the residue by prep-TLC (8% MeOH in CH₂Cl₂) afforded the entitled salt LW-IV-50 as a yellow gel (98%). IR (cm⁻¹) 3453, 2957, 2872, 1739, 1538, 1513, 1347, 1223, 756; [α]_{D}²⁰−17.34 (c 1.96, CHCl₃); ¹H NMR (400 MHz, CDCl₃) δ 8.73 (d, J=2.0 Hz, 1H), 8.41 (dd, J=9.2, 2.0 Hz, 1H), 7.90 (d, J=9.2 Hz, 1H), 5.57 (d, J=18.4 Hz, 1H), 5.49 (d, J=18.4 Hz, 1H), 4.78 (ddd, J=10.8, 10.8, 4.4 Hz, 1H), 4.26 (s, 3H), 3.53 (m, 2H), 1.96 (m, 1H), 1.79 (m, 1H), 1.70-1.60 (m, 2H), 1.49-1.40 (m, 2H), 1.38 (t, J=8.0 Hz, 3H), 1.09 (m, 1H), 1.00 (m, 1H), 0.89 (d, J=6.8 Hz, 3H), 0.88 (d, J=6.8 Hz, 3H), 0.84 (m, 1H), 0.70 (d, J=6.8 Hz, 3H); ¹³C NMR (100 MHz, CDCl₃) δ 165.3, 160.5, 146.2, 135.1, 131.6, 122.4, 114.2, 110.2, 78.3, 49.0, 46.8, 40.7, 34.7, 33.9, 31.6, 26.5, 23.2, 22.0, 21.2, 20.9, 16.2, 11.3; ES-API MS: m/z calcd for C₂₂H₃₂IN₃O₄, found 402.2 [M−I].

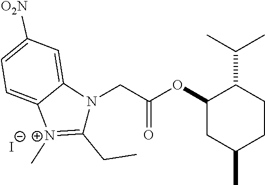 [SMILES: CCc1n(CC(=O)O[C@@H]2C[C@H](C)CC[C@H]2C(C)C)c2cc([N+](=O)[O-])ccc2[n+]1C ; [I-]]

The title compounds were obtained following the general procedure (Step C) described above. Purification of the residue by prep-TLC (8% MeOH in CH₂Cl₂) afforded the entitled salt LW-IV-51 as a yellow gel (65%). IR (cm⁻¹) 3454, 2957, 2928, 2872, 1740, 1534, 1347, 1223, 751; [α]_{D}²⁰−25.52 (c 0.76, CHCl₃); ¹H NMR (400 MHz, CDCl₃) δ 8.50 (dd, J=8.8, 2.0 Hz, 1H), 8.48 (d, J=1.6 Hz, 1H), 8.06 (d, J=9.2 Hz, 1H), 5.46 (d, J=18.4 Hz, 1H), 5.39 (d, J=18.4 Hz, 1H), 4.84 (ddd, J=10.8, 10.8, 4.4 Hz, 1H), 4.24 (s, 3H), 3.55 (m, 2H), 2.01 (m, 1H), 1.84 (m, 1H), 1.75-1.67 (m, 2H), 1.53-1.45 (m, 2H), 1.41 (t, J=7.6 Hz, 3H), 1.15 (m, 1H), 1.04 (m, 1H), 0.94 (d, J=6.8 Hz, 3H), 0.93 (d, J=6.8 Hz, 3H), 0.89 (m, 1H), 0.75 (d, J=7.2 Hz, 3H); ¹³C NMR (100 MHz, CDCl₃) δ 165.2, 160.9, 146.6, 135.4, 131.5, 122.5, 114.6, 109.5, 78.6, 48.8, 47.0, 40.8, 34.6, 34.0, 31.7, 26.8, 23.3, 22.1, 21.7, 20.9, 16.2, 11.4; ES-API MS: m/z calcd for C₂₂H₃₂IN₃O₄, found 402.2 [M−I].

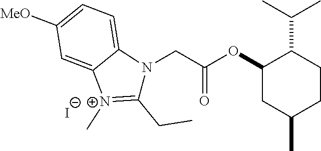 [SMILES: CCc1n(CC(=O)O[C@@H]2C[C@H](C)CC[C@H]2C(C)C)c2ccc(OC)cc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-32 as a yellow solid (71%). IR (cm⁻¹) 2958, 2872, 1743, 1627, 1524, 1500, 1456, 1266, 1217; [α]_{D}²⁰−22.979 (c 0.992 CHCl₃); ¹H NMR (400 MHz, CDCl₃) δ 7.39 (d, J=8.8 Hz, 1H), 7.28 (d, J=2.4 Hz, 1H), 7.12 (dd, J=9.2, 2.4 Hz, 1H), 5.37 (d, J=18.0 Hz, 1H), 5.31 (d, J=18.0 Hz, 1H), 4.75 (ddd, J=10.8, 10.8, 4.4 Hz, 1H), 4.14 (s, 3H), 3.93 (s, 3H), 3.45 (m, 2H), 1.95 (m, 1H), 1.74 (m, 1H), 1.66 (m, 2H), 1.43 (m, 2H), 1.35 (t, J=7.6 Hz, 3H), 1.06 (m, 1H), 0.98 (m, 1H), 0.89 (d, J=6.4 Hz, 3H), 0.86 (d, J=6.4 Hz, 3H), 0.84 (m, 1H), 0.68 (d, J=6.8 Hz, 3H); ¹³C NMR (100 MHz, CDCl₃) δ 165.6, 159.7, 154.7, 132.9, 125.4, 117.4, 113.0, 96.0, 77.9, 57.1, 48.2, 46.9, 40.7, 34.0, 31.6, 29.8, 26.5, 23.2, 22.0, 20.9, 20.2, 16.2, 11.6; ES-API MS: m/z calcd for C₂₃H₃₅IN₂O₃, found 387.3 [M−I].

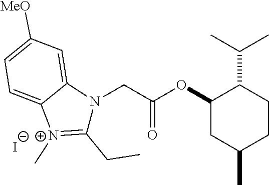 [SMILES: CCc1n(CC(=O)O[C@@H]2C[C@H](C)CC[C@H]2C(C)C)c2cc(OC)ccc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-31 as a white solid (61%). IR (cm⁻¹) 2950, 2352, 1739, 1694, 1982, 1652, 1626, 1496, 1455, 1372, 1254, 1216; [α]_{D}²⁰−22.904 (c 1.004 CHCl₃); ¹H NMR (400 MHz, CDCl₃) δ 7.63 (d, J=9.2 Hz, 1H), 7.14 (dd, J=9.2, 2.0 Hz, 1H), 7.02 (d, J=2.0 Hz, 1H), 5.48 (d, J=18.0 Hz, 1H), 5.38 (d, J=18.0 Hz, 1H), 4.77 (ddd, J=10.8, 10.8, 4.4 Hz, 1H), 3.10 (s, 3H), 3.87 (s, 3H), 3.45 (m, 2H), 1.98 (m, 1H), 1.76 (m, 1H), 1.66 (m, 2H), 1.42 (m, 2H), 1.35 (t, J=8.0 Hz, 3H), 1.08 (m, 1H), 1.00 (m, 1H), 0.89 (d, J=6.8 Hz, 3H), 0.86 (d, J=7.2 Hz, 3H), 0.84 (m, 1H), 0.69 (d, J=7.2 Hz, 3H); ¹³C NMR (100 MHz, CDCl₃) δ 165.7, 159.7, 154.7, 132.6, 125.8, 117.0, 113.8, 95.4, 77.8, 56.7, 48.4, 46.9, 40.8, 34.0, 33.7, 31.6, 26.5, 23.2, 22.0, 20.9, 20.2, 16.2, 11.7; ES-API MS: m/z calcd for C₂₃H₃₅IN₂O₃, found 387.3 [M−I].

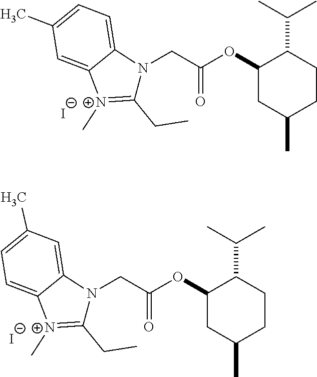 [SMILES: CCc1n(CC(=O)O[C@@H]2C[C@H](C)CC[C@H]2C(C)C)c2ccc(C)cc2[n+]1C ; [I-] ; CCc1n(CC(=O)O[C@@H]2C[C@H](C)CC[C@H]2C(C)C)c2cc(C)ccc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-37, an inseparable mixture, as a white-yellow solid (80%). IR (cm⁻¹) 2958, 2198, 1738, 1732, 1538, 1520, 1496, 1470, 1455, 1372, 1224; ¹H NMR (400 MHz, CDCl₃) δ 7.61 (d, J=8.4 Hz, 1H), 7.53 (s, 1H), 7.32 (d, J=8.4 Hz, 1H), 7.26 (d, J=9.6 Hz, 1H), 7.23 (d, J=9.6 Hz, 1H), 7.20 (s, 1H), 5.32 (dd, J=18.0, 3.6 Hz, 2H), 5.25 (dd, J=18.0, 8.0 Hz, 2H), 4.60 (m, 2H), 4.02 (s, 3H), 4.01 (s, 3H), 3.36 (m, 4H), 2.38 (s, 3H), 2.35 (s, 3H), 1.82 (m, 2H), 1.58 (m, 2H), 1.50 (m, 4H), 1.27 (m, 4H), 1.23 (t, J=8.0 Hz, 6H), 0.91 (m, 2H), 0.84 (m, 2H), 0.74 (d, J=6.8 Hz, 6H), 0.70 (d, J=6.8 Hz, 6H), 0.68 (m, 2H), 0.53 (d, J=7.2 Hz, 6H); ¹³C NMR (100 MHz, CDCl₃) δ 165.09, 165.07, 154.62, 154.58, 137.63, 137.58, 131.4, 131.0, 129.3, 129.0, 128.35, 128.28, 112.54, 112.49, 111.5 (2C), 77.26, 77.23, 47.9, 47.8, 46.32, 46.31, 40.2, 33.6, 33.5 (2C), 31.0 (2C), 25.90, 25.87, 22.70, 22.67, 21.54 (2C), 21.45, 21.40, 20.39, 20.37, 19.4, 15.68, 15.65, 11.31, 11.28; ES-API MS: m/z calcd for C₂₃H₃₅IN₂O₂, found 371.3 [M−I].

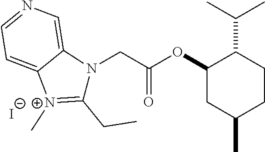 [SMILES: CCc1n(CC(=O)O[C@@H]2C[C@H](C)CC[C@H]2C(C)C)c2cnccc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-20 as a yellow solid (74%). IR (cm⁻¹) 2955, 2870, 1741, 1654, 1522, 1479, 1370, 1318, 1222; [α]_{D}²⁰−10.46 (c 0.994 CHCl₃); ¹H NMR (400 MHz, CDCl₃) δ 10.47 (s, 1H), 8.54 (dd, J=6.8, 0.8 Hz, 1H), 7.93 (d, J=6.8 Hz, 1H), 5.58 (d, J=18.4 Hz, 1H), 5.43 (d, J=18.4 Hz, 1H), 4.70 (ddd, J=10.8, 10.8, 4.4 Hz, 1H), 4.56 (s, 3H), 2.88 (qd, J=7.6, 3.2 Hz, 2H), 1.99 (m, 1H), 1.79 (m, 1H), 1.60 (m, 2H), 1.41 (t, J=7.6 Hz, 3H), 1.34 (m, 2H), 1.03 (m, 1H), 0.95 (m, 1H), 0.83 (d, J=6.8 Hz, 3H), 0.82 (d, J=6.8 Hz, 3H), 0.78 (m, 1H), 0.66 (d, J=6.8 Hz, 3H); ¹³C NMR (100 MHz, CDCl₃) δ 168.2, 166.1, 151.5, 136.0, 134.0, 132.0, 116.6, 77.6, 48.2, 47.7, 46.7, 40.6, 33.9, 31.4, 26.2, 23.1, 21.9, 21.4, 20.8, 16.2, 10.7; ES-API MS: m/z calcd for C₂₁H₃₂IN₃O₂, found 358.3 [M−I].

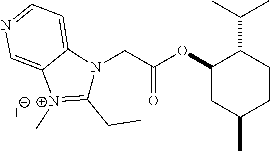 [SMILES: CCc1n(CC(=O)O[C@@H]2C[C@H](C)CC[C@H]2C(C)C)c2ccncc2[n+]1C ; [I-]]

The title compounds were obtained following the general procedure (Step

C) described above. Purification of the residue by flash chromatography on silica gel (gradient elution, 015% MeOH in CH₂Cl₂) afforded the entitled salt AP-I-21 as a yellow solid (72%). IR (cm⁻¹) 2955, 2870, 1740, 1647, 1534, 1494, 1458, 1372, 1309, 1222; [α]_{D}²⁰−24.04 (c 0.990 CHCl₃); ¹H NMR (400 MHz, CDCl₃) δ 9.22 (s, 1H), 8.86 (d, J=6.8 Hz, 1H), 8.22 (d, J=6.8 Hz, 1H), 5.37 (d, J=18.4 Hz, 1H), 5.28 (d, J=18.4 Hz, 1H), 4.70 (ddd, J=10.8, 10.8, 4.4 Hz, 1H), 4.60 (s, 3H), 2.90 (q, J=7.6 Hz, 2H), 1.94 (m, 1H), 1.73 (m, 1H), 1.62 (m, 2H), 1.39 (t, J=7.6 Hz, 3H), 1.35 (m, 2H), 1.05-0.92 (m, 2H), 0.85 (d, J=6.8 Hz, 3H), 0.83 (d, J=6.8 Hz, 3H), 0.80 (m, 1H), 0.66 (d, J=6.8 Hz, 3H); ¹³C NMR (100 MHz, CDCl₃) δ 166.0, 165.2, 145.0, 140.1, 138.0, 136.4, 109.8, 77.6, 49.0, 47.3, 46.7, 40.7, 33.9, 31.5, 26.3, 23.2, 21.9, 21.5, 20.8, 16.2, 10.6; ES-API MS: m/z calcd for C₂₁H₃₂IN₃O₂, found 358.3 [M−I].

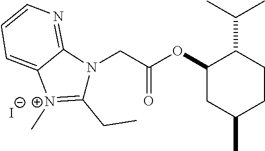 [SMILES: CCc1n(CC(=O)O[C@@H]2C[C@H](C)CC[C@H]2C(C)C)c2ncccc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-29 as a yellow solid (75%). IR (cm⁻¹) 2955, 2870, 1741, 1225; [α]_{D}²⁰−21.00 (c 0.95 CHCl₃); ¹H NMR (400 MHz, CDCl₃) δ 9.15 (d, J=6.0 Hz, 1H), 8.87 (d, J=8.0 Hz, 1H), 7.68 (dd, J=8.0, 6.8 Hz, 1H), 5.50 (d, J=18.4 Hz, 1H), 5.38 (d, J=18.4 Hz, 1H), 4.70 (ddd, J=11.2, 10.8, 4.4 Hz, 1H), 4.55 (s, 3H), 2.95 (q, J=7.2 Hz, 2H), 1.93 (m, 1H), 1.78 (m, 1H), 1.73 (m, 1H), 1.61 (m, 2H), 1.42 (t, J=7.2 Hz, 3H), 1.35 (m, 1H), 1.00 (m, 2H), 0.84 (d, J=6.8 Hz, 3H), 0.82 (d, J=6.8 Hz, 3H), 0.79 (m, 1H), 0.66 (d, J=6.8 Hz, 3H); ¹³C NMR (100 MHz, CDCl₃) δ 166.0, 165.2, 145.0, 140.1, 138.0, 136.4, 109.8, 77.6, 49.0, 47.3, 46.7, 40.7, 33.9, 31.5, 26.3, 23.2, 21.9, 21.5, 20.8, 16.2, 10.6; ES-API MS: m/z calcd for C₂₁H₃₂IN₃O₂, found 358.3 [M−I].

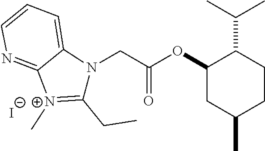 [SMILES: CCc1n(CC(=O)O[C@@H]2C[C@H](C)CC[C@H]2C(C)C)c2cccnc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-39 as a yellow solid (78%). IR (cm⁻¹) 2954, 2871, 1745, 1446, 1229, 954; [α]_{D}²⁰−19.73 (c 0.97 CHCl₃); ¹H NMR (400 MHz, CDCl₃) δ 8.62 (d, J=4.8 Hz, 1H), 8.41 (d, J=8.4 Hz, 1H), 7.60 (dd, J=8.0, 4.8 Hz, 1H), 5.46 (d, J=18.0 Hz, 1H), 5.37 (d, J=18.0 Hz, 1H), 4.78 (ddd, J=11.2, 10.8, 4.4 Hz, 1H), 4.25 (s, 3H), 3.51 (m, 2H), 1.97 (m, 1H), 1.83 (m, 1H), 1.68 (m, 2H), 1.44 (m, 2H), 1.41 (t, J=7.6 Hz, 3H), 1.09 (m, 1H), 0.99 (m, 1H), 0.90 (d, J=6.4 Hz, 3H), 0.89 (d, J=6.8 Hz, 3H), 0.86 (m, 1H), 0.71 (d, J=7.2 Hz, 3H); ¹³C NMR (100 MHz, CDCl₃) δ 165.8, 157.8, 148.5, 143.0, 125.1, 122.82, 122.77, 77.9, 47.9, 45.7, 40.7, 34.6, 34.0, 31.6, 26.4, 23.3, 22.1, 21.3, 20.9, 16.3, 11.3; ES-API MS: m/z calcd for C₂₁H₃₂IN₃O₂, found 358.3 [M−I].

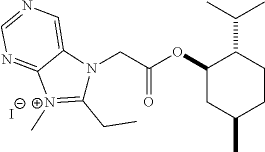 [SMILES: CCc1n(CC(=O)O[C@@H]2C[C@H](C)CC[C@H]2C(C)C)c2cncnc2[n+]1C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-34 as a yellow solid (64%). IR (cm⁻¹) 3446, 2959, 2872, 2199, 1747, 1732, 1652, 1646, 1586, 1538, 1505, 1480, 1456, 1372, 1293, 1224; [α]_{D}²⁰−2.042 (c 0.979 CHCl₃); ¹H NMR (400 MHz, CDCl₃) δ 9.78 (s, 1H), 9.57 (s, 1H), 5.26 (d, J=18.4 Hz, 1H), 5.21 (d, J=18.4 Hz, 1H), 4.73 (ddd, J=10.8, 10.8, 4.4 Hz, 1H), 4.62 (s, 3H), 3.03 (q, J=7.2 Hz, 2H), 1.94 (m, 1H), 1.78 (m, 1H), 1.65 (m, 2H), 1.45 (t, J=7.2 Hz, 3H), 1.38 (m, 2H), 1.02 (m, 2H), 0.88 (d, J=6.8 Hz, 6H), 0.83 (m, 1H), 0.70 (d, J=7.2 Hz, 3H); ¹³C NMR (100 MHz, CDCl₃) δ 167.2, 165.8, 155.3, 148.3, 141.1, 132.3, 77.6, 46.8, 46.2, 45.1, 40.7, 34.0, 31.5, 26.4, 23.3, 22.4, 22.0, 20.8, 16.3, 10.2; ES-API MS: m/z calcd for C₂₀H₃₁IN₄O₂, found 359.3 [M−I].

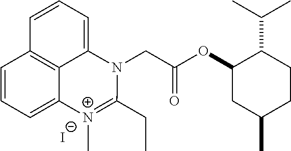 [SMILES: CCC1=[N+](C)c2cccc3cccc(c23)N1CC(=O)O[C@@H]1C[C@H](C)CC[C@H]1C(C)C ; [I-]]

The title compounds were obtained following the general procedure (Step C) described above. Purification of the residue by flash chromatography on silica gel (gradient elution, 015% MeOH in CH₂Cl₂) afforded the entitled salt AP-I-30 as a green solid (47%). IR (cm⁻¹) 2957, 2871, 2359, 1738, 1643, 1588, 1562, 1482, 1378, 1217; [α]_{D}²⁰−12.67 (c 1.010 CHCl₃); ¹H NMR (400 MHz, CDCl₃) δ 7.53 (t, J=8.0 Hz, 2H), 7.46 (t, J=8.0 Hz, 1H), 7.37 (t, J=8.8 Hz, 1H), 7.05 (d, J=8.0 Hz, 1H), 6.65 (d, J=8.0 Hz, 1H), 5.46 (br, 1H), 4.80 (ddd, J=10.8, 10.8, 4.4 Hz, 1H), 4.71 (br, 1H), 3.81 (s, 3H), 3.39 (br, 2H), 1.99 (m, 1H), 1.67 (m, 2H), 1.46 (m, 6H), 1.10 (m, 1H), 0.99 (m, 1H), 0.90 (d, J=6.4 Hz, 3H), 0.90 (m, 1H), 0.86 (m, 3H), 0.69 (m, 3H); ¹³C NMR (100 MHz, CDCl₃) δ 166.5, 166.1, 134.3, 133.2, 133.1, 128.6, 128.1, 124.9, 124.6, 120.4, 109.6, 108.1, 77.9, 53.4, 46.9, 40.7, 39.2, 34.0, 31.6, 29.8, 28.3, 23.1, 22.0, 20.9, 16.1, 10.8; ES-API MS: m/z calcd for C₂₆H₃₅IN₂O₂, found 407.3 [M−I].

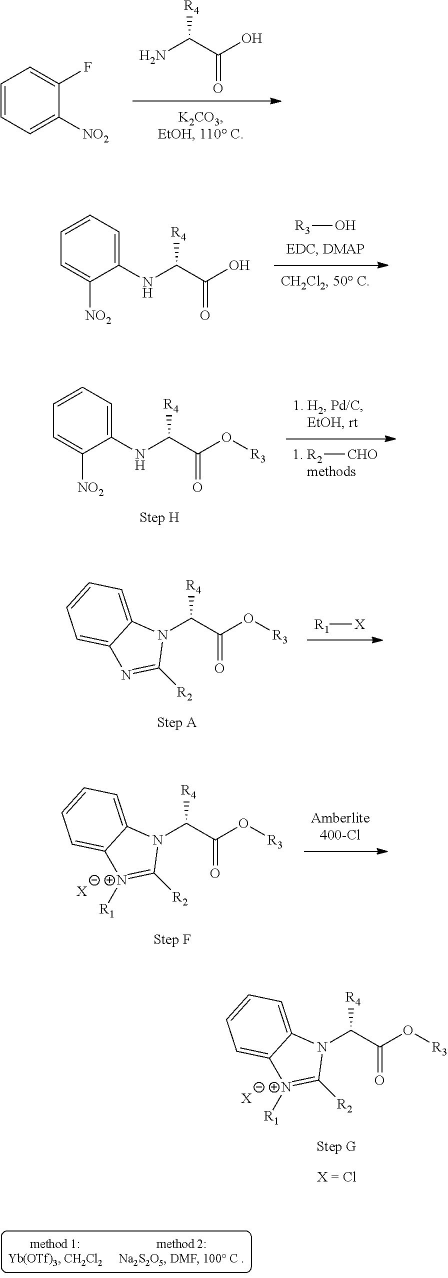

Step H: General Procedure for the Preparation of Mentholated Nitrobenzene from 2-fluoro-nitrobenzne

A mixture of 2-fluoro-nitrobenzene (10.2 mmol, 1 equiv.) and the corresponding amino acid (11.2 mmol, 1.1 equiv.) in EtOH (20 mL) was added K₂CO₃ (20.4 mmol, 2 equiv.). The resulting mixture was heated at 100° C. in a pressure-sealed tube for overnight. The solvent was removed under reduced pressure and quenched with 1N HCl. The mixture was extracted with EtOAc (×3), and the combined organic extracts was washed with brine and dried over anhydrous MgSO₄ and filtrated. The solvent was concentrated under reduced pressure, and the residue was used directly to the subsequent reaction. A mixture of the corresponding acid residue (6.76 mmol, 1.0 equiv.) and the corresponding menthol (7.43 mmol, 1.1 equiv.) was dissolved in anhydrous CH₂Cl₂ (13 mL). N-(3-Dimethylaminopropyl)-N-ethylcarbodiimide hydrochloride (EDC) (7.43 mmol, 1.1 equiv.) and 4-(dimethylamino)pyridine (DMAP) (3.38 mmol, 0.5 equiv.) were added sequentially to the reaction mixture. The resulting mixture was heated at 50° C. and stirred overnight. The solvent was removed under reduced pressure and the residue was purified by flash chromatography as indicated to give the corresponding mentholated nitrobenzene.

Step A: General Procedure for the Preparation of Benzimidazole from Aniline

Method 1: The corresponding aniline (1.0 mmol, 1 equiv.) was dissolved in anhydrous CH₂Cl₂ (3.3 mL) and cooled to 0° C. The corresponding aldehyde (1.3 mmol, 1.3 equiv.) and Yb(OTf)₃ (0.1 mmol, 0.10 equiv.) were added sequentially to the reaction. The mixture was raised to rt and stirred for overnight. The solvent was removed under reduced pressure and purified by flash chromatography on silica gel as indicated to give the desired benzimidazole.

Method 2: The corresponding aniline (0.75 mmol, 1 equiv.) was dissolved in anhydrous DMF (1.5 mL) and added the corresponding aldehyde (0.83 mmol, 1.1 equiv.). The mixture was stirred at rt for 10 min and then added Na₂S₂O₅ (0.83 mmol, 1.1 equiv.). The resulting mixture was heated at 100° C. for overnight. The solvent was removed under reduced pressure and purified by flash chromatography on silica gel as indicated to give the desired benzimidazole.

Step F: General Procedure for the Preparation of Alkyl Iodide Salt from Benzimidazole

The corresponding benzimidazole (0.10 mmol, 1 equiv.) was dissolved in the corresponding alkyl iodide (1.0 mL). The resulting mixture was stirred at 65° C. from 6 h to overnight depending upon TLC analysis. The excess alkyl iodide was removed under reduced pressure and the resulting residue was purified by either prep-TLC or flash chromatography on silica gel as indicated to give the salt.

Step G: General Procedure for the Preparation of Alkyl Chloride Salt from the Iodide Salt

The corresponding alkly iodide salt of benzimidazole (0.10 mmol, 1 equiv.) was dissolved in H₂O. This solution was added to Amberlite® IRA-400 Cl-exchange resin, and eluted with H₂O. Water was finally removed under reduced pressure and afforded the product as indicated.

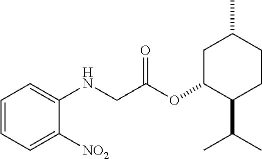 [SMILES: CC(C)[C@@H]1CC[C@@H](C)C[C@H]1OC(=O)CNc1ccccc1[N+](=O)[O-]]

The title compound was obtained following the general procedure (Step H) described above. Purification of the residue by flash chromatography on silica gel (gradient elution, 20% EtOAc in hexanes) afforded Lqr-5-067 as a yellow gel (84%). IR (cm⁻¹) 2924, 1724, 1650, 1537, 1425, 1369, 1273, 1205, 1149, 745; [α]_{D}²⁰−92.913 (c 1.3, CHCl₃); ¹H NMR (400 MHz, CDCl₃) δ 8.41 (t, J=4.4 Hz, 1H), 8.19 (dd, J=8.8, 1.6 Hz, 1H), 7.51-7.36 (m, 1H), 6.81-6.61 (m, 2H), 4.80 (td, J=11.2, 4.4 Hz, 1H), 4.07 (dd, J=5.2, 0.8 Hz, 2H), 2.03-1.85 (m, 1H), 1.85-1.73 (m, 1H), 1.73-1.62 (m, 2H), 1.59-1.35 (m, 2H), 1.35-1.24 (m, 1H), 1.13-0.95 (m, 2H), 0.90 (d, J=6.8 Hz, 3H), 0.87 (d, J=7.2 Hz, 3H), 0.73 (d, J=7.2 Hz, 3H); ¹³C NMR (100 MHz, CDCl₃) δ 168.7, 144.1, 136.1, 126.9, 116.1, 113.6, 76.0, 46.9, 45.1, 40.7, 34.0, 31.3, 26.2, 23.3, 21.9, 20.7, 16.2; ES-API MS: m/z calcd for C₁₈H₂₇N₂O₄, found 335.1 [M+H].

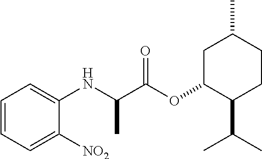 [SMILES: CC(C)[C@@H]1CC[C@@H](C)C[C@H]1OC(=O)[C@@H](C)Nc1ccccc1[N+](=O)[O-]]

The title compound was obtained following the general procedure (Step H) described above. Purification of the residue by flash chromatography on silica gel (gradient elution, 5% EtOAc in hexanes) afforded LW-III-235 as a yellow gel (98%). IR (cm⁻¹) 3363, 2957, 2931, 2871, 1738, 1619, 1574, 1271, 1165, 1041, 743; [α]_{D}²⁰−283.3 (c 1.00, CHCl₃); ¹H NMR (400 MHz, CDCl₃) δ 8.27 (d, J=6.4 Hz, 1H), 8.17 (dd, J=8.4, 1.6 Hz, 1H), 7.41 (ddd, J=7.2, 7.2, 1.6 Hz, 1H), 6.72 (d, J=8.8 Hz, 1H), 6.69 (ddd, J=7.2, 7.2, 1.2 Hz, 1H), 4.67 (ddd, J=10.8, 10.8, 4.4 Hz, 1H), 4.26 (pent, J=6.8 Hz, 1H), 1.99 (d, J=12.0 Hz, 1H), 1.65 (m, 2H), 1.59 (d, J=6.8 Hz, 3H), 1.55 (m, 1H), 1.47 (m, 1H), 1.34 (m, 1H), 0.98 (m, 2H), 0.89 (d, J=6.8 Hz, 3H), 0.84 (m, 1H), 0.76 (d, J=6.8 Hz, 3H), 0.60 (d, J=6.8 Hz, 3H); ¹³C NMR (100 MHz, CDCl₃) δ 172.4, 143,9, 136.1, 132.6, 126.9, 116.1, 113.8, 75.7, 51.9, 46.8, 40.6, 34.1, 31.3, 25.9, 23.0, 21.9, 20.7, 18.5, 15.7; ES-API MS: m/z calcd for C₁₉H₂₈N₂O₄, found 349.2 [M+H].

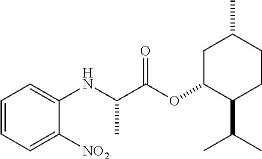

The title compound was obtained following the general procedure (Step H) described above. Purification of the residue by flash chromatography on silica gel (gradient elution, 15% EtOAc in hexanes) afforded LW-III-234 as a yellow gel (98%). IR (cm⁻¹) 3362, 2930, 2956, 2871, 1738, 1619, 1574, 1514, 1165, 1041, 743; [α]_{D}²⁰+84.32 (c 1.2, CHCl₃); ¹H NMR (400 MHz, CDCl₃) δ 8.31 (d, J=6.0 Hz, 1H), 8.19 (dd, J=8.4, 1.6 Hz, 1H), 7.40 (ddd, J=7.2, 7.2, 1.6 Hz, 1H), 6.71 (d, J=8.4 Hz, 1H), 6.69 (ddd, J=7.2, 7.2, 1.2 Hz, 1H), 4.74 (ddd, J=10.8, 10.8, 4.4 Hz, 1H), 4.26 (pent, J=6.4 Hz, 1H), 2.00-1.83 (m, 3H), 1.67 (m, 3H), 1.58 (d, J=6.8 Hz, 3H), 1.51-1.35 (m, 3H), 0.90 (d, J=6.8 Hz, 3H), 0.88 (d, J=6.8 Hz, 3H), 0.75 (d, J=6.8 Hz, 3H); ¹³C NMR (100 MHz, CDCl₃) δ 172.2, 143,8, 136.0, 132.6, 127.0, 116.1, 113.8, 75.7, 51.5, 46.8, 40.5, 34.1, 31.3, 26.4, 23.2, 21.9, 20.7, 18.5, 16.2; ES-API MS: m/z calcd for C₁₉H₂₈N₂O₄, found 349.2 [M+H].

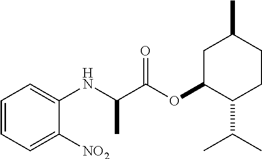

The title compound was obtained following the general procedure (Step H) described above. Purification of the residue by flash chromatography on silica gel (gradient elution, 10% EtOAc in hexanes) afforded LW-III-260 as a yellow gel (94%). IR (cm⁻¹) 3361, 2957, 1738, 1620, 1513, 1352, 1271, 1164, 1040, 742; [α]_{D}²⁰−125.3 (c 1.17, CHCl₃); ¹H NMR (400 MHz, CDCl₃) δ 8.31 (d, J=4.4 Hz, 1H), 8.18 (d, J=8.8 Hz, 1H), 7.40 (ddd, J=7.8, 7.6, 1.6 Hz, 1H), 6.71 (d, J=8.4 Hz, 1H), 6.69 (dd, J=7.6, 8.4 Hz, 2H), 4.74 (ddd, J=11.2, 10.8, 4.4 Hz, 1H), 4.26 (m, 1H), 1.93-1.84 (m, 2H), 1.69 (m, 1H), 1.66 (m, 1H), 1.58 (d, J=7.2 Hz, 3H), 1.52-1.38 (m, 2H), 1.09-0.94 (m, 2H), 0.90 (d, J=7.2 Hz, 3H), 0.88 (d, J=6.4 Hz, 3H), 0.75 (d, J=7.2 Hz, 3H); ¹³C NMR (100 MHz, CDCl₃) δ 172.4, 144.0, 136.2, 132.8, 127.2, 116.3, 114.0, 75.9, 51.7, 47.0, 40.7, 34.3, 31.5, 26.6, 23.5, 22.1, 20.9, 18.7, 16.4; ES-API MS: m/z calcd for C₁₉H₂₈N₂O₄, found 349.2 [M+H].

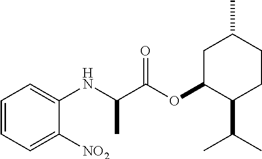

The title compound was obtained following the general procedure (Step H) described above. Purification of the residue by flash chromatography on silica gel (gradient elution, 10% EtOAc in hexanes) afforded LW-III-261 as a yellow gel (90%). IR (cm⁻¹) 3370, 2951, 2925, 2870, 1732, 1619, 1513, 1270, 1166, 743; [α]_{D}²⁰−157.62 (c 1.02, CHCl₃); ¹H NMR (400 MHz, CDCl₃) δ 8.27 (d, J=6.8 Hz, 1H), 8.14 (d, J=8.4 Hz, 1H), 7.38 (dd, J=8.4, 8.4 Hz, 1H), 6.72 (d, J=8.8 Hz, 1H), 6.66 (dd, J=8.4, 7.2 Hz, 1H), 5.21 (m, 1H), 4.28 (pent, J=6.8 Hz, 1H), 1.79 (dd, J=14.0, 2.4 Hz, 1H), 1.69 (dd, J=12.8, 3.2 Hz, 1H), 1.62 (d, J=12.8 Hz, 1H), 1.57 (d, J=6.8 Hz, 3H), 1.39 (m, 1H), 1.27-1.16 (m, 2H), 1.03-0.92 (m, 2H), 0.87 (d, J=6.8 Hz, 3H), 0.82 (d, J=6.8 Hz, 3H), 0.73 (m, 1H), 0.68 (d, J=6.8 Hz, 3H); ¹³C NMR (100 MHz, CDCl₃) δ 172.2, 144.1, 136.2, 132.7, 127.0, 116.3, 114.0, 72.8, 52.0, 46.6, 39.0, 34.6, 29.4, 26.5, 25.2, 22.1, 21.1, 20.7, 18.7; ES-API MS: m/z calcd for C₁₉H₂₈N₂O₄, found 349.2 [M+H].

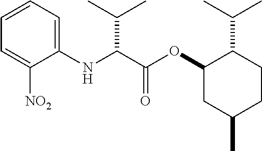

The title compound was obtained following the general procedure (Step H) described above. Purification of the residue by flash chromatography on silica gel (gradient elution, 8% EtOAc in hexanes) afforded LW-III-286 as a yellow gel (93%). IR (cm⁻¹) 3379, 2959, 2931, 2872, 1732, 1618, 1576, 1513, 1265, 1162, 743; [α]_{D}²⁰−300.19 (c 0.78, CHCl₃); NMR (400 MHz, CDCl₃) δ 8.33 (d, J=8.0 Hz, 1H), 8.18 (dd, J=8.8, 1.2 Hz, 1H), 7.40 (ddd, J=6.8, 6.8, 1.6 Hz, 1H), 6.77 (d, J=8.4 Hz, 1H), 6.68 (ddd, J=6.8, 6.8, 1.2 Hz, 1H), 4.64 (ddd, J=10.8, 10.8, 4.4 Hz, 1H), 3.99 (dd, J=8.0, 6.0 Hz, 1H), 2.30 (oct, J=6.0 Hz, 1H), 1.99 (m, 1H), 1.68-1.58 (m, 2H), 1.52-1.40 (m, 2H), 1.36-1.23 (m, 2H), 1.12 (d, J=6.8 Hz, 3H), 1.09 (d, J=6.8 Hz, 3H), 1.01-0.95 (m, 2H), 0.88 (d, J=6.8 Hz, 3H), 0.70 (d, J=7.2 Hz, 3H), 0.51 (d, J=6.8 Hz, 3H); ¹³C NMR (100 MHz, CDCl₃) δ 171.5, 145.0, 136.4, 132.8, 127.1, 116.3, 114.2, 75.9, 62.7, 46.9, 40.9, 34.3, 31.6, 31.4, 25.9, 23.0, 22.2, 20.9, 19.6, 18.6, 15.7; ES-API MS: m/z calcd for C₂₁H₃₂N₂O₄, found 377.2 [M+H].

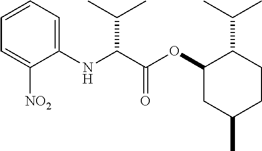

The title compound was obtained following the general procedure (Step H) described above. Purification of the residue by flash chromatography on silica gel (gradient elution, 8% EtOAc in hexanes) afforded LW-III-287 as a yellow gel (81%). IR (cm⁻¹) 3385, 2958, 2871, 1728, 1618, 1577, 1512, 1268, 1152, 744; [α]_{D}²⁰−359.04 (c 0.68, CHCl₃); ¹H NMR (400 MHz, CDCl₃) δ 8.49 (d, J=8.8 Hz, 1H), 8.18 (dd, J=8.4, 1.6 Hz, 1H), 7.40 (ddd, J=6.8, 6.8, 1.6 Hz, 1H), 6.81 (dd, J=8.8, 0.4 Hz, 1H), 6.68 (ddd, J=7.2, 7.2, 1.2 Hz, 1H), 4.59 (ddd, J=10.8, 10.8, 4.4 Hz, 1H), 3.92 (d, J=8.4 Hz, 1H), 1.99 (m, 1H), 1.66-1.55 (m, 2H), 1.44 (m, 1H), 1.34-1.25 (m, 2H), 1.15 (s, 9H), 0.99-0.90 (m, 2H), 0.88 (d, J=6.4 Hz, 3H), 0.82 (m, 1H), 0.62 (d, J=6.8 Hz, 3H), 0.42 (d, J=6.8 Hz, 3H); ¹³C NMR (100 MHz, CDCl₃) δ 171.2, 145.1, 136.5, 132.8, 127.1, 116.3, 114.2, 75.8, 66.1, 46.9, 40.9, 34.3, 34.2, 31.6, 27.2 (3C), 25.7, 22.9, 22.2, 20.8, 15.6; ES-API MS: m/z calcd for C₂₂H₃₄N₂O₄, found 391.3 [M+H].

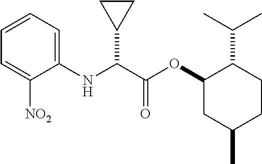

The title compound was obtained following the general procedure (Step H) described above. Purification of the residue by flash chromatography on silica gel (gradient elution, 8% EtOAc in hexanes) afforded LW-III-288 as a yellow gel (91%). IR (cm⁻¹) 3387, 2957, 2929, 2871, 1733, 1618, 1574, 1512, 1353, 1272, 1164, 743; [α]_{D}²⁰−215.73 (c 0.90, CHCl₃); ¹H NMR (400 MHz, CDCl₃) δ 8.35 (d, J=6.0 Hz, 1H), 8.18 (dd, J=9.2, 1.2 Hz, 1H), 7.40 (ddd, J=6.8, 6.8, 1.6 Hz, 1H), 6.69 (m, 1H), 6.67 (m, 1H), 4.69 (ddd, J=11.2, 11.2, 4.4 Hz, 1H), 3.67 (dd, J=7.2, 6.4 Hz, 1H), 2.01 (m, 1H), 1.70-1.57 (m, 4H), 1.48 (m, 1H), 1.40-1.24 (m, 5H), 1.00 (m, 1H), 0.90 (d, J=6.4 Hz, 3H), 0.84 (m, 1H), 0.76 (d, J=7.2 Hz, 3H), 0.70-0.66 (m, 1H), 0.59 (d, J=7.2 Hz, 3H); ¹³C NMR (100 MHz, CDCl₃) δ 171.6, 144.4, 136.3, 132.8, 127.1, 116.4, 114.2, 76.0, 60.2, 47.1, 40.9, 34.3, 31.6, 26.0, 23.1, 22.2, 20.9, 15.9, 13.9, 3.6, 3.3; ES-API MS: m/z calcd for C₂₁H₃₀N₂O₄, found 375.2 [M+H].

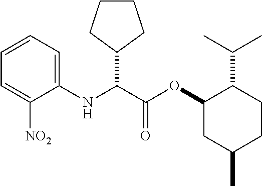

The title compound was obtained following the general procedure (Step H) described above. Purification of the residue by flash chromatography on silica gel (gradient elution, 8% EtOAc in hexanes) afforded LW-III-289 as a yellow gel (96%). IR (cm⁻¹) 3381, 2956, 2871, 1732, 1616, 1576, 1266, 1169, 744; [α]_{D}²⁰−230.24 (c 0.68, CHCl₃); ¹H NMR (400 MHz, CDCl₃) δ 8.26 (d, J=7.2 Hz, 1H), 8.17 (dd, J=8.8, 1.6 Hz, 1H), 7.40 (ddd, J=6.8, 7.2, 1.6 Hz, 1H), 6.80 (dd, J=8.8, 0.8 Hz, 1H), 6.68 (ddd, J=6.8, 7.2, 1.2 Hz, 1H), 4.61 (ddd, J=10.8, 10.8, 4.4 Hz, 1H), 4.00 (dd, J=7.6, 7.6 Hz, 1H), 2.43 (hex, J=8.0 Hz, 1H), 1.99-1.88 (m, 2H), 1.73-1.57 (m, 5H), 1.54-1.38 (m, 4H), 1.34-1.23 (m, 3H), 1.00-0.91 (m, 2H), 0.88 (d, J=6.4 Hz, 3H), 0.84 (m, 1H), 0.67 (d, J=7.2 Hz, 3H), 0.47 (d, J=6.8 Hz, 3H); ¹³C NMR (100 MHz, CDCl₃) δ 171.8, 144.7, 136.2, 132.5, 126.9, 116.2, 114.1, 75.5, 61.0, 46.7, 42.4, 40.6, 34.1, 31.3, 29.3, 29.0, 25.7, 25.4, 25.3, 22.8, 21.9, 20.6, 15.5; ES-API MS: m/z calcd for C₂₃H₃₄N₂O₄, found 403.3 [M+H].

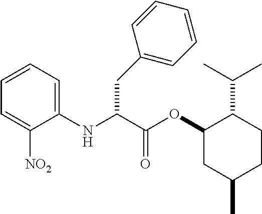

The title compound was obtained following the general procedure (Step H) described above. Purification of the residue by flash chromatography on silica gel (gradient elution, 10% EtOAc in hexanes) afforded LW-III-298 as a yellow gel (71%). IR (cm⁻¹) 2952, 1731, 1614, 1503, 1348, 1268, 1152, 744; [α]_{D}²⁰−67.68 (c 1.17, CHCl₃); ¹H NMR (400 MHz, CDCl₃) δ 8.32 (d, J=7.6 Hz, 1H), 8.16 (dd, J=8.8, 1.2 Hz, 1H), 7.38 (ddd, J=6.8, 6.8, 1.2 Hz, 1H), 7.34-7.24 (m, 5H), 6.72 (d, J=8.8 Hz, 1H), 6.68 (ddd, J=7.2, 7.2, 1.2 Hz, 1H), 4.65 (ddd, J=11.2, 10.8, 4.4 Hz, 1H), 4.44 (ddd, J=8.8, 7.6, 7.2 Hz, 1H), 3.30 (dd, J=13.6, 5.6 Hz, 1H), 3.17 (dd, J=14.0, 7.6 Hz, 1H), 1.95 (m, 1H), 1.68-1.59 (m, 2H), 1.51-1.26 (m, 3H), 1.03-0.92 (m, 2H), 0.90 (d, J=6.4 Hz, 3H), 0.83 (m, 1H), 0.70 (d, J=6.8 Hz, 3H), 0.54 (d, J=7.2 Hz, 3H); ¹³C NMR (100 MHz, CDCl₃) δ 171.5, 144.3, 136.3, 135.8, 132.9, 129.5 (2C), 129.0 (2C), 127.6, 127.1, 116.6, 114.2, 76.2, 58.2, 46.9, 40.8, 38.9, 34.2, 31.5, 25.8, 23.1, 22.2, 20.9, 15.9; ES-API MS: m/z calcd for C₂₅H₃₂N₂O₄, found 425.2 [M+H].

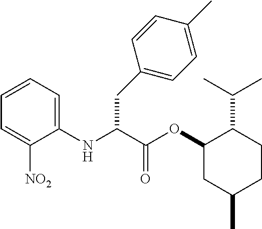

The title compound was obtained following the general procedure (Step H) described above. Purification of the residue by flash chromatography on silica gel (gradient elution, 10% EtOAc in hexanes) afforded LW-III-299 as a yellow gel (78%). IR (cm⁻¹) 2957, 2358, 1728, 1613, 1503, 1263; [α]_{D}²⁰−58.08 (c 1.05, CHCl₃); ¹H NMR (400 MHz, CDCl₃) δ 8.31 (d, J=7.2 Hz, 1H), 8.16 (dd, J=8.8, 1.6 Hz, 1H), 7.38 (ddd, J=6.8, 6.8, 1.2 Hz, 1H), 7.17 (d, J=8.4 Hz, 2H), 7.12 (d, J=8.0 Hz, 2H), 6.72 (d, J=8.8 Hz, 1H), 6.68 (ddd, J=7.2, 7.2, 1.2 Hz, 1H), 4.64 (ddd, J=10.8, 10.8, 4.4 Hz, 1H), 4.41 (ddd, J=7.6, 7.2, 6.0 Hz, 1H), 3.26 (dd, J=14.0, 5.6 Hz, 1H), 3.13 (dd, J=13.6, 7.6 Hz, 1H), 2.32 (s, 3H), 1.94 (m, 1H), 1.68-1.58 (m, 2H), 1.45 (m, 1H), 1.38-1.26 (m, 2H), 1.01-0.91 (m, 2H), 0.89 (d, J=6.4 Hz, 3H), 0.84 (m, 1H), 0.69 (d, J=6.8 Hz, 3H), 0.53 (d, J=6.8 Hz, 3H); ¹³C NMR (100 MHz, CDCl₃) δ 171.6, 144.3, 137.2, 136.3, 132.9, 132.6, 129.7 (2C), 129.4 (2C), 127.1, 116.5, 114.2, 76.1, 58.4, 46.9, 40.8, 38.5, 34.2, 31.6, 25.8, 23.1, 22.2, 21.3, 20.9, 15.9; ES-API MS: m/z calcd for C₂₆H₃₄N₂O₄, found 439.3 [M+H].

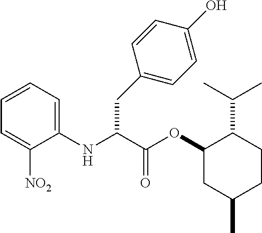

The title compound was obtained following the general procedure (Step H) described above. Purification of the residue by flash chromatography on silica gel (gradient elution, 10% EtOAc in hexanes) afforded LW-III-300 as a yellow gel (6%). IR (cm⁻¹) 3374, 2962, 2871, 1727, 1614, 1574, 1351, 1262, 1039, 801; [α]_{D}²⁰−46.90 (c 1.33, CHCl₃); ¹H NMR (400 MHz, CDCl₃) δ 8.30 (d, J=7.2 Hz, 1H), 8.15 (dd, J=8.4, 0.8 Hz, 1H), 7.38 (dd, J=8.0, 8.0 Hz, 1H), 7.13 (d, J=8.0 Hz, 2H), 6.77 (d, J=8.4 Hz, 2H), 6.71 (d, J=8.4 Hz, 1H), 6.68 (dd, J=8.0, 7.6 Hz, 1H), 5.28 (s, 1H), 4.64 (ddd, J=10.8, 11.2, 4.8 Hz, 1H), 4.39 (m, 1H), 3.22 (dd, J=14.0, 5.6 Hz, 1H), 3.11 (dd, J=13.6, 7.6 Hz, 1H), 1.94 (m, 1H), 1.72-1.59 (m, 2H), 1.48-1.24 (m, 3H), 1.01-0.92 (m, 2H), 0.88 (d, J=6.4 Hz, 3H), 0.83 (m, 1H), 0.70 (d, J=6.8 Hz, 3H), 0.53 (d, J=6.8 Hz, 3H); ¹³C NMR (100 MHz, CDCl₃) δ 171.8, 155.3, 144.3, 136.4, 132.8, 130.7 (2C), 127.6, 127.1, 116.6, 115.9 (2C), 114.2, 76.3, 58.4, 46.9, 40.8, 38.1, 34.2, 31.5, 25.8, 23.1, 22.2, 20.9, 15.9; ES-API MS: m/z calcd for C₂₅H₃₂N₂O₅, found 441.2 [M+H].

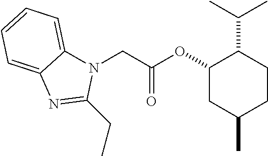

The title compound was obtained following the general procedure (Step A, method 1) described above. Purification of the residue by flash chromatography on silica gel (gradient elution, 60% EtOAc in hexanes) afforded LW-III-242 as a pale yellow gel (63%). IR (cm⁻¹) 2949, 2360, 1738, 1463, 1195, 742; [α]_{D}²⁰+19.07 (c 1.08, CHCl₃); ¹H NMR (400 MHz, CDCl₃) δ 7.72 (m, 1H), 7.23-7.17 (m, 3H), 5.18 (m, 1H), 4.80 (s, 2H), 2.85 (q, J=7.6 Hz, 2H), 1.81 (d, J=14.0 Hz, 1H), 1.54-1.46 (m, 2H), 1.46 (t, J=7.6 Hz, 3H), 1.08 (m, 1H), 0.99-0.76 (m, 5H), 0.72-0.70 (m, 9H); ¹³C NMR (100 MHz, CDCl₃) δ 166.9, 156.0, 142.5, 135.3, 122.4, 122.2, 119.4, 108.6, 73.5, 46.4, 45.4, 38.9, 34.3, 28.9, 26.3, 24.8, 21.9, 20.8, 20.7, 20.5, 11.5; ES-API MS: m/z calcd for C₂₁H₃₀N₂O₂, found 343.2 [M+H].

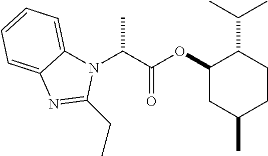

The title compound was obtained following the general procedure (Step A, method 1) described above. Purification of the residue by flash chromatography on silica gel (gradient elution, 45% EtOAc in hexanes) afforded LW-III-236 as a pale yellow gel (65%). IR (cm⁻¹) 2956, 2871, 2360, 1736, 1459, 1221, 742; [α]_{D}²⁰−55.87 (c 1.02, CHCl₃); ¹H NMR (400 MHz, CDCl₃) δ 7.71 (dd, J=6.8, 1.6 Hz, 1H), 7.27 (dd, J=6.8, 1.6 Hz, 1H), 7.20 (ddd, J=7.6, 7.6, 1.2 Hz, 1H), 7.16 (ddd, J=7.6, 7.6, 1.2 Hz, 1H), 5.09 (q, J=7.2 Hz, 1H), 4.71 (ddd, J=10.8, 10.8, 4.4 Hz, 1H), 2.89 (q, J=7.6 Hz, 2H), 1.79 (d, J=7.2 Hz, 3H), 1.73 (m, 2H), 1.62-1.57 (m, 2H), 1.45 (t, J=7.6 Hz, 3H), 1.37 (m, 1H), 1.24 (m, 1H), 1.20 (m, 1H), 0.98 (m, 1H), 0.83 (d, J=6.8 Hz, 3H), 0.77 (d, J=6.4 Hz, 3H), 0.74 (d, J=6.8 Hz, 3H), 0.60 (m, 1H); ¹³C NMR (100 MHz, CDCl₃) δ 169.3, 155.6, 142.8, 133.7, 122.0, 121.9, 119.4, 110.5, 76.4, 53.0, 46.6, 40.1, 33.8, 31.2, 26.4, 23.3, 21.8, 21.3, 20.6, 16.32, 16.28, 12.0; ES-API MS: m/z calcd for C₂₂H₃₂N₂O₂, found 357.3 [M+H].

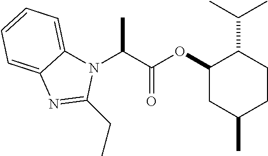

The title compound was obtained following the general procedure (Step A, method 1) described above. Purification of the residue by flash chromatography on silica gel (gradient elution, 50% EtOAc in hexanes) afforded LW-III-237 as a pale yellow gel (46%). IR (cm⁻¹) 2956, 2870, 1737, 1459, 1215, 742; [α]_{D}²⁰−35.64 (c 1.24, CHCl₃); ¹H NMR (400 MHz, CDCl₃) δ 7.68 (d, J=8.4 Hz, 1H), 7.27 (d, J=7.2 Hz, 1H), 7.16 (ddd, J=7.2, 7.2, 1.2 Hz, 1H), 7.13 (ddd, J=7.6, 7.6, 1.2 Hz, 1H), 5.05 (q, J=7.6 Hz, 1H), 4.56 (ddd, J=10.8, 11.2, 4.4 Hz, 1H), 2.90 (q, J=7.6 Hz, 2H), 2.00 (d, J=12.4 Hz, 1H), 1.78 (d, J=7.6 Hz, 3H), 1.58 (d, J=12.8 Hz, 1H), 1.49 (m, 1H), 1.43 (t, J=7.6 Hz, 3H), 1.05 (m, 1H), 0.89 (m, 3H), 0.85 (d, J=6.8 Hz, 3H), 0.82-0.69 (m, 2H), 0.44 (d, J=6.8 Hz, 3H), 0.25 (d, J=6.8 Hz, 3H); ¹³C NMR (100 MHz, CDCl₃) δ 169.2, 155.6, 142.8, 133.6, 122.1, 121.8, 119.4, 110.6, 76.2, 53.1, 46.6, 40.6, 33.9, 31.3, 25.2, 22.8, 21.9, 21.2, 20.3, 15.8, 15.4, 12.0; ES-API MS: m/z calcd for C₂₂H₃₂N₂O₂, found 357.3 [M+H].

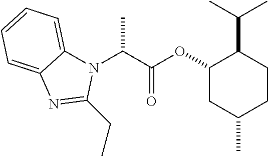

The title compound was obtained following the general procedure (Step A, method 1) described above. Purification of the residue by flash chromatography on silica gel (gradient elution, 50% EtOAc in hexanes) afforded LW-III-262 as a pale yellow gel (60%). IR (cm⁻¹) 2959, 1738, 1692, 1679, 1462, 1383, 1258, 1032, 747; [α]_{D}²⁰+27.37 (c 1.03, CHCl₃); ¹H NMR (400 MHz, CDCl₃) δ 7.70 (dd, J=7.2, 1.2 Hz, 1H), 7.29 (dd, J=7.2, 1.6 Hz, 1H), 7.19 (ddd, J=6.8, 6.8, 1.2 Hz, 1H), 7.16 (ddd, J=6.8, 6.8, 1.2 Hz, 1H), 5.08 (q, J=7.2 Hz, 1H), 4.58 (ddd, J=10.8, 10.8, 4.0 Hz, 1H), 2.93 (q, J=7.6 Hz, 2H), 2.02 (m, 1H), 1.81 (d, J=7.6 Hz, 3H), 1.61 (d, J=12.8 Hz, 1H), 1.51 (m, 1H), 1.46 (t, J=7.6 Hz, 3H), 1.07 (m, 1H), 0.96-0.89 (m, 2H), 0.88 (d, J=6.4 Hz, 3H), 0.84-0.71 (m, 3H), 0.46 (d, J=6.8 Hz, 3H), 0.27 (d, J=6.8 Hz, 3H); ¹³C NMR (100 MHz, CDCl₃) δ 169.4, 155.9, 142.9, 133.8, 122.4, 122.1, 119.6, 110.8, 76.5, 53.4, 46.9, 40.8, 34.2, 31.5, 25.4, 23.0, 22.2, 21.4, 20.6, 16.1, 15.6, 12.3; ES-API MS: m/z calcd for C₂₂H₃₂N₂O₂, found 357.3 [M+H].

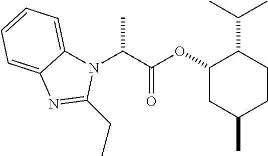

The title compound was obtained following the general procedure (Step A, method 1) described above. Purification of the residue by flash chromatography on silica gel (gradient elution, 50% EtOAc in hexanes) afforded LW-III-263 as a pale yellow gel (31%). IR (cm⁻¹) 2950, 2870, 1738, 1460, 1403, 1382, 1260, 1216, 1102, 742; [α]_{D}²⁰+16.81 (c 1.13, CHCl₃); ¹H NMR (400 MHz, CDCl₃) δ 7.67 (dd, J=7.2, 1.2 Hz, 1H), 7.26 (ddd, J=7.2, 7.2, 2.0 Hz, 1H), 7.16 (ddd, J=7.2, 7.2, 1.2 Hz, 1H), 7.12 (ddd, J=7.2, 7.2, 1.2 Hz, 1H), 5.10 (m, 1H), 5.08 (q, J=7.6 Hz, 1H), 2.90 (q, J=7.6 Hz, 2H), 1.94 (dd, J=14.4, 2.4 Hz, 1H), 1.82 (d, J=7.2 Hz, 3H), 1.61 (m, 1H), 1.51-1.37 (m, 2H), 1.45 (t, J=7.6 Hz, 3H), 0.99 (ddd, J=13.4, 13.4, 2.0 Hz, 1H), 0.81 (d, J=6.8 Hz, 3H), 0.81 (m, 1H), 0.77-0.68 (m, 2H), 0.46 (d, J=6.4 Hz, 3H), 0.37 (d, J=6.4 Hz, 3H), 0.26 (m, 1H); ¹³C NMR (100 MHz, CDCl₃) δ 169.1, 155.6, 142.8, 133.8, 122.1, 121.9, 119.4, 110.6, 73.2, 53.2, 46.5, 38.8, 34.4, 28.1, 26.7, 24.8, 22.1, 21.2, 20.5, 20.3, 15.8, 12.0; ES-API MS: m/z calcd for C₂₂H₃₂N₂O₂, found 357.3 [M+H].

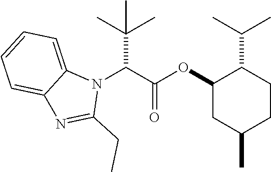

The title compound was obtained following the general procedure (Step A, method 1) described above. Purification of the residue by flash chromatography on silica gel (gradient elution, 45% EtOAc in hexanes) afforded LW-III-290 as a pale yellow gel (25%). IR (cm⁻¹) 2962, 2874, 1741, 1460, 1409, 1261, 1034, 802; [α]_{D}²⁰−80.16 (c 1.14, CHCl₃); NMR (400 MHz, CDCl₃) δ 7.69 (dd, J=6.8, 2.0 Hz, 1H), 7.64 (dd, J=7.2, 1.6 Hz, 1H), 7.18 (m, 2H), 4.69 (ddd, J=10.8, 10.8, 4.4 Hz, 1H), 4.43 (d, J=11.6 Hz, 1H), 2.99 (m, 1H), 2.93 (m, 2H), 1.67-1.59 (m, 4H), 1.47 (t, J=7.6 Hz, 3H), 1.36 (m, 1H), 1.26 (m, 1H), 1.15 (d, J=6.4 Hz, 3H), 0.98 (m, 1H), 0.80 (m, 1H), 0.77 (d, J=6.4 Hz, 3H), 0.76 (d, J=6.0 Hz, 3H), 0.73 (m, 1H), 0.68 (d, J=6.8 Hz, 3H), 0.63 (d, J=6.4 Hz, 3H); ¹³C NMR (100 MHz, CDCl₃) δ 168.6, 156.6, 142.9, 133.9, 122.3, 122.1, 119.4, 112.4, 76.1, 65.7, 46.9, 40.4, 34.1, 31.4, 28.2, 26.3, 23.3, 22.0, 21.6, 20.8, 20.3, 18.9, 16.2, 12.2; ES-API MS: m/z calcd for C₂₄H₃₆N₂O₂, found 385.3 [M+H].

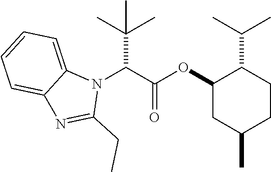

The title compound was obtained following the general procedure (Step A, method 1) described above. Purification of the residue by flash chromatography on silica gel (gradient elution, 45% EtOAc in hexanes) afforded LW-III-291 as a pale yellow gel (10%). IR (cm⁻¹) 2961, 2873, 1742, 1261, 1039, 802; [α]_{D}²⁰−51.21 (c 1.23, CHCl₃); ¹H NMR (400 MHz, CDCl₃) δ 7.69 (d, J=8.0 Hz, 1H), 7.61 (d, J=8.0 Hz, 1H), 7.18 (dd, J=6.8, 7.6 Hz, 1H), 7.14 (dd, J=8.4, 6.8 Hz, 1H), 4.69 (ddd, J=11.0, 11.0, 4.4 Hz, 1H), 4.69 (s, 1H), 3.01 (dq, J=15.6, 7.6 Hz, 1H), 2.87 (dq, J=14.8, 7.2 Hz, 1H), 1.75 (m, 1H), 1.62-1.55 (m, 3H), 1.47 (t, J=7.6 Hz, 3H), 1.33 (m, 1H), 1.22 (m, 1H), 1.18 (s, 9H), 1.03-0.91 (m, 2H), 0.83 (d, J=7.2 Hz, 3H), 0.75 (d, J=6.8 Hz, 3H), 0.72 (d, J=6.4 Hz, 3H), 0.67 (m, 1H); ¹³C NMR (100 MHz, CDCl₃) δ 167.5, 157.5, 143.0, 134.6, 122.01, 121.99, 119.2, 114.1, 75.9, 66.4, 46.8, 40.3, 37.3, 34.1, 31.4, 28.3 (3C), 26.7, 23.5, 21.9 (2C), 20.8, 16.5, 12.3; ES-API MS: m/z calcd for C₂₅H₃₈N₂O₂, found 399.3 [M+H].

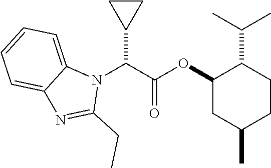

The title compound was obtained following the general procedure (Step A, method 1) described above. Purification of the residue by flash chromatography on silica gel (gradient elution, 45% EtOAc in hexanes) afforded LW-III-292 as a pale yellow gel (35%). IR (cm⁻¹) 3374, 2957, 2871, 1738, 1457, 1274, 1026, 804, 741; [α]_{D}²⁰−57.58 (c 1.00, CHCl₃); ¹H NMR (400 MHz, CDCl₃) δ 7.70 (dd, J=6.8, 1.6 Hz, 1H), 7.44 (d, J=8.0 Hz, 1H), 7.17 (m, 2H), 4.69 (ddd, J=10.8, 10.8, 4.4 Hz, 1H), 4.13 (d, J=10.0 Hz, 1H), 2.84 (dq, J=15.6, 8.0 Hz, 1H), 2.82 (dq, J=15.6, 8.0 Hz, 1H), 1.89 (m, 1H), 1.78 (d, J=12.0 Hz, 1H), 1.68 (m, 1H), 1.59 (m, 2H), 1.42 (t, J=7.6 Hz, 3H), 1.36 (m, 1H), 1.23 (m, 1H), 0.97 (m, 1H), 0.88 (m, 2H), 0.78 (d, J=6.8 Hz, 3H), 0.77 (d, J=6.8 Hz, 3H), 0.72 (m, 1H), 0.69 (d, J=6.8 Hz, 3H), 0.63-0.50 (m, 2H), 0.25 (m, 1H); ¹³C NMR (100 MHz, CDCl₃) δ 168.5, 155.8, 142.7, 134.4, 122.1, 121.8, 119.2, 111.0, 76.3, 63.2, 46.7, 40.3, 33.9, 31.2, 26.2, 23.2, 21.8, 21.4, 20.6, 16.1, 12.3, 12.0, 6.1, 3.9; ES-API MS: m/z calcd for C₂₄H₃₄N₂O₂, found 383.3 [M+H].

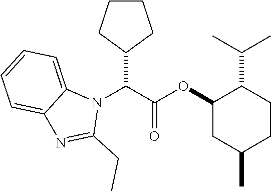

The title compound was obtained following the general procedure (Step A, method 1) described above. Purification of the residue by flash chromatography on silica gel (gradient elution, 45% EtOAc in hexanes) afforded LW-III-293 as a pale yellow gel (30%). IR (cm⁻¹) 2959, 2871, 1738, 1460, 1260, 1027, 803, 742; [α]_{D}²⁰−54.38 (c 1.00, CHCl₃); NMR (400 MHz, CDCl₃) δ 7.70 (d, J=6.8 Hz, 1H), 7.58 (d, J=4.4 Hz, 1H), 7.19 (m, 2H), 4.69 (ddd, J=10.0, 10.0, 3.6 Hz, 1H), 4.58 (d, J=11.2 Hz, 1H), 3.19 (m, 1H), 2.93 (m, 2H), 2.08 (m, 2H), 1.71-1.59 (m, 7H), 1.47 (t, J=6.8 Hz, 3H), 1.39-0.87 (m, 8H), 0.79 (d, J=7.2 Hz, 3H), 0.77 (d, J=6.4 Hz, 3H), 0.70 (d, J=6.4 Hz, 3H); ¹³C NMR (100 MHz, CDCl₃) δ 168.9, 156.4, 142.9, 134.2, 122.3, 122.1, 119.4, 112.1, 76.2, 63.9, 46.9, 40.4, 39.9, 34.1, 31.4, 31.1, 29.9, 26.5, 25.6, 25.0, 23.4, 22.0, 21.6, 20.8, 16.3, 12.2; ES-API MS: m/z calcd for C₂₆H₃₈N₂O₂, found 411.3 [M+H].

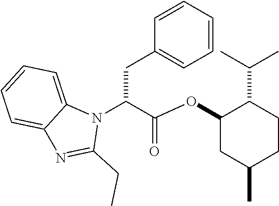

The title compound was obtained following the general procedure (Step A, method 1) described above. Purification of the residue by flash chromatography on silica gel (gradient elution, 45% EtOAc in hexanes) afforded LW-III-301 as a pale yellow gel (43%). IR (cm⁻¹) 2957, 2871, 1737, 1460, 1276, 1215, 743; [α]_{D}²⁰ +42.49 (c 1.12, CHCl₃); ¹H NMR (400 MHz, CDCl₃) δ 7.75 (m, 1H), 7.44 (m, 1H), 7.29-7.23 (m, 2H), 7.20-7.13 (m, 3H), 6.80-6.78 (m, 2H), 5.02 (dd, J=10.8, 4.4 Hz, 1H), 4.78 (ddd, J=10.8, 10.8, 4.4 Hz, 1H), 3.65 (dd, J=14.0, 4.8 Hz, 1H), 3.50 (dd, J=14.0, 10.8 Hz, 1H), 2.42 (dq, J=15.6, 7.6 Hz, 1H), 2.26 (dq, J=15.2, 7.2 Hz, 1H), 1.79 (m, 1H), 1.71 (m, 1H), 1.64 (m, 1H), 1.61 (m, 1H), 1.42 (m, 1H), 1.52 (m, 1H), 1.18 (t, J=7.2 Hz, 3H), 1.02 (m, 1H), 0.84 (d, J=7.2 Hz, 3H), 0.81 (d, J=6.4 Hz, 3H), 0.77 (d, J=6.8 Hz, 3H), 0.72 (m, 1H), 0.64 (m, 1H); ¹³C NMR (100 MHz, CDCl₃) δ 168.7, 156.6, 143.0, 136.7, 133.6, 129.0 (2C), 128.9 (2C), 127.4, 122.4, 122.2, 119.7, 111.3, 76.8, 60.1, 46.8, 40.3, 36.0, 34.1, 31.4, 26.6, 23.5, 22.0, 20.8, 20.7, 16.6, 11.6; ES-API MS: m/z calcd for C₂₈H₃₆N₂O₂, found 433.3 [M+H].

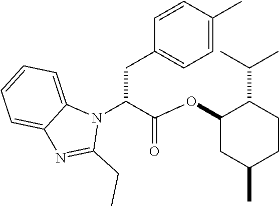

The title compound was obtained following the general procedure (Step A, method 1) described above. Purification of the residue by flash chromatography on silica gel (gradient elution, 45% EtOAc in hexanes) afforded LW-III-302 as a pale yellow gel (44%). IR (cm⁻¹) 2957, 2871, 1738, 1460, 1276, 1214, 1170, 742; [α]_{D}²⁰+60.85 (c 1.16, CHCl₃); ¹H NMR (400 MHz, CDCl₃) δ 7.75 (d, J=9.2 Hz, 1H), 7.45 (d, J=8.4 Hz, 1H), 7.27 (ddd, J=9.6, 9.6, 2.0 Hz, 1H), 7.25 (ddd, J=9.6, 9.6, 1.6 Hz, 1H), 6.96 (d, J=8.0 Hz, 2H), 6.68 (d, J=8.0 Hz, 2H), 5.01 (dd, J=10.4, 4.8 Hz, 1H), 4.78 (ddd, J=11.2, 10.8, 4.4 Hz, 1H), 3.61 (dd, J=14.0, 4.8 Hz, 1H), 3.46 (dd, J=13.6, 10.4 Hz, 1H), 2.44 (dq, J=15.2, 7.6 Hz, 1H), 2.33 (dq, J=15.6, 8.0 Hz, 1H), 2.26 (s, 3H), 1.80 (m, 1H), 1.71 (m, 1H), 1.64 (m, 1H), 1.61 (m, 1H), 1.42 (m, 1H), 1.24 (m, 1H), 1.20 (t, J=7.6 Hz, 3H), 1.02 (m, 1H), 0.90 (m, 1H), 0.84 (d, J=7.2 Hz, 3H), 0.81 (d, J=6.4 Hz, 3H), 0.77 (d, J=6.8 Hz, 3H), 0.75 (m, 1H); ¹³C NMR (100 MHz, CDCl₃) δ 168.7, 156.6, 143.0, 136.9, 133.4, 129.5 (3C), 128.8 (2C), 122.3, 122.1, 119.6, 111.3, 76.7, 60.2, 46.8, 40.3, 35.5, 34.0, 31.4, 26.5, 23.5, 22.0, 21.1, 20.8, 20.7, 16.5, 11.6; ES-API MS: m/z calcd for C₂₉H₃₈N₂O₂, found 447.3 [M+H].

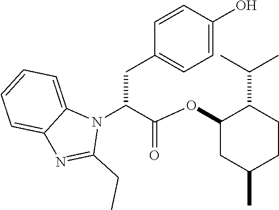

The title compound was obtained following the general procedure (Step A, method 1) described above. Purification of the residue by flash chromatography on silica gel (gradient elution, 45% EtOAc in hexanes) afforded LW-III-303 as a pale yellow gel (22%). IR (cm⁻¹) 2959, 1738, 1613, 1514, 1463, 1415, 1260, 1030, 802, 743; [α]_{D}²⁰+90.65 (c 0.30, CHCl₃); ¹H NMR (400 MHz, CDCl₃) δ 11.9 (br, 1H), 7.57 (d, J=8.0 Hz, 1H), 7.33 (d, J=8.0 Hz, 1H), 7.22 (ddd, J=8.4, 6.8, 1.2 Hz, 1H), 7.16 (dd, J=7.2, 7.2 Hz, 1H), 6.55 (d, J=8.4 Hz, 2H), 6.28 (d, J=8.0 Hz, 2H), 4.86-4.80 (m, 2H), 3.43 (dd, J=14.4, 3.2 Hz, 1H), 3.36 (dd, J=16.4, 11.6 Hz, 1H), 2.22 (dq, J=15.2, 7.6 Hz, 1H), 1.99 (dq, J=15.2, 7.2 Hz, 1H), 1.87 (m, 1H), 1.82 (m, 1H), 1.66-1.61 (m, 2H), 1.44 (m, 1H), 1.32-1.25 (m, 3H), 1.06 (m, 1H), 1.02 (t, J=7.6 Hz, 3H), 0.87 (d, J=7.2 Hz, 1H), 0.81 (d, J=7.2 Hz, 6H), 0.76 (m, 1H), 0.69 (m, 1H); ¹³C NMR (100 MHz, CDCl₃) δ 168.5, 158.0, 157.3, 132.7, 129.9 (2C), 126.2, 123.1, 123.0, 118.8, 115.9 (2C), 111.7, 110.2, 77.0, 60.7, 46.8, 40.4, 35.0, 34.1, 31.5, 26.8, 23.6, 22.1, 20.9, 20.2, 16.7, 11.2; ES-API MS: m/z calcd for C₂₈H₃₆N₂O₃, found 449.3 [M+H].

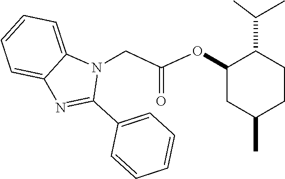

The title compound was obtained following the general procedure (Step A, method 1) described above. Purification of the residue by flash chromatography on silica gel (gradient elution, 40% EtOAc in hexanes) afforded Lqr-5-069 as a yellow solid (40%). IR (cm⁻¹) 2954, 1734, 1458, 1208, 981, 740; [α]_{D}²⁰−24.663 (c 0.6, CHCl₃); ¹H NMR (400 MHz, CDCl₃) δ 7.93-7.78 (m, 1H), 7.74-7.68 (m, 2H), 7.59-7.44 (m, 3H), 7.44-7.21 (m, 3H), 4.88 (d, J=3.6 Hz, 2H), 4.78 (td, J=11.2, 4.4 Hz, 1H), 1.96 (d, J=12.0 Hz, 1H), 1.77-1.53 (m, 3H), 1.52-1.40 (m, 1H), 1.34-1.21 (m, 1H), 1.10-0.74 (m, 3H), 0.90 (d, J=6.8 Hz, 3H), 0.80 (d, J=7.2 Hz, 3H), 0.69 (d, J=7.2 Hz, 3H); ¹³C NMR (100 MHz, CDCl₃) δ 167.4, 153.9, 142.9, 136.0, 130.0, 129.7, 129.3 (2C), 128.7 (2C), 123.2, 122.8, 120.1, 109.4, 76.4, 46.8, 46.6, 40.5, 33.9, 31.3, 26.0, 23.0, 21.9, 20.6, 15.9; ES-API MS: m/z calcd for C₂₅H₃₀N₂O₂, found 391.2 [M+H].

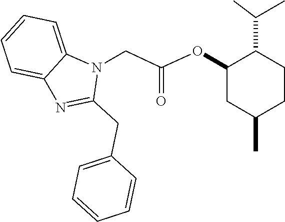

The title compound was obtained following the general procedure (Step A, method 1) described above. Purification of the residue by flash chromatography on silica gel (gradient elution, 40% EtOAc in hexanes) afforded Lqr-5-070 as a yellow solid (30%). IR (cm⁻¹) 2954, 1739, 1455, 1211, 959, 743; [α]_{D}²⁰−26.442 (c 0.4, CHCl₃); ¹H NMR (400 MHz, CDCl₃) δ 7.87-7.68 (m, 1H), 7.45-7.09 (m, 8H), 4.74-4.54 (m, 1H), 4.65 (s, 2H), 4.42-4.18 (m, 2H), 1.86 (d, J=12.0 Hz, 1H), 1.68-1.55 (m, 2H), 1.54-1.36 (m, 2H), 1.33-1.15 (m, 1H), 0.97 (qd, J=12.8, 3.2 Hz, 1H), 0.88 (d, J=6.8 Hz, 3H), 0.83-0.76 (m, 2H), 0.77 (d, J=7.2 Hz, 3H), 0.64 (d, J=7.2 Hz, 3H); ¹³C NMR (100 MHz, CDCl₃) δ 166.7, 153.1, 135.6, 129.3, 128.9 (2C), 128.5, 128.4 (2C), 127.1, 122.8, 122.4, 119.6, 108.8, 76.4, 46.6, 45.3, 40.4, 34.3, 33.9, 31.2, 26.0, 23.1, 21.9, 20.6, 16.0; ES-API MS: m/z calcd for C₂₆H₃₂N₂O₂, found 405.2 [M+H].

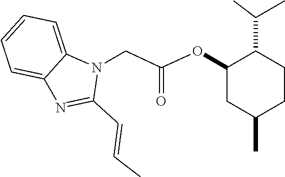

The title compound was obtained following the general procedure (Step A, method 1) described above. Purification of the residue by flash chromatography on silica gel (gradient elution, 40% EtOAc in hexanes) afforded Lqr-5-071 as a yellow gel (23%). IR (cm⁻¹) 2919, 2342, 1733, 1646, 1540, 1472, 1019, 787; [α]_{D}²⁰−14.929 (c 0.2, CHCl₃); ¹H NMR (400 MHz, CDCl₃) δ 7.75-7.65 (m, 1H), 7.33-7.15 (m, 3H), 7.13-6.97 (m, 1H), 6.34 (d, J=15.6 Hz, 1H), 4.83 (s, 2H), 4.70 (td, J=11.2, 4.4 Hz, 1H), 1.98-1.88 (m, 1H), 1.98 (d, J=6.8 Hz, 3H), 1.72-1.50 (m, 3H), 1.50-1.33 (m, 1H), 1.30-1.17 (m, 1H), 1.06-0.73 (m, 3H), 0.85 (d, J=6.8 Hz, 3H), 0.75 (d, J=7.2 Hz, 3H), 0.64 (d, J=6.8 Hz, 3H); ¹³C NMR (100 MHz, CDCl₃) δ 166.9, 151.1, 142.8, 137.2, 135.2, 122.6, 122.5, 119.3, 116.7, 108.6, 76.4, 46.6, 45.1, 40.5, 33.9, 31.3, 26.1, 23.1, 21.8, 20.5, 18.9, 16.0; ES-API MS: m/z calcd for C₂₂H₃₀N₂O₂, found 355.2 [M+H].

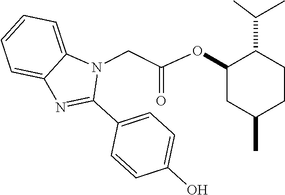

The title compound was obtained following the general procedure (Step A, method 1) described above. Purification of the residue by flash chromatography on silica gel (gradient elution, 40% EtOAc in hexanes) afforded Lqr-5-084 as a yellow solid (50%). IR (cm⁻¹) 2955, 1740, 1609, 1458, 1387, 1283, 1216; [α]_{D}²⁰−29.595 (c 0.5, CHCl₃); ¹H NMR (400 MHz, CDCl₃) δ 7.93-7.84 (m, 1H), 7.41 (d, J=8.4 Hz, 2H), 7.37 (dd, J=6.4, 3.2 Hz, 2H), 7.34-7.28 (m, 1H), 6.87 (d, J=8.8 Hz, 2H), 4.89 (d, J=4.8 Hz, 2H), 4.78 (td, J=10.8, 4.4 Hz, 1H), 1.97 (d, J=11.6 Hz, 1H), 1.72-1.56 (m, 3H), 1.53-1.39 (m, 1H), 1.38-1.22 (m, 1H), 1.08-0.78 (m, 3H), 0.89 (d, J=6.8 Hz, 3H), 0.80 (d, J=7.2 Hz, 3H), 0.68 (d, J=6.8 Hz, 3H); ¹³C NMR (100 MHz, CDCl₃) δ 166.9, 160.5, 158.7, 154.1, 145.5, 134.8, 132.3, 130.8 (2C), 123.9, 118.4, 116.7 (2C), 109.8, 46.8, 46.7, 40.5, 33.9, 31.3, 26.1, 23.1, 21.9, 20.6, 15.9; ES-API MS: m/z calcd for C₂₅H₃₀N₂O₃, found 407.2 [M+H].

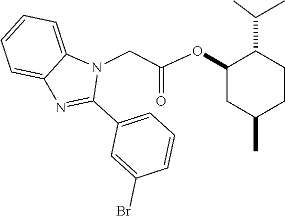

The title compound was obtained following the general procedure (Step A, method 1) described above. Purification of the residue by flash chromatography on silica gel (gradient elution, 40% EtOAc in hexanes) afforded Lqr-5-085 as a yellow solid (43%). IR (cm⁻¹) 2954, 1739, 1455, 1211, 959, 743; [α]_{D}²⁰−22.996 (c 0.8, CHCl₃); ¹H NMR (400 MHz, CDCl₃) δ 7.91 (t, J=2.0 Hz, 1H), 7.89-7.84 (m, 1H), 7.72-7.63 (m, 2H), 7.40 (d, J=7.6 Hz, 1H), 7.38-7.33 (m, 2H), 7.33-7.29 (m, 1H), 4.89 (d, J=3.2 Hz, 2H), 4.78 (td, J=10.8, 4.4 Hz, 1H), 2.02-1.93 (m, 1H), 1.72-1.38 (m, 4H), 1.34-1.22 (m, 1H), 1.09-0.76 (m, 3H), 0.90 (d, J=6.8 Hz, 3H), 0.79 (d, J=6.8 Hz, 3H), 0.68 (d, J=6.8 Hz, 3H); ¹³C NMR (100 MHz, CDCl₃) δ 166.9, 152.0, 135.7, 135.6, 133.3, 133.3, 132.2, 130.4, 127.9, 123.8, 123.4, 122.9, 120.0, 109.5, 76.7, 46.7, 46.7, 40.6, 33.9, 31.3, 26.1, 23.1, 21.9, 20.6, 16.0; ES-API MS: m/z calcd for C₂₅H₂₉BrN₂O₂, found 469.1 [M+H].

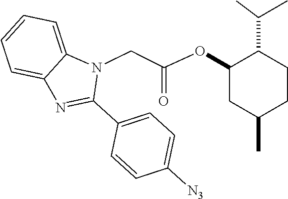

The title compound was obtained following the general procedure (Step A, method 1) described above. Purification of the residue by flash chromatography on silica gel (gradient elution, 40% EtOAc in hexanes) afforded Lqr-5-086 as a yellow solid (90%). IR (cm⁻¹) 2954, 2119, 1739, 1481, 1385, 1284, 1213, 745; [α]_{D}²⁰−24.669 (c 0.8, CHCl₃); ¹H NMR (400 MHz, CDCl₃) δ 7.94-7.84 (m, 1H), 7.77 (d, J=7.6, 2H), 7.41-7.35 (m, 2H), 7.35-7.28 (m, 1H), 7.18 (d, J=8.4 Hz, 2H), 4.90 (s, 2H), 4.78 (td, J=10.8, 4.4 Hz, 1H), 1.96 (d, J=12.0 Hz, 1H), 1.75-1.61 (m, 2H), 1.61-1.52 (m, 1H), 1.52-1.40 (m, 1H), 1.31-1.25 (m, 1H), 1.09-0.73 (m, 3H), 0.90 (d, J=6.0 Hz, 3H), 0.81 (d, J=7.6 Hz, 3H), 0.70 (d, J=7.6 Hz, 3H); ¹³C NMR (100 MHz, CDCl₃) δ 167.0, 151.4, 148.6, 142.9, 136.2, 136.1, 130.3 (2C), 124.2, 124.0 (2C), 123.5, 120.6, 109.6, 76.9, 46.8, 46.79, 40.6, 33.9, 31.3, 26.2, 23.1, 21.9, 20.6, 16.0; ES-API MS: m/z calcd for C₂₅H₂₉N₅O₂, found 432.2 [M+H].

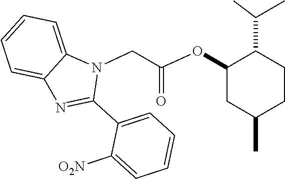

The title compound was obtained following the general procedure (Step A, method 1) described above. Purification of the residue by flash chromatography on silica gel (gradient elution, 40% EtOAc in hexanes) afforded Lqr-5-108 as a yellow solid (38%). IR (cm⁻¹) 2955, 2360, 1736, 1578, 1533, 1458, 1348, 1212, 744; [α]_{D}²⁰−30.110 (c 0.5, CHCl₃); ¹H NMR (400 MHz, CDCl₃) δ 8.28-8.13 (m, 1H), 7.85-7.78 (m, 1H), 7.77-7.71 (m, 2H), 7.71-7.65 (m, 1H), 7.40-7.29 (m, 3H), 4.79-4.63 (m, 3H), 1.89 (d, J=12.0 Hz, 1H), 1.76-1.55 (m, 2H), 1.54-1.35 (m, 2H), 1.33-1.20 (m, 1H), 1.08-0.73 (m, 3H), 0.88 (d, J=6.4 Hz, 3H), 0.77 (d, J=7.2 Hz, 3H), 0.63 (d, J=7.2 Hz, 3H); ¹³C NMR (100 MHz, CDCl₃) δ 166.8, 161.0, 149.4, 142.8, 135.0, 133.2, 133.1, 131.3, 125.4, 124.8, 123.6, 122.8, 120.3, 109.4, 85.9, 46.7, 46.1, 40.5, 33.9, 31.3, 26.0, 23.1, 21.9, 20.6, 16.0; ES-API MS: m/z calcd for C₂₅H₂₉N₃O₄, found 436.2 [M+H].

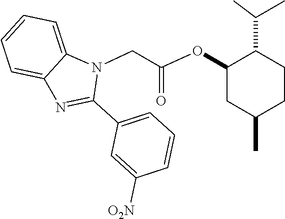

The title compound was obtained following the general procedure (Step A, method 1) described above. Purification of the residue by flash chromatography on silica gel (gradient elution, 40% EtOAc in hexanes) afforded Lqr-5-109 as a yellow solid (58%). IR (cm⁻¹) 2956, 1738, 1538, 1458, 1349, 1214, 739; [α]_{D}²⁰−24.668 (c 0.7, CHCl₃); ¹H NMR (400 MHz, CDCl₃) δ 8.63 (t, J=2.0 Hz, 1H), 8.44-8.34 (m, 1H), 8.13 (d, J=7.6 Hz, 1H), 7.89-7.83 (m, 1H), 7.72 (t, J=8.0 Hz, 1H), 7.43-7.29 (m, 3H), 4.91 (d, J=1.2 Hz, 2H), 4.78 (td, J=10.8, 4.4 Hz, 1H), 1.98 (d, J=12.0 Hz, 1H), 1.72-1.58 (m, 2H), 1.57-1.38 (m, 2H), 1.35-1.23 (m, 1H), 1.08-0.72 (m, 3H), 0.89 (d, J=6.4 Hz, 3H), 0.76 (d, J=6.8 Hz, 3H), 0.65 (d, J=6.8 Hz, 3H); ¹³C NMR (100 MHz, CDCl₃) δ 166.9, 151.2, 148.3, 142.7, 136.0, 135.3, 131.6, 130.0, 124.7, 124.0, 124.0, 123.4, 120.4, 109.6, 76.9, 46.8, 46.7, 40.5, 33.9, 31.3, 26.1, 23.1, 21.8, 20.5, 15.9; ES-API MS: m/z calcd for C₂₅H₂₉N₃O₄, found 436.2 [M+H].

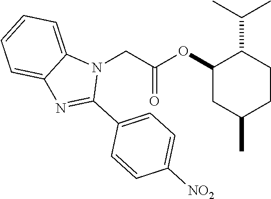

The title compound was obtained following the general procedure (Step A, method 1) described above. Purification of the residue by flash chromatography on silica gel (gradient elution, 40% EtOAc in hexanes) afforded Lqr-5-110 as a yellow solid (38%). IR (cm⁻¹) 2956, 1739, 1603, 1525, 1457, 1300, 1215, 855, 741; [α]_{D}²⁰−26.996 (c 0.5, CHCl₃); ¹H NMR (400 MHz, CDCl₃) δ 8.37 (d, J=7.6 Hz, 2H), 7.95 (d, J=7.6 Hz, 2H), 7.91-7.80 (m, 1H), 7.44-7.29 (m, 3H), 4.89 (s, 2H), 4.78 (td, J=10.8, 4.4 Hz, 1H), 1.95 (d, J=11.6 Hz, 1H), 1.74-1.62 (m, 2H), 1.62-1.40 (m, 2H), 1.38-1.20 (m, 1H), 1.09-0.75 (m, 3H), 0.90 (d, J=6.8 Hz, 3H), 0.81 (d, J=7.2 Hz, 3H), 0.68 (d, J=6.8 Hz, 3H); ¹³C NMR (100 MHz, CDCl₃) δ 167.0, 151.3, 148.5, 142.9, 136.2, 136.0, 130.2 (2C), 124.1, 124.0 (2C), 123.4, 120.5, 109.6, 76.8, 46.8, 46.7, 40.6, 33.9, 31.3, 26.2, 23.1, 21.9, 20.6, 16.0; ES-API MS: m/z calcd for C₂₅H₂₉N₃O₄, found 436.2 [M+H].

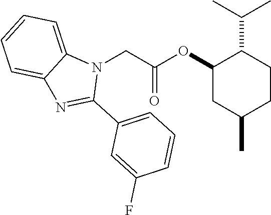

The title compound was obtained following the general procedure (Step A, method 1) described above. Purification of the residue by flash chromatography on silica gel (gradient elution, 40% EtOAc in hexanes) afforded Lqr-5-111 as a yellow solid (27%). IR (cm⁻¹) 2956, 1739, 1587, 1458, 1214, 890, 743; [α]_{D}²⁰−27.679 (c 0.35, CHCl₃); ¹H NMR (400 MHz, CDCl₃) δ 7.89-7.80 (m, 1H), 7.53-7.42 (m, 3H), 7.39-7.27 (m, 3H), 7.26-7.18 (m, 1H), 4.88 (d, J=2.4 Hz, 2H), 4.78 (td, J=10.8, 4.4 Hz, 1H), 1.97 (d, J=11.6 Hz, 1H), 1.73-1.52 (m, 3H), 1.51-1.39 (m, 1H), 1.35-1.21 (m, 1H), 1.09-0.76 (m, 3H), 0.90 (d, J=6.8 Hz, 3H), 0.80 (d, J=7.2 Hz, 3H), 0.68 (d, J=6.8 Hz, 3H); ¹³C NMR (100 MHz, CDCl₃) δ 167.2, 163.9, 152.5, 142.8, 136.0, 131.8, 130.5 (d, J=9.3 Hz, 1C), 125.0 (d, J=3.1 Hz, 1C), 123.5, 123.0, 120.2, 117.1 (d, J=20.8 Hz, 1C), 116.4 (d, J=22.9 Hz, 1C), 109.4, 76.5, 46.8, 46.6, 40.5, 33.9, 31.3, 26.1, 23.1, 21.9, 20.6, 15.9; ES-API MS: m/z calcd for C₂₅H₂₉FN₂O₂, found 409.2 [M+H].

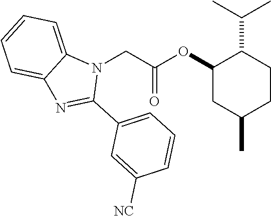

The title compound was obtained following the general procedure (Step A, method 1) described above. Purification of the residue by flash chromatography on silica gel (gradient elution, 40% EtOAc in hexanes) afforded Lqr-5-112 as a yellow solid (44%). IR (cm⁻¹) 2955, 1736, 1456, 1215, 743, 743; [α]_{D}²⁰−29.990 (c 0.6, CHCl₃); ¹H NMR (400 MHz, CDCl₃) δ 8.06 (t, J=1.2 Hz, 1H), 8.02-7.96 (m, 1H), 7.89-7.83 (m, 1H), 7.81 (dt, J=8.0, 1.2 Hz, 1H), 7.64 (t, J=8.0 Hz, 1H), 7.40-7.35 (m, 2H), 7.35-7.29 (m, 1H), 4.87 (s, 2H), 4.78 (td, J=10.8, 4.4 Hz, 1H), 2.01-1.91 (m, 1H), 1.74-1.61 (m, 2H), 1.61-1.38 (m, 2H), 1.37-1.22 (m, 2H), 1.06-0.94 (m, 2H), 0.90 (d, J=6.8 Hz, 3H), 0.81 (d, J=7.2 Hz, 3H), 0.68 (d, J=7.2 Hz, 3H); ¹³C NMR (100 MHz, CDCl₃) δ 167.0, 151.4, 142.7, 136.02, 133.4, 133.3, 132.7, 131.3, 129.7, 123.9, 123.3, 120.4, 117.8, 113.4, 109.5, 76.8, 46.8, 46.6, 40.6, 33.9, 31.3, 26.2, 23.1, 21.8, 20.6, 16.0; ES-API MS: m/z calcd for C₂₆H₂₉N₃O₂, found 416.2 [M+H].

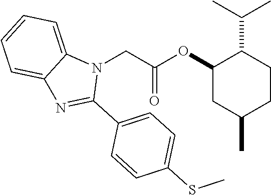

The title compound was obtained following the general procedure (Step A, method 1) described above. Purification of the residue by flash chromatography on silica gel (gradient elution, 40% EtOAc in hexanes) afforded Lqr-5-113 as a yellow solid (36%). IR (cm⁻¹) 2958, 2360, 1738, 1455, 1385, 1261, 1210, 1100, 799, 744; [α]_{D}²⁰−32.668 (c 0.5, CHCl₃); ¹H NMR (400 MHz, CDCl₃) δ 7.89-7.77 (m, 1H), 7.68-7.57 (m, 2H), 7.40-7.25 (m, 5H), 4.87 (d, J=3.2 Hz, 2H), 4.77 (td, J=10.8, 4.4 Hz, 1H), 2.54 (s, 3H), 2.02-1.92 (m, 1H), 1.74-1.54 (m, 3H), 1.53-1.40 (m, 1H), 1.35-1.20 (m, 2H), 1.09-0.92 (m, 2H), 0.90 (d, J=6.8 Hz, 3H), 0.81 (d, J=7.2 Hz, 3H), 0.69 (d, J=7.2 Hz, 3H); ¹³C NMR (100 MHz, CDCl₃) δ 167.4, 153.6, 142.9, 141.6, 136.0, 129.5 (2C), 126.0 (2C), 125.9, 123.1, 122.8, 120.0, 109.3, 76.4, 46.8, 46.7, 40.6, 33.9, 31.3, 26.1, 23.0, 21.9, 20.6, 16.0, 15.1; ES-API MS: m/z calcd for C₂₆H₃₂N₂O₂S, found 437.2 [M+H].

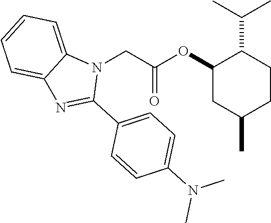

The title compound was obtained following the general procedure (Step A, method 1) described above. Purification of the residue by flash chromatography on silica gel (gradient elution, 40% EtOAc in hexanes) afforded Lqr-5-114 as a yellow solid (39%). IR (cm⁻¹) 2926, 1742, 1613, 1490, 1459, 1195, 745; [α]_{D}²⁰−29.887 (c 0.5, CHCl₃); ¹H NMR (400 MHz, CDCl₃) δ 7.86-7.76 (m, 1H), 7.64-7.54 (m, 2H), 7.37-7.19 (m, 3H), 6.82-6.74 (m, 2H), 4.87 (q, J=18.0 Hz, 2H), 4.79 (td, J=10.8, 4.4 Hz, 1H), 3.04 (s, 6H), 2.03-1.95 (m, 1H), 1.74-1.60 (m, 3H), 1.56-1.39 (m, 1H), 1.36-1.22 (m, 2H), 1.12-0.76 (m, 2H), 0.90 (d, J=6.8 Hz, 3H), 0.81 (d, J=7.2 Hz, 3H), 0.71 (d, J=7.2 Hz, 3H); ¹³C NMR (100 MHz, CDCl₃) δ 167.7, 154.9, 151.3, 143.0, 136.1, 130.3 (2C), 122.5, 122.4, 119.5 (2C), 116.6, 111.7, 109.1, 76.2, 46.9, 46.7, 40.6, 40.1 (2C), 33.9, 31.3, 26.0, 23.0, 21.9, 20.7, 16.0; ES-API MS: m/z calcd for C₂₇H₃₅N₃O₂, found 434.2 [M+H].

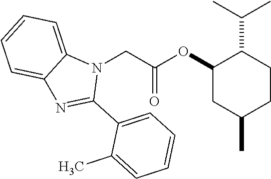

The title compound was obtained following the general procedure (Step A, method 1) described above. Purification of the residue by flash chromatography on silica gel (gradient elution, 40% EtOAc in hexanes) afforded Lqr-5-115 as a yellow solid (27%). IR (cm⁻¹) 2955, 1753, 1612, 1459, 1384, 1209, 981, 744; [α]_{D}²⁰−31.668 (c 0.3, CHCl₃); ¹H NMR (400 MHz, CDCl₃) δ 7.91-7.79 (m, 1H), 7.44-7.27 (m, 7H), 4.68 (s, 2H), 4.67 (td, J=10.8, 4.4 Hz, 1H), 2.29 (s, 3H), 1.93-1.83 (m, 1H), 1.75-1.55 (m, 2H), 1.48-1.35 (m, 2H), 1.30-1.15 (m, 1H), 1.03-0.90 (m, 1H), 0.90-0.77 (m, 2H), 0.87 (d, J=6.8 Hz, 3H), 0.74 (d, J=7.2 Hz, 3H), 0.61 (d, J=6.8 Hz, 3H); ¹³C NMR (100 MHz, CDCl₃) δ 167.0, 153.5, 142.9, 138.4, 134.9, 130.6, 130.1, 130.0, 129.2, 125.6, 123.0, 122.5, 120.1, 109.3, 76.2, 46.8, 45.9, 40.5, 33.9, 31.3, 25.8, 23.0, 21.9, 20.6, 19.7, 15.9; ES-API MS: m/z calcd for C₂₆H₃₂N₂O₂, found 405.2 [M+H].

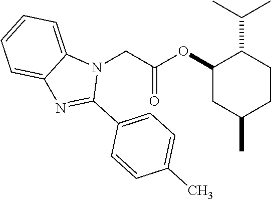

The title compound was obtained following the general procedure (Step A, method 1) described above. Purification of the residue by flash chromatography on silica gel (gradient elution, 40% EtOAc in hexanes) afforded Lqr-5-116 as a yellow solid (46%). IR (cm⁻¹) 2955, 2360, 1738, 1613, 1484, 1459, 1384, 1209, 961, 822, 743; [α]_{D}²⁰−38.776 (c 0.6, CHCl₃); ¹H NMR (400 MHz, CDCl₃) δ 7.87-7.79 (m, 1H), 7.60 (d, J=8.0 Hz, 2H), 7.36-7.26 (m, 5H), 4.87 (d, J=4.0 Hz, 2H), 4.78 (td, J=10.8, 4.4 Hz, 1H), 2.44 (s, 3H), 2.00-1.91 (m, 1H), 1.72-1.54 (m, 2H), 1.53-1.38 (m, 1H), 1.35-1.22 (m, 2H), 1.09-0.72 (m, 3H), 0.89 (d, J=6.4 Hz, 3H), 0.80 (d, J=7.2 Hz, 3H), 0.69 (d, J=7.2 Hz, 3H); ¹³C NMR (100 MHz, CDCl₃) δ 167.4, 154.1, 142.9, 140.1, 136.0, 129.4 (2C), 129.2 (2C), 126.8, 123.0, 122.7, 119.9, 109.3, 76.3, 46.8, 46.7, 40.5, 33.9, 31.3, 26.0, 23.0, 21.9, 21.4, 20.6, 15.9; ES-API MS: m/z calcd for C₂₆H₃₂N₂O₂, found 405.2 [M+H].

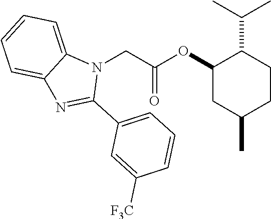

The title compound was obtained following the general procedure (Step A, method 1) described above. Purification of the residue by flash chromatography on silica gel (gradient elution, 40% EtOAc in hexanes) afforded Lqr-5-117 as a yellow solid (53%). IR (cm⁻¹) 2957, 1738, 1460, 1326, 1213, 1129, 1072, 743; [α]_{D}²⁰−29.886 (c 0.3, CHCl₃); ¹H NMR (400 MHz, CDCl₃) δ 8.04 (s, 1H), 7.93 (d, J=7.6 Hz, 1H), 7.89-7.82 (m, 1H), 7.79 (d, J=8.0 Hz, 1H), 7.66 (t, J=7.6 Hz, 1H), 7.40-7.33 (m, 2H), 7.33-7.29 (m, 1H), 4.87 (d, J=2.4 Hz, 2H), 4.77 (td, J=10.8, 4.4 Hz, 1H), 2.00-1.91 (m, 1H), 1.73-1.58 (m, 2H), 1.58-1.39 (m, 2H), 1.34-1.21 (m, 1H), 1.05-0.75 (m, 3H), 0.90 (d, J=6.8 Hz, 3H), 0.77 (d, J=7.2 Hz, 3H), 0.66 (d, J=7.2 Hz, 3H); ¹³C NMR (100 MHz, CDCl₃) δ 167.0, 152.3, 142.8, 136.0, 132.5, 131.6, 131.3, 130.77, 129.4, 126.7 (q, J=3.6 Hz, 1C), 126.3 (q, J=3.8 Hz, 1C), 123.7, 123.2, 120.3, 109.5, 77.2, 46.7, 46.7, 40.5, 33.9, 31.3, 26.1, 23.1, 21.8, 20.5, 15.9; ES-API MS: m/z calcd for C₂₆H₂₉F₃N₂O₂, found 459.2 [M+H].

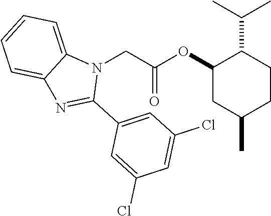

The title compound was obtained following the general procedure (Step A, method 1) described above. Purification of the residue by flash chromatography on silica gel (gradient elution, 40% EtOAc in hexanes) afforded Lqr-5-118 as a yellow solid (45%). IR (cm⁻¹) 2956, 1738, 1598, 1460, 1384, 1212, 861, 806, 742; [α]_{D}²⁰−38.667 (c 0.5, CHCl₃); ¹H NMR (400 MHz, CDCl₃) δ 7.90-7.80 (m, 1H), 7.65 (d, J=2.0 Hz, 2H), 7.51 (t, J=2.0 Hz, 1H), 7.40-7.33 (m, 2H), 7.33-7.27 (m, 1H), 4.87 (d, J=3.6 Hz, 2H), 4.79 (dt, J=10.8, 4.4 Hz, 1H), 2.03-1.93 (m, 1H), 1.74-1.53 (m, 3H), 1.53-1.39 (m, 1H), 1.35-1.22 (m, 1H), 1.09-0.73 (m, 3H), 0.89 (d, J=6.8 Hz, 3H), 0.80 (d, J=6.8 Hz, 3H), 0.68 (d, J=7.2 Hz, 3H); ¹³C NMR (100 MHz, CDCl₃) δ 166.9, 151.0, 142.7, 136.0, 135.6, 132.6, 130.0 (2C), 127.6 (2C), 123.9, 123.3, 120.4, 109.5, 76.8, 46.7, 46.7, 40.6, 33.9, 31.3, 26.1, 23.1, 21.8, 20.6, 16.0; ES-API MS: m/z calcd for C₂₅H₂₈Cl₂N₂O₂, found 459.1 [M+H].

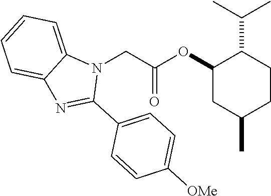

The title compound was obtained following the general procedure (Step A, method 1) described above. Purification of the residue by flash chromatography on silica gel (gradient elution, 40% EtOAc in hexanes) afforded Lqr-5-119 as a yellow solid (31%). IR (cm⁻¹) 2955, 1739, 1612, 1484, 1459, 1387, 1210, 1032, 838, 746; [α]_{D}²⁰−30.666 (c 0.3, CHCl₃); ¹H NMR (400 MHz, CDCl₃) δ 7.88-7.76 (m, 1H), 7.69-7.62 (m, 2H), 7.37-7.23 (m, 3H), 7.08-6.95 (m, 2H), 4.86 (d, J=3.6 Hz, 2H), 4.78 (td, J=10.8, 4.4 Hz, 1H), 3.88 (s, 3H), 2.01-1.91 (m, 1H), 1.73-1.54 (m, 3H), 1.53-1.38 (m, 1H), 1.36-1.21 (m, 1H), 1.09-0.75 (m, 3H), 0.90 (d, J=6.8 Hz, 3H), 0.80 (d, J=7.2 Hz, 3H), 0.69 (d, J=7.2 Hz, 3H); ¹³C NMR (100 MHz, CDCl₃) δ 167.5, 160.9, 154.0, 142.9, 136.0, 130.7 (2C), 122.9, 122.7, 122.0, 119.8, 114.2 (2C), 109.3, 76.3, 55.3, 46.8, 46.7, 40.6, 33.9, 31.3, 26.0, 23.0, 21.9, 20.6, 16.0; ES-API MS: m/z calcd for C₂₆H₃₂N₂O₃, found 421.2 [M+H].

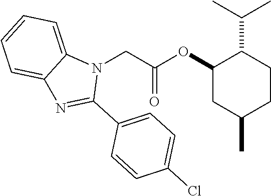

The title compound was obtained following the general procedure (Step A, method 2) described above. Purification of the residue by flash chromatography on silica gel (gradient elution, 015% EtOAc in hexanes) afforded LW-V-240 as a white solid (45%). IR (cm⁻¹) 2956, 2870, 1738, 1458, 1212, 1095, 744; [α]_{D}²⁰−33.32 (c 0.45, CHCl₃); ¹H NMR (400 MHz, CDCl₃) δ 7.82 (m, 1H), 7.65 (d, J=8.4 Hz, 2H), 7.47 (d, J=8.4 Hz, 2H), 7.32 (m, 2H), 7.27 (m, 1H), 4.86 (d, J=17.6 Hz, 1H), 4.81 (d, J=18.0 Hz, 1H), 4.76 (ddd, J=11.2, 10.8, 4.4 Hz, 1H), 1.94 (m, 1H), 1.68-1.61 (m, 2H), 1.56 (m, 1H), 1.45 (m, 1H), 1.28 (m, 1H), 1.00 (m, 1H), 0.93 (m, 1H), 0.89 (d, J=6.4 Hz, 3H), 0.84 (m, 1H), 0.80 (d, J=6.8 Hz, 3H), 0.67 (d, J=6.8 Hz, 3H); ¹³C NMR (100 MHz, CDCl₃) δ 167.4, 153.0, 143.0, 136.5, 136.2, 130.8 (2C), 129.3 (2C), 128.5, 123.6, 123.2, 120.3, 109.6, 76.7, 47.0, 46.8, 40.8, 34.1, 31.5, 26.3, 23.3, 22.1, 20.8, 16.2; ES-API MS: m/z calcd for C₂₅H₂₉ClN₂O₂, found 425.1 [M+H].

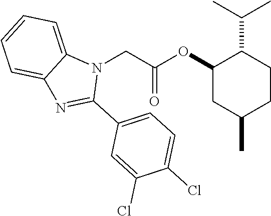

The title compound was obtained following the general procedure (Step A, method 2) described above. Purification of the residue by flash chromatography on silica gel (gradient elution, 15% EtOAc in hexanes) afforded LW-V-241 as a white solid (36%). IR (cm⁻¹) 2956, 2870, 1738, 1455, 1213, 743; [α]_{D}²⁰−30.86 (c 0.46, CHCl₃); ¹H NMR (400 MHz, CDCl₃) δ 7.83 (dd, J=1.2, 1.2 Hz, 1H), 7.80 (m, 1H), 7.54 (m, 2H), 7.31 (m, 2H), 7.26 (m, 1H), 4.85 (d, J=18.0 Hz, 1H), 4.81 (d, J=18.0 Hz, 1H), 4.76 (ddd, J=10.8, 10.8, 4.4 Hz, 1H), 1.94 (m, 1H), 1.67-1.60 (m, 2H), 1.55 (m, 1H), 1.44 (m, 1H), 1.28 (m, 1H), 1.04-0.93 (m, 2H), 0.88 (d, J=6.4 Hz, 3H), 0.83 (m, 1H), 0.79 (d, J=7.2 Hz, 3H), 0.66 (d, J=6.8 Hz, 3H); ¹³C NMR (100 MHz, CDCl₃) δ 167.2, 151.5, 142.9, 136.2, 134.7, 133.4, 131.2, 131.0, 129.9, 128.5, 123.9, 123.3, 120.4, 109.6, 76.8, 46.9, 46.8, 40.8, 34.1, 31.5, 26.3, 23.3, 22.0, 20.8, 16.2; ES-API MS: m/z calcd for C₂₅H₂₈Cl₂N₂O₂, found 459.1 [M+H].

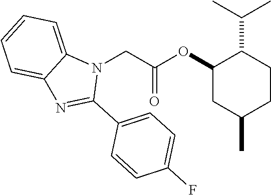

The title compound was obtained following the general procedure (Step A, method 2) described above. Purification of the residue by flash chromatography on silica gel (gradient elution, 15% EtOAc in hexanes) afforded LW-V-242 as a white solid (36%). IR (cm⁻¹) 2956, 2870, 1741, 1484, 1458, 1212, 1158, 745; [α]_{D}²⁰−31.99 (c 0.45, CHCl₃); ¹H NMR (400 MHz, CDCl₃) δ 7.81 (m, 1H), 7.70 (d, J=8.8 Hz, 1H), 7.69 (d, J=8.8 Hz, 1H), 7.31 (m, 2H), 7.27 (m, 1H), 7.19 (d, J=8.8 Hz, 1H), 7.17 (d, J=8.8 Hz, 1H), 4.85 (d, J=18.0 Hz, 1H), 4.81 (d, J=18.0 Hz, 1H), 4.76 (ddd, J=10.8, 11.2, 4.4 Hz, 1H), 1.94 (m, 1H), 1.68-1.61 (m, 2H), 1.57 (m, 1H), 1.45 (m, 1H), 1.28 (m, 1H), 1.00 (m, 1H), 0.95 (m, 1H), 0.89 (d, J=6.4 Hz, 3H), 0.84 (m, 1H), 0.79 (d, J=6.8 Hz, 3H), 0.67 (d, J=6.8 Hz, 3H); ¹³C NMR (100 MHz, CDCl₃) δ 167.3, 163.8 (d, J=249.5 Hz, 1C), 153.0, 142.8, 136.0, 131.4 (d, J=8.5 Hz, 2C), 126.0 (d, J=3.4 Hz, 1C), 123.3, 122.9, 120.1, 116.0 (d, J=21.8 Hz, 2C), 109.4, 76.5, 46.8, 46.6, 40.6, 33.9, 31.3, 26.1, 23.1, 21.9, 20.6, 16.0; ES-API MS: m/z calcd for C₂₅H₂₉FN₂O₂, found 409.2 [M+H].

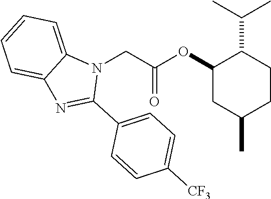

The title compound was obtained following the general procedure (Step A, method 2) described above. Purification of the residue by flash chromatography on silica gel (gradient elution, 15% EtOAc in hexanes) afforded LW-V-243 as a white solid (55%). IR (cm⁻¹) 2958, 2872, 1739, 1324, 1213, 1168, 1129, 742; [α]_{D}²⁰−28.45 (c 0.52, CHCl₃); ¹H NMR (400 MHz, CDCl₃) δ 7.83-7.79 (m, 3H), 7.72 (m, 2H), 7.32-7.24 (m, 3H), 4.83 (s, 2H), 4.73 (ddd, J=10.8, 11.2, 4.4 Hz, 1H), 1.91 (m, 1H), 1.66-1.58 (m, 2H), 1.54 (m, 1H), 1.42 (m, 1H), 1.26 (m, 1H), 0.98 (m, 1H), 0.91 (m, 1H), 0.86 (d, J=6.4 Hz, 3H), 0.82 (m, 1H), 0.77 (d, J=7.2 Hz, 3H), 0.64 (d, J=6.8 Hz, 3H); ¹³C NMR (100 MHz, CDCl₃) δ 167.3, 152.4, 143.0, 136.2, 133.6, 132.0 (q, J=32.6 Hz, 1C), 129.8 (3C), 125.9 (q, J=3.7 Hz, 1C), 123.9 (q, J=271.1 Hz, 1C), 123.9, 123.3, 120.5, 109.7, 76.7, 46.9, 46.7, 40.7, 34.1, 31.5, 26.3, 23.2, 22.0, 20.7, 16.1; ES-API MS: m/z calcd for C₂₆H₂₉F₃N₂O₂, found 459.2 [M+H].

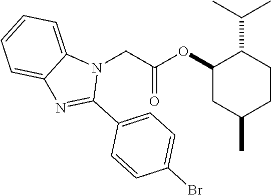

The title compound was obtained following the general procedure (Step A, method 2) described above. Purification of the residue by flash chromatography on silica gel (gradient elution, 15% EtOAc in hexanes) afforded LW-V-244 as a white solid (59%). IR (cm⁻¹) 2955, 2870, 1737, 1457, 1211, 1012, 743; [α]_{D}²⁰−28.83 (c 0.43, CHCl₃); ¹H NMR (400 MHz, CDCl₃) δ 7.79 (m, 1H), 7.60 (d, J=8.4 Hz, 2H), 7.55 (d, J=8.8 Hz, 2H), 7.28 (m, 2H), 7.24 (m, 1H), 4.82 (d, J=18.0 Hz, 1H), 4.78 (d, J=18.0 Hz, 1H), 4.73 (ddd, J=10.8, 10.8, 4.4 Hz, 1H), 1.91 (m, 1H), 1.66-1.98 (m, 2H), 1.54 (m, 1H), 1.42 (m, 1H), 1.26 (m, 1H), 0.97 (m, 1H), 0.91 (m, 1H), 0.87 (d, J=6.4 Hz, 3H), 0.81 (m, 1H), 0.78 (d, J=6.8 Hz, 3H), 0.65 (d, J=6.8 Hz, 3H); ¹³C NMR (100 MHz, CDCl₃) δ 167.3, 152.9, 143.0, 136.1, 132.2 (2C), 130.9 (2C), 128.9, 124.8, 123.6, 123.1, 120.3, 109.6, 76.6, 46.9, 46.7, 40.7, 34.1, 31.5, 26.3, 23.2, 22.1, 20.8, 16.1; ES-API MS: m/z calcd for C₂₅H₂₉BrN₂O₂, found 469.1 [M+H].

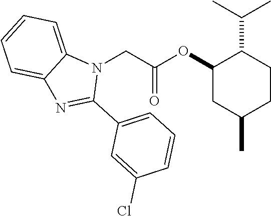 [SMILES: CC(C)[C@@H]1CC[C@@H](C)C[C@H]1OC(=O)Cn1c(-c2cccc(Cl)c2)nc2ccccc21]

The title compound was obtained following the general procedure (Step A, method 2) described above. Purification of the residue by flash chromatography on silica gel (gradient elution, 15% EtOAc in hexanes) afforded LW-V-245 as a white solid (60%). IR (cm⁻¹) 2956, 2870, 1739, 1456, 1212, 742; [α]_{D}²⁰−30.36 (c 0.54, CHCl₃); ¹H NMR (400 MHz, CDCl₃) δ 7.79 (m, 1H), 7.70 (m, 1H), 7.55 (ddd, J=7.6, 2.0, 1.2 Hz, 1H), 7.44 (ddd, J=8.8, 2.0, 1.2 Hz, 1H), 7.38 (dd, J=7.6, 8.0 Hz, 1H), 7.28 (m, 2H), 7.24 (m, 1H), 4.84 (d, J=18.0 Hz, 1H), 4.79 (d, J=18.0 Hz, 1H), 4.74 (ddd, J=10.8, 10.8, 4.4 Hz, 1H), 1.93 (m, 1H), 1.65-1.57 (m, 2H), 1.54 (m, 1H), 1.41 (m, 1H), 1.25 (m, 1H), 0.96 (m, 1H), 0.92 (m, 1H), 0.86 (d, J=6.4 Hz, 3H), 0.81 (m, 1H), 0.76 (d, J=7.2 Hz, 3H), 0.64 (d, J=6.8 Hz, 3H); ¹³C NMR (100 MHz, CDCl₃) δ 167.3, 152.5, 142.9, 136.1, 135.0, 131.7, 130.23, 130.17, 129.5, 127.5, 123.6, 123.1, 120.3, 109.6, 76.6, 46.9, 46.7, 40.7, 34.0, 31.4, 26.2, 23.2, 22.0, 20.7, 16.1; ES-API MS: m/z calcd for C₂₅H₂₉ClN₂O₂, found 425.1 [M+H].

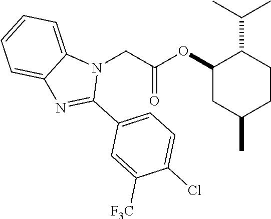 [SMILES: CC(C)[C@@H]1CC[C@@H](C)C[C@H]1OC(=O)Cn1c(-c2ccc(Cl)c(C(F)(F)F)c2)nc2ccccc21]

The title compound was obtained following the general procedure (Step A, method 2) described above. Purification of the residue by flash chromatography on silica gel (gradient elution, 15% EtOAc in hexanes) afforded LW-V-246 as a white solid (62%). IR (cm⁻¹) 2957, 2872, 1739, 1456, 1313, 1214, 1180, 1144, 1037, 744; [α]_{D}²⁰−33.77 (c 0.45, CHCl₃); ¹H NMR (400 MHz, CDCl₃) δ 8.08 (d, J=2.0 Hz, 1H), 7.81-7.77 (m, 2H), 7.59 (d, J=8.4 Hz, 1H), 7.30 (m, 2H), 7.26 (m, 1H), 4.83 (d, J=18.0 Hz, 1H), 4.78 (d, J=18.0 Hz, 1H), 4.73 (ddd, J=10.8, 10.8, 4.4 Hz, 1H), 1.91 (m, 1H), 1.66-1.57 (m, 2H), 1.50 (m, 1H), 1.42 (m, 1H), 1.26 (m, 1H), 0.97 (m, 1H), 0.92 (m, 1H), 0.86 (d, J=6.4 Hz, 3H), 0.82 (m, 1H), 0.75 (d, J=6.8 Hz, 3H), 0.62 (d, J=6.8 Hz, 3H); ¹³C NMR (100 MHz, CDCl₃) δ 167.0, 151.3, 142.7, 136.0, 134.2 (q, J=1.6 Hz, 1C), 133.3, 132.0, 129.1 (q, J=31.7 Hz, 1C), 128.9, 128.5 (q, J=5.3 Hz, 1C), 123.9, 123.3, 122.4 (q, J=272.3 Hz, 1C), 120.3, 109.5, 76.8, 46.8, 46.6, 40.5, 33.9, 31.3, 26.2, 23.1, 21.8, 20.5, 15.9; ES-API MS: m/z calcd for C₂₆H₂₈Cl F₃N₂O₂, found 493.1 [M+H].

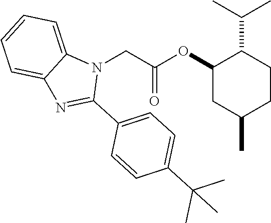 [SMILES: CC(C)[C@@H]1CC[C@@H](C)C[C@H]1OC(=O)Cn1c(-c2ccc(C(C)(C)C)cc2)nc2ccccc21]

The title compound was obtained following the general procedure (Step A, method 2) described above. Purification of the residue by flash chromatography on silica gel (gradient elution, 15% EtOAc in hexanes) afforded LW-V-247 as a white solid (55%). IR (cm⁻¹) 2957, 2870, 1741, 1458, 1210, 741; [α]_{D}²⁰−29.43 (c 0.36, CHCl₃); ¹H NMR (400 MHz, CDCl₃) δ 7.81 (m, 1H), 7.62 (d, J=8.4 Hz, 2H), 7.49 (d, J=8.4 Hz, 2H), 7.31-7.23 (m, 3H), 4.88 (d, J=18.0 Hz, 1H), 4.83 (d, J=18.0 Hz, 1H), 4.76 (ddd, J=11.2, 11.2, 4.4 Hz, 1H), 1.96 (m, 1H), 1.67-1.56 (m, 3H), 1.44 (m, 1H), 1.35 (s, 9H), 1.27 (m, 1H), 0.99 (m, 1H), 0.93 (m, 1H), 0.88 (d, J=6.4 Hz, 3H), 0.82 (m, 1H), 0.78 (d, J=6.8 Hz, 3H), 0.68 (d, J=6.8 Hz, 3H); ¹³C NMR (100 MHz, CDCl₃) δ 167.7, 154.2, 153.4, 143.1, 136.2, 129.2 (2C), 127.0, 125.9 (2C), 123.1, 122.8, 120.1, 109.5, 76.4, 46.93, 46.86, 40.7, 35.0, 34.1, 31.5, 31.4 (3C), 26.2, 23.2, 22.1, 20.8, 16.2; ES-API MS: m/z calcd for C₂₉H₃₈N₂O₂, found 447.3 [M+H].

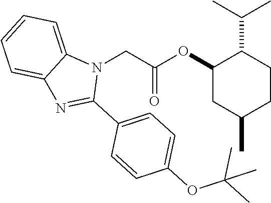 [SMILES: CC(C)[C@@H]1CC[C@@H](C)C[C@H]1OC(=O)Cn1c(-c2ccc(OC(C)(C)C)cc2)nc2ccccc21]

The title compound was obtained following the general procedure (Step A, method 2) described above. Purification of the residue by flash chromatography on silica gel (gradient elution, 15% EtOAc in hexanes) afforded LW-V-248 as a white solid (62%). IR (cm⁻¹) 2956, 2871, 1740, 1483, 1458, 1209, 1160, 743; [α]_{D}²⁰−28.62 (c 0.58, CHCl₃); NMR (400 MHz, CDCl₃) δ 7.75 (m, 1H), 7.56 (d, J=8.8 Hz, 2H), 7.26-7.18 (m, 3H), 7.05 (d, J=8.8 Hz, 2H), 4.83 (d, J=17.6 Hz, 1H), 4.77 (d, J=17.6 Hz, 1H), 4.72 (ddd, J=10.8, 10.8, 4.4 Hz, 1H), 1.92 (m, 1H), 1.63-1.50 (m, 3H), 1.41 (m, 1H), 1.34 (s, 9H), 1.23 (m, 1H), 0.96 (m, 1H), 0.89 (m, 1H), 0.83 (d, J=6.4 Hz, 3H), 0.78 (m, 1H), 0.75 (d, J=6.8 Hz, 3H), 0.64 (d, J=6.8 Hz, 3H); ¹³C NMR (100 MHz, CDCl₃) δ 167.5, 157.3, 153.9, 143.0, 136.1, 130.2 (2C), 124.4, 124.0 (2C), 123.0, 122.8, 119.9, 109.4, 79.3, 76.3, 46.8, 46.7, 40.6, 34.0, 31.4, 28.9 (3C), 26.1, 23.2, 22.0, 20.7, 16.1; ES-API MS: m/z calcd for C₂₉H₃₈N₂O₃, found 463.2 [M+H].

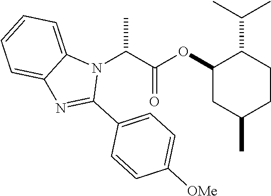 [SMILES: COc1ccc(-c2nc3ccccc3n2[C@H](C)C(=O)O[C@@H]2C[C@H](C)CC[C@H]2C(C)C)cc1]

The title compound was obtained following the general procedure (Step A, method 1) described above. Purification of the residue by flash chromatography on silica gel (gradient elution, 40% EtOAc in hexanes) afforded LW-III-308 as a pale yellow gel (28%). IR (cm⁻¹) 3056, 2957, 2871, 1732, 1614, 1486, 1455, 1253, 1033, 747; [α]_{D}²⁰+14.03 (c 0.67, CHCl₃); ¹H NMR (400 MHz, CDCl₃) δ 7.80 (d, J=7.6 Hz, 1H), 7.63 (d, J=8.8 Hz, 2H), 7.36 (d, J=7.2 Hz, 1H), 7.28 (dd, J=7.2, 6.4 Hz, 1H), 7.23 (ddd, J=8.0, 7.6, 1.6 Hz, 1H), 7.02 (d, J=8.8 Hz, 2H), 5.24 (q, J=7.2 Hz, 1H), 4.72 (ddd, J=10.8, 10.8, 4.4 Hz, 1H), 3.87 (s, 3H), 1.75 (d, J=7.6 Hz, 3H), 1.77 (m, 2H), 1.54-1.58 (m, 2H), 1.39 (m, 1H), 1.25 (m, 1H), 0.99 (m, 1H), 0.86 (d, J=7.2 Hz, 3H), 0.78 (d, J=6.4 Hz, 3H), 0.72 (m, 1H), 0.73 (d, J=6.8 Hz, 3H), 0.57 (m, 1H); ¹³C NMR (100 MHz, CDCl₃) δ 169.8, 161.1, 154.3, 143.4, 134.0, 131.1 (2C), 122.8, 122.7, 122.4, 120.1, 114.4 (2C), 111.6, 76.5, 55.6, 54.4, 46.9, 40.3, 34.0, 31.4, 26.5, 23.4, 22.0, 20.9, 16.4, 16.3; ES-API MS: m/z calcd for C₂₇H₃₄N₂O₃, found 435.3 [M+H].

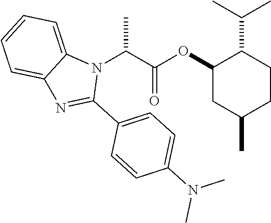 [SMILES: CC(C)[C@@H]1CC[C@@H](C)C[C@H]1OC(=O)[C@@H](C)n1c(-c2ccc(N(C)C)cc2)nc2ccccc21]

The title compound was obtained following the general procedure (Step A, method 1) described above. Purification of the residue by flash chromatography on silica gel (gradient elution, 40% EtOAc in hexanes) afforded LW-III-309 as a pale yellow gel (30%). IR (cm⁻¹) 2955, 1738, 1614, 1494, 1454, 1368, 1224, 1198, 821, 746; [α]_{D}²⁰+49.20 (c 0.89, CHCl₃); ¹H NMR (400 MHz, CDCl₃) δ 7.79 (d, J=7.6 Hz, 1H), 7.58 (d, J=8.8 Hz, 2H), 7.34 (d, J=8.0 Hz, 2H), 7.26 (dd, J=7.6, 6.4 Hz, 1H), 7.21 (dd, J=7.6, 7.2 Hz, 1H), 6.79 (d, J=8.4 Hz, 2H), 5.32 (q, J=7.2 Hz, 1H), 4.72 (ddd, J=11.2, 10.8, 4.4 Hz, 1H), 3.04 (s, 6H), 1.83-1.75 (m, 2H), 1.74 (d, J=7.2 Hz, 3H), 1.54-1.60 (m, 2H), 1.41 (m, 1H), 1.25 (m, 1H), 1.00 (m, 1H), 0.87 (d, J=7.2 Hz, 3H), 0.79 (d, J=6.4 Hz, 3H), 0.74 (d, J=7.2 Hz, 3H), 0.59 (m, 1H); ¹³C NMR (100 MHz, CDCl₃) δ 169.9, 155.0, 151.3, 143.4, 133.9, 130.5 (2C), 122.24, 122.18, 119.7, 116.9, 111.8 (2C), 111.3, 76.2, 54.3, 46.7, 40.2 (2C), 40.1, 33.9, 31.2, 26.3, 23.2, 21.8, 20.7, 16.22, 16.16; ES-API MS: m/z calcd for C₂₈H₃₇N₃O₂, found 448.3 [M+H].

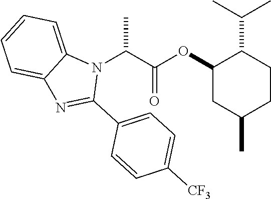 [SMILES: CC(C)[C@@H]1CC[C@@H](C)C[C@H]1OC(=O)[C@@H](C)n1c(-c2ccc(C(F)(F)F)cc2)nc2ccccc21]

The title compound was obtained following the general procedure (Step A, method 1) described above. Purification of the residue by flash chromatography on silica gel (gradient elution, 15% EtOAc in hexanes) afforded LW-III-310 as a pale yellow gel (54%). IR (cm⁻¹) 2958, 2872, 1738, 1454, 1324, 1128, 850, 745; [α]_{D}²⁰+5.33 (c 0.30, CHCl₃); NMR (400 MHz, CDCl₃) δ 7.85 (d, J=7.6 Hz, 2H), 7.85 (m, 1H), 7.79 (d, J=8.4 Hz, 2H), 7.41 (dd, J=7.2, 1.6 Hz, 1H), 7.33 (ddd, J=7.2, 7.2, 1.6 Hz, 1H), 7.30 (ddd, J=7.2, 7.2, 1.6 Hz, 1H), 5.19 (q, J=7.2 Hz, 1H), 4.74 (ddd, J=11.2, 10.8, 4.4 Hz, 1H), 1.79 (d, J=7.2 Hz, 3H), 1.74 (m, 1H), 1.86-1.61 (m, 2H), 1.40 (m, 1H), 1.26 (m, 1H), 1.01 (m, 1H), 0.88 (d, J=7.2 Hz, 3H), 0.79 (d, J=6.4 Hz, 3H), 0.77 (m, 1H), 0.74 (d, J=7.2 Hz, 3H), 0.73 (m, 1H), 0.59 (m, 1H); ¹³C NMR (100 MHz, CDCl₃) δ 169.3, 152.6, 143.4, 134.1, 133.9, 132.2 (q, J=32.6 Hz, 1C), 130.2 (2C), 126.0 (q, J=3.7 Hz, 1C), 124.0 (q, J=271 Hz, 1C), 123.6, 123.2, 120.6, 111.9, 76.8, 54.6, 47.0, 40.3, 34.0, 31.4, 26.7, 23.4, 22.0, 20.9, 16.4 (2C); ES-API MS: m/z calcd for C₂₇H₃₁F₃N₂O₂, found 473.3 [M+H].

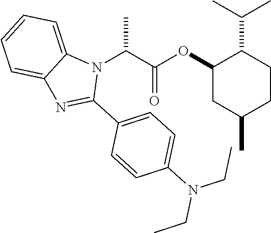 [SMILES: CCN(CC)c1ccc(-c2nc3ccccc3n2[C@H](C)C(=O)O[C@@H]2C[C@H](C)CC[C@H]2C(C)C)cc1]

The title compound was obtained following the general procedure (Step A, method 1) described above. Purification of the residue by flash chromatography on silica gel (gradient elution, 45% EtOAc in hexanes) afforded LW-IV-55 as a pale yellow gel (21%). IR (cm⁻¹) 2957, 2929, 2870, 1735, 1614, 1489, 1455, 1198, 746; [α]_{B}²⁰+39.99 (c 1.09, CHCl₃); NMR (400 MHz, CDCl₃) δ 7.78 (d, J=7.6 Hz, 1H), 7.54 (d, J=8.8 Hz, 2H), 7.33 (d, J=8.0 Hz, 1H), 7.26-7.18 (m, 2H), 6.74 (d, J=8.8 Hz, 2H), 5.36 (q, J=7.2 Hz, 1H), 4.72 (ddd, J=11.2, 10.8, 4.0 Hz, 1H), 3.42 (q, J=7.2 Hz, 4H), 1.79 (m, 1H), 1.75 (d, J=7.2 Hz, 3H), 1.61 (m, 2H), 1.39 (m, 1H), 1.25 (m, 1H), 1.21 (t, J=6.8 Hz, 6H), 1.15 (m, 1H), 1.00 (m, 1H), 0.87 (d, J=6.8 Hz, 3H), 0.79 (d, J=6.8 Hz, 3H), 0.75 (d, J=6.8 Hz, 3H), 0.70 (m, 1H), 0.60 (m, 1H); ¹³C NMR (100 MHz, CDCl₃) δ 170.2, 155.5, 148.9, 143.8, 134.1, 130.9 (2C), 122.3, 122.2, 119.8, 116.2, 111.5, 111.3 (2C), 76.3, 54.5, 46.9, 44.6 (2C), 40.3, 34.1, 31.4, 26.5, 23.4, 22.0, 20.9, 16.41, 16.36, 12.7 (2C); ES-API MS: m/z calcd for C₃₀H₄₁N₃O₂, found 476.3 [M+H].

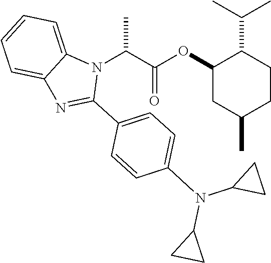 [SMILES: CC(C)[C@@H]1CC[C@@H](C)C[C@H]1OC(=O)[C@@H](C)n1c(-c2ccc(N(C3CC3)C3CC3)cc2)nc2ccccc21]

The title compound was obtained following the general procedure (Step A, method 1) described above. Purification of the residue by flash chromatography on silica gel (gradient elution, 30% EtOAc in hexanes) afforded LW-IV-92 as a pale yellow gel (17%). IR (cm⁻¹) 3087, 3009, 2956, 2870, 1738, 1614, 1486, 1455, 1371, 1346, 746; [α]_{D}²⁰+43.90 (c 1.69, CHCl₃); ¹H NMR (400 MHz, CDCl₃) δ 7.79 (d, J=7.6 Hz, 1H), 7.55 (d, J=8.8 Hz, 2H), 7.34 (d, J=8.0 Hz, 1H), 7.26 (dd, J=5.2, 8.8 Hz, 1H), 7.21 (dd, J=8.0, 7.2 Hz, 1H), 7.12 (d, J=8.8 Hz, 2H), 5.26 (q, J=7.2 Hz, 1H), 4.72 (ddd, J=10.8, 10.8, 4.4 Hz, 1H), 2.53 (m, 2H), 1.79 (m, 2H), 1.75 (d, J=7.2 Hz, 3H), 1.66-1.57 (m, 2H), 1.40 (m, 1H), 1.30-1.22 (m, 2H), 1.01 (m, 1H), 0.95-0.88 (m, 4H), 0.87 (d, J=6.8 Hz, 3H), 0.79 (d, J=6.4 Hz, 3H), 0.76-0.73 (m, 6H), 0.71 (m, 1H), 0.60 (m, 1H); ¹³C NMR (100 MHz, CDCl₃) δ 170.2, 155.5, 151.3, 143.8, 134.1, 129.9 (2C), 122.4, 122.3, 119.9, 117.9, 114.1 (2C), 111.5, 76.4, 54.5, 46.9, 40.3, 34.1, 31.4, 30.8 (2C), 26.5, 23.4, 22.0, 21.0, 16.5, 16.4, 9.5 (4C); ES-API MS: m/z calcd for C₃₂H₄₁N₃O₂, found 500.3 [M+H].

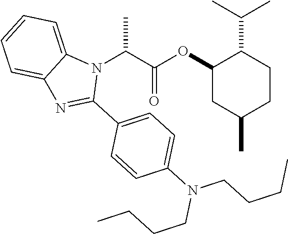 [SMILES: CCCCN(CCCC)c1ccc(-c2nc3ccccc3n2[C@H](C)C(=O)O[C@@H]2C[C@H](C)CC[C@H]2C(C)C)cc1]

The title compound was obtained following the general procedure (Step A, method 1) described above. Purification of the residue by flash chromatography on silica gel (gradient elution, 30% EtOAc/hexanes) afforded LW-IV-93 as a pale yellow gel (12%). IR (cm⁻¹) 2956, 2872, 1738, 1614, 1489, 1455, 1369, 1221, 1198, 818, 746; [α]_{D}²⁰+50.73 (c 1.36, CHCl₃); ¹H NMR (400 MHz, CDCl₃) δ 7.78 (d, J=8.0 Hz, 1H), 7.53 (d, J=8.8 Hz, 2H), 7.33 (d, J=8.0 Hz, 1H), 7.25 (dd, J=8.0, 7.2 Hz, 1H), 7.19 (dd, J=8.0, 7.2 Hz, 1H), 6.69 (d, J=8.8 Hz, 2H), 5.36 (q, J=7.2 Hz, 1H), 4.72 (ddd, J=10.8, 10.8, 4.0 Hz, 1H), 3.33 (m, 4H), 1.79 (m, 2H), 1.74 (d, J=7.6 Hz, 3H), 1.66-1.55 (m, 5H), 1.42-1.33 (m, 4H), 1.30-1.22 (m, 2H), 1.02 (m, 1H), 0.97 (m, 6H), 0.91 (m, 1H), 0.87 (d, J=7.2 Hz, 3H), 0.79 (d, J=6.4 Hz, 3H), 0.75 (d, J=6.8 Hz, 3H), 0.72 (m, 1H), 0.60 (m, 1H); ¹³C NMR (100 MHz, CDCl₃) δ 170.0, 155.3, 149.1, 143.6, 133.9, 130.6 (2C), 122.1, 122.0, 119.6, 115.8, 111.3, 111.2 (2C), 76.2, 54.3, 50.7 (2C), 46.7, 40.1, 33.9, 31.2, 29.3 (2C), 26.3, 23.2, 21.8, 20.8, 20.3 (2C), 16.2, 16.1, 14.0 (2C); ES-API MS: m/z calcd for C₃₄H₄₉N₃O₂, found 532.4 [M+H].

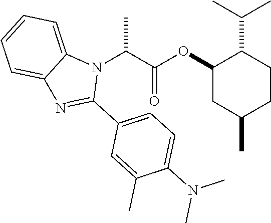 [SMILES: Cc1cc(-c2nc3ccccc3n2[C@H](C)C(=O)O[C@@H]2C[C@H](C)CC[C@H]2C(C)C)ccc1N(C)C]

The title compound was obtained following the general procedure (Step A, method 1) described above. Purification of the residue by flash chromatography on silica gel (gradient elution, 45% EtOAc in hexanes) afforded LW-IV-74 as a pale yellow gel (36%). IR (cm⁻¹) 2954, 2870, 2781, 1738, 1614, 1486, 1455, 1220, 747; [α]_{D}²⁰+21.80 (c 2.00, CHCl₃); ¹H NMR (400 MHz, CDCl₃) δ 7.79 (d, J=7.6 Hz, 1H), 7.50 (s, 1H), 7.44 (dd, J=8.4, 1.6 Hz, 1H), 7.35 (d, J=7.6 Hz, 1H), 7.23 (m, 2H), 7.08 (d, J=8.4 Hz, 1H), 5.32 (q, J=7.2 Hz, 1H), 4.73 (ddd, J=10.8, 10.8, 4.4 Hz, 1H), 2.76 (s, 6H), 2.37 (s, 3H), 1.85-1.74 (m, 2H), 1.73 (d, J=7.2 Hz, 3H), 1.65-1.56 (m, 2H), 1.39 (m, 1H), 1.28-1.22 (m, 2H), 0.99 (m, 1H), 0.86 (d, J=6.8 Hz, 3H), 0.78 (d, J=6.4 Hz, 3H), 0.74 (d, J=6.8 Hz, 3H), 0.59 (m, 1H); ¹³C NMR (100 MHz, CDCl₃) δ 169.8, 154.5, 154.3, 143.5, 133.9, 132.4, 132.0, 127.6, 123.4, 122.4, 122.3, 119.9, 118.1, 111.4, 76.2, 54.3, 46.7, 43.8 (2C), 40.1, 33.9, 31.2, 26.3, 23.1, 21.8, 20.8, 18.8, 16.18, 16.17; ES-API MS: m/z calcd for C₂₉H₃₉N₃O₂, found 462.3 [M+H].

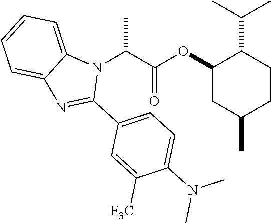 [SMILES: CC(C)[C@@H]1CC[C@@H](C)C[C@H]1OC(=O)[C@@H](C)n1c(-c2ccc(N(C)C)c(C(F)(F)F)c2)nc2ccccc21]

The title compound was obtained following the general procedure (Step A, method 1) described above. Purification of the residue by flash chromatography on silica gel (gradient elution, 45% EtOAc in hexanes) afforded LW-IV-68 as a pale yellow gel (38%). IR (cm⁻¹-) 3368, 2956, 2872, 2360, 1739, 1622, 1456, 1128, 1109, 1050, 748; [α]_{D}²⁰+16.44 (c 1.07, CHCl₃); ¹H NMR (400 MHz, CDCl₃) δ 7.94 (d, J=2.0 Hz, 1H), 7.82 (dd, J=6.8, 1.2 Hz, 1H), 7.80 (dd, J=8.4, 2.0 Hz, 1H), 7.41 (dd, J=7.2, 1.2 Hz, 1H), 7.34 (d, J=8.8 Hz, 1H), 7.30 (ddd, J=7.2, 7.6, 1.2 Hz, 1H), 7.26 (ddd, J=8.0, 7.2, 1.2 Hz, 1H), 5.21 (q, J=7.6 Hz, 1H), 4.75 (ddd, J=10.8, 10.8, 4.4 Hz, 1H), 2.87 (s, 6H), 1.77 (d, J=7.2 Hz, 3H), 1.73 (m, 1H), 1.66-1.58 (m, 2H), 1.40 (m, 1H), 1.30-1.23 (m, 2H), 1.00 (m, 1H), 0.86 (d, J=6.8 Hz, 3H), 0.79 (d, J=6.8 Hz, 3H), 0.77 (m, 1H), 0.74 (d, J=6.8 Hz, 3H), 0.61 (q, J=11.2 Hz, 1H); ¹³C NMR (100 MHz, CDCl₃) δ 169.4, 154.3, 152.8, 143.3, 133.8, 133.5, 129.3 (q, J=5.0 Hz, 1C), 123.2 (q, J=135.0 Hz, 1C), 123.6, 122.9, 122.7, 121.7, 120.2, 111.7, 76.5, 54.4, 46.6, 45.0, 40.1, 33.8, 31.2, 26.3, 23.1, 21.8, 20.6, 16.2, 16.1; ES-API MS: m/z calcd for C₂₉H₃₆F₃N₃O₂, found 516.3 [M+H].

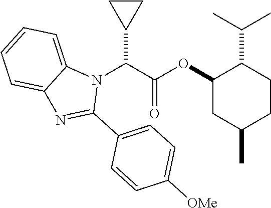 [SMILES: COc1ccc(-c2nc3ccccc3n2[C@@H](C(=O)O[C@@H]2C[C@H](C)CC[C@H]2C(C)C)C2CC2)cc1]

The title compound was obtained following the general procedure (Step A, method 1) described above. Purification of the residue by flash chromatography on silica gel (gradient elution, 45% EtOAc in hexanes) afforded LW-IV-3 as a pale yellow gel (41%). IR (cm⁻¹) 2957, 2871, 1739, 1614, 1486, 1455, 1371, 1253, 1176, 1029, 747; [α]D²⁰+18.87 (c 1.25, CHCl₃); ¹H NMR (400 MHz, CDCl₃) δ 7.83 (dd, J=8.4, 1.6 Hz, 1H), 7.62-7.59 (m, 3H), 7.31 (ddd, J=7.2, 6.8, 1.2 Hz, 1H), 7.27 (ddd, J=7.2, 6.8, 1.2 Hz, 1H), 7.00 (d, J=8.8 Hz, 2H), 4.80 (ddd, J=10.8, 10.8, 4.4 Hz, 1H), 4.23 (d, J=10.0 Hz, 1H), 3.88 (s, 3H), 1.91-1.79 (m, 3H), 1.70-1.64 (m, 2H), 1.45 (m, 1H), 1.35 (m, 1H), 1.26 (m, 1H), 1.05 (m, 1H), 0.89 (d, J=6.8 Hz, 3H), 0.84 (d, J=6.4 Hz, 3H), 0.79 (m, 2H), 0.78 (d, J=7.2 Hz, 3H), 0.42-0.33 (m, 2H), -0.11 (m, 1H); ¹³C NMR (100 MHz, CDCl₃) δ 169.1, 161.1, 154.3, 134.4, 131.3 (3C), 123.0, 122.9, 120.0, 114.4 (3C), 112.3, 76.6, 64.7, 55.6, 47.1, 40.5, 34.2, 31.5, 26.6, 23.4, 22.1, 21.0, 16.4, 12.4, 6.3, 3.9; ES-API MS: m/z calcd for C₂₉H₃₆N₂O₃, found 461.3 [M+H].

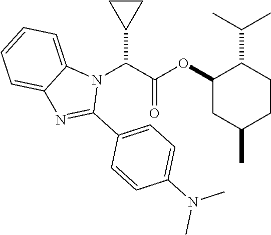 [SMILES: CC(C)[C@@H]1CC[C@@H](C)C[C@H]1OC(=O)[C@@H](C1CC1)n1c(-c2ccc(N(C)C)cc2)nc2ccccc21]

The title compound was obtained following the general procedure (Step A, method 1) described above. Purification of the residue by flash chromatography on silica gel (gradient elution, 50% EtOAc in hexanes) afforded LW-IV-4 as a pale yellow gel (40%). IR (cm⁻¹) 2956, 2870, 1738, 1678, 1614, 1492, 1455, 1367, 1276, 1197, 748; [α]_{D}²⁰+37.30 (c 0.81, CHCl₃); ¹H NMR (400 MHz, CDCl₃) δ 7.81 (d, J=8.4 Hz, 1H), 7.58 (dd, J=7.2, 1.2 Hz, 1H), 7.53 (d, J=8.8 Hz, 2H), 7.28 (ddd, J=7.6, 7.2, 1.2 Hz, 1H), 7.24 (ddd, J=7.6, 7.2, 1.2 Hz, 1H), 6.75 (d, J=9.2 Hz, 2H), 4.81 (ddd, J=10.8, 10.8, 4.0 Hz, 1H), 4.32 (d, J=10.0 Hz, 1H), 3.04 (s, 6H), 1.94-1.82 (m, 3H), 1.70-1.62 (m, 2H), 1.46 (m, 1H), 1.35 (m, 1H), 1.05 (m, 1H), 0.89 (d, J=7.2 Hz, 3H), 0.84 (d, J=6.8 Hz, 3H), 0.81 (m, 1H), 0.78 (d, J=6.8 Hz, 3H), 0.78 (m, 1H), 0.75 (m, 1H), 0.42-0.31 (m, 2H), -0.09 (m, 1H); ¹³C NMR (100 MHz, CDCl₃) δ 169.4, 155.4, 151.5, 134.5, 130.9 (2C), 128.9, 124.2, 122.6, 119.7, 113.0, 112.3, 112.0 (2C), 76.5, 64.7, 47.1, 40.9, 40.5, 40.4, 34.2, 31.5, 26.5, 23.4, 22.1, 21.0, 16.4, 12.5, 6.3, 3.8; ES-API MS: m/z calcd for C₃₀H₃₉N₃O₂, found 474.3 [M+H].

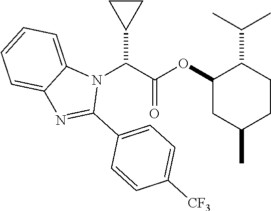 [SMILES: CC(C)[C@@H]1CC[C@@H](C)C[C@H]1OC(=O)[C@@H](C1CC1)n1c(-c2ccc(C(F)(F)F)cc2)nc2ccccc21]

The title compound was obtained following the general procedure (Step A, method 1) described above. Purification of the residue by flash chromatography on silica gel (gradient elution, 15% EtOAc in hexanes) afforded LW-IV-5 as a pale yellow gel (63%). IR (cm⁻¹) 2958, 2930, 2872, 1741, 1454, 1371, 1325, 1170, 1131, 1019, 743; [α]_{D}²⁰+7.52 (c 1.01, CHCl₃); ¹H NMR (400 MHz, CDCl₃) δ 7.85 (m, 1H), 7.83 (d, J=8.0 Hz, 2H), 7.76 (d, J=8.0 Hz, 2H), 7.64 (m, 1H), 7.33 (m, 2H), 4.82 (ddd, J=11.2, 10.8, 4.8 Hz, 1H), 4.16 (d, J=9.6 Hz, 1H), 1.91-1.77 (m, 3H), 1.71-1.62 (m, 2H), 1.46 (m, 1H), 1.36 (m, 1H), 1.05 (m, 1H), 0.89 (d, J=7.2 Hz, 3H), 0.84 (d, J=6.4 Hz, 3H), 0.84 (m, 1H), 0.81 (m, 2H), 0.77 (d, J=7.2 Hz, 3H), 0.45-0.36 (m, 2H), −0.11 (m, 1H); ¹³C NMR (100 MHz, CDCl₃) δ 168.7, 152.7, 143.3, 134.6, 134.0, 132.1 (q, J=32.6 Hz, 1C), 130.3 (3C), 125.9 (q, J=3.7 Hz, 1C), 124.0 (q, J=271.1 Hz, 1C), 123.6, 123.2, 120.5, 112.5, 76.8, 64.8, 47.1, 40.6, 34.1, 31.5, 26.6, 23.3, 22.1, 21.0, 16.3, 12.5, 6.4, 4.0; ES-API MS: m/z calcd for C₂₉H₃₃F₃N₂O₂, found 499.3 [M+H].

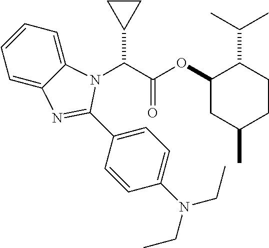

The title compound was obtained following the general procedure (Step A, method 1) described above. Purification of the residue by flash chromatography on silica gel (gradient elution, 45% EtOAc in hexanes) afforded LW-IV-56 as a pale yellow gel (34%). IR (cm⁻¹) 2958, 2929, 2870, 1738, 1614, 1489, 1455, 1359, 1270, 1198, 746; [α]_{D}²⁰+52.32 (c 1.33, CHCl₃); ¹H NMR (400 MHz, CDCl₃) δ 7.78 (d, J=7.6 Hz, 1H), 7.58 (d, J=7.6 Hz, 1H), 7.49 (d, J=8.8 Hz, 2H), 7.28-7.20 (m, 2H), 6.70 (d, J=8.8 Hz, 2H), 4.80 (ddd, J=10.8, 10.8, 4.4 Hz, 1H), 4.35 (d, J=10.0 Hz, 1H), 3.45 (q, J=7.2 Hz, 4H), 1.93-1.82 (m, 3H), 1.68-1.64 (m, 2H), 1.46 (m, 1H), 1.35 (m, 1H), 1.25 (m, 1H), 1.21 (t, J=7.2 Hz, 6H), 1.05 (m, 1H), 0.88 (d, J=7.2 Hz, 3H), 0.84 (d, J=6.4 Hz, 3H), 0.81 (m, 1H), 0.78 (d, J=7.2 Hz, 3H), 0.75 (m, 1H), 0.44-0.31 (m, 2H), -0.04 (m, 1H); ¹³C NMR (100 MHz, CDCl₃) δ 169.5, 155.7, 148.9, 143.8, 134.7, 131.0 (2C), 122.34, 122.29, 119.8, 116.2, 112.2, 111.3 (2C), 76.4, 64.6, 47.1, 44.5 (2C), 40.5, 34.2, 31.5, 26.5, 23.4, 22.1, 21.0, 16.4, 12.8 (2C), 12.5, 6.3, 3.8; ES-API MS: m/z calcd for C₃₂H₄₃N₃O₂, found 502.3 [M+H].

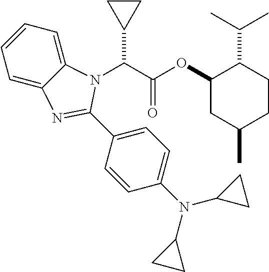

The title compound was obtained following the general procedure (Step A, method 1) described above. Purification of the residue by flash chromatography on silica gel (gradient elution, 30% EtOAc in hexanes) afforded LW-IV-94 as a pale yellow gel (20%). IR (cm⁻¹) 3086, 3009, 2956, 2870, 1739, 1613, 1485, 1455, 1370, 1346, 1277, 1187, 824, 746; [α]_{D}²⁰+45.28 (c 0.68, CHCl₃); ¹H NMR (400 MHz, CDCl₃) δ 7.79 (d, J=7.2 Hz, 1H), 7.58 (d, J=7.2 Hz, 1H), 7.51 (d, J=8.8 Hz, 2H), 7.27 (ddd, J=7.2, 7.6, 0.8 Hz, 1H), 7.23 (ddd, J=7.2, 7.6, 0.8 Hz, 1H), 7.09 (d, J=8.8 Hz, 2H), 4.80 (ddd, J=11.2, 10.8, 4.4 Hz, 1H), 4.35 (d, J=10.0 Hz, 1H), 2.52 (m, 2H), 1.95-1.82 (m, 3H), 1.73-1.59 (m, 3H), 1.45 (m, 1H), 1.35 (m, 1H), 1.05 (m, 1H), 0.94-0.88 (m, 6H), 0.87-0.82 (m, 4H), 0.80-0.72 (m, 8H), 0.67 (m, 1H), 0.44-0.32 (m, 2H), -0.05 (m, 1H); ¹³C NMR (100 MHz, CDCl₃) δ 169.5, 155.8, 151.2, 143.8, 134.7, 130.1 (2C), 122.4 (2C), 119.9, 117.9, 114.0 (2C), 112.2, 76.4, 64.5, 47.1, 40.5, 34.2, 31.5, 30.8 (2C), 26.5, 23.4, 22.1, 21.0, 16.4, 12.5, 9.5 (4C), 6.3, 3.8; ES-API MS: m/z calcd for C₃₄H₄₃N₃O₂, found 526.3 [M+H].

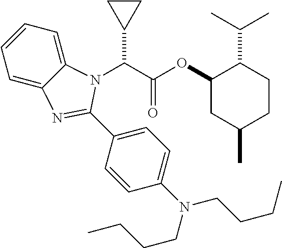

The title compound was obtained following the general procedure (Step A, method 1) described above. Purification of the residue by flash chromatography on silica gel (gradient elution, 30% EtOAc in hexanes) afforded LW-IV-95 as a pale yellow gel (14%). IR (cm⁻¹) 3316, 2956, 2871, 1740, 1614, 1489, 1455, 1369, 1198, 818, 746; [α]_{D}²⁰+42.61 (c 1.07, CHCl₃); ¹H NMR (400 MHz, CDCl₃) δ 7.78 (d, J=7.2 Hz, 1H), 7.57 (d, J=7.2 Hz, 1H), 7.48 (d, J=8.8 Hz, 2H), 7.27-7.20 (m, 2H), 6.65 (d, J=9.2 Hz, 2H), 4.80 (ddd, J=10.8, 10.8, 4.4 Hz, 1H), 4.35 (d, J=9.6 Hz, 1H), 3.31 (m, 4H), 1.97-1.81 (m, 3H), 1.68-1.56 (m, 6H), 1.45 (m, 1H), 1.41-1.31 (m, 5H), 0.97 (t, J=7.2 Hz, 6H), 0.92-0.87 (m, 5H), 0.84 (d, J=6.8 Hz, 3H), 0.80-0.74 (m, 5H), 0.44-0.31 (m, 2H), -0.04 (m, 1H); ¹³C NMR (100 MHz, CDCl₃) δ 169.3, 155.5, 149.1, 143.6, 134.5, 130.7, 122.11, 122.06, 119.5, 115.8, 112.0, 111.1, 76.1, 64.3, 50.7 (2C), 46.9, 40.3, 34.0, 31.3, 29.3 (2C), 26.3, 23.2, 21.9, 20.8, 20.3 (2C), 16.2, 14.0 (2C), 12.2, 6.1, 3.5; ES-API MS: m/z calcd for C₃₆H₅₁N₃O₂, found 558.4 [M+H].

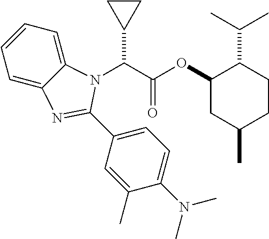

The title compound was obtained following the general procedure (Step A, method 1) described above. Purification of the residue by flash chromatography on silica gel (gradient elution, 45% EtOAc in hexanes) afforded LW-IV-75 as a pale yellow gel (20%). IR (cm⁻¹) 2955, 2870, 2782, 1739, 1614, 1487, 1455, 1273, 1166, 752; [α]_{D}²⁰+25.63 (c 2.20, CHCl₃); ¹H NMR (400 MHz, CDCl₃) δ 7.79 (d, J=8.4 Hz, 1H), 7.59 (d, J=8.4 Hz, 1H), 7.44 (s, 1H), 7.41 (d, J=8.4 Hz, 1H), 7.25 (m, 2H), 7.04 (d, J=8.4 Hz, 1H), 4.80 (ddd, J=10.8, 10.8, 4.4 Hz, 1H), 4.31 (d, J=10.0 Hz, 1H), 2.75 (s, 6H), 2.35 (s, 3H), 1.94-1.82 (m, 3H), 1.70-1.60 (m, 2H), 1.45 (m, 1H), 1.34 (m, 1H), 1.25 (m, 1H), 1.03 (m, 1H), 0.87 (d, J=7.2 Hz, 3H), 0.82 (d, J=6.8 Hz, 3H), 0.80 (m, 1H), 0.77 (d, J=7.2 Hz, 3H), 0.74 (m, 1H), 0.43-0.31 (m, 2H), −0.08 (m, 1H); ¹³C NMR (100 MHz, CDCl₃) δ 169.3, 154.9, 154.3, 143.6, 134.5, 132.6, 131.9, 127.9, 123.4, 122.6, 122.5, 120.0, 118.2, 112.3, 76.3, 64.4, 47.0, 43.9 (2C), 40.5, 34.1, 31.4, 26.4, 23.2, 22.0, 21.0, 18.9, 16.2, 12.5, 6.2, 3.7; ES-API MS: m/z calcd for C₃₁H₄₁N₃O₂, found 488.3 [M+H].

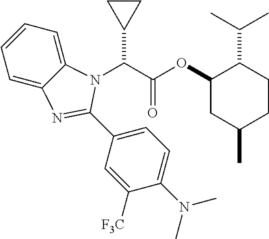

The title compound was obtained following the general procedure (Step A, method 1) described above. Purification of the residue by flash chromatography on silica gel (gradient elution, 45% EtOAc in hexanes) afforded LW-IV-69 as a pale yellow gel (24%). IR (cm⁻¹) 3367, 2956, 2872, 1740, 1621, 1455, 1144, 1050, 750; [α]_{D}²⁰+6.66 (c 0.93, CHCl₃); ¹H NMR (400 MHz, CDCl₃) δ 7.89 (d, J=2.0 Hz, 1H), 7.82 (dd, J=5.6, 2.0 Hz, 1H), 7.78 (dd, J=8.4, 2.0 Hz, 1H), 7.66 (dd, J=6.8, 2.0 Hz, 1H), 7.33-7.27 (m, 3H), 4.82 (ddd, J=10.8, 10.8, 4.4 Hz, 1H), 4.19 (d, J=10.0 Hz, 1H), 2.87 (s, 6H), 1.92-1.83 (m, 2H), 1.80 (m, 1H), 1.70-1.64 (m, 2H), 1.45 (m, 1H), 1.35 (m, 1H), 1.04 (m, 1H), 0.92 (m, 1H), 0.87 (d, J=7.2 Hz, 3H), 0.83 (d, J=6.4 Hz, 3H), 0.81 (m, 2H), 0.76 (d, J=6.8 Hz, 3H), 0.44-0.38 (m, 2H), −0.06 (m, 1H); ¹³C NMR (100 MHz, CDCl₃) δ 168.7, 154.1, 153.0, 143.3, 134.4, 133.7, 129.4 (q, J=5.0 Hz, 1C), 125.3, 123.5, 123.0, 122.7, 122.6, 121.6, 120.0, 112.4, 76.5, 64.6, 46.7, 44.94, 44.92, 40.3, 33.9, 31.3, 26.3, 23.1, 21.8, 20.7, 16.0, 12.3, 6.0, 3.8; ES-API MS: m/z calcd for C₃₁H₃₈F₃N₃O₂, found 542.3 [M+H].

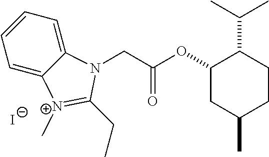

The title compounds were obtained following the general procedure (Step F) described above. Purification of the residue by prep-TLC (8% MeOH in CH₂Cl₂) afforded the entitled salt LW-III-244 as a yellow gel (80%). IR (cm⁻¹) 2926, 1738, 1472, 1224, 1200, 766; [α]_{D}²⁰+15.33 (c 1.33, CHCl₃); ¹H NMR (400 MHz, CDCl₃) δ 7.77 (dd, J=6.4, 2.0 Hz, 1H), 7.61-7.52 (m, 3H), 5.49 (d, J=18.4 Hz, 1H), 5.38 (d, J=18.4 Hz, 1H), 5.31 (m, 1H), 4.14 (s, 3H), 3.53 (m, 2H), 1.90 (dd, J=14.4, 2.4 Hz, 1H), 1.76-1.70 (m, 2H), 1.57 (m, 1H), 1.37 (t, J=8.0 Hz, 3H), 1.32 (m, 1H), 1.22 (ddd, J=12.8, 13.2, 3.2 Hz, 1H), 1.06 (ddd, J=12.4, 12.4, 2.4 Hz, 1H), 0.98 (dddd, J=9.2, 9.2, 3.6, 3.6 Hz, 1H), 0.90 (ddd, J=12.4, 12.4, 3.2 Hz, 1H), 0.87 (d, J=6.8 Hz, 3H), 0.84 (d, J=6.4 Hz, 3H), 0.80 (d, J=6.8 Hz, 3H); ¹³C NMR (100 MHz, CDCl₃) δ 165.5, 155.8, 131.5, 131.3, 127.2, 127.1, 113.0, 112.2, 75.0, 48.2, 46.5, 39.0, 34.4, 33.5, 29.2, 26.5, 22.0, 20.9, 20.7, 11.5; ES-API MS: m/z calcd for C₂₂H₃₃IN₂O₂, found 357.2 [M−I].

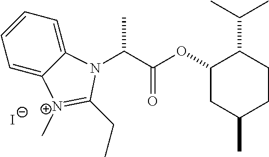

The title compounds were obtained following the general procedure (Step F) described above. Purification of the residue by prep-TLC (8% MeOH in CH₂Cl₂) afforded the entitled salt LW-III-238 as a yellow gel (90%). IR (cm⁻¹) 3446, 3227, 2956, 2930, 2871, 1739, 1473, 1227, 1089, 752; [α]_{D}²⁰−18.21 (c 1.12, CHCl₃); ¹H NMR (400 MHz, CDCl₃) δ 7.84 (d, J=8.4 Hz, 1H), 7.57 (ddd, J=6.4, 6.4, 2.0 Hz, 1H), 7.52-7.46 (m, 2H), 5.74 (q, J=7.6 Hz, 1H), 4.74 (ddd, J=10.8, 10.8, 4.4 Hz, 1H), 4.20 (s, 3H), 3.76 (dq, J=15.6, 8.0 Hz, 1H), 3.57 (dq, J=16.0, 8.0 Hz, 1H), 2.00 (d, J=7.2 Hz, 3H), 1.81 (m, 1H), 1.75 (m, 1H), 1.64-1.57 (m, 2H), 1.36 (t, J=8.0 Hz, 3H), 1.36-1.28 (m, 2H), 1.30 (m, 1H), 0.96 (m, 1H), 0.85 (d, J=7.2 Hz, 3H), 0.77 (d, J=6.4 Hz, 3H), 0.73 (d, J=7.2 Hz, 3H), 0.70 (m, 1H); ¹³C NMR (100 MHz, CDCl₃) δ 167.3, 155.3, 132.0, 129.4, 126.9, 126.7, 113.60, 113.57, 77.6, 56.0, 46.6, 40.1, 34.0, 33.7, 31.3, 26.2, 22.9, 21.8, 20.7, 20.4, 17.0, 15.9, 11.9; ES-API MS: m/z calcd for C₂₃H₃₅IN₂O₂, found 371.3 [M−I].

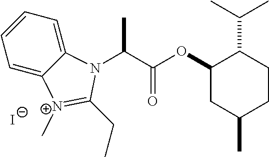

The title compounds were obtained following the general procedure (Step F) described above. Purification of the residue by prep-TLC (8% MeOH in CH₂Cl₂) afforded the entitled salt LW-III-239 as a yellow gel (85%). IR (cm⁻¹) 3455, 2956, 2871, 1739, 1473, 1225, 1090, 755; [α]_{D}²⁰−29.82 (c 1.18, CHCl₃); ⁱH NMR (400 MHz, CDCl₃) δ 7.81 (d, J=8.4 Hz, 1H), 7.59 (ddd, J=6.4, 6.4, 2.0 Hz, 1H), 7.56-7.50 (m, 2H), 5.73 (q, J=7.2 Hz, 1H), 4.66 (ddd, J=10.8, 10.4, 4.0 Hz, 1H), 4.22 (s, 3H), 3.82 (dq, J=16.4, 8.4 Hz, 1H), 3.66 (dq, J=16.4, 8.0 Hz, 1H), 2.05 (d, J=7.2 Hz, 3H), 2.02 (d, J=12.0 Hz, 1H), 1.62 (d, J=12.4 Hz, 1H), 1.55 (dd, J=13.6, 2.4 Hz, 1H), 1.40 (m, 1H), 1.40 (t, J=7.6 Hz, 3H), 1.21 (d, J=13.6 Hz, 1H), 1.04-0.89 (m, 3H), 0.88 (d, J=6.4 Hz, 3H), 0.85-0.75 (m, 1H), 0.57 (d, J=6.8 Hz, 3H), 0.37 (d, J=6.8 Hz, 3H); ¹³C NMR (100 MHz, CDCl₃) δ 167.2, 155.5, 132.0, 129.6, 126.9, 126.7, 113.8, 113.3, 77.6, 56.1, 46.6, 40.5, 33.9, 33.8, 31.3, 25.8, 22.9, 21.8, 20.7, 20.4, 16.6, 15.6, 11.9; ES-API MS: m/z calcd for C₂₃H₃₅IN₂O₂, found 371.3 [M−I].

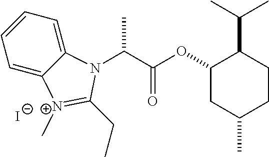

The title compounds were obtained following the general procedure (Step F) described above. Purification of the residue by prep-TLC (8% MeOH in CH₂Cl₂) afforded the entitled salt LW-III-264 as a yellow gel (83%). IR (cm⁻¹) 2956, 1738, 1727, 1484, 1469, 1225; [α]_{D}²⁰+34.92 (c 1.38, CHCl₃); ¹H NMR (400 MHz, CDCl₃) δ 7.79 (d, J=8.0 Hz, 1H), 7.60 (ddd, J=6.4, 6.4, 2.4 Hz, 1H), 7.55 (m, 2H), 5.70 (q, J=7.6 Hz, 1H), 4.67 (ddd, J=10.8, 11.2, 4.4 Hz, 1H), 4.21 (s, 3H), 3.81 (dq, J=15.2, 7.6 Hz, 1H), 3.67 (dq, J=15.6, 8.0 Hz, 1H), 2.06 (d, J=7.2 Hz, 3H), 2.03 (m, 1H), 1.63 (m, 1H), 1.57 (m, 1H), 1.46 (m, 1H), 1.40 (t, J=7.6 Hz, 3H), 1.22 (m, 1H), 1.05-0.92 (m, 3H), 0.89 (d, J=6.8 Hz, 3H), 0.81 (m, 1H), 0.58 (d, J=6.8 Hz, 3H), 0.38 (d, J=6.8 Hz, 3H); ¹³C NMR (100 MHz, CDCl₃) δ 167.4, 155.8, 132.2, 129.8, 127.1, 126.9, 114.0, 113.4, 77.9, 56.2, 46.8, 40.7, 33.99, 33.95, 31.5, 26.1, 23.1, 22.0, 21.0, 20.6, 16.7, 15.8, 12.0; ES-API MS: m/z calcd for C₂₃H₃₅IN₂O₂, found 371.3 [M−I].

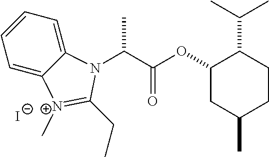

The title compounds were obtained following the general procedure (Step F) described above. Purification of the residue by prep-TLC (8% MeOH in CH₂Cl₂) afforded the entitled salt LW-III-265 as a yellow gel (75%). IR (cm⁻¹) 2924, 2361, 1738, 1469, 1230, 1089, 751; [α]_{D}²⁰+24.43 (c 1.08, CHCl₃); ¹H NMR (400 MHz, CDCl₃) δ 7.79 (d, J=8.4 Hz, 1H), 7.60 (ddd, J=8.0, 4.8, 4.8 Hz, 1H), 7.54 (d, J=0.8 Hz, 1H), 7.53 (dd, J=2.0, 0.8 Hz, 1H), 5.73 (q, J=7.2 Hz, 1H), 5.25 (m, 1H), 4.22 (s, 3H), 3.79 (dq, J=16.0, 8.0 Hz, 1H), 3.68 (dq, J=16.0, 8.0 Hz, 1H), 2.08 (d, J=7.2 Hz, 3H), 2.02 (dd, J=14.4, 2.4 Hz, 1H), 1.76-1.49 (m, 4H), 1.42 (t, J=7.6 Hz, 3H), 1.10 (ddd, J=13.8, 13.8, 2.0 Hz, 1H), 0.99 (ddd, J=12.4, 12.4, 2.8 Hz, 1H), 0.92 (m, 1H), 0.90 (m, 1H), 0.89 (d, J=6.4 Hz, 3H), 0.65 (d, J=6.0 Hz, 3H), 0.58 (d, J=6.4 Hz, 3H); ¹³C NMR (100 MHz, CDCl₃) δ 167.5, 155.7, 132.2, 129.8, 127.1, 126.9, 114.0, 113.4, 75.2, 56.4, 46.6, 39.0, 34.5, 34.0, 29.0, 27.0, 25.1, 22.3, 21.0, 20.83, 20.82, 17.0, 12.2; ES-API MS: m/z calcd for C₂₃H₃₅IN₂O₂, found 371.3 [M−I].

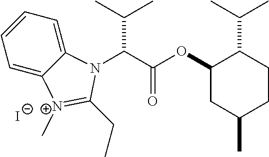

The title compounds were obtained following the general procedure (Step F) described above. Purification of the residue by prep-TLC (10% MeOH in CH₂Cl₂) afforded the entitled salt LW-III-294 as a yellow gel (99%). IR (cm⁻¹) 3418, 2961, 2872, 1738, 1470, 1261, 1038, 802; [α]_{D}²⁰−25.94 (c 1.11, CHCl₃); ¹H NMR (400 MHz, CDCl₃) δ 7.92 (d, J=9.6 Hz, 2H), 7.64 (dd, J=7.6, 8.8 Hz, 1H), 7.56 (dd, J=8.8, 7.6 Hz, 1H), 4.79 (d, J=11.2 Hz, 1H), 4.74 (ddd, J=11.2, 11.2, 4.4 Hz, 1H), 4.39 (s, 3H), 3.83 (dq, J=15.6, 7.6 Hz, 1H), 3.60 (dq, J=15.6, 8.0 Hz, 1H), 3.50 (m, 1H), 1.73 (m, 1H), 1.68-1.59 (m, 3H), 1.47 (t, J=8.0 Hz, 3H), 1.42-1.29 (m, 2H), 1.27 (d, J=6.4 Hz, 3H), 0.98 (m, 1H), 0.84 (d, J=6.8 Hz, 3H), 0.82 (d, J=6.8 Hz, 3H), 0.77 (d, J=6.4 Hz, 3H), 0.73 (m, 1H), 0.71 (d, J=6.8 Hz, 3H), 0.69 (m, 1H); ¹³C NMR (100 MHz, CDCl₃) δ 166.5, 155.6, 132.0, 129.7, 127.5, 127.2, 115.1, 113.8, 77.9, 67.3, 46.7, 40.4, 35.1, 33.8, 31.5, 29.0, 26.3, 23.0, 21.9, 20.9, 20.2, 20.0, 19.6, 16.0, 12.2; ES-API MS: m/z calcd for C₂₅H₃₉IN₂O₂, found 400.3 [M−I].

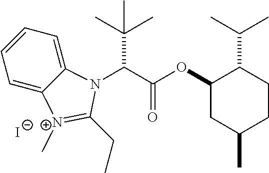

The title compounds were obtained following the general procedure (Step F) described above. Purification of the residue by prep-TLC (10% MeOH in CH₂Cl₂) afforded the entitled salt LW-III-295 as a yellow gel (97%). IR (cm⁻¹) 3410, 2961, 1743, 1469, 1261, 1045, 802; [α]_{D}²⁰−18.41 (c 1.26, CHCl₃); ⁱH NMR (400 MHz, CDCl₃) δ 7.92 (d, J=8.4 Hz, 1H), 7.83 (d, J=8.4 Hz, 1H), 7.62 (dd, J=7.6, 8.0 Hz, 1H), 7.52 (dd, J=8.0, 7.6 Hz, 1H), 4.96 (s, 1H), 4.76 (ddd, J=10.8, 10.8, 4.0 Hz, 1H), 4.44 (s, 3H), 4.09 (dq, J=15.6, 8.0 Hz, 1H), 3.44 (dq, J=14.8, 8.0 Hz, 1H), 1.79 (m, 1H), 1.65-1.51 (m, 3H), 1.44 (t, J=7.6 Hz, 3H), 1.35 (m, 1H), 1.27 (s, 9H), 1.25-1.19 (m, 2H), 0.98 (m, 1H), 0.86 (d, J=6.8 Hz, 3H), 0.76 (d, J=6.8 Hz, 3H), 0.73 (d, J=6.4 Hz, 3H), 0.46 (m, 1H); ¹³C NMR (100 MHz, CDCl₃) δ 165.3, 156.5, 132.2, 130.6, 127.3, 126.7, 116.4, 113.6, 77.5, 68.4, 46.7, 40.2, 37.3, 35.3, 33.8, 31.4, 28.5 (3C), 26.5, 23.1, 21.9, 20.9, 20.4, 16.1, 12.0; ES-API MS: m/z calcd for C₂₆H₄₁IN₂O₂, found 414.3 [M−I+H].

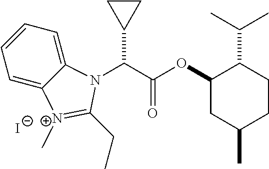

The title compounds were obtained following the general procedure (Step F) described above. Purification of the residue by prep-TLC (10% MeOH in CH₂Cl₂) afforded the entitled salt LW-III-296 as a yellow gel (98%). IR (cm⁻¹) 3439, 2957, 2871, 1738, 1470, 1242, 1036, 753; [α]_{D}−37.85 (c 1.03, CHCl₃); ¹H NMR (400 MHz, CDCl₃) δ 7.86 (d, J=8.0 Hz, 1H), 7.74 (d, J=8.4 Hz, 1H), 7.60 (ddd, J=7.2, 7.6, 1.2 Hz, 1H), 7.53 (ddd, J=7.6, 7.2, 1.2 Hz, 1H), 4.91 (d, J=9.6 Hz, 1H), 4.73 (ddd, J=10.8, 10.8, 4.4 Hz, 1H), 4.25 (s, 3H), 3.62 (m, 2H), 1.89-1.79 (m, 3H), 1.67-1.59 (m, 2H), 1.39 (m, 2H), 1.35 (t, J=7.6 Hz, 3H), 1.14 (m, 1H), 1.03-0.88 (m, 4H), 0.85 (d, J=6.8 Hz, 3H), 0.80 (d, J=6.4 Hz, 3H), 0.72 (d, J=6.8 Hz, 3H), 0.70-0.60 (m, 2H); ¹³C NMR (100 MHz, CDCl₃) δ 167.0, 155.2, 132.0, 130.3, 127.1, 126.9, 114.4, 113.5, 77.9, 65.3, 46.8, 40.3, 34.3, 33.9, 31.4, 26.3, 23.0, 21.9, 20.9, 20.2, 16.0, 12.9, 12.2, 7.6, 5.1; ES-API MS: m/z calcd for C₂₅H₃₇IN₂O₂, found 397.3 [M−I].

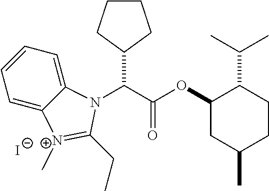

The title compounds were obtained following the general procedure (Step F) described above. Purification of the residue by prep-TLC (10% MeOH in CH₂Cl₂) afforded the entitled salt LW-III-297 as a yellow gel (81%). IR (cm⁻¹) 3022, 2955, 2870, 1739, 1470, 1262, 1083, 749; [α]_{D}²⁰−26.78 (c 1.06, CHCl₃); ¹H NMR (400 MHz, CDCl₃) δ 7.90 (d, J=8.0 Hz, 1H), 7.83 (d, J=8.4 Hz, 1H), 7.64 (dd, J=7.2, 8.4 Hz, 1H), 7.56 (dd, J=8.4, 7.6 Hz, 1H), 4.99 (d, J=11.2 Hz, 1H), 4.75 (ddd, J=10.8, 10.8, 4.4 Hz, 1H), 4.38 (s, 3H), 3.86 (m, 1H), 3.61 (m, 1H), 3.12 (m, 1H), 2.15 (m, 1H), 1.84-1.50 (m, 9H), 1.46 (t, J=8.0 Hz, 3H), 1.42-1.31 (m, 3H), 1.20 (m, 2H), 0.99 (m, 1H), 0.86 (d, J=7.2 Hz, 3H), 0.79 (d, J=6.4 Hz, 3H), 0.76 (m, 1H), 0.72 (d, J=6.8 Hz, 3H); ¹³C NMR (100 MHz, CDCl₃) δ 166.9, 155.4, 132.0, 129.9, 127.4, 127.2, 114.7, 113.8, 77.9, 77.4, 65.8, 46.8, 40.4, 35.0, 33.9, 31.5, 31.3, 30.7, 26.4, 25.5, 25.0, 23.0, 21.9, 20.9, 20.2, 16.0, 12.2; ES-API MS: m/z calcd for C₂₇H₄₁IN₂O₂, found 425.3 [M−I].

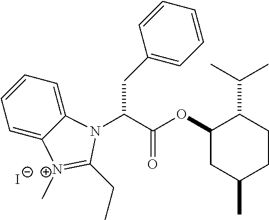

The title compounds were obtained following the general procedure (Step F) described above. Purification of the residue by prep-TLC (7% MeOH in CH₂Cl₂) afforded the entitled salt LW-III-304 as a yellow gel (91%). IR (cm⁻¹) 3030, 2957, 2870, 1738, 1260, 1080, 750, 702; [α]_{D}²⁰−23.03 (c 1.12, CHCl₃); ⁱH NMR (400 MHz, CDCl₃) δ 7.96 (d, J=8.4 Hz, 1H), 7.69-7.65 (m, 2H), 7.60 (dd, J=8.0, 7.6 Hz, 1H), 7.18-7.17 (m, 3H), 7.02-7.00 (m, 2H), 5.77 (dd, J=9.6, 5.6 Hz, 1H), 4.82 (ddd, J=11.2, 10.8, 4.0 Hz, 1H), 4.17 (s, 3H), 3.86 (dd, J=14.4, 5.6 Hz, 1H), 3.55 (dd, J=14.4, 9.6 Hz, 1H), 3.27-3.15 (m, 2H), 1.81 (m, 1H), 1.74 (m, 1H), 1.66-1.59 (m, 2H), 1.44-1.30 (m, 2H), 1.11 (t, J=7.6 Hz, 3H), 0.99 (m, 1H), 0.84 (d, J=6.8 Hz, 3H), 0.81 (m, 1H), 0.80 (d, J=6.8 Hz, 3H), 0.76 (m, 1H), 0.74 (d, J=7.2 Hz, 3H); NMR (100 MHz, CDCl₃) δ 166.2, 155.8, 134.7, 132.0, 129.6, 129.3 (2C), 129.1 (3C), 128.2, 127.5, 127.2, 114.1, 78.2, 62.5, 46.7, 40.3, 35.8, 34.4, 33.8, 31.5, 26.4, 23.1, 21.9, 20.9, 19.2, 16.2, 11.8; ES-API MS: m/z calcd for C₂₉H₃₉IN₂O₂, found 447.3 [M−I].

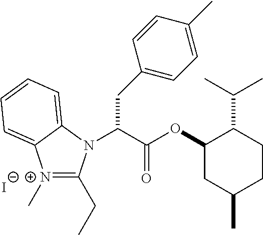

The title compounds were obtained following the general procedure (Step F) described above. Purification of the residue by prep-TLC (7% MeOH in CH₂Cl₂) afforded the entitled salt LW-III-305 as a yellow gel (86%). IR (cm⁻¹) 2956, 2870, 1738, 1712, 1470, 1261, 1178, 1038, 750; [α]_{D}²⁰+38.05 (c 1.03, CHCl₃); ⁱH NMR (400 MHz, CDCl₃) δ 7.97 (d, J=8.8 Hz, 1H), 7.69-7.65 (m, 2H), 7.59 (ddd, J=8.4, 8.0, 0.8 Hz, 1H), 6.96 (d, J=8.0 Hz, 2H), 6.85 (d, J=8.0 Hz, 2H), 5.68 (dd, J=10.4, 5.6 Hz, 1H), 4.80 (ddd, J=11.2, 10.8, 4.4 Hz, 1H), 4.19 (s, 3H), 4.80 (dd, J=14.4, 5.6 Hz, 1H), 3.49 (dd, J=14.4, 10.0 Hz, 1H), 3.23 (dq, J=15.6, 7.6 Hz, 1H), 3.11 (dq, J=14.8, 7.6 Hz, 1H), 2.22 (s, 3H), 1.80 (m, 1H), 1.72 (m, 1H), 1.66-1.58 (m, 2H), 1.38 (m, 1H), 1.32 (m, 1H), 1.12 (t, J=7.6 Hz, 3H), 0.98 (m, 1H), 0.83 (d, J=7.2 Hz, 3H), 0.78 (m, 1H), 0.79 (d, J=6.8 Hz, 3H), 0.75 (m, 1H), 0.73 (d, J=6.8 Hz, 3H); ¹³C NMR (100 MHz, CDCl₃) δ 166.2, 155.6, 137.9, 131.9, 131.4, 130.0 (2C), 129.5, 128.9 (2C), 127.4, 127.1, 114.14, 114.08, 78.2, 62.5, 46.7, 40.3, 35.3, 34.5, 33.8, 31.5, 26.3, 23.0, 21.9, 21.2, 20.9, 19.1, 16.1, 11.8; ES-API MS: m/z calcd for C₃₀H₄₁IN₂O₂, found 461.3 [M−I].

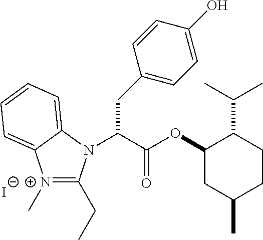

The title compounds were obtained following the general procedure (Step F) described above. Purification of the residue by prep-TLC (7% MeOH in CH₂Cl₂) afforded the entitled salt LW-III-306 as a yellow gel (85%). IR (cm⁻¹) 3204, 2963, 1738, 1514, 1470, 1445, 1261, 1031, 801; [α]_{D}²⁰+65.48 (c 0.40, CHCl₃); ⁱH NMR (400 MHz, CDCl₃) δ 7.86 (d, J=8.0 Hz, 1H), 7.71-7.60 (m, 3H), 7.56 (br, 1H), 6.87 (d, J=8.4 Hz, 2H), 6.61 (d, J=8.4 Hz, 2H), 5.57 (dd, J=11.2, 4.4 Hz, 1H), 4.87 (ddd, J=10.8, 10.8, 4.4 Hz, 1H), 4.11 (s, 3H), 3.74 (dd, J=14.4, 4.0 Hz, 1H), 3.38 (dd, J=14.4, 11.6 Hz, 1H), 3.02 (dq, J=15.6, 7.6 Hz, 1H), 2.81 (m, 1H), 1.89-1.83 (m, 2H), 1.78-1.63 (m, 3H), 1.48-1.36 (m, 2H), 1.25 (m, 2H), 1.03 (m, 1H), 0.91 (d, J=7.2 Hz, 3H), 0.88 (m, 1H), 0.84 (d, J=6.8 Hz, 3H), 0.81 (d, J=6.8 Hz, 3H), 0.77 (m, 1H); ¹³C NMR (100 MHz, CDCl₃) δ 166.3, 156.9, 155.8, 131.9, 129.7 (2C), 127.6, 127.3, 125.2, 117.4 (2C), 114.1, 114.0, 78.3, 62.9, 46.8, 40.4, 35.0, 34.1, 34.0, 31.6, 29.9, 26.5, 23.2, 22.0, 21.0, 18.3, 16.3, 11.8; ES-API MS: m/z calcd for C₂₉H₃₉IN₂O₃, found 463.3 [M−I].

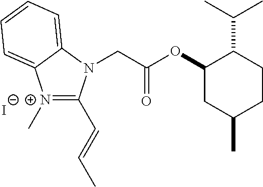

The title compounds were obtained following the general procedure (Step F) described above. Purification of the residue by flash chromatography on silica gel (gradient elution, 5% MeOH in CH₂Cl₂) afforded the entitled salt Lqr-7-075 as a yellow solid (54%). IR (cm⁻¹) 2919, 2342, 1733, 1646, 1540, 1472, 1019, 787; [α]_{D}²⁰−11.998 (c 0.05, CHCl₃); ¹H NMR (400 MHz, CDCl₃) δ 7.73 (d, J=8.4 Hz, 1H), 7.33-7.15 (m, 3H), 7.09 (dd, J=15.2, 6.8 Hz, 1H), 6.37 (d, J=15.2 Hz, 1H), 4.83 (s, 2H), 4.81-4.65 (m, 1H), 3.70 (s, 3H), 2.08-1.92 (m, 4H), 1.82-1.16 (m, 5H), 1.14-0.72 (m, 6H), 0.78 (d, J=7.2 Hz, 3H), 0.66 (d, J=7.2 Hz, 3H); ES-API MS: m/z calcd for C₂₃H₃₃IN₂O₂, found 369.2 [M−I].

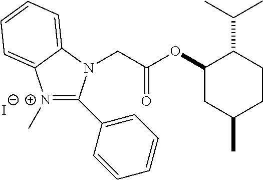

The title compounds were obtained following the general procedure (Step F) described above. Purification of the residue by flash chromatography on silica gel (gradient elution, 5% MeOH in CH₂Cl₂) afforded the entitled salt Lqr-5-073 as a yellow solid (77%). IR (cm⁻¹) 2921, 1734, 1466, 1208, 1036, 981, 759; [α]_{D}²⁰−53.998 (c 0.1, CHCl₃); ¹H NMR (400 MHz, CDCl₃) δ 8.06 (m, 2H), 7.85-7.74 (m, 2H), 7.74-7.66 (m, 4H), 7.66-7.60 (m, 1H), 5.19 (d, J=18.4 Hz, 1H), 5.13 (d, J=18.0 Hz, 1H), 4.76 (td, J=10.8, 4.4 Hz, 1H), 4.02 (s, 3H), 1.95-1.84 (m, 1H), 1.75-1.59 (m, 3H), 1.52-1.42 (m, 1H), 1.42-1.31 (m, 1H), 1.09-0.95 (m, 2H), 0.94-0.82 (m, 1H), 0.92 (d, J=6.8 Hz, 3H), 0.86 (d, J=7.2 Hz, 3H), 0.69 (d, J=7.2 Hz, 3H); ¹³C NMR (100 MHz, CDCl₃) δ 165.8, 151.3, 133.6, 131.9, 131.7, 131.2, 129.8, 127.8 (2C), 127.6 (2C), 120.2, 113.3, 112.9, 77.6, 48.7, 46.7, 40.5, 33.8, 33.7, 31.4, 26.3, 23.1, 21.8, 20.6, 16.1; ES-API MS: m/z calcd for C₂₆H₃₃IN₂O₂, found 405.2 [M−I].

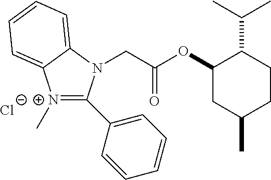

The title compounds were obtained following the general procedure (Step G) described above. Purification of the residue by flash chromatography on silica gel (gradient elution, 5% MeOH in CH₂Cl₂) afforded the entitled salt Lqr-5-077 as a yellow solid (90%). IR (cm⁻¹) 2921, 1734, 1466, 1208, 1036, 981, 759; [α]_{D}²⁰−17.998 (c 0.1, CHCl₃); ¹H NMR (400 MHz, CDCl₃) δ 8.17-7.95 (m, 2H), 7.88-7.80 (m, 1H), 7.79-7.73 (m, 2H), 7.73-7.63 (m, 4H), 5.36 (d, J=18.0 Hz, 1H), 5.21 (d, J=18.0 Hz, 1H), 4.75 (td, J=10.8, 4.0 Hz, 1H), 4.06 (s, 3H), 1.89 (d, J=12.4 Hz, 1H), 1.67 (d, J=10.4 Hz, 2H), 1.64-1.54 (m, 1H), 1.52-1.31 (m, 2H), 1.09-0.94 (m, 2H), 0.91 (d, J=6.8 Hz, 3H), 0.85 (d, J=7.2 Hz, 3H), 0.89-0.78 (m, 1H), 0.68 (d, J=6.8 Hz, 3H); ¹³C NMR (100 MHz, CDCl₃) δ 166.1, 151.5, 133.4, 131.9, 131.6, 130.9, 129.9, 127.7 (2C), 127.4 (2C), 120.3, 113.2, 113.0, 77.4, 48.3, 46.7, 40.5, 33.8, 33.5, 31.4, 26.2, 23.1, 21.8, 20.6, 16.1; ES-API MS: m/z calcd for C₂₆H₃₃ClN₂O₂, found 405.2 [M-Cl].

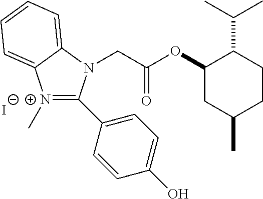

The title compounds were obtained following the general procedure (Step F) described above. Purification of the residue by flash chromatography on silica gel (gradient elution, 5% MeOH in CH₂Cl₂) afforded the entitled salt Lqr-5-088 as a yellow solid (67%). IR (cm⁻¹) 2955, 1740, 1608, 1469, 1287, 1226, 1035, 910, 851; [α]_{D}²⁰−47.668 (c 0.2, CHCl₃); ¹H NMR (400 MHz, CDCl₃) δ 10.40 (s, 1H), 7.82-7.72 (m, 1H), 7.72-7.60 (m, 2H), 7.60-7.46 (m, 3H), 7.46-7.34 (m, 2H), 5.10 (s, 2H), 4.86-4.72 (m, 1H), 4.06 (s, 3H), 1.95 (d, J=10.8 Hz, 1H), 1.77-1.54 (m, 3H), 1.53-1.31 (m, 2H), 1.14-0.81 (m, 3H), 0.92 (d, J=6.0 Hz, 3H), 0.88 (d, J=6.4 Hz, 3H), 0.73 (d, J=6.4 Hz, 3H); ¹³C NMR (100 MHz, CDCl₃) δ 165.8, 163.3, 152.2, 131.9, 131.8 (2C), 131.4, 127.5, 127.4, 117.9, 113.1, 112.5, 108.6, 77.8, 77.2, 48.2, 46.8, 40.5, 33.8, 31.4, 26.3, 23.1, 21.9, 20.7, 16.0; ES-API MS: m/z calcd for C₂₆H₃₃IN₂O₃, found 421.2 [M−I].

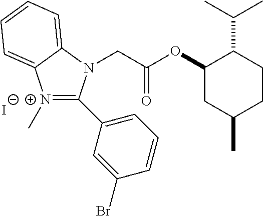

The title compounds were obtained following the general procedure (Step F) described above. Purification of the residue by flash chromatography on silica gel (gradient elution, 5% MeOH in CH₂Cl₂) afforded the entitled salt Lqr-5-089 as a yellow solid (67%). IR (cm⁻¹) 2953, 1739, 1516, 1438, 1457, 1224, 1035; [α]_{D}²⁰−32.668 (c 0.2, CHCl₃); ¹H NMR (400 MHz, CDCl₃) δ 8.39-8.20 (m, 1H), 8.02-7.92 (m, 1H), 7.90 (d, J=8.4 Hz, 1H), 7.88-7.81 (m, 1H), 7.77-7.57 (m, 4H), 5.23 (s, 2H), 4.78 (td, J=10.8, 4.4 Hz, 1H), 4.08 (s, 3H), 1.93 (d, J=11.2 Hz, 1H), 1.70-1.65 (m, 2H), 1.53-1.33 (m, 2H), 1.27-1.16 (m, 1H), 1.12-0.96 (m, 2H), 0.92 (d, J=6.0 Hz, 3H), 0.91-0.81 (m, 1H), 0.87 (d, J=4.2 Hz, 3H), 0.70 (d, J=7.2 Hz, 3H); ¹³C NMR (100 MHz, CDCl₃) δ 165.8, 149.5, 136.9, 132.8, 131.9, 131.8, 131.6, 130.4, 128.1, 127.8, 123.7, 122.1, 113.4, 113.0, 77.8, 77.2, 48.5, 46.6, 40.5, 33.8, 31.4, 26.4, 23.2, 21.8, 20.6, 16.1; ES-API MS: m/z calcd for C₂₆H₃₂BrIN₂O₂, found 483.1 [M−I].

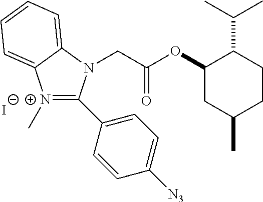

The title compounds were obtained following the general procedure (Step F) described above. Purification of the residue by flash chromatography on silica gel (gradient elution, 5% MeOH in CH₂Cl₂) afforded the entitled salt Lqr-5-090 as a yellow solid (50%). IR (cm⁻¹) 2957, 2385, 2121, 1734, 1603, 1489, 1281, 1225, 1094; [α]_{D}²⁰−12.008 (c 0.1, CHCl₃); ¹H NMR (400 MHz, CDCl₃) δ 8.08-7.90 (m, 2H), 7.87-7.79 (m, 1H), 7.81-7.70 (m, 2H), 7.70-7.61 (m, 1H), 7.41-7.31 (m, 2H), 5.32-5.11 (m, 2H), 4.86-4.71 (m, 1H), 4.08 (s, 3H), 1.96-1.87 (m, 1H), 1.76-1.60 (m, 3H), 1.56-1.36 (m, 2H), 1.13-0.96 (m, 2H), 0.96-0.85 (m, 1H), 0.93 (d, J=6.8 Hz, 3H), 0.89 (d, J=7.2 Hz, 3H), 0.72 (d, J=6.8 Hz, 3H); ¹³C NMR (100 MHz, CDCl₃) δ 165.8, 163.3, 152.4, 131.8 (2C), 131.7, 131.4, 127.5, 127.4, 117.9 (2C), 113.1, 112.5, 108.6, 77.8, 48.1, 46.8, 40.5, 33.8 (2C), 31.4, 26.3, 23.1, 21.9, 20.7, 16.0; ES-API MS: m/z calcd for C₂₆H₃₂IN₅O₂, found 446.2 [M−I].

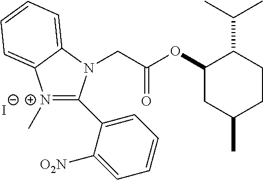

The title compounds were obtained following the general procedure (Step F) described above. Purification of the residue by flash chromatography on silica gel (gradient elution, 5% MeOH in CH₂Cl₂) afforded the entitled salt Lqr-5-120 as a yellow solid (80%). IR (cm⁻¹) 2955, 2342, 1736, 1533, 1458, 1348, 1212, 788; [α]_{D}²⁰−30.220 (c 0.4, CHCl₃); ¹H NMR (400 MHz, CDCl₃) δ 9.27-9.10 (m, 1H), 8.49 (td, J=8.4, 1.2 Hz, 1H), 8.14 (tdd, J=7.6, 2.8, 1.2 Hz, 1H), 8.10-8.00 (m, 1H), 7.89-7.79 (m, 1H), 7.79-7.66 (m, 3H), 5.29 (d, J=18.0 Hz, 1H), 5.26 (d, J=17.6 Hz, 1H), 4.80 (dd, J=17.6, 1.2 Hz, 1H), 4.72-4.53 (m, 1H), 3.94 (d, J=1.2 Hz, 3H), 1.89-1.79 (m, 1H), 1.76-1.54 (m, 3H), 1.49-1.14 (m, 2H), 1.05-0.74 (m, 3H), 0.87 (d, J=7.2 Hz, 3H), 0.83 (d, J=6.8 Hz, 3H), 0.66 (d, J=6.8 Hz, 2H), 0.51 (d, J=6.8 Hz, 1H); ¹³C NMR (100 MHz, CDCl₃) δ 164.4, 147.3, 136.1, 135.7, 135.1, 131.9, 131.8, 128.0, 127.9, 125.9, 115.2, 113.3, 113.2, 77.9, 77.7, 48.9, 46.5, 40.3, 33.7, 33.7, 31.3, 26.2, 23.0, 21.8, 20.6, 16.0; ES-API MS: m/z calcd for C₂₆H₃₂IN₃O₄, found 450.2 [M−I].

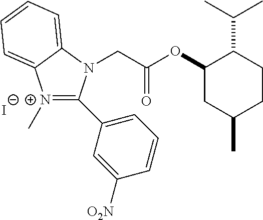

The title compounds were obtained following the general procedure (Step F) described above. Purification of the residue by flash chromatography on silica gel (gradient elution, 5% MeOH in CH₂Cl₂) afforded the entitled salt Lqr-5-121 as a yellow solid (80%). IR (cm⁻¹) 2956, 1738, 1583, 1519, 1458, 1349, 1214, 740; [α]_{D}²⁰−24.668 (c 0.35, CHCl₃); ¹H NMR (400 MHz, CDCl₃) δ 9.06 (d, J=6.8 Hz, 1H), 8.66 (s, 1H), 8.64-8.57 (m, 1H), 7.98 (t, J=8.0 Hz, 1H), 7.90-7.81 (m, 1H), 7.81-7.70 (m, 2H), 7.67-7.61 (m, 1H), 5.14 (s, 2H), 4.76 (td, J=10.8, 4.4 Hz, 1H), 4.04 (s, 3H), 1.90 (d, J=11.2 Hz, 1H), 1.67 (d, J=12.0 Hz, 2H), 1.53-1.42 (m, 1H), 1.37 (t, J=11.6 Hz, 1H), 1.10-0.93 (m, 3H), 0.89 (d, J=6.8 Hz, 3H), 0.94-0.86 (m, 1H), 0.83 (d, J=6.4 Hz, 3H), 0.65 (d, J=4.4 Hz, 3H); ¹³C NMR (100 MHz, CDCl₃) δ 165.4, 148.7, 148.2, 138.8, 132.0, 131.8, 131.6, 128.3, 128.1, 128.0, 125.7, 121.9, 113.5, 112.9, 78.1, 48.9, 46.6, 40.4, 34.0, 33.7, 31.3, 26.4, 23.2, 21.8, 20.5, 16.0; ES-API MS: m/z calcd for C₂₆H₃₂IN₃O₄, found 450.2 [M−I].

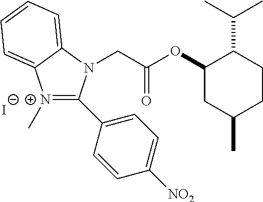

The title compounds were obtained following the general procedure (Step F) described above. Purification of the residue by flash chromatography on silica gel (gradient elution, 5% MeOH in CH₂Cl₂) afforded the entitled salt Lqr-5-122 as a yellow solid (80%). IR (cm⁻¹) 2956, 1739, 1603, 1525, 1457, 1294, 1215, 741; [α]_{D}²⁰−34.660 (c 0.35, CHCl₃); ¹H NMR (400 MHz, CDCl₃) δ 8.55-8.45 (m, 4H), 7.86-7.80 (m, 1H), 7.80-7.70 (m, 2H), 7.68-7.61 (m, 1H), 5.13 (d, J=6.4 Hz, 2H), 4.75 (td, J=10.8, 4.4 Hz, 1H), 4.02 (s, 3H), 1.93-1.83 (m, 1H), 1.74-1.64 (m, 2H), 1.63-1.55 (m, 1H), 1.51-1.34 (m, 2H), 1.10-0.95 (m, 2H), 0.95-0.78 (m, 1H), 0.91 (d, J=6.4 Hz, 3H), 0.86 (d, J=7.2 Hz, 3H), 0.68 (d, J=7.2 Hz, 3H); ¹³C NMR (100 MHz, CDCl₃) δ 165.5, 150.7, 149.0, 133.5, 132.1, 131.9, 128.4 (2C), 128.1 (2C), 126.2, 124.5, 113.5, 112.8, 78.0, 48.8, 46.7, 40.5, 34.0, 33.7, 31.4, 26.4, 23.1, 21.8, 20.5, 16.1; ES-API MS: m/z calcd for C₂₆H₃₂IN₃O₄, found 450.2 [M−I].

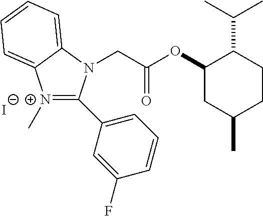

The title compounds were obtained following the general procedure (Step F) described above. Purification of the residue by flash chromatography on silica gel (gradient elution, 5% MeOH in CH₂Cl₂) afforded the entitled salt Lqr-5-123 as a yellow solid (60%). IR (cm⁻¹) 2956, 1739, 1587, 1458, 1386, 1214, 961, 891, 743; [α]_{D}²⁰−41.228 (c 0.3, CHCl₃); ¹H NMR (400 MHz, CDCl₃) δ 8.15-7.99 (m, 1H), 7.87-7.78 (m, 1H), 7.79-7.67 (m, 4H), 7.67-7.60 (m, 1H), 7.53-7.42 (m, 1H), 5.17 (d, J=18.4 Hz, 1H), 5.10 (d, J=18.0 Hz, 1H), 4.78 (td, J=10.8, 4.4 Hz, 1H), 4.02 (s, 3H), 1.91 (d, J=12.4 Hz, 1H), 1.68 (d, J=11.2 Hz, 2H), 1.54-1.33 (m, 2H), 1.28-1.12 (m, 1H), 1.10-0.95 (m, 1H), 0.94-0.76 (m, 2H), 0.92 (d, J=6.8 Hz, 3H), 0.86 (d, J=6.8 Hz, 3H), 0.70 (d, J=7.2 Hz, 3H); ¹³C NMR (100 MHz, CDCl₃) δ 165.7, 149.7, 132.3 (d, J=7.5 Hz, 1C), 132.1, 132.0, 131.7, 128.0, 127.9 (d, J=5.7 Hz, 1C), 127.8, 121.9 (d, J=8.2 Hz, 1C), 121.1, 120.9, 113.4, 112.9, 77.7, 48.8, 46.7, 40.5, 33.8, 33.7, 31.4, 26.4, 23.1, 21.8, 20.5, 16.0; ES-API MS: m/z calcd for C₂₆H₃₂FIN₂O₂, found 423.2 [M−I].

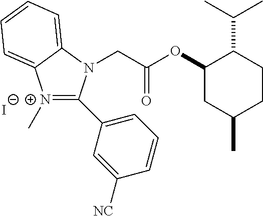

The title compounds were obtained following the general procedure (Step F) described above. Purification of the residue by flash chromatography on silica gel (gradient elution, 5% MeOH in CH₂Cl₂) afforded the entitled salt Lqr-5-124 as a yellow solid (62%). IR (cm⁻¹) 2955, 2231, 1736, 1589, 1456, 1385, 1215, 961, 743; [α]_{D}²⁰−47.660 (c 0.5, CHCl₃); ¹H NMR (400 MHz, CDCl₃) δ 8.81 (s, 1H), 8.19 (s, 1H), 8.05 (d, J=8.0 Hz, 1H), 7.89 (t, J=8.0 Hz, 1H), 7.86-7.81 (m, 1H), 7.79-7.69 (m, 2H), 7.67-7.58 (m, 1H), 5.11 (s, 2H), 4.77 (td, J=10.8, 4.4 Hz, 1H), 4.01 (s, 3H), 1.90 (d, J=12.0 Hz, 1H), 1.68 (d, J=11.2, 2H), 1.55-1.33 (m, 2H), 1.31-1.15 (m, 2H), 1.11-0.95 (m, 2H), 0.92 (d, J=6.8 Hz, 3H), 0.87 (d, J=7.2 Hz, 3H), 0.70 (d, J=6.8 Hz, 3H); ¹³C NMR (100 MHz, CDCl₃) δ 165.5, 158.9, 148.8, 136.7, 133.9, 132.0, 131.7, 131.1, 128.3, 128.0, 122.0, 116.6, 114.2, 113.5, 112.8, 78.1, 48.8, 46.7, 40.5, 34.0, 33.7, 31.4, 26.5, 23.2, 21.8, 20.5, 16.2; ES-API MS: m/z calcd for C₂₇H₃₂IN₃O₂, found 430.2 [M−I].

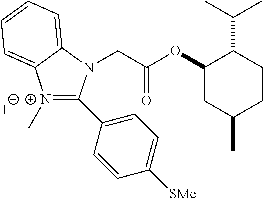

The title compounds were obtained following the general procedure (Step F) described above. Purification of the residue by flash chromatography on silica gel (gradient elution, 5% MeOH in CH₂Cl₂) afforded the entitled salt Lqr-5-125 as a yellow solid (60%). IR (cm⁻¹) 2958, 2360, 1738, 1602, 1455, 1261, 1201, 1100, 799, 744; [α]_{D}²⁰−34.220 (c 0.3, CHCl₃); ¹H NMR (400 MHz, CDCl₃) δ 7.95 (d, J=7.2 Hz, 2H), 7.83-7.76 (m, 1H), 7.72-7.65 (m, 2H), 7.64-7.58 (m, 1H), 7.46 (d, J=8.8 Hz, 2H), 5.18 (d, J=18.0 Hz, 1H), 5.12 (d, J=18.0 Hz, 1H), 4.76 (td, J=10.8, 4.4 Hz, 1H), 4.03 (s, 3H), 2.56 (s, 3H), 1.96-1.86 (m, 1H), 1.74-1.55 (m, 3H), 1.52-1.33 (m, 2H), 1.20 (t, J=7.2 Hz, 1H), 1.10-0.95 (m, 2H), 0.92 (d, J=6.8 Hz, 3H), 0.87 (d, J=7.2 Hz, 3H), 0.70 (d, J=7.2 Hz, 3H); ¹³C NMR (100 MHz, CDCl₃) δ 165.8, 151.3, 147.3, 131.9, 131.7, 131.2, 127.7, 127.5 (2C), 125.9 (2C), 115.2, 113.3, 112.8, 77.6, 77.2, 48.8, 46.7, 40.5, 33.8, 31.4, 26.3, 23.1, 21.8, 20.6, 16.1, 14.6; ES-API MS: m/z calcd for C₂₇H₃₅IN₂O₂S, found 451.2 [M−I].

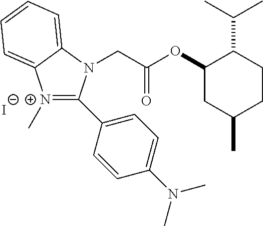

The title compounds were obtained following the general procedure (Step F) described above. Purification of the residue by flash chromatography on silica gel (gradient elution, 5% MeOH in CH₂Cl₂) afforded the entitled salt Lqr-5-126 as a yellow solid (87%). IR (cm⁻¹) 2954, 1739, 1600, 1512, 1475, 1382, 1226, 1200, 757; [α]_{D}²⁰−43.996 (c 0.7, CHCl₃); ¹H NMR (400 MHz, CDCl₃) δ 7.85-7.78 (m, 1H), 7.70 (d, J=8.8 Hz, 2H), 7.67-7.57 (m, 3H), 6.84 (d, J=8.8 Hz, 2H), 5.19 (d, J=18.0 Hz, 1H), 5.16 (d, J=18.0 Hz, 1H), 4.78 (td, J=10.8, 4.4 Hz, 1H), 4.07 (s, 3H), 3.10 (s, 6H), 1.98-1.89 (m, 1H), 1.74-1.62 (m, 3H), 1.53-1.33 (m, 2H), 1.19 (t, J=7.2 Hz, 1H), 1.11-0.95 (m, 2H), 0.91 (d, J=6.8 Hz, 3H), 0.87 (d, J=7.2 Hz, 3H), 0.71 (d, J=7.2 Hz, 3H); ¹³C NMR (100 MHz, CDCl₃) δ 166.1, 153.1, 152.7, 132.1 (2C), 132.0, 131.7, 127.2 (2C), 127.1, 113.2, 112.5, 111.9, 104.5, 48.7, 46.7 (2C), 40.5, 40.0, 34.0, 33.8, 31.4, 26.2, 23.0, 21.8, 20.7, 16.1; ES-API MS: m/z calcd for C₂₈H₃₈IN₃O₂, found 448.3 [M−I].

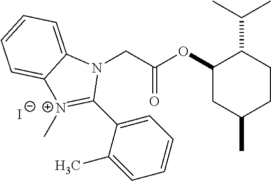 [SMILES: Cc1ccccc1-c1n(CC(=O)O[C@@H]2C[C@H](C)CC[C@H]2C(C)C)c2ccccc2[n+]1C ; [I-]]

The title compounds were obtained following the general procedure (Step F) described above. Purification of the residue by flash chromatography on silica gel (gradient elution, 5% MeOH in CH₂Cl₂) afforded the entitled salt Lqr-5-127 as a yellow solid (82%). IR (cm⁻¹) 2954, 1753, 1610, 1459, 1384, 1209, 981, 744; [α]_{D}²⁰−30.445 (c 0.4, CHCl₃); ¹H NMR (400 MHz, CDCl₃) δ 8.15 (d, J=7.2 Hz, 1H), 7.92-7.85 (m, 1H), 7.80-7.69 (m, 3H), 7.69-7.62 (m, 1H), 7.56-7.43 (m, 2H), 5.30 (dd, J=18.4, 5.2 Hz, 1H), 5.12 (dd, J=18.4, 1.6 Hz, 1H), 4.75-4.62 (m, 1H), 3.93 (d, J=1.6 Hz, 3H), 2.25 (s, 3H), 1.93-1.75 (m, 1H), 1.72-1.55 (m, 2H), 1.49-1.23 (m, 3H), 1.08-0.75 (m, 3H), 0.92-0.83 (m, 5H), 0.78-0.66 (m, 3H), 0.56 (d, J=6.8 Hz, 1H); ¹³C NMR (100 MHz, CDCl₃) δ 165.34, 151.07, 145.08, 138.75, 133.69, 131.87, 131.71, 131.55, 131.38, 127.92, 127.64, 127.47, 119.52, 113.56, 46.6, 40.4, 40.3, 33.7, 31.3, 26.2, 23.1, 21.8, 20.6, 20.5, 19.8, 19.5, 16.0; ES-API MS: m/z calcd for C₂₇H₃₅IN₂O₂, found 419.2 [M−I].

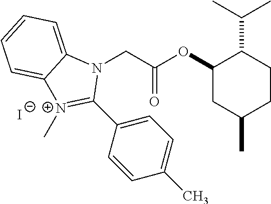 [SMILES: Cc1ccc(-c2n(CC(=O)O[C@@H]3C[C@H](C)CC[C@H]3C(C)C)c3ccccc3[n+]2C)cc1 ; [I-]]

The title compounds were obtained following the general procedure (Step F) described above. Purification of the residue by flash chromatography on silica gel (gradient elution, 5% MeOH in CH₂Cl₂) afforded the entitled salt Lqr-5-128 as a yellow solid (80%). IR (cm⁻¹) 2955, 2360, 1738, 1613, 1484, 1559, 1384, 1206, 961, 822, 743; [α]_{D}²⁰−40.226 (c 0.4, CHCl₃); ¹H NMR (400 MHz, CDCl₃) δ 7.89 (d, J=6.8 Hz, 2H), 7.84-7.77 (m, 1H), 7.73-7.65 (m, 2H), 7.65-7.60 (m, 1H), 7.48 (d, J=8.0 Hz, 2H), 5.18 (d, J=18.4 Hz, 1H), 5.14 (d, J=18.0 Hz, 1H), 4.76 (td, J=10.8, 4.4 Hz, 1H), 4.02 (s, 3H), 2.50 (s, 3H), 1.97-1.85 (m, 1H), 1.75-1.54 (m, 3H), 1.52-1.31 (m, 2H), 1.28-1.15 (m, 1H), 1.11-0.95 (m, 2H), 0.91 (d, J=6.8 Hz, 3H), 0.87 (d, J=7.2 Hz, 3H), 0.69 (d, J=7.2 Hz, 3H); ¹³C NMR (100 MHz, CDCl₃) δ 165.8, 151.6, 144.6, 131.9, 131.7, 130.9, 130.5 (2C), 127.7 (2C), 127.5, 117.0, 113.3, 112.8, 77.5, 48.7, 46.7, 40.5, 33.8, 33.7, 31.4, 26.3, 23.1, 21.8, 21.8, 20.6, 16.1; ES-API MS: m/z calcd for C₂₇H₃₅IN₂O₂, found 419.2 [M−I].

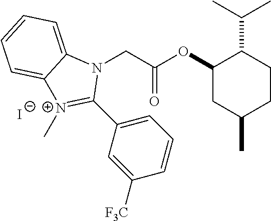 [SMILES: CC(C)[C@@H]1CC[C@@H](C)C[C@H]1OC(=O)Cn1c(-c2cccc(C(F)(F)F)c2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt Lqr-5-129 as a yellow solid (60%). IR (cm⁻¹) 2957, 1738, 1597, 1460, 1389, 13226, 1213, 1169, 1129, 743; [α]_{D}²⁰−38.667 (c 0.3, CHCl₃); ¹H NMR (400 MHz, CDCl₃) δ 8.72-8.56 (m, 1H), 8.12-8.01 (m, 1H), 7.99 (d, J=8.0 Hz, 1H), 7.94-7.82 (m, 2H), 7.74-7.57 (m, 3H), 5.25-4.91 (m, 2H), 4.71 (td, J=10.8, 4.4 Hz, 1H), 3.99 (s, 3H), 1.93-1.78 (m, 1H), 1.70-1.48 (m, 3H), 1.48-1.26 (m, 2H), 1.06-0.90 (m, 2H), 0.90-0.80 (m, 1H), 0.87 (d, J=6.8 Hz, 3H), 0.84-0.72 (m, 3H), 0.63 (d, J=6.4 Hz, 3H); ¹³C NMR (100 MHz, CDCl₃) δ 165.2, 149.2, 135.6, 131.9 (q, J=33.5 Hz, 1C), 131.8, 131.5, 131.0, 130.2 (q, J=3.3 Hz, 1C), 128.0, 127.9, 127.4 (q, J=2.7 Hz, 1C), 124.2, 121.1, 114.0, 112.8, 77.8, 48.8, 46.5, 40.2, 34.2 (d, J=95.8 Hz, 1C), 31.3 (d, J=20.3 Hz, 1C), 26.2, 22.7 (d, J=51.0 Hz, 1C), 21.7, 20.4, 15.9, 14.0; ES-API MS: m/z calcd for C₂₇H₃₂F₃IN₂O₂, found 473.2 [M−I].

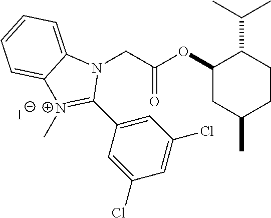 [SMILES: CC(C)[C@@H]1CC[C@@H](C)C[C@H]1OC(=O)Cn1c(-c2cc(Cl)cc(Cl)c2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt Lqr-5-130 as a yellow solid (60%). IR (cm⁻¹) 2956, 1738, 1598, 1565, 1460, 1384, 1212, 861, 806, 742; [α]_{D}²⁰−48.224 (c 0.3, CHCl₃); ¹H NMR (400 MHz, CDCl₃) δ 8.18-8.02 (m, 2H), 7.85-7.78 (m, 1H), 7.77-7.70 (m, 3H), 7.66-7.60 (m, 1H), 5.13 (d, J=2.8 Hz, 2H), 4.79 (td, J=10.8, 4.4 Hz, 1H), 4.02 (s, 3H), 1.98-1.89 (m, 1H), 1.75-1.60 (m, 3H), 1.54-1.35 (m, 2H), 1.29-1.17 (m, 1H), 1.12-0.96 (m, 2H), 0.92 (d, J=6.4 Hz, 3H), 0.87 (d, J=7.2 Hz, 3H), 0.71 (d, J=7.2 Hz, 3H); ¹³C NMR (100 MHz, CDCl₃) δ 165.5, 148.2, 136.9, 133.9 (2C), 132.0, 131.8, 129.6, 128.3 (2C), 128.0, 123.2, 113.4, 112.9, 78.0, 48.8, 46.7, 40.5, 33.8, 33.8, 31.4, 26.5, 23.2, 21.8, 20.6, 16.1; ES-API MS: m/z calcd for C₂₆H₃₁Cl₂IN₂O₂, found 473.1 [M−I].

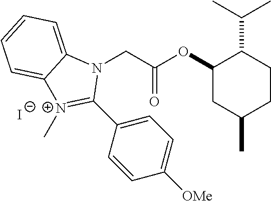 [SMILES: COc1ccc(-c2n(CC(=O)O[C@@H]3C[C@H](C)CC[C@H]3C(C)C)c3ccccc3[n+]2C)cc1 ; [I-]]

The title compounds were obtained following the general procedure (Step F) described above. Purification of the residue by flash chromatography on silica gel (gradient elution, 5% MeOH in CH₂Cl₂) afforded the entitled salt Lqr-5-131 as a yellow solid (72%). IR (cm⁻¹) 2229, 1757, 1621, 1486, 1427, 1384, 1331, 1165, 714; [α]_{D}²⁰−40.668 (c 0.5, CHCl₃); ¹H NMR (400 MHz, CDCl₃) δ 7.95 (d, J=7.6 Hz, 2H), 7.85-7.76 (m, 1H), 7.71-7.63 (m, 2H), 7.63-7.58 (m, 1H), 7.16 (d, J=8.8 Hz, 2H), 5.23-5.07 (m, 2H), 4.76 (td, J=10.8, 4.4 Hz, 1H), 4.02 (s, 3H), 3.92 (s, 3H), 1.95-1.86 (m, 1H), 1.86-1.80 (m, 1H), 1.74-1.57 (m, 3H), 1.53-1.32 (m, 2H), 1.09-0.94 (m, 2H), 0.91 (d, J=6.8 Hz, 3H), 0.86 (d, J=7.2 Hz, 3H), 0.69 (d, J=7.2 Hz, 3H); ¹³C NMR (100 MHz, CDCl₃) δ 165.9, 163.5, 151.6, 133.0, 131.9, 131.7, 127.6 (2C), 127.4, 115.4 (2C), 113.3, 112.7, 111.4, 77.5, 55.7, 48.7, 46.7, 40.5, 33.8, 33.8, 31.4, 26.3, 23.1, 21.8, 20.6, 16.1; ES-API MS: m/z calcd for C₂₇H₃₅IN₂O₃, found 435.2 [M−I].

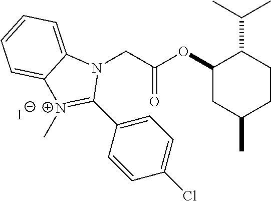 [SMILES: CC(C)[C@@H]1CC[C@@H](C)C[C@H]1OC(=O)Cn1c(-c2ccc(Cl)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-249 as a yellow solid (89%). IR (cm⁻¹) 2957, 2871, 1741, 1482, 1469, 1224, 1095, 756; [α]_{D}²⁰−31.15 (c 0.43, CHCl₃); ¹H NMR (400 MHz, CDCl₃) δ 7.98 (m, 2H), 7.85 (m, 1H), 7.66-7.59 (m, 5H), 5.15 (d, J=18.0 Hz, 1H), 5.05 (d, J=18.0 Hz, 1H), 4.67 (ddd, J=10.8, 10.8, 4.4 Hz, 1H), 3.96 (s, 3H), 1.81 (m, 1H), 1.61 (m, 2H), 1.51 (m, 1H), 1.39 (m, 1H), 1.31 (m, 1H), 0.95 (m, 2H), 0.85 (d, J=6.4 Hz, 3H), 0.81 (m, 1H), 0.80 (d, J=6.8 Hz, 3H), 0.61 (d, J=7.2 Hz, 3H); ¹³C NMR (100 MHz, CDCl₃) δ 165.5, 150.2, 140.4, 132.8, 130.3 (4C), 128.0, 127.8, 118.4, 113.8, 112.8, 77.7, 67.1, 48.8, 46.7, 40.5, 34.4, 33.8, 31.4, 26.3, 23.1, 21.9, 20.6, 16.1; ES-API MS: m/z calcd for C₂₆H₃₂ClIN₂O₂, found 439.2 [M−I].

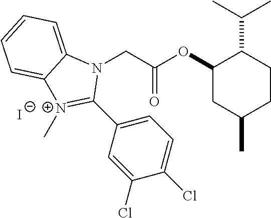 [SMILES: CC(C)[C@@H]1CC[C@@H](C)C[C@H]1OC(=O)Cn1c(-c2ccc(Cl)c(Cl)c2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-250 as a yellow solid (88%). IR (cm⁻¹) 2957, 2928, 2871, 1740, 1484, 1454, 1225, 1035, 755; [α]_{D}²⁰−25.87 (c 0.51, CHCl₃); ¹H NMR (400 MHz, CDCl₃) δ 8.07 (m, 1H), 8.01 (m, 1H), 7.86 (m, 1H), 7.71 (d, J=8.4 Hz, 1H), 7.66-7.58 (m, 3H), 5.11 (d, J=18.4 Hz, 1H), 5.04 (d, J=18.4 Hz, 1H), 4.67 (ddd, J=10.8, 10.8, 4.4 Hz, 1H), 3.96 (s, 3H), 1.82 (m, 1H), 1.60 (m, 2H), 1.51 (m, 1H), 1.38 (m, 1H), 1.30 (m, 1H), 0.95 (m, 2H), 0.84 (d, J=6.4 Hz, 3H), 0.81 (m, 1H), 0.78 (d, J=6.8 Hz, 3H), 0.60 (d, J=6.8 Hz, 3H); ¹³C NMR (100 MHz, CDCl₃) δ 165.3, 148.6, 138.8, 134.4, 132.4, 132.1, 131.9, 131.6, 131.2, 128.1, 127.9, 119.8, 113.9, 112.8, 77.8, 48.8, 46.6, 40.5, 34.6, 33.7, 31.3, 26.3, 23.1, 21.8, 20.6, 16.1; ES-API MS: m/z calcd for C₂₆H₃₁Cl₂IN₂O₂, found 473.1 [M−I].

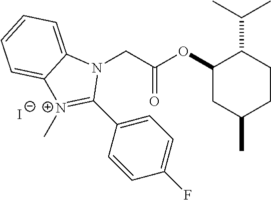 [SMILES: CC(C)[C@@H]1CC[C@@H](C)C[C@H]1OC(=O)Cn1c(-c2ccc(F)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-251 as a yellow solid (80%). IR (cm⁻¹) 2956, 2872, 1741, 1606, 1483, 1469, 1229, 758; [α]_{D}²⁰−31.57 (c 0.38, CHCl₃); ¹H NMR (400 MHz, CDCl₃) δ 7.95 (m, 2H), 7.81 (m, 1H), 7.55 (m, 3H), 7.26 (d, J=8.4 Hz, 1H), 7.24 (d, J=8.4 Hz, 1H), 5.09 (d, J=18.0 Hz, 1H), 4.98 (d, J=18.0 Hz, 1H), 4.59 (ddd, J=11.2, 10.8, 4.4 Hz, 1H), 3.90 (s, 3H), 1.75 (m, 1H), 1.54 (m, 2H), 1.43 (m, 1H), 1.31 (m, 1H), 1.23 (m, 1H), 0.88 (m, 2H), 0.77 (d, J=6.4 Hz, 3H), 0.75 (m, 1H), 0.72 (d, J=6.8 Hz, 3H), 0.53 (d, J=6.8 Hz, 3H); ¹³C NMR (100 MHz, CDCl₃) δ 165.3 (d, J=256.1 Hz, 1C), 165.2, 150.0, 133.8, 133.7, 131.5 (d, J=29.8 Hz, 2C), 127.7, 127.5, 117.3 (d, J=22.3 Hz, 2C), 115.8 (d, J=3.5 Hz, 1C), 113.7, 112.6, 77.4, 48.5, 46.4, 40.3, 34.4, 33.5, 31.1, 26.1, 22.9, 21.7, 20.4, 15.9; ES-API MS: m/z calcd for C₂₆H₃₂FIN₂O₂, found 423.2 [M−I].

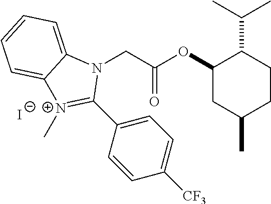 [SMILES: CC(C)[C@@H]1CC[C@@H](C)C[C@H]1OC(=O)Cn1c(-c2ccc(C(F)(F)F)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-252 as a yellow solid (87%). IR (cm⁻¹) 2929, 2957, 2873, 1741, 1323, 1225, 1136, 757; [α]_{D}²⁰−29.99 (c 0.52, CHCl₃); ¹H NMR (400 MHz, CDCl₃) δ 8.19 (m, 2H), 7.87-7.83 (m, 3H), 7.64-7.58 (m, 3H), 5.13 (d, J=18.4 Hz, 1H), 5.00 (d, J=18.4 Hz, 1H), 4.63 (ddd, J=10.8, 10.8, 4.4 Hz, 1H), 3.94 (s, 3H), 1.76 (m, 1H), 1.58 (m, 2H), 1.45 (m, 1H), 1.34 (m, 1H), 1.26 (m, 1H), 0.91 (m, 2H), 0.81 (d, J=6.4 Hz, 3H), 0.78 (m, 1H), 0.74 (d, J=6.8 Hz, 3H), 0.56 (d, J=6.8 Hz, 3H); ¹³C NMR (100 MHz, CDCl₃) δ 165.2, 149.4, 135.0 (q, J=33.2 Hz, 1C), 132.2 (2C), 131.8, 131.5, 128.0, 127.8, 126.6 (q, J=3.5 Hz, 2C), 123.7 (d, J=0.8 Hz, 1C), 122.9 (q, J=271.8 Hz, 1C), 113.9, 112.8, 77.7, 48.7, 46.5, 40.4, 34.6, 33.6, 31.2, 26.2, 23.0, 21.7, 20.4, 16.0; ES-API MS: m/z calcd for C₂₇H₃₂F₃IN₂O₂, found 473.2 [M−I].

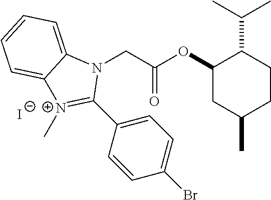 [SMILES: CC(C)[C@@H]1CC[C@@H](C)C[C@H]1OC(=O)Cn1c(-c2ccc(Br)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-253 as a yellow solid (82%). IR (cm⁻¹) 2955, 2926, 2870, 1740, 1482, 1467, 1225, 756; [α]_{D}²⁰−30.72 (c 0.41, CHCl₃); ¹-H NMR (400 MHz, CDCl₃) δ 7.88-7.82 (m, 3H), 7.72 (m, 2H), 7.62-7.56 (m, 3H), 5.13 (d, J=18.0 Hz, 1H), 5.02 (d, J=18.0 Hz, 1H), 4.63 (ddd, J=10.8, 10.8, 4.4 Hz, 1H), 3.93 (s, 3H), 1.77 (m, 1H), 1.57 (m, 2H), 1.46 (m, 1H), 1.34 (m,1H), 1.27 (m, 1H), 0.91 (m, 2H), 0.81 (d, J=6.8 Hz, 3H), 0.78 (m, 1H), 0.76 (d, J=6.8 Hz, 3H), 0.57 (d, J=7.2 Hz, 3H); ¹³C NMR (100 MHz, CDCl₃) δ 165.3, 150.0, 133.1 (3C), 132.6, 131.8, 131.5, 128.8, 127.8, 127.6, 118.7, 113.8, 112.7, 77.5, 48.6, 46.5, 40.4, 34.5, 33.6, 31.2, 26.2, 23.0, 21.8, 20.5, 16.0; ES-API MS: m/z calcd for C₂₆H₃₂BrIN₂O₂, found 483.1 [M−I].

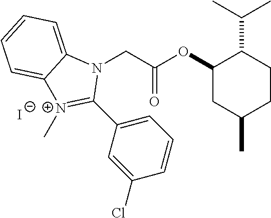 [SMILES: CC(C)[C@@H]1CC[C@@H](C)C[C@H]1OC(=O)Cn1c(-c2cccc(Cl)c2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-254 as a yellow solid (93%). IR (cm⁻¹) 2955, 2926, 2870, 1740, 1484, 1460, 1225, 750; [α]_{D}²⁰−32.18 (c 0.41, CHCl₃); ¹H NMR (400 MHz, CDCl₃) δ 7.96 (m, 1H), 7.87 (m, 1H), 7.78 (m, 1H), 7.66 (m, 1H), 7.63-7.57 (m, 4H), 5.11 (d, J=18.4 Hz, 1H), 5.02 (d, J=18.4 Hz, 1H), 4.65 (ddd, J=11.2, 11.2, 4.4 Hz, 1H), 3.94 (s, 3H), 1.81 (m, 1H), 1.57 (m, 2H), 1.49 (m, 1H), 1.35 (m, 1H), 1.27 (m, 1H), 0.92 (m, 2H), 0.81 (d, J=6.8 Hz, 3H), 0.78 (m, 1H), 0.74 (d, J=6.8 Hz, 3H), 0.58 (d, J=7.2 Hz, 3H); ¹³C NMR (100 MHz, CDCl₃) δ 165.3, 149.3, 135.7, 133.8, 131.7, 131.4, 130.2, 129.8, 127.9, 127.7, 121.6, 113.9, 112.8, 77.6, 53.6, 48.7, 46.5, 40.4, 34.5, 33.6, 31.2, 26.2, 23.0, 21.7, 20.5, 16.0; ES-API MS: m/z calcd for C₂₆H₃₂ClIN₂O₂, found 439.2 [M−I].

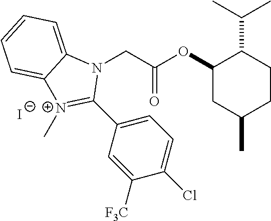 [SMILES: CC(C)[C@@H]1CC[C@@H](C)C[C@H]1OC(=O)Cn1c(-c2ccc(Cl)c(C(F)(F)F)c2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-255 as a white solid (86%). IR (cm⁻¹) 2957, 2872, 1740, 1466, 1318, 1225, 1141, 1037, 756; [α]_{D}²⁰−21.45 (c 0.41, CHCl₃); ¹H NMR (400 MHz, CDCl₃) δ 8.42 (m, 1H), 8.13 (m, 1H), 7.85 (m, 1H), 7.75 (d, J=8.4 Hz, 1H), 7.60-7.54 (m, 3H), 5.04 (d, J=18.4 Hz, 1H), 4.97 (d, J=18.4 Hz, 1H), 4.60 (ddd, J=10.8, 10.8, 4.4 Hz, 1H), 3.90 (s, 3H), 1.74 (m, 1H), 1.55 (m, 2H), 1.45 (m, 1H), 1.32 (m, 1H), 1.24 (m, 1H), 0.88 (m, 2H), 0.78 (d, J=6.4 Hz, 3H), 0.75 (m, 1H), 0.71 (d, J=6.8 Hz, 3H), 0.52 (d, J=7.2 Hz, 3H); ¹³C NMR (100 MHz, CDCl₃) δ 165.0, 148.3, 138.1 (q, J=1.2 Hz, 1C), 136.8, 133.2, 131.7, 131.3, 129.7 (q, J=2.6 Hz, 1C), 129.5 (q, J=32.4 Hz, 1C), 128.0, 127.8, 121.6 (q, J=272.9 Hz, 1C), 118.9, 113.9, 112.6, 77.7, 48.6, 46.3, 40.2, 34.6, 33.5, 31.1, 26.1, 22.9, 21.5, 20.3, 15.8; ES-API MS: m/z calcd for C₂₇H₃₁ClF₃IN₂O₂, found 507.2 [M−I].

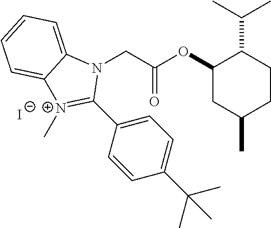 [SMILES: CC(C)[C@@H]1CC[C@@H](C)C[C@H]1OC(=O)Cn1c(-c2ccc(C(C)(C)C)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-256 as a white solid (95%). IR (cm⁻¹) 2957, 2870, 1741, 1480, 1366, 1225, 754; [α]_{D}²⁰−36.57 (c 0.41, CHCl₃); NMR (400 MHz, CDCl₃) δ 7.83 (m, 1H), 7.72 (m, 2H), 7.61-7.52 (m, 5H), 5.18 (d, J=18.4 Hz, 1H), 4.99 (d, J=18.4 Hz, 1H), 4.64 (ddd, J=10.8, 10.8, 4.4 Hz, 1H), 3.93 (s, 3H), 1.79 (m, 1H), 1.55 (m, 2H), 1.50 (m, 1H), 1.33 (m, 1H), 1.27 (s, 9H), 1.24 (m, 1H), 0.89 (m, 2H), 0.79 (d, J=6.4 Hz, 3H), 0.76 (m, 1H), 0.72 (d, J=6.8 Hz, 3H), 0.57 (d, J=7.2 Hz, 3H); ¹³C NMR (100 MHz, CDCl₃) δ 165.5, 157.2, 151.2, 131.6, 131.4, 130.4 (2C), 127.5, 127.3, 126.7 (2C), 116.7, 113.6, 112.8, 77.2, 48.6, 46.5, 40.3, 35.2, 34.3, 33.6, 31.2, 30.8 (3C), 26.0, 22.9, 21.7, 20.5, 15.9; ES-API MS: m/z calcd for C₃₀H₄₁IN₂O₂, found 461.3 [M−I].

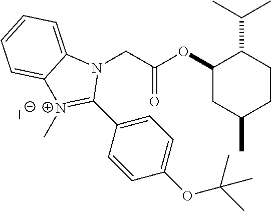 [SMILES: CC(C)[C@@H]1CC[C@@H](C)C[C@H]1OC(=O)Cn1c(-c2ccc(OC(C)(C)C)cc2)[n+](C)c2ccccc21 ; [I-]]

The title compounds were obtained following the general procedure (Step F) described above. Purification of the residue by flash chromatography on silica gel (gradient elution, 5% MeOH in CH₂Cl₂) afforded the entitled salt LW-V-257 as a white solid (88%). IR (cm⁻¹) 2956, 2871, 1740, 1605, 1469, 1369, 1225, 1160; [α]_{D}²⁰−33.16 (c 0.41, CHCl₃); ¹H NMR (400 MHz, CDCl₃) δ 7.81 (m, 1H), 7.71 (d, J=8.4 Hz, 2H), 7.58-7.52 (m, 3H), 7.11 (d, J=8.8 Hz, 2H), 5.15 (d, J=18.0 Hz, 1H), 5.02 (d, J=18.0 Hz, 1H), 4.63 (ddd, J=10.8, 10.8, 4.4 Hz, 1H), 3.93 (s, 3H), 1.78 (m, 1H), 1.55 (m, 2H), 1.51 (m, 1H), 1.38 (s, 9H), 1.33 (m, 1H), 1.25 (m, 1H), 0.89 (m, 2H), 0.79 (d, J=6.8 Hz, 3H), 0.76 (m, 1H), 0.73 (d, J=6.8 Hz, 3H), 0.57 (d, J=7.2 Hz, 3H); ¹³C NMR (100 MHz, CDCl₃) δ 165.5, 160.6, 151.1, 132.0 (2C), 131.6, 131.3, 127.5, 127.3, 122.4 (2C), 113.6, 112.7, 112.2, 80.2, 77.3, 48.6, 46.5, 40.3, 32.3, 33.6, 31.2, 28.7 (3C), 26.1, 22.9, 21.7, 20.5, 15.9; ES-API MS: m/z calcd for C₃₀H₄₁IN₂O₃, found 477.3 [M−I].

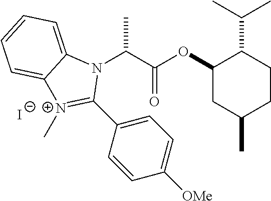 [SMILES: COc1ccc(-c2n([C@H](C)C(=O)O[C@@H]3C[C@H](C)CC[C@H]3C(C)C)c3ccccc3[n+]2C)cc1 ; [I-]]

The title compounds were obtained following the general procedure (Step F) described above. Purification of the residue by prep-TLC (8% MeOH in CH₂Cl₂) afforded the entitled salt LW-III-311 as a yellow gel (87%). IR (cm⁻¹) 3440, 2956, 2871, 1738, 1609, 1469, 1302, 1260, 1182, 843, 754; [α]_{D}²⁰+33.51 (c 1.08, CHCl₃); ¹H NMR (400 MHz, CDCl₃) δ 8.39 (d, J=4.0 Hz, 1H), 7.89 (d, J=8.0 Hz, 1H), 7.64 (ddd, J=8.4, 7.2, 0.8 Hz, 1H), 7.58 (dd, J=8.0, 6.8 Hz, 1H), 7.53 (d, J=8.4 Hz, 1H), 7.45 (m, 1H), 7.21 (m, 1H), 7.11 (m, 1H), 5.16 (q, J=7.2 Hz, 1H), 4.76 (ddd, J=11.2, 10.8, 4.4 Hz, 1H), 4.01 (s, 3H), 3.90 (s, 3H), 1.92 (d, J=7.6 Hz, 3H), 1.78 (m, 1H), 1.74 (m, 1H), 1.67-1.60 (m, 2H), 1.41 (m, 1H), 1.35 (m, 1H), 0.99 (m, 1H), 0.89 (d, J=6.8 Hz, 3H), 0.82 (m, 1H), 0.81 (d, J=6.8 Hz, 3H), 0.76 (m, 1H), 0.72 (d, J=7.2 Hz, 3H); ¹³C NMR (100 MHz, CDCl₃) δ 167.7, 163.6, 151.1, 134.5, 132.6, 131.5, 129.7, 127.5, 127.3, 115.9, 115.6, 114.4, 113.8, 111.7, 77.7, 56.7, 56.0, 46.8, 40.3, 34.6, 33.8, 31.4, 26.5, 23.1, 21.9, 20.9, 16.7, 16.1; ES-API MS: m/z calcd for C₂₈H₃₇IN₂O₃, found 449.3 [M−I].

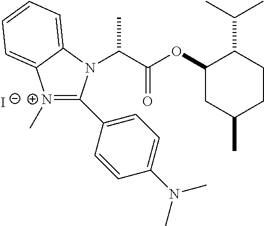

The title compounds were obtained following the general procedure (Step F) described above. Purification of the residue by prep-TLC (7% MeOH in CH₂Cl₂) afforded the entitled salt LW-III-312 as a yellow gel (80%). IR (cm⁻¹) 3429, 2955, 2870, 1738, 1605, 1514, 1470, 1372, 1201, 753; [α]_{D}²⁰+89.60 (c 1.06, CHCl₃); ¹H NMR (400 MHz, CDCl₃) δ 7.93 (d, J=8.4 Hz, 1H), 7.94-7.30 (br, 2H), 7.63 (ddd, J=8.4, 7.2, 1.2 Hz, 1H), 7.55 (ddd, J=8.4, 7.2, 1.2 Hz, 1H), 7.50 (d, J=8.4 Hz, 1H), 6.85 (br, 2H), 5.27 (q, J=7.6 Hz, 1H), 4.78 (ddd, J=11.2, 10.8, 4.4 Hz, 1H), 4.08 (s, 3H), 3.09 (s, 6H), 1.90 (d, J=7.6 Hz, 3H), 1.83-1.72 (m, 2H), 1.69-1.62 (m, 2H), 1.47-1.33 (m, 2H), 1.01 (m, 1H), 0.90 (d, J=6.8 Hz, 3H), 0.82 (d, J=6.8 Hz, 3H), 0.78 (m, 1H), 0.74 (d, J=6.8 Hz, 3H), 0.72 (m, 1H); ¹³C NMR (100 MHz, CDCl₃) δ 167.9, 153.2, 152.3, 132.6, 129.7, 127.2, 127.0, 114.4, 113.6, 104.7, 77.7, 56.6, 46.8, 40.3, 40.2, 34.8, 33.9, 31.4, 26.5, 23.2, 22.0, 20.9, 16.7, 16.2; ES-API MS: m/z calcd for C₂₉H₄₀IN₃O₂, found 462.3 [M−I].

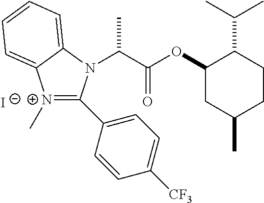

The title compounds were obtained following the general procedure (Step F) described above. Purification of the residue by prep-TLC (8% MeOH in CH₂Cl₂) afforded the entitled salt LW-IV-1 as a yellow gel (82%). IR (cm⁻¹) 3440, 2960, 2872, 1738, 1469, 1323, 1261, 1135, 802, 753, 701; [α]_{D}²⁰+8.75 (c 2.01, CHCl₃); ¹H NMR (400 MHz, CDCl₃) δ 8.86 (d, J=8.0 Hz, 1H), 7.94 (d, J=8.0 Hz, 1H), 7.89 (d, J=7.6 Hz, 1H), 7.86 (d, J=8.4 Hz, 1H), 7.80 (d, J=7.6 Hz, 1H), 7.66 (ddd, J=8.4, 7.6, 1.6 Hz, 1H), 7.61 (d, J=8.4, 7.2, 1.6 Hz, 1H), 7.57 (d, J=8.4 Hz, 1H), 5.05 (q, J=7.6 Hz, 1H), 4.73 (ddd, J=11.2, 10.8, 4.4 Hz, 1H), 3.95 (s, 3H), 1.95 (d, J=7.6 Hz, 3H), 1.76 (m, 1H), 1.72-1.59 (m, 3H), 1.44-1.32 (m, 2H), 0.97 (m, 1H), 0.87 (d, J=6.8 Hz, 3H), 0.82 (m, 1H), 0.79 (d, J=6.8 Hz, 3H), 0.76 (m, 1H), 0.69 (d, J=7.2 Hz, 3H); ¹³C NMR (100 MHz, CDCl₃) δ 167.4, 149.0, 135.2 (q, J=33.2 Hz, 1C), 134.0, 132.7, 131.1, 129.9, 127.8, 127.6, 127.2, 126.4, 124.4, 121.7, 114.5, 113.9, 77.9, 56.8, 46.7, 40.2, 34.8, 33.7, 31.3, 26.4, 23.0, 21.8, 20.8, 16.7, 16.0; ES-API MS: m/z calcd for C₂₈H₃₄F₃IN₂O₂, found 487.3 [M−I].

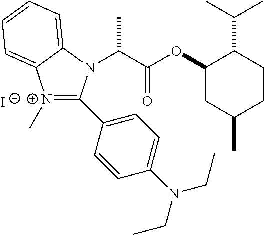

The title compounds were obtained following the general procedure (Step F) described above. Purification of the residue by prep-TLC (8% MeOH in CH₂Cl₂) afforded the entitled salt LW-IV-57 as a yellow gel (80%). IR (cm⁻¹) 3432, 2958, 2872, 1738, 1603, 1471, 1202, 752; [α]_{D}²⁰−25.52 (c 0.76, CHCl₃); ¹H NMR (400 MHz, CDCl₃) δ 8.12-7.30 (m, 2H), 7.92 (d, J=8.4 Hz, 1H), 7.61 (dd, J=8.0, 7.6 Hz, 1H), 7.54 (dd, J=7.6, 7.6 Hz, 1H), 7.48 (d, J=8.0 Hz, 1H), 6.80 (m, 2H), 5.28 (q, J=7.2 Hz, 1H), 4.77 (ddd, J=10.8, 10.8, 4.4 Hz, 1H), 4.08 (s, 3H), 3.43 (q, J=7.2 Hz, 4H), 1.89 (d, J=7.2 Hz, 3H), 1.83-1.74 (m, 2H), 1.68-1.58 (m, 2H), 1.46-1.32 (m, 2H), 1.21 (t, J=6.8 Hz, 6H), 1.00 (m, 1H), 0.89 (d, J=7.2 Hz, 3H), 0.81 (d, J=6.8 Hz, 3H), 0.77 (m, 1H), 0.74 (d, J=6.8 Hz, 3H), 0.73 (m, 1H); ¹³C NMR (100 MHz, CDCl₃) δ 167.9, 152.4, 151.1, 132.6, 132.1, 129.6, 127.1, 126.9, 114.3, 113.5, 111.7, 103.8, 77.6, 56.6, 46.8, 44.7 (2C), 40.3, 34.8, 33.8, 26.5, 23.2, 21.9, 20.9, 16.6, 16.2, 12.5 (2C); ES-API MS: m/z calcd for C₃₁H₄₄IN₃O₂, found 492.3 [M−I+H].

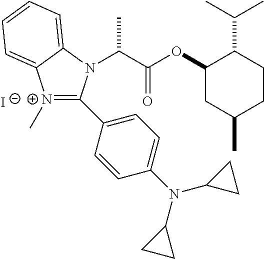

The title compounds were obtained following the general procedure (Step F) described above. Purification of the residue by prep-TLC (8% MeOH in CH₂Cl₂) afforded the entitled salt LW-IV-96 as a yellow gel (93%). IR (cm⁻¹) 3427, 2957, 2871, 1738, 1605, 1470, 1455, 1357, 1190, 752; [α]_{D}²⁰+84.84 (c 0.99, CHCl₃); ¹H NMR (400 MHz, CDCl₃) δ 8.04 (br, 1H), 7.93 (d, J=8.4 Hz, 1H), 7.64 (ddd, J=8.0, 7.2, 0.8 Hz, 1H), 7.56 (ddd, J=8.4, 7.2, 0.8 Hz, 1H), 7.51 (d, J=8.0 Hz, 1H), 7.46-7.07 (br, 3H), 5.27 (q, J=7.6 Hz, 1H), 4.79 (ddd, J=10.8, 10.8, 4.4 Hz, 1H), 4.09 (s, 3H), 2.55 (m, 2H), 1.92 (d, J=7.6 Hz, 3H), 1.85-1.75 (m, 2H), 1.71-1.60 (m, 2H), 1.44 (m, 1H), 1.38 (m, 1H), 1.02 (m, 1H), 0.98 (m, 4H), 0.92 (d, J=6.8 Hz, 3H), 0.85 (m, 1H), 0.83 (d, J=6.8 Hz, 3H), 0.80-0.72 (m, 8H); ¹³C NMR (100 MHz, CDCl₃) δ 168.0, 153.8, 152.4, 132.7, 129.7, 127.2, 127.0, 114.4, 113.6, 106.1, 77.7, 56.6, 46.9, 40.4, 34.9, 33.9, 31.5, 30.7 (2C), 26.6, 23.2, 22.0, 21.0, 16.7, 16.3, 9.70 (2C), 9.65 (2C); ES-API MS: m/z calcd for C₃₃H₄₄IN₃O₂, found 514.4 [M−I].

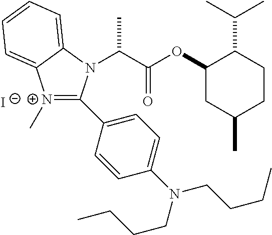

The title compounds were obtained following the general procedure (Step F) described above. Purification of the residue by prep-TLC (8% MeOH in CH₂Cl₂) afforded the entitled salt LW-IV-97 as a yellow gel (91%). IR (cm⁻¹) 3427, 2956, 2930, 2872, 1739, 1605, 1511, 1470, 1369, 1202, 825, 752; [α]_{D}²⁰+71.66 (c 1.08, CHCl₃); ¹H NMR (400 MHz, CDCl₃) δ 8.00 (br, 1H), 7.91 (d, J=8.4 Hz, 1H), 7.63 (ddd, J=8.4, 7.2, 1.2 Hz, 1H), 7.55 (ddd, J=8.4, 7.2, 0.8 Hz, 1H), 7.50 (d, J=8.0 Hz, 1H), 7.35 (br, 1H), 6.78 (br, 2H), 5.29 (q, J=7.2 Hz, 1H), 4.80 (ddd, J=10.8, 10.8, 4.4 Hz, 1H), 4.10 (s, 3H), 3.35 (m, 4H), 1.92 (d, J=7.6 Hz, 3H), 1.86-1.77 (m, 2H), 1.72-1.57 (m, 7H), 1.50-1.34 (m, 6H), 1.03 (m, 1H), 0.98 (t, J=7.2 Hz, 6H), 0.92 (d, J=7.2 Hz, 3H), 0.84 (d, J=6.4 Hz, 3H), 0.80 (m, 1H), 0.76 (d, J=7.2 Hz, 3H); ¹³C NMR (100 MHz, CDCl₃) δ 168.1, 152.5, 151.4, 132.8, 129.7, 127.1, 126.9, 114.4, 114.1, 113.6, 111.8, 104.0, 77.7, 56.7, 51.0 (2C), 46.9, 40.4, 34.8, 33.9, 31.5, 29.3 (2C), 26.6, 23.2, 22.0, 21.0, 20.4 (2C), 16.7, 16.3, 14.1 (2C); ES-API MS: m/z calcd for C₃₅H₅₂IN₃O₂, found 546.4 [M−I].

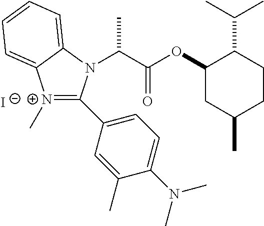

The title compounds were obtained following the general procedure (Step F) described above. Purification of the residue by prep-TLC (10% MeOH in CH₂Cl₂) afforded the entitled salt LW-IV-76 as a yellow gel (88%). IR (cm⁻¹) 3426, 2955, 2870, 2790, 1738, 1605, 1470, 1220, 1118, 754; [α]_{D}²⁰+64.72 (c 2.24, CHCl₃); ¹H NMR (400 MHz, CDCl₃) δ 8.03-7.85 (m, 1H), 7.91 (d, J=8.4 Hz, 1H), 7.58 (ddd, J=7.8, 7.6, 1.2 Hz, 1H), 7.52 (ddd, J=8.4, 7.2, 0,8 Hz, 1H), 7.47 (d, J=8.0 Hz, 1H), 7.24-6.97 (m, 2H), 5.16 (q, J=7.2 Hz, 1H), 4.71 (ddd, J=8.0, 7.2 Hz, 1H), 4.00 (s, 3H), 2.78 (s, 6H), 2.30 (s, 3H), 1.84 (d, J=7.6 Hz, 3H), 1.76-1.65 (m, 2H), 1.61-1.52 (m, 2H), 1.36 (m, 1H), 1.30 (m, 1H), 0.94 (m, 1H), 0.83 (d, J=6.8 Hz, 3H), 0.77 (m, 1H), 0.75 (d, J=6.8 Hz, 3H), 0.71 (m, 1H), 0.67 (d, J=6.8 Hz, 3H); ¹³C NMR (100 MHz, CDCl₃) δ 167.5, 157.1, 151.3, 132.3, 130.4, 129.4, 128.1, 127.2, 127.0, 119.0, 118.1, 114.4, 113.5, 111.2, 77.5, 56.3, 46.5, 43.1 (2C), 40.1, 34.7, 33.6, 31.2, 26.3, 22.9, 21.7, 20.7, 19.6, 16.5, 16.0; ES-API MS: m/z calcd for C₃₅H₅₂IN₃O₂, found 476.3 [M−I].

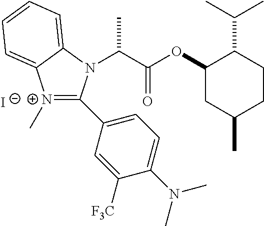

The title compounds were obtained following the general procedure (Step F) described above. Purification of the residue by prep-TLC (8% MeOH in CH₂Cl₂) afforded the entitled salt LW-IV-70 as a yellow gel (99%). IR (cm⁻¹) 3420, 2957, 2928, 2873, 1739, 1616, 1470, 1116, 753; [α]_{D}²⁰+1.2 (c 1.17, CHCl₃); ¹H NMR (400 MHz, CDCl₃) δ 8.85 (d, J=6.8 Hz, 1H), 7.83 (d, J=6.8 Hz, 1H), 7.69-7.60 (m, 4H), 7.38 (d, J=8.0 Hz, 1H), 5.16 (d, J=6.8 Hz, 1H), 4.82 (ddd, J=10.0, 9.2, 2.0 Hz, 1H), 4.06 (s, 3H), 3.04 (s, 6H), 2.02 (d, J=6.0 Hz, 3H), 1.85 (m, 1H), 1.82 (m, 1H), 1.71-1.65 (m, 2H), 1.51-1.36 (m, 3H), 1.03 (m, 1H), 0.90 (d, J=6.8 Hz, 3H), 0.85 (d, J=6.4 Hz, 3H), 0.81 (m, 1H), 0.76 (d, J=6.4 Hz, 3H); ¹³C NMR (100 MHz, CDCl₃) δ 168.1, 155.2, 150.3, 137.4, 132.8, 130.6, 129.9, 127.6, 127.4, 125.3, 122.6, 121.0, 114.5, 114.2, 110.2, 109.7, 78.0, 57.2, 46.8, 44.0, 40.4, 34.6, 34.0, 31.5, 26.6, 23.2, 22.0, 20.9, 17.0, 16.2; ES-API MS: m/z calcd for C₃₅H₅₂IN₃O₂, found 530.3 [M−I].

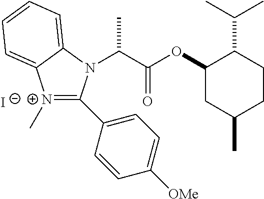

The title compounds were obtained following the general procedure (Step

F) described above. Purification of the residue by prep-TLC (8% MeOH in CH₂Cl₂) afforded the entitled salt LW-IV-6 as a yellow gel (87%). IR (cm⁻¹) 3430, 3016, 2957, 2871, 1738, 1732, 1609, 1470, 1260, 1036, 844, 732; [α]_{D}²⁰+27.83 (c 1.25, CHCl₃); ¹H NMR (400 MHz, CDCl₃) δ 8.15 (m, 1H), 7.96 (d, J=8.4 Hz, 1H), 7.78 (d, J=8.0 Hz, 1H), 7.66 (ddd, J=8.4, 7.2, 0.8 Hz, 1H), 7.60 (ddd, J=8.4, 7.2, 0.8 Hz, 1H), 7.42 (m, 1H), 7.21 (m, 1H), 7.09 (m, 1H), 4.75 (ddd, J=11.2, 10.8, 4.4 Hz, 1H), 4.24 (d, J=10.0 Hz, 1H), 4.07 (s, 3H), 3.89 (s, 3H), 1.83 (m, 1H), 1.76 (m, 1H), 1.67-1.61 (m, 3H), 1.47-1.35 (m, 2H), 0.99 (m, 1H), 0.90 (m, 1H), 0.86 (d, J=7.2 Hz, 3H), 0.84 (m, 1H), 0.83 (d, J=6.4 Hz, 3H), 0.80 (m, 1H), 0.71 (d, J=6.8 Hz, 3H), 0.62-0.51 (m, 2H), 0.30 (m, 1H); ¹³C NMR (100 MHz, CDCl₃) δ 167.1, 163.5, 150.8, 133.7, 132.3, 131.6, 130.2, 127.6, 127.5, 115.9, 115.6, 114.4, 114.2, 111.5, 77.9, 66.6, 56.0, 46.8, 40.4, 35.1, 33.8, 31.4, 26.4, 23.0, 21.9, 20.8, 16.0, 12.4, 7.7, 5.4; ES-API MS: m/z calcd for C₃₀H₃₉IN₂O₃, found 475.3 [M−I].

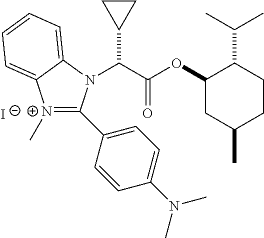

The title compounds were obtained following the general procedure (Step F) described above. Purification of the residue by prep-TLC (8% MeOH in CH₂Cl₂) afforded the entitled salt LW-IV-7 as a yellow gel (86%). IR (cm⁻¹) 3427, 2955, 2870, 1738, 1606, 1469, 1372, 1201, 1037, 732; [α]_{D}²⁰+84.89 (c 1.14, CHCl₃); ¹H NMR (400 MHz, CDCl₃) δ 8.01 (d, J=8.0 Hz, 1H), 7.85-7.26 (br, 2H), 7.74 (d, J=8.4 Hz, 1H), 7.65 (dd, J=7.6, 7.6 Hz, 1H), 7.58 (dd, J=7.6, 7.6 Hz, 1H), 6.82 (br, 2H), 4.80 (ddd, J=11.2, 10.8, 4.4 Hz, 1H), 4.29 (d, J=10.4 Hz, 1H), 4.12 (s, 3H), 3.40 (s, 1H), 3.09 (s, 5H), 1.88 (m, 1H), 1.82 (m, 1H), 1.69-1.63 (m, 3H), 1.49-1.37 (m, 2H), 1.02 (m, 1H), 0.93 (m, 1H), 0.89 (d, J=7.2 Hz, 3H), 0.87 (m, 1H), 0.85 (d, J=6.4 Hz, 3H), 0.82 (m, 1H), 0.75 (d, J=7.2 Hz, 3H), 0.60-0.49 (m, 2H), 0.13 (m, 1H); ¹³C NMR (100 MHz, CDCl₃) δ 167.3, 153.2, 152.2, 132.5, 130.1, 127.4, 127.2, 114.4, 114.1, 112.2, 110.1, 104.3, 77.9, 66.7, 50.7, 46.9, 40.4, 40.2 (2C), 35.1, 33.9, 31.4, 29.8, 26.5, 23.1, 22.0, 20.9, 16.1, 12.5, 7.5, 4.9; ES-API MS: m/z calcd for C₃₁H₄₂IN₃O₂, found 488.3 [M−I].

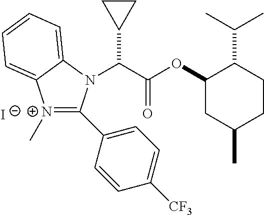

The title compounds were obtained following the general procedure (Step F) described above. Purification of the residue by prep-TLC (7% MeOH in CH₂Cl₂) afforded the entitled salt LW-IV-8 as a yellow gel (83%). IR (cm⁻¹) 3453, 3014, 2958, 2931, 2872, 1738, 1323, 1174, 1135, 754; [α]_{D}²⁰−3.25 (c 1.29, CHCl₃); ¹H NMR (400 MHz, CDCl₃) δ 8.65 (d, J=6.4 Hz, 1H), 7.94 (m, 1H), 7.92 (d, J=8.4 Hz, 2H), 7.85 (d, J=8.0 Hz, 2H), 7.68 (dd, J=8.4, 6.8 Hz, 1H), 7.64 (d, J=8.4, 6.8 Hz, 1H), 4.73 (ddd, J=10.8, 10.8, 4.4 Hz, 1H), 4.29 (d, J=9.6 Hz, 1H), 4.01 (s, 3H), 1.81 (m, 1H), 1.75-1.62 (m, 3H), 1.48-1.36 (m, 3H), 0.99 (m, 1H), 0.89 (m, 1H), 0.85 (d, J=6.4 Hz, 3H), 0.84 (d, J=6.4 Hz, 3H), 0.80 (m, 1H), 0.68 (d, J=6.8 Hz, 3H), 0.65-0.55 (m, 3H); ¹³C NMR (100 MHz, CDCl₃) δ 167.1, 148.8, 135.2 (q, J=33.3 Hz, 1C), 133.2, 132.5, 131.5, 130.6, 127.94, 127.90, 127.1, 126.5, 124.7, 124.5, 121.7, 114.5, 114.3, 78.2, 66.8, 46.8, 40.4, 35.1, 33.8, 31.4, 26.3, 23.0, 21.9, 20.8, 16.0, 12.5, 8.2, 6.1; ES-API MS: m/z calcd for C₃₀H₃₆F₃IN₂O₂, found 513.3 [M−I].

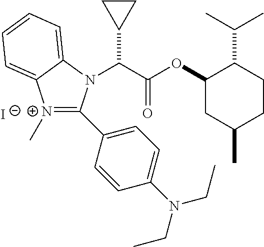

The title compounds were obtained following the general procedure (Step F) described above. Purification of the residue by prep-TLC (8% MeOH in CH₂Cl₂) afforded the entitled salt LW-IV-58 as a yellow gel (86%). IR (cm⁻¹) 3424, 2958, 2928, 2872, 1738, 1604, 1511, 1470, 1276, 1202, 752; [α]_{D}²⁰+105.32 (c 1.20, CHCl₃); ¹H NMR (400 MHz, CDCl₃) δ 8.01 (d, J=8.4 Hz, 1H), 7.74 (d, J=8.4 Hz, 1H), 7.66 (dd, J=8.0, 7.6 Hz, 1H), 7.58 (dd, J=8.4, 7.6 Hz, 1H), 7.75-7.15 (br, 2H), 6.80 (br, 2H), 4.82 (ddd, J=11.2, 10.8, 4.4 Hz, 1H), 4.32 (d, J=10.0 Hz, 1H), 4.15 (s, 3H), 3.44 (q, J=7.2 Hz, 4H), 1.90 (m, 1H), 1.84 (m, 1H), 1.78-1.66 (m, 3H), 1.51-1.39 (m, 2H), 1.23 (t, J=6.8 Hz, 6H), 1.04 (m, 1H), 0.92 (m, 1H), 0.91 (d, J=6.8 Hz, 3H), 0.89 (m, 1H), 0.87 (d, J=6.8 Hz, 3H), 0.84 (m, 1H), 0.77 (d, J=6.8 Hz, 3H), 0.63-0.52 (m, 2H), 0.17 (m, 1H); ¹³C NMR (100 MHz, CDCl₃) δ 167.4, 152.3, 151.2, 132.6, 132.0, 130.1, 127.4 (2C), 127.1 (2C), 114.5, 114.2, 111.7, 103.6, 77.9, 66.7, 47.0, 44.8 (2C), 40.5, 35.3, 33.9, 31.5, 26.6, 23.2, 22.0, 21.0, 16.2, 12.5 (2C), 7.6, 5.0; ES-API MS: m/z calcd for C₃₃H₄₆IN₃O₂, found 518.4 [M−I+H].

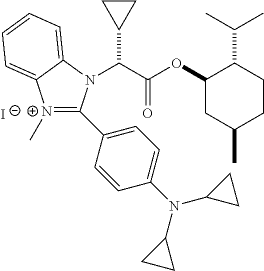

The title compounds were obtained following the general procedure (Step F) described above. Purification of the residue by prep-TLC (8% MeOH in CH₂Cl₂) afforded the entitled salt LW-IV-98 as a yellow gel (97%). IR (cm⁻¹) 3010, 2956, 2925, 2871, 1738, 1604, 1470, 1357, 1190, 752; [α]_{D}²⁰+99.71 (c 0.73, CHCl₃); ¹H NMR (400 MHz, CDCl₃) δ 8.03 (d, J=8.4 Hz, 1H), 7.96 (br, 1H), 7.76 (d, J=8.0 Hz, 1H), 7.68 (ddd, J=8.4, 7.6, 1.2 Hz, 1H), 7.61 (ddd, J=8.4, 8.4, 0.8 Hz, 1H), 7.56-7.11 (br, 3H), 4.84 (ddd, J=10.8, 10.8, 4.4 Hz, 1H), 4.33 (d, J=10.0 Hz, 1H), 4.18 (s, 3H), 2.56 (m, 2H), 1.92 (m, 1H), 1.86 (m, 1H), 1.80-1.65 (m, 4H), 1.54-1.40 (m, 2H), 1.24 (m, 1H), 1.06 (m, 1H), 1.00 (d, J=6.8 Hz, 3H), 0.97-0.75 (m, 15H), 0.66-0.52 (m, 2H), 0.26 (m, 1H); ¹³C NMR (100 MHz, CDCl₃) δ 167.5, 153.9, 152.3, 132.7, 130.2, 127.5, 127.2, 114.7, 114.2, 105.9, 77.9, 66.7, 47.0, 40.6, 35.4, 34.0, 31.6, 30.8 (2C), 26.6, 23.3, 22.1, 21.0, 16.3, 12.6, 9.8 (2C), 9.7 (2C), 7.7, 5.2; ES-API MS: m/z calcd for C₃₅H₄₆IN₃O₂, found 540.4 [M−I].

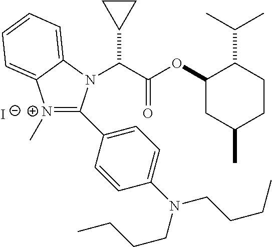

The title compounds were obtained following the general procedure (Step F) described above. Purification of the residue by prep-TLC (8% MeOH in CH₂Cl₂) afforded the entitled salt LW-IV-99 as a yellow gel (85%). IR (cm⁻¹) 3391, 2956, 2930, 2871, 1738, 1605, 1512, 1470, 1369, 1202, 753; [α]_{D}²⁰+112.32 (c 1.20, CHCl₃); ¹H NMR (400 MHz, CDCl₃) δ 8.01 (d, J=8.0 Hz, 1H), 7.85 (br, 1H), 7.74 (d, J=8.0 Hz, 1H), 7.66 (dd, J=7.2, 8.0 Hz, 1H), 7.58 (dd, J=8.0, 7.6 Hz, 1H), 7.43-7.09 (br, 1H), 6.75 (br, 2H), 4.83 (ddd, J=11.2, 10.8, 4.4 Hz, 1H), 4.33 (d, J=10.0 Hz, 1H), 4.16 (s, 3H), 3.35 (m, 4H), 1.91 (m, 1H), 1.84 (m, 1H), 1.78-1.57 (m, 6H), 1.52-1.34 (m, 5H), 1.05 (m, 1H), 0.98 (t, J=7.6 Hz, 6H), 0.95-0.83 (m, 8H), 0.78 (d, J=7.2 Hz, 3H), 0.64-0.52 (m, 2H), 0.22 (m, 1H); ¹³C NMR (100 MHz, CDCl₃) δ 167.5, 152.3, 151.5, 132.7, 132.0, 130.2, 127.3, 127.1, 114.5, 114.1, 111.8, 103.7, 77.8, 66.7, 51.0 (2C), 47.0, 40.6, 35.3, 34.0, 31.5, 29.2 (2C), 26.6, 23.2, 22.1, 21.0, 20.4 (2C), 16.2, 14.1 (2C), 12.6, 7.7, 5.1; ES-API MS: m/z calcd for C₃₇H₅₄IN₃O₂, found 572.4 [M−I].

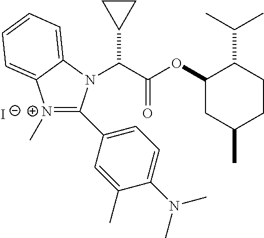

The title compounds were obtained following the general procedure (Step F) described above. Purification of the residue by prep-TLC (10% MeOH in CH₂Cl₂) afforded the entitled salt LW-IV-77 as a yellow gel (80%). IR (cm⁻¹) 3423, 2955, 2870, 1736, 1605, 1469, 1217, 1038, 753; [α]_{D}²⁰+57.47 (c 1.51, CHCl₃); ¹H NMR (400 MHz, CDCl₃) δ 8.01 (d, J=8.0 Hz, 1H), 7.90-7.68 (br, 1H), 7.77 (d, J=8.0 Hz, 1H), 7.65 (dd, J=7.2, 7.2 Hz, 1H), 7.59 (d, J=8.4, 7.2 Hz, 1H), 7.23-6.95 (m, 2H), 4.77 (ddd, J=10.4, 10.4, 3.2 Hz, 1H), 4.25 (d, J=10.0 Hz, 1H), 4.09 (s, 3H), 2.83 (s, 6H), 2.35 (m, 3H), 1.96-1.61 (m, 5H), 1.49-1.33 (m, 2H), 0.99 (m, 1H), 0.88 (m, 1H), 0.86 (d, J=6.8 Hz, 3H), 0.83 (m, 1H), 0.82 (d, J=6.4 Hz, 3H), 0.76 (m, 1H), 0.71 (d, J=6.8 Hz, 3H), 0.55 (m, 2H), 0.17 (m, 1H); ¹³C NMR (100 MHz, CDCl₃) δ 167.0, 157.3, 151.3, 132.3, 132.0, 130.2, 130.0, 127.5, 127.4, 119.1, 118.2, 114.5, 114.2, 110.1, 77.8, 66.5, 46.7, 43.2 (2C), 40.4, 35.2, 33.8, 31.4, 26.3, 23.0, 21.9, 20.8, 19.8, 16.0, 12.4, 7.5, 5.0; ES-API MS: m/z calcd for C₃₂H₄₄IN₃O₂, found 502.4 [M−I].

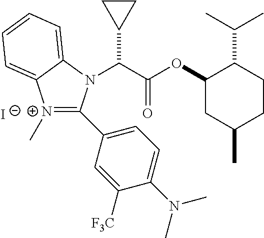

The title compounds were obtained following the general procedure (Step F) described above. Purification of the residue by prep-TLC (8% MeOH in CH₂Cl₂) afforded the entitled salt LW-IV-71 as a yellow gel (84%). IR (cm⁻¹) 3407, 2927, 2956, 2872, 1738, 1616, 1470, 1116, 753; [α]_{D}²⁰−17.36 (c 0.91, CHCl₃); ¹H NMR (400 MHz, CDCl₃) δ 8.75 (m, 1H), 7.90 (m, 2H), 7.70 (dd, J=8.0, 7.2 Hz, 1H), 7.64 (dd, J=8.4, 7.2 Hz, 1H), 7.51 (m, 1H), 7.38 (m, 1H), 4.82 (m, 1H), 4.25 (m, 1H), 4.13 (s, 3H), 3.06 (s, 6H), 1.88 (m, 1H), 1.82 (m, 1H), 1.74-1.63 (m, 3H), 1.50-1.39 (m, 2H), 1.04 (m, 1H), 0.94-0.83 (m, 9H), 0.77 (m, 2H), 0.70 (m, 2H), 0.59 (m, 1H), 0.42 (m, 1H); ¹³C NMR (100 MHz, CDCl₃) δ 167.2, 154.8, 150.0, 136.7, 132.5, 130.4, 127.5, 127.4, 125.1, 122.4, 121.0, 115.0, 114.0, 108.7, 78.1, 66.9, 46.7, 43.8, 43.7, 40.3, 35.0, 33.8, 31.4, 29.7, 26.3, 23.0, 21.8, 20.7, 15.9, 12.6, 7.6, 5.6; ES-API MS: m/z calcd for C₃₂H₄₁F₃IN₃O₂, found 556.3 [M−I].

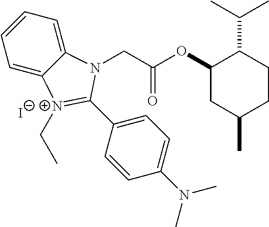

The title compounds were obtained following the general procedure (Step F) described above. Purification of the residue by flash chromatography on silica gel (gradient elution, 5% MeOH in CH₂Cl₂) afforded the entitled salt LW-V-229 as a yellow gel (73%). IR (cm⁻¹) 2955, 2923, 2870, 1740, 1606, 1505, 1471, 1372, 1224, 1199, 755; [α]_{D}²⁰−36.61 (c 1.30, CHCl₃); ¹H NMR (400 MHz, CDCl₃) δ 7.82 (m, 1H), 7.68-7.59 (m, 5H), 6.83 (d, J=9.2 Hz, 2H), 5.28 (d, J=18.4 Hz, 1H), 5.16 (d, J=18.4 Hz, 1H), 4.74 (ddd, J=10.8, 11.2, 4.4 Hz, 1H), 4.45 (q, J=7.6 Hz, 2H), 3.10 (s, 6H), 1.91 (m, 1H), 1.70-1.59 (m, 3H), 1.54 (t, J=7.2 Hz, 3H), 1.44 (m, 1H), 1.36 (m, 1H), 1.06-0.95 (m, 2H), 0.90 (d, J=6.4 Hz, 3H), 0.87 (m, 1H), 0.84 (d, J=7.2 Hz, 3H), 0.68 (d, J=7.2 Hz, 3H); ¹³C NMR (100 MHz, CDCl₃) δ 166.3, 153.2, 152.6, 132.2, 131.7 (2C), 130.9, 127.5, 127.3, 113.4, 113.3, 112.2 (2C), 105.0, 77.5, 49.1, 46.9, 42.6, 40.7, 40.2 (2C), 34.0, 31.6, 26.3, 23.3, 22.1, 20.9, 16.3, 15.2; ES-API MS: m/z calcd for C₂₉H₄₀IN₃O₂, found 462.3 [M−I].

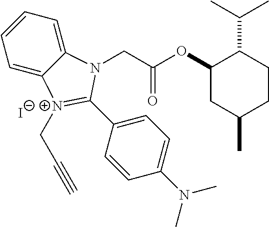

The title compounds were obtained following the general procedure (Step F) described above. Purification of the residue by flash chromatography on silica gel (gradient elution, 10% MeOH in CH₂Cl₂) afforded the entitled salt LW-V-230 as a yellow gel (77%). IR (cm⁻¹) 3305, 3192, 2955, 2925, 2871, 1740, 1605, 1505, 1471, 1374, 1228, 1202, 753; [α]_{D}²⁰−35.65 (c 1.75, CHCl₃); ¹H NMR (400 MHz, CDCl₃) δ 7.90 (m, 1H), 7.67-7.58 (m, 5H), 6.79 (d, J=9.2 Hz, 2H), 5.28 (d, J=18.0 Hz, 1H), 5.19 (d, J=2.0 Hz, 2H), 5.14 (d, J=18.0 Hz, 1H), 4.67 (ddd, J=11.2, 10.8, 4.4 Hz, 1H), 3.06 (s, 6H), 2.57 (t, J=2.4 Hz, 1H), 1.84 (m, 1H), 1.60 (m, 2H), 1.52 (m, 1H), 1.37 (m, 1H), 1.29 (m, 1H), 0.99-0.89 (m, 2H), 0.83 (d, J=6.4 Hz, 3H), 0.80 (m, 1H), 0.77 (d, J=6.8 Hz, 3H), 0.60 (d, J=6.8 Hz, 3H); ¹³C NMR (100 MHz, CDCl₃) δ 166.6, 153.3, 152.6, 131.8 (2C), 131.7, 130.7, 127.5, 127.4, 113.7, 113.0, 112.1 (2C), 103.8, 77.4, 76.8, 74.6, 48.8, 46.6, 40.5, 40.1 (2C), 37.8, 33.8, 31.4, 26.1, 23.0, 21.9, 20.7, 16.0; ES-API MS: m/z calcd for C₃₀H₃₈IN₃O₂, found 472.3 [M−I].

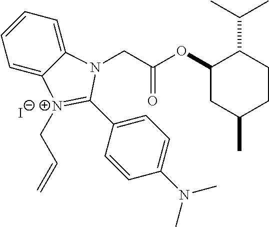

The title compounds were obtained following the general procedure (Step F) described above. Purification of the residue by flash chromatography on silica gel (gradient elution, 5% MeOH in CH₂Cl₂) afforded the entitled salt LW-V-231 as a yellow gel (86%). IR (cm⁻¹) 2955, 2925, 2870, 1741, 1606, 1504, 1471, 1373, 1227, 1201, 753; [α]_{D}²⁰−37.25 (c 2.55, CHCl₃); ¹H NMR (400 MHz, CDCl₃) δ 7.73-7.66 (m, 2H), 7.61-7.55 (m, 4H), 6.78 (d, J=8.8 Hz, 2H), 6.06 (m, 1H), 5.37 (m, 1H), 5.34 (d, J=18.4 Hz, 1H), 5.23 (m, 1H), 5.21 (d, J=18.0 Hz, 1H), 5.00 (m, 2H), 4.70 (ddd, J=11.2, 10.8, 4.4 Hz, 1H), 3.07 (s, 6H), 1.87 (m, 1H), 1.63 (m, 2H), 1.58 (m, 1H), 1.41 (m, 1H), 1.32 (m, 1H), 1.02-0.92 (m, 2H), 0.87 (d, J=6.8 Hz, 3H), 0.84 (m, 1H), 0.81 (d, J=7.2 Hz, 3H), 0.64 (d, J=6.8 Hz, 3H); ¹³C NMR (100 MHz, CDCl₃) δ 166.1, 153.3, 153.1, 132.0, 131.6 (2C), 131.3, 129.9, 127.4, 127.3, 119.9, 113.7, 113.2, 112.1 (2C), 104.6, 77.4, 49.4, 49.3, 46.8, 40.6, 40.2 (2C), 33.9, 31.5, 26.2, 23.1, 22.0, 20.8, 16.2; ES-API MS: m/z calcd for C₃₀H₄₀IN₃O₂, found 474.3 [M−I].

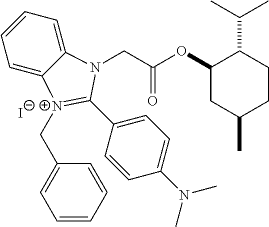

The title compounds were obtained following the general procedure (Step F) described above. Purification of the residue by flash chromatography on silica gel (gradient elution, 5% MeOH in CH₂Cl₂) and re-purified again by prep-TLC (8% MeOH in CH₂Cl₂) afforded the entitled salt LW-V-232 as a yellow gel (59%). IR (cm⁻¹) 2924, 1739, 1606, 1503, 1470, 1444, 1372, 1227, 1201, 751; [α]_{D}²⁰−21.33 (c 1.05, CHCl₃); ¹H NMR (400 MHz, CDCl₃) δ 7.69 (d, J=8.4 Hz, 1H), 7.63 (m, 2H), 7.57 (m, 1H), 7.50-7.44 (m, 2H), 7.35-7.28 (m, 3H), 7.19 (m, 2H), 6.76 (d, J=9.2 Hz, 2H), 5.63 (s, 2H), 5.39 (d, J=18.0 Hz, 1H), 5.27 (d, J=18.0 Hz, 1H), 4.74 (ddd, J=10.8, 10.8, 4.4 Hz, 1H), 3.06 (s, 6H), 1.89 (m, 1H), 1.66 (m, 2H), 1.61 (m, 1H), 1.44 (m, 1H), 1.35 (m, 1H), 1.05-0.93 (m, 2H), 0.89 (d, J=6.4 Hz, 3H), 0.87 (m, 1H), 0.82 (d, J=6.8 Hz, 3H), 0.66 (d, J=7.2 Hz, 3H); ¹³C NMR (100 MHz, CDCl₃) δ 166.2, 153.6, 153.3, 133.2, 132.2, 131.8 (2C), 131.4, 129.6 (2C), 128.8, 127.5, 127.3, 126.7 (2C), 113.9, 113.3, 112.2 (2C), 104.7, 77.5, 50.7, 49.4, 46.8, 40.7, 40.2 (2C), 34.0, 31.5, 26.3, 23.2, 22.0, 20.9, 16.2; ES-API MS: m/z calcd for C₃₄H₄₂IN₃O₂, found 524.3 [M−I].

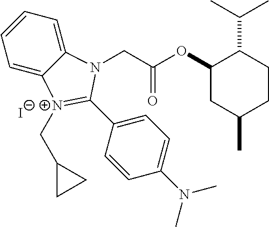

The title compounds were obtained following the general procedure (Step F) described above. Purification of the residue by flash chromatography on silica gel (gradient elution, 5% MeOH in CH₂Cl₂) afforded the entitled salt LW-V-234 as a yellow gel (95%). IR (cm⁻¹) 2955, 2924, 2871, 1741, 1606, 1504, 1470, 1372, 1224, 1199, 752; [α]_{D}²⁰−40.77 (c 1.80, CHCl₃); ¹H NMR (400 MHz, CDCl₃) δ 7.88 (dd, J=6.4, 2.4 Hz, 1H), 7.66 (dd, J=6.8, 2.0 Hz, 1H), 7.56 (m, 2H), 7.49 (d, J=8.4 Hz, 2H), 6.75 (d, J=9.2 Hz, 2H), 5.23 (d, J=18.0 Hz, 1H), 5.07 (d, J=18.4 Hz, 1H), 4.65 (ddd, J=11.2, 10.8, 4.4 Hz, 1H), 4.28 (dd, J=6.8, 2.8 Hz, 2H), 3.02 (s, 6H), 1.82 (m, 1H), 1.57 (m, 2H), 1.50 (m, 1H), 1.35 (m, 1H), 1.26 (m, 1H), 1.12 (m, 1H), 0.97-0.86 (m, 2H), 0.81 (d, J=6.4 Hz, 3H), 0.78 (m, 1H), 0.74 (d, J=7.2 Hz, 3H), 0.58 (d, J=7.2 Hz, 3H), 0.49 (m, 2H), 0.20 (m, 2H); ¹³C NMR (100 MHz, CDCl₃) δ 165.8, 152.9, 152.3, 131.6, 131.3 (2C), 131.1, 127.2, 127.1, 113.7, 113.0, 111.9 (2C), 104.5, 77.2, 51.6, 48.6, 46.6, 40.4, 40.0 (2C), 33.7, 31.3, 26.0, 22.9, 21.8, 20.6, 16.0, 10.7, 4.80, 4.79; ES-API MS: m/z calcd for C₃₁H₄₂IN₃O₂, found 488.3 [M−I].

### Procedure for Chemical Probes Synthesis:

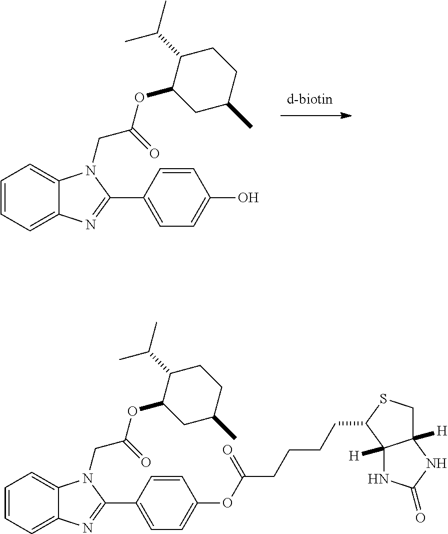

A mixture of starting material 2-phenyl-benzimidazole compound (2.6 mg, 0.006 mmol) and d-biotin (5 mg, 0.018 mmol) was dissolved in CH₂Cl₂ (1 mL) in a round-bottom flask. To this solution was added EDCI (3 mg, 0.018 mmol) and DMAP (2 mg, 0.018 mmol). The resulting mixture was stirred at rt for overnight. The solvent was removed under reduced pressure, and the resulting residue was purified by flash chromatography on silica gel (gradient elution, 10% MeOH in CH₂Cl₂) afford the entitled compound Lqr-5-091 as a colorless gel (2 mg, 0.003 mmol, 50%). IR (cm⁻¹) 2926, 2360, 1741, 1700, 1466, 1223, 1094; [α]_{D}²⁰+18.990 (c 0.10, CHCl₃); ¹H NMR (400 MHz, CDCl₃) δ 7.87-7.80 (m, 1H), 7.76 (d, J=8.4 Hz, 2H), 7.38-7.30 (m, 2H), 7.30-7.22 (m, 3H), 5.12 (s, 1H), 4.89 (d, J=2.4 Hz, 2H), 4.86-4.73 (m, 2H), 4.57-4.49 (m, 1H), 4.39-4.29 (m, 1H), 3.28-3.13 (m, 1H), 2.95 (dd, J=12.8, 5.2 Hz, 1H), 2.74 (dd, J=12.8, 8.4 Hz, 1H), 2.64 (t, J=7.2 Hz, 2H), 2.34 (t, J=7.2 Hz, 1H), 2.02-1.95 (m, 1H), 1.89-1.19 (m, 10H), 1.09-0.77 (m, 3H), 0.90 (d, J=6.4 Hz, 3H), 0.81 (d, J=7.2 Hz, 3H), 0.70 (d, J=7.2 Hz, 3H); ¹³C NMR (100 MHz, CDCl₃) δ 171.6, 167.3, 163.6, 153.0, 151.9, 142.7, 135.9, 130.5 (2C), 127.1, 123.3, 122.9, 122.1 (2C), 120.0, 109.4, 76.5, 61.9, 60.0, 55.4, 46.7, 40.5, 33.9, 31.5, 31.3, 28.3, 26.1, 25.2, 24.6, 23.0, 22.6, 21.9, 20.6, 15.9, 14.1; ES-API MS: m/z calcd for C₃₅H₄₄N₄O₅S, found 633.2 [M+H].

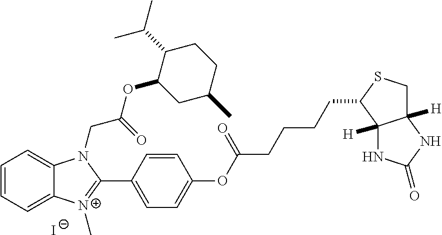

The title compounds were obtained following the general procedure (Step F) described above. Purification of the residue by prep-TLC (10% MeOH/CH₂Cl₂) afforded the entitled salt Lqr-5-092 as a yellow gel (54%). IR (cm⁻¹) 2926, 2362, 1742, 1700, 1470, 1233, 1094; [α]_{D}²⁰+15.997 (c 0.10, CHCl₃); ¹H NMR (400 MHz, CDCl₃) δ 8.13-7.99 (m, 2H), 7.89-7.79 (m, 1H), 7.74-7.55 (m, 3H), 7.45 (d, J=8.4 Hz, 2H), 5.97-5.79 (m, 1H), 5.65-5.48 (m, 1H), 5.24-5.06 (m, 2H), 4.75 (td, J=10.8, 4.4 Hz, 1H), 4.58-4.45 (m, 1H), 4.42-4.32 (m, 1H), 4.04 (s, 3H), 3.24-3.12 (m, 1H), 2.95-2.84 (m, 1H), 2.83-2.72 (m, 1H), 2.66 (t, J=7.6 Hz, 2H), 1.96-1.10 (m, 14H), 1.08-0.93 (m, 1H), 0.89 (d, J=6.4 Hz, 3H), 0.84 (d, J=6.8 Hz, 3H), 0.67 (d, J=7.2 Hz, 3H); ¹³C NMR (100 MHz, CDCl₃) δ 171.1, 165.8, 154.6, 151.5, 150.7, 135.7, 132.9, 131.9, 131.7, 128.3, 127.9, 127.7, 125.5, 123.4, 113.3, 112.8, 77.2, 46.7, 46.1, 40.5, 34.2, 33.9, 33.8, 31.4, 30.3, 29.7, 28.4, 28.3, 26.3, 24.5, 23.1, 21.8, 21.1, 20.6, 16.1, 8.5; ES-API MS: m/z calcd for C₃₆H₄₇IN₄O₅S, found 647.3 [M−I].

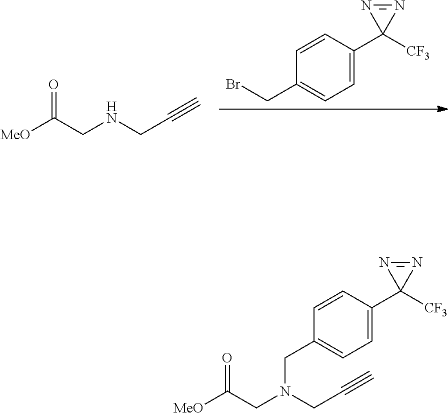

To a solution of methyl prop-2-yn-1-ylglycinate (50 mg, 0.39 mmol) in anhydrous DMF (1.3 mL) at 0° C. under argon atmosphere was added the Et₃N (100 mg, 0.34 mmol), followed by addition of 3-(4-(bromomethyl)phenyl)-3-(trifluoromethyl)-3H-diazirine (115 mg, 0.41 mmol). The resulting yellow mixture was stirred for overnight without replenish the ice bath. The solvent was removed under reduced pressure, and the resulting residue was purified by flash chromatography on silica gel (gradient elution, 15% EtOAc in hexanes) to obtain the entitled compound LW-III-89 as a pale yellow gel (97 mg, 0.30 mmol, 76%). IR (cm⁻¹) 3305, 2955, 2842, 1748, 1614, 1436, 1346, 1232, 1155, 939, 807, 666; NMR (400 MHz, CDCl₃) δ 7.42 (d, J=8.4 Hz, 2H), 7.14 (d, J=8.4 Hz, 2H), 3.75 (s, 2H), 3.70 (s, 3H), 3.45 (d, J=2.4 Hz, 2H), 3.39 (s, 2H), 2.27 (t, J=2.4 Hz, 1H); ¹³C NMR (100 MHz, CDCl₃) δ 171.2, 140.0, 129.6 (2C), 128.4, 126.7 (2C), 123.7, 120.9, 78.2, 74.0, 57.2, 54.1, 51.8, 42.5; ES-API MS: m/z calcd for C₁₅H₁₄F₃N₃O₂, found 326.1 [M+H].

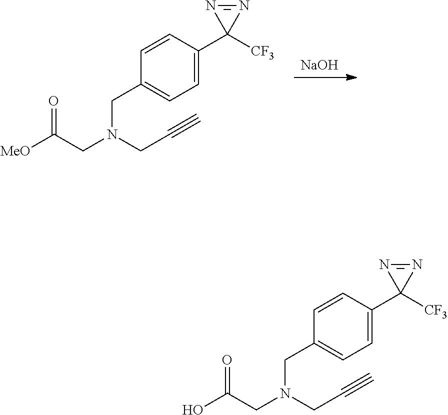

To a solution of methyl glycinate LW-III-89 (97 mg, 0.30 mmol) in MeOH (1.0 mL) at 0° C. was added 1M NaOH (0.6 mL). The resulting solution was raised to rt and stirred for 4 h. The reaction was quenched with 1N HCl (0.5 mL) then with pH6 phosphate buffer solution (0.3 mL) until it reached pH6. The majority of the solvent was removed under reduced pressure azerotropically with toluene, and the resulting residue was dried using Kugelrohr apparatus to afford the acid LW-III-90 as a pale yellow solid (122 mg, 0.39 mmol). IR (cm⁻¹) 3307, 2834, 1591, 1404, 1346, 1232, 1186, 1154, 939; ¹H NMR (400 MHz, CDCl₃) δ 7.32 (d, J=8.4 Hz, 2H), 7.08 (d, J=8.0 Hz, 2H), 3.67 (m, 2H), 3.29 (m, 2H), 3.24 (m, 2H), 2.24 (m, 1H); ¹³C NMR (100 MHz, CDCl₃) δ 169.1, 133.6, 124.9 (2C), 123.9, 121.9 (2C), 118.6, 72.4, 69.9, 52.4, 50.9, 37.3, 23.5 (d, J=40 Hz, 1C); ES-API MS: m/z calcd for C₁₄H₁₂F₃N₃O₂, found 312.1 [M+H].

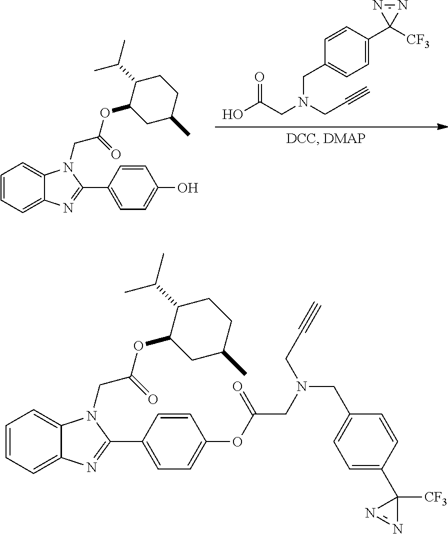

To a solution of phenol (30 mg, 0.07 mmol) in CH₂Cl₂ (0.8 mL) was added DCC (17.3 mg, 0.08 mmol) and DMAP (4.6 mg, 0.038 mmol). Finally, the acid LW-III-90 (26 mg, 0.08 mmol) was added in one portion. The resulting mixture was stirred at rt over 48 h. The solvent was removed under reduced pressured and purified by flash chromatography on silica gel (gradient elution, 040% EtOAc in hexanes) to obtain the desired compound LW-III-92, which was re-purified by prep-TLC (50% EtOAc in hexanes) to obtain the pure product as a colorless gel (15 mg, 0.021 mmol, 28%). IR (cm⁻¹) 3307, 2959, 2872, 1748, 1615, 1484, 1457, 1386, 1346, 1161, 1019, 984, 938, 911, 805, 743; [α]_{D}²⁰−16.7 (c 0.67, CHCl₃); ¹H NMR (400 MHz, CDCl₃) δ 7.84 (m, 1H), 7.77 (d, J=8.4 Hz, 2H), 7.48 (d, J=8.0 Hz, 2H), 7.34 (m, 2H), 7.29 (m, 1H), 7.26 (d, J=8.4 Hz, 2H), 7.18 (d, J=8.0 Hz, 2H), 4.91 (d, J=18.0 Hz, 1H), 4.86 (d, J=18.0 Hz, 1H), 4.79 (ddd, J=10.8, 10.8, 4.4 Hz, 1H), 3.87 (s, 2H), 3.71 (s, 2H), 3.55 (d, J=2.0 Hz, 2H), 2.34 (t, J=2.0 Hz, 1H), 1.98 (d, J=12.0 Hz, 1H), 1.69-1.56 (m, 4H), 1.47 (m, 1H), 1.34-1.24 (m, 3H), 0.91 (d, J=6.4 Hz, 3H), 0.81 (d, J=7.2 Hz, 3H), 0.70 (d, J=6.8 Hz, 3H); ¹³C NMR (100 MHz, CDCl₃) δ 168.8, 167.3, 152.9, 151.6, 139.5, 136.0, 130.6 (2C), 129.5 (2C), 128.4, 126.6 (2C), 123.4, 123.0, 121.9 (2C), 120.7, 120.1, 109.4, 77.8, 77.2, 76.6, 74.1, 57.0, 54.1, 46.8, 46.7, 42.4, 40.6, 33.9, 31.4, 28.3 (d, J=40.2 Hz, 1C), 26.1, 23.1, 21.9, 20.6, 16.0, 1.0; ES-API MS: m/z calcd for C₃₉H₄₀F₃N₅O₄, found 700.3 [M+H].

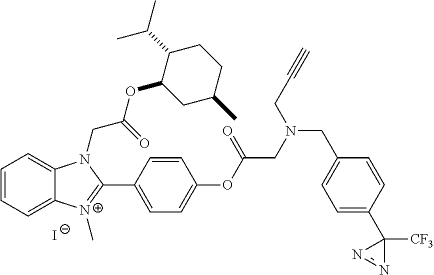

The title compounds were obtained following the general procedure (Step F) described above. Purification of the residue by prep-TLC (10% MeOH/CH₂Cl₂) afforded the entitled salt LW-III-97 as a yellow gel (54%). IR (cm⁻¹) 2959, 1738, 1607, 1483, 1469, 1346, 1229; [α]_{D}²⁰−16.26 (c 0.75, CHCl₃); ¹H NMR (400 MHz, CDCl₃) δ 8.15 (d, J=6.4 Hz, 2H), 7.81 (m, 1H), 7.70 (m, 2H), 7.61 (m, 1H), 7.47 (d, J=8.4 Hz, 2H), 7.45 (d, J=9.2 Hz, 2H), 7.18 (d, J=8.0 Hz, 2H), 5.18 (d, J=18.0 Hz, 1H), 5.11 (d, J=18.0 Hz, 1H), 4.77 (ddd, J=10.8, 10.8, 4.4 Hz, 1H), 4.02 (s, 3H), 3.87 (s, 2H), 3.74 (s, 2H), 3.54 (d, J=1.6 Hz, 2H), 2.35 (t, J=1.6 Hz, 1H), 1.91 (d, J=12.0 Hz, 1H), 1.70-1.64 (m, 4H), 1.40 (m, 2H), 1.03 (m, 2H), 0.92 (d, J=6.4 Hz, 3H), 0.87 (d, J=7.2 Hz, 3H), 0.70 (d, J=6.8 Hz, 3H); ¹³C NMR (100 MHz, CDCl₃) δ 168.4, 165.8, 154.2, 150.5, 139.4, 133.1, 132.0, 131.7, 129.4 (4C), 128.5, 128.0, 127.8, 126.7 (2C), 123.2 (2C), 117.5, 113.4, 112.8, 77.8, 77.7, 77.2, 74.3, 57.1, 54.1, 48.8, 46.7, 42.4, 40.5, 33.83, 33.80, 31.4, 26.4, 23.2, 21.9, 20.6, 16.1; ES-API MS: m/z calcd for C₄₀H₄₃F₃IN₅O₄, found 421.2 [M−I-probe].

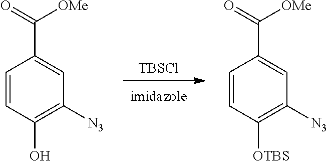 [SMILES: COC(=O)c1ccc(O)c(N=[N+]=[N-])c1 ; "TBSCl" ; COC(=O)c1ccc(O[Si](C)(C)C(C)(C)C)c(N=[N+]=[N-])c1]

To a solution of 3-azido-4-hydroxybenzoate (50 mg, 0.26 mmol) in anhydrous DMF (0.5 mL) at rt was added imidazole (35.2 mg, 0.52 mmol) and TBSCl (58.5 g, 0.38 mmol). The resulting mixture was stirred at rt overnight. The reaction mixture was quenched with 1N HCl (2 mL) and extracted with EtOAc (2×10 mL). The organic extract was washed with saturated NaHCO₃ solution (5 mL) and brine, and dried over with anhydrous MgSO₄ and filtrated. The solvent was removed under reduced pressure, and the resulting residue was purified by flash chromatography on silica gel (gradient elution, 12% EtOAc in hexanes) to afford the entitled compound LW-II-288 as a yellow gum (77 mg, 0.25 mmol, 97%). IR (cm⁻¹) 2954, 2932, 2860, 2105, 1725, 1508, 1437, 1317, 1260, 1115, 926, 843; ¹H NMR (400 MHz, CDCl₃) δ 7.70 (s, 1H), 7.69 (dd, J=8.8, 2.0 Hz, 1H), 6.84 (d, J=8.8 Hz, 1H), 3.87 (s, 3H), 0.99 (s, 9H), 0.25 (s, 6H); ¹³C NMR (100 MHz, CDCl₃) δ 166.1, 151.9, 130.9, 127.3, 124.0, 122.0, 120.2, 52.1, 25.6 (3C), 18.5-4.3 (2C); ES-API MS: m/z calcd for C₁₄H₂₁N₃O₃Si, found 282.0 [M−N2+H].

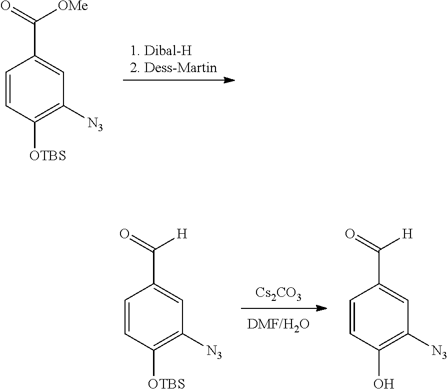

To a solution of ester LW-II-288 (77 mg, 0.25 mmol) in anhydrous CH₂Cl₂ (1.0 mL) at −78° C. was added Dibal-H (2 mL, 2 mmol, 1M solution in THF) over a course of 1 h. The reaction was raised to rt and stirred for 4 h. TLC indicated starting material remained, thus the reaction mixture was heated to 50° C. and stirred for additional 2 h. The reaction was cooled to 0° C. and quenched with MeOH with continuous stirring for 10 min. The mixture was then poured into a flask containing saturated Na/K-tartrate solution, the resulting mixture was stirred vigorously at rt for 1 h. The mixture was extracted with EtOAc (3×10 mL), and the organic extract was washed with brine and dried over with anhydrous MgSO₄. The solvent was removed under reduced pressure, and the resulting residue was purified by flash chromatography on silica gel (gradient elution, 030% EtOAc in hexanes) to afford the benzyl alcohol as a colorless gel (54 mg, 0.19 mmol, 70%). The alcohol was dissolved in anhydrous CH₂Cl₂ (0.64 mL) and added a solution of Dess-Martin periodinane (98 mg, 0.23 mmol) in anhydrous CH₂Cl₂ (1.0 mL) at 0° C. The resulting mixture was slowly raised to rt. After 30 min, the reaction was quenched with saturated NaHCO₃ solution at 0° C. and extracted with EtOAc. The mixture was extracted with EtOAc (3×10 mL), and the organic extract was washed with brine and dried over with anhydrous MgSO₄. The solvent was removed under reduced pressure, and the resulting residue was purified by flash chromatography on silica gel (gradient elution, 10% EtOAc in hexanes) to afford the entitled aldehyde LW-II-290 as a yellow gel (40 mg, 0.14 mmol, 74%). IR (cm⁻¹) 2957, 2932, 2860, 2117, 1698, 1594, 1505, 1428, 1311, 1256, 1199, 1090, 897, 843, 806, 786; ¹H NMR (400 MHz, CDCl₃) δ 9.84 (s, 1H), 7.55 (d, J=2.0 Hz, 1H), 7.53 (d, J=8.0, 2.0 Hz, 1H), 6.93 (d, J=8.4 Hz, 1H), 1.00 (s, 9H), 0.27 (s, 6H); ¹³C NMR (100 MHz, CDCl₃) δ 186.1, 149.5, 128.1, 126.9, 124.3, 117.2, 116.6, 21.6 (3C), 14.6 (2C), −8.3.

To a solution of aldehyde LW-II-290 (153 mg, 0.55 mmol) in DMF/H₂O (0.6 mL, v:v, 10:1) at rt was added Cs₂CO₃ (90 mg, 0.28 mmol). The reaction was stirred at this temperature for 15 min. The reaction mixture was filtered through a pad of celite and washed the residue with EtOAc. The solvent was removed under reduced pressure, and the resulting residue was purified by flash chromatography on silica gel (gradient elution, 35% EtOAc in hexanes) to afford the entitled compound LW-II-296 as a yellow solid (75 mg, 0.46 mmol, 83%). IR (cm⁻¹) 3159, 2962, 2111, 1667, 1580, 1234, 828; ¹H NMR (400 MHz, CDCl₃) δ 9.86 (s, 1H), 7.67 (d, J=1.6 Hz, 1H), 7.59 (dd, J=8.0, 1.6 Hz, 1H), 7.06 (d, J=8.4 Hz, 1H), 5.87 (br, 1H); ¹³C NMR (100 MHz, CDCl₃) δ 190.0, 152.7, 130.3, 129.8, 127.6, 118.3, 116.1; ES-API MS: m/z calcd for C₇H₅N₃O₂, found 162.9 [M−H].

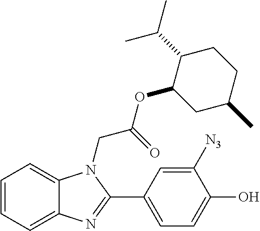

The title compound was obtained following the general procedure (Step A, method 1) described above. Purification of the residue by flash chromatography on silica gel (gradient elution, 050% EtOAc in hexanes) to give the entitled compound LW-III-1 as a pale yellow gel (82 mg, 0.183 mmol, 58%). IR (cm⁻¹) 2957, 2926, 2871, 2120, 1739, 1463, 1386, 1318, 1288, 1215, 735; [α]_{D}²⁰−21.60 (c 0.50, CHCl₃); ¹1-1 NMR (400 MHz, CDCl₃) δ 7.82 (m, 1H), 7.35-7.26 (m, 4H), 7.15 (dd, J=8.4, 2.0 Hz, 1H), 6.79 (d, J=8.4 Hz, 1H), 4.88 (d, J=17.6 Hz, 1H), 4.83 (d, J=17.6 Hz, 1H), 4.76 (ddd, J=11.2, 10.8, 4.4 Hz, 1H), 1.97 (m, 1H), 1.67-1.53 (m, 3H), 1.45 (m, 1H), 1.29 (m, 1H), 1.05-0.92 (m, 2H), 0.88 (d, J=6.4 Hz, 3H), 0.83 (m, 1H), 0.77 (d, J=6.8 Hz, 3H), 0.66 (d, J=6.8 Hz, 3H); ¹³C NMR (100 MHz, CDCl₃) δ 167.1, 153.5, 152.2, 141.4, 135.4, 127.9, 126.8, 123.6, 123.4, 120.8, 120.0, 119.2, 117.0, 109.6, 46.8, 46.6, 40.6, 33.9, 31.4, 26.2, 23.1, 21.9, 20.6, 16.0; ES-API MS: m/z calcd for C₂₅H₂₉N₅O₃, found 448.0 [M+H].

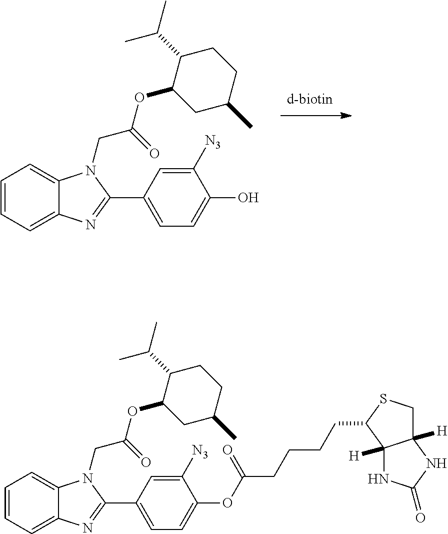

Biotin (27 mg, 0.11 mmol) was dissolved in pyridine (1 mL) at 50° C. in a round-bottom flask. Phenol LW-III-1 (50 mg, 0.11 mmol) was added to the reaction in one portion, followed by addition of DCC (23 mg, 0.11 mmol) solution in pyridine (1 mL), and the resulting mixture was stirred at 50° C. for overnight. The solvent was removed under reduced pressure, and the resulting residue was purified by flash chromatography on silica gel (gradient elution, 80% EtOAc in hexanes to remove excess pyridine, and switched to gradient elution, 06% MeOH in CH₂Cl₂) to afford the entitled compound LW-III-16 as a colorless gel (58 mg, 0.086 mmol, 77%). IR (cm⁻¹) 3228, 2929, 2870, 2123, 1739, 1704, 1485, 1458, 1212, 1101, 744; [α]_{D}²⁰+4.54 (c 1.19, CHCl₃); ¹H NMR (400 MHz, CDCl₃) δ 7.84 (m, 1H), 7.64 (m, 1H), 7.46 (d, J=8.0 Hz, 1H), 7.35-7.26 (m, 3H), 7.22 (d, J=8.4 Hz, 1H), 5.90 (br, 1H), 5.33 (br, 1H), 4.92 (d, J=18.4 Hz, 1H), 4.87 (d, J=18.4 Hz, 1H), 4.78 (ddd, J=10.8, 11.2, 4.4 Hz, 1H), 4.52 (dd, J=6.4, 5.2 Hz, 1H), 4.34 (dd, J=6.4, 5.2 Hz, 1H), 3.20 (m, 1H), 2.93 (dd, J=12.8, 4.8 Hz, 1H), 2.75 (d, J=13.2 Hz, 1H), 2.66 (dd, J=7.6, 7.2 Hz, 1H), 1.99 (m, 1H), 1.88-1.43 (m, 9H), 1.34-1.25 (m, 2H), 1.07-0.93 (m, 2H), 0.90 (d, J=6.4 Hz, 3H), 0.84 (m, 1H), 0.80 (d, J=7.2 Hz, 3H), 0.68 (d, J=6.8 Hz, 3H); ¹³C NMR (100 MHz, CDCl₃) δ 171.2, 167.3, 163.6, 152.1, 143.1, 142.7, 133.4, 128.8, 126.0, 124.1, 123.6, 123.2, 121.2, 120.2, 109.5, 76.7, 62.0, 60.1, 55.4, 46.8 (2C), 40.6, 40.5, 33.9, 33.5, 31.4, 28.32, 28.30, 26.2, 24.6, 23.1, 21.9, 20.6, 16.0; ES-API MS: m/z calcd for C₃₅H₄₃N₇O₅S, found 674.0 [M+H].

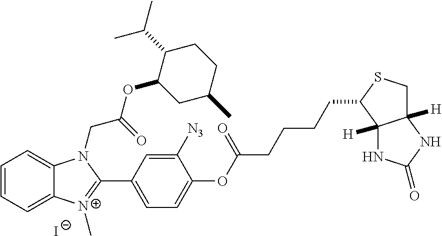

The title compounds were obtained following the general procedure (Step F) described above. Purification of the residue by prep-TLC (10% MeOH/CH₂Cl₂) afforded the entitled salt LW-III-17 as a yellow gel (36%). IR (cm⁻¹) 3264, 2928, 2870, 2360, 2342, 2125, 1739, 1694, 1484, 1469, 1222, 1100, 733; [α]_{D}²⁰−2.58 (c 0.31, CHCl₃); ¹H NMR (400 MHz, CDCl₃) δ 7.95 (m, 1H), 7.86 (d, J=8.0 Hz, 2H), 7.68 (m, 2H), 7.59 (d, J=8.0 Hz, 1H), 7.45 (d, J=8.4 Hz, 1H), 5.70 (br, 2H), 5.14 (m, 2H), 4.75 (ddd, J=11.2, 10.8, 4.4 Hz, 1H), 4.55 (m, 1H), 4.41 (m, 1H), 4.03 (s, 3H), 3.18 (m, 1H), 2.89 (m, 2H), 2.70 (dd, J=7.2, 6.8 Hz, 2H), 1.99 (m, 1H), 1.72-1.53 (m, 9H), 1.46-1.34 (m, 2H), 1.08-0.98 (m, 2H), 0.90 (d, J=6.8 Hz, 3H), 0.90 (m, 1H), 0.85 (d, J=7.2 Hz, 3H), 0.68 (d, J=7.2 Hz, 3H); ¹³C NMR (100 MHz, CDCl₃) δ 170.7, 165.7, 149.7, 145.9, 134.9, 132.0, 131.6, 128.5, 128.0, 127.8, 125.7, 123.2, 118.5, 113.7, 112.8, 77.9, 77.2, 62.0, 60.4, 55.6, 48.8, 46.7, 40.5, 34.4, 33.8, 33.6, 31.4, 28.6, 28.4, 26.4, 24.5, 23.2, 21.9, 20.6, 16.1; ES-API MS: m/z calcd for C₃₆H₄₆IN₇O₅S, found 688.0 [M−I].

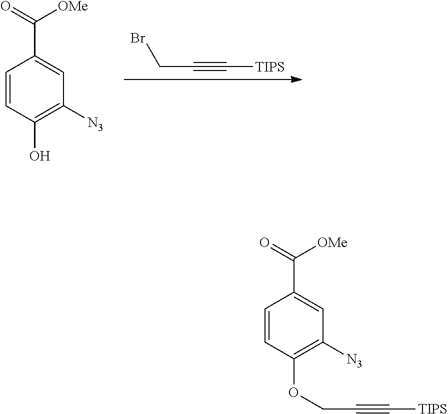

To a solution of 3-azido-4-hydroxybenzoate (300 mg, 1.55 mmol) in anhydrous DMF (3.1 mL) at rt was added K₂CO₃ (644 mg, 4.66 mmol) and TIPS-protected propargyl bromide (641 mg, 2.33 mmol). The resulting mixture was stirred at rt for 4 h. The reaction mixture was passed through a pad of celite and washed the remaining residue with EtOAc. The solvent was removed under reduced pressure, and the resulting residue was purified by flash chromatography on silica gel (gradient elution, 6% EtOAc in hexanes) to afford the entitled compound LW-II-257 as a yellow gel (575 mg, 1.48 mmol, 96%). IR (cm⁻¹) 2945, 2892, 2867, 2120, 1728, 1602, 1505, 1464, 1436, 1418, 1368, 1315, 1253, 1121, 1031, 997, 884, 763, 680; ¹H NMR (400 MHz, CDCl₃) δ 7.74 (dd, J=8.8, 2.4 Hz, 1H), 7.64 (d, J=2.0 Hz, 1H), 7.08 (d, J=8.8 Hz, 1H), 4.81 (s, 2H), 3.84 (s, 3H), 0.97 (s, 21H); ¹³C NMR (100 MHz, CDCl₃) δ 165.9, 153.4, 129.1, 127.2, 123.9, 121.7, 113.5, 100.2, 90.9, 57.6, 52.0, 18.4 (6C), 11.0 (3C); ES-API MS: m/z calcd for C₂₀H₂₉N₃O₃Si, found 388.2 [M+H].

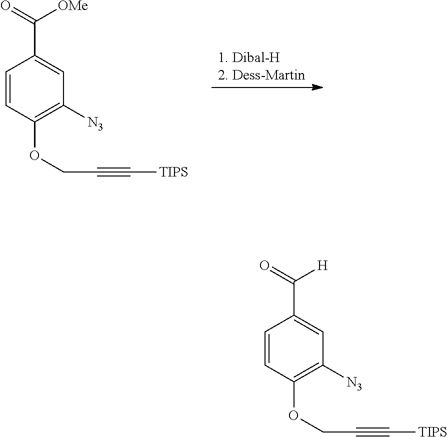

To a solution of ester LW-II-257 (675 mg, 1.74 mmol) in anhydrous CH₂Cl₂ (5.8 mL) at −78° C. was added Dibal-H (10 mL, 10 mmol) over a course of 1 h. Without replenishing the ice bath, the reaction was slowly raised to rt overnight. The reaction was cooled to 0° C. and quenched with excess amount of MeOH with continuous stirring for 10 min. The mixture was then poured into a flask containing saturated Na/K-tartrate solution, and the resulting mixture was stirred vigorously at rt for 1 h. The mixture was extracted with EtOAc (3×10 mL), and the organic extract was washed with brine and dried over with anhydrous MgSO₄. The solvent was removed under reduced pressure, and the resulting residue was purified by flash chromatography on silica gel (gradient elution, 025% EtOAc in hexanes) to afford the benzyl alcohol as a colorless gel (288 mg, 0.80 mmol, 46%). The alcohol was dissolved in anhydrous CH₂Cl₂ (2.67 mL) and added a solution of Dess-Martin periodinane (510 mg, 1.2 mmol) in anhydrous CH₂Cl₂ (1.0 mL) at 0° C. The resulting mixture was raised to rt and stirred for 30 min. The reaction was quenched with saturated NaHCO₃ solution at 0° C. and extracted with Et₂O. The organic extract was washed with brine and dried over with anhydrous MgSO₄, and filtrated. The solvent was removed under reduced pressure, and the resulting residue was purified by flash chromatography on silica gel (gradient elution, 15% EtOAc in hexanes) to afford the entitled compound LW-II-270 as a colorless gel (205 mg, 0.57 mmol, 72%). IR (cm⁻¹) 2944, 2892, 2866, 2125, 1694, 1597, 1582, 1505, 1463, 1431, 1309, 1230, 1198, 1089, 1030, 997, 883, 680; ¹H NMR (400 MHz, CDCl₃) δ 9.84 (s, 1H), 7.60 (dd, J=8.5, 2.0 Hz, 1H), 7.52 (d, J=2.0 Hz, 1H), 7.21 (d, J=8.5 Hz, 1H), 4.88 (s, 2H), 1.00 (br, 21H); ¹³C NMR (100 MHz, CDCl₃) δ 190.1, 154.6, 130.8, 130.2, 128.3, 120.7, 113.9, 99.8, 91.4, 57.7, 18.4 (6C), 11.0 (3C); ES-API MS: m/z calcd for C₁₉H₂₇N₃O₂Si, found 358.2 [M+H].

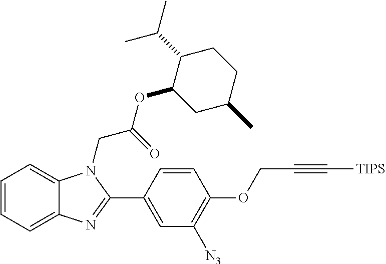

The title compound was obtained following the general procedure (Step A, method 1) described above. Purification of the residue by flash chromatography on silica gel (gradient elution, 30% EtOAc in hexanes) to give the entitled compound LW-II-280 as a pale yellow gel (29.4 mg, 0.046 mmol, 50%). IR (cm⁻¹) 2957, 2118, 1739, 1456, 1372, 1211, 1031, 806, 744, 680; [α]_{D}²⁰−14.28 (c 0.42, CHCl₃); ¹H NMR (400 MHz, CDCl₃) δ 7.84 (m, 1H), 7.44 (m, 2H), 7.33 (m, 2H), 7.29 (m, 1H), 7.23 (d, J=8.5 Hz, 1H), 4.89 (s, 2H), 4.86 (d, J=4.5 Hz, 2H), 4.79 (ddd, J=11.5, 11.0, 4.5 Hz, 1H), 2.00 (d, J=11.0 Hz, 1H), 1.69-1.64 (m, 3H), 1.59 (ddd, J=7.0, 7.0, 2.0 Hz, 1H), 1.49 (m, 1H), 1.33-1.26 (m, 3H), 1.05 (s, 21H), 0.92 (d, J=6.5 Hz, 3H), 0.81 (d, J=7.0 Hz, 3H), 0.69 (d, J=7.0 Hz, 3H); ¹³C NMR (100 MHz, CDCl₃) δ 167.4, 152.9, 151.3, 142.8, 136.0, 129.9, 126.2, 123.7, 123.3, 123.0, 121.7, 120.0, 114.5, 109.4, 100.4, 90.9, 76.6, 57.8, 46.8 (2C), 40.6, 34.0, 31.4, 26.1, 23.1, 21.9, 20.7, 18.5 (6C), 16.0, 11.0 (3C).

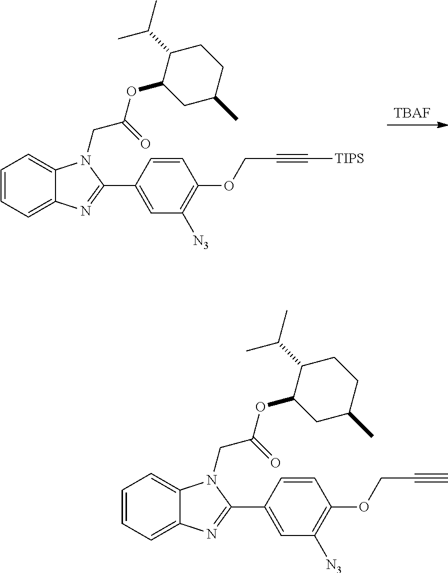

To a solution of TIPS-protected alkyne (23 mg, 0.036 mmol) in anhydrous THF (0.36 mL) at 0° C. was added TBAF (0.05 mL, 1M solution in THF). The reaction was stirred for 2 h and slowly raised to rt. The reaction was quenched with saturated NaHCO₃ solution (2 mL). The mixture was extracted with EtOAc (3×10 mL), and the organic extract was washed with brine and dried over with anhydrous MgSO₄. The solvent was removed under reduced pressure, and the resulting residue was purified by flash chromatography on silica gel (gradient elution, 35% EtOAc in hexanes) to afford the entitled compound LW-II-282 (9 mg, 0.019 mmol, 53%). IR (cm⁻¹) 3295, 2922, 2118, 1736, 1484, 1459, 1299, 1215, 1017, 745; [α]_{D}²⁰−29.38 (c 0.81, CHCl₃); ¹H NMR (400 MHz, CDCl₃) δ 7.82 (m, 1H), 7.45 (m, 2H), 7.33 (m, 2H), 7.29 (m, 1H), 7.15 (d, J=9.2 Hz, 1H), 4.87 (d, J=3.6 Hz, 2H), 4.84 (d, J=2.4 Hz, 2H), 4.78 (ddd, J=11.2, 10.8, 4.8 Hz, 1H), 2.59 (t, J=2.4 Hz, 1H), 1.99 (d, J=11.0 Hz, 1H), 1.89-1.42 (m, 5H), 1.33-1.24 (m, 3H), 0.90 (d, J=6.4 Hz, 3H), 0.80 (d, J=7.2 Hz, 3H), 0.68 (d, J=7.2 Hz, 3H); ¹³C NMR (100 MHz, CDCl₃) δ 167.4, 152.7, 151.0, 142.8, 136.0, 135.0, 129.9, 126.4, 124.0, 123.3, 123.0, 121.6, 120.1, 114.0, 109.4, 76.76, 76,75, 56.8, 46.80, 46.76, 40.6, 34.0, 31.4, 26.2, 23.1, 21.9, 20.7, 16.0; ES-API MS: m/z calcd for C₂₈F₃₁H₅O₃, found 486.0 [M+H].

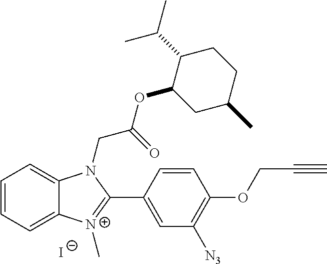

The title compounds were obtained following the general procedure (Step F) described above. Purification of the residue by prep-TLC (5% MeOH in CH₂Cl₂) afforded the entitled salt LW-II-291 as a yellow gel (60%). IR (cm⁻¹) 2957, 2926, 2870, 2360, 2342, 2121, 1739, 1493, 1482, 1471, 1228, 1009, 759, 732; [α]_{D}²⁰−22.08 (c 0.83, CHCl₃); ¹H NMR (400 MHz, CDCl₃) δ 8.13 (d, J=8.8 Hz, 1H), 7.80 (m, 1H), 7.67 (m, 2H), 7.60 (m, 1H), 7.43 (m, 1H), 7.36 (d, J=8.8 Hz, 1H), 5.14 (m, 2H), 4.90 (d, J=2.4 Hz, 2H), 4.78 (ddd, J=10.8, 10.8, 4.4 Hz, 1H), 4.02 (s, 3H), 2.66 (t, J=2.4 Hz, 1H), 1.93 (d, J=12.0 Hz, 1H), 1.71-1.35 (m, 8H), 0.92 (d, J=6.8 Hz, 3H), 0.87 (d, J=7.2 Hz, 3H), 0.71 (d, J=7.2 Hz, 3H); ¹³C NMR (100 MHz, CDCl₃) δ 166.1, 154.2, 150.6, 132.1, 131.9, 130.9, 130.1, 128.1, 127.9, 122.9, 114.8, 113.6, 113.5, 113.0, 78.0, 77.9, 76.7, 57.2, 49.0, 46.9, 40.8, 34.1, 34.0, 31.6, 26.7, 23.4, 22.1, 20.9, 16.3; ES-API MS: m/z calcd for C₂₉H₃₄IN₅O₃, found 500.0 [M−I].

### Example 2

### Biological Activity

1. Methods

A. Cell Culture

Mut6 tumor cells were cultured in DMEM/F12 media supplemented by EGF (10 ng/mL), basic FGF (10 ng/mL), heparin (4 ug/mL), glucose (total 21 mM), NaHCO₃ (113 μg/mL), Hepes (119 μg/mL), glutamine (2 mM), sodium pyruvate (1 mM), N₂ supplement (ThermoFisher Gibco 17502-048, 100×, at 100-fold dilution), B27 supplement (ThermoFisher Gibco 12587-010, 50×, at 100-fold dilution), and penicillin/streptomycin (ThermoFisher, final 1%). Cells were maintained in Ultra-low attachment plate (Corning) as sphere-forming cells. For ATP assays and protein/RNA isolation, cells were plated into 96-well plates or 6-well plated that were coated with poly-lysine (Sigma P6407, 10 μg/mL in PBS, 4 hr at 37° C.) and laminin (ThermoFisher 23017015, ˜5 mg/L in PBS, overnight at 37° C.). MEFs and Astrocytes were cultured in DMEM supplemented with 10% FBS and 1% Penicillin/Streptomycin.

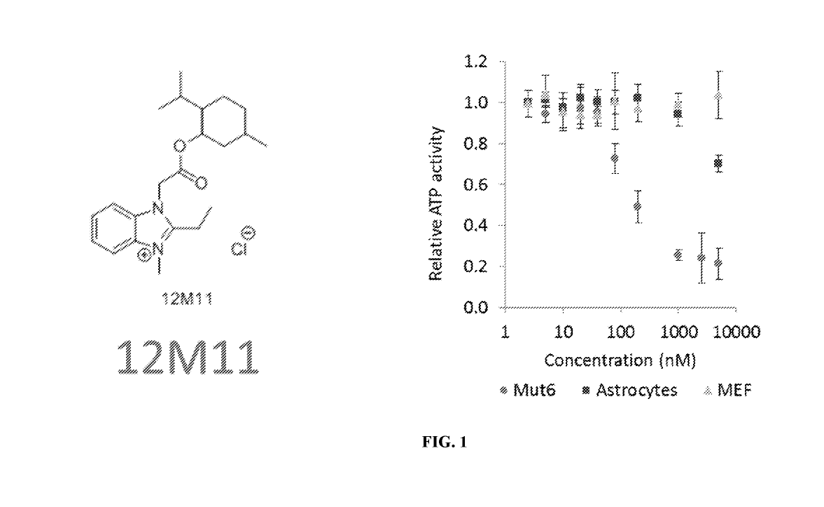

B. Cytotox Testing for 12M11 and Analogs in Mut6, MEF, and Astrocytes (FIG. 1)

2,000 cells were plated into each well of 96-well plates. About 3 hours later, cells were treated by 12M11, at 800-fold dilution (4 mM stock was diluted into 5 μM final concentration). Cells were further incubated for 4 days. CellTiter-Glo assay (Promega) was performed according to manufacturer's instructions, by adding 20 μL of reagent into each well. Luminescence signal was detected by the PolarStar plate reader (BMG Labtech). Experiments were performed in quadruplicate, and results are presented with mean average −/+ standard deviation. EC₅₀₅ were calculated by GraphPad Prism6 software.

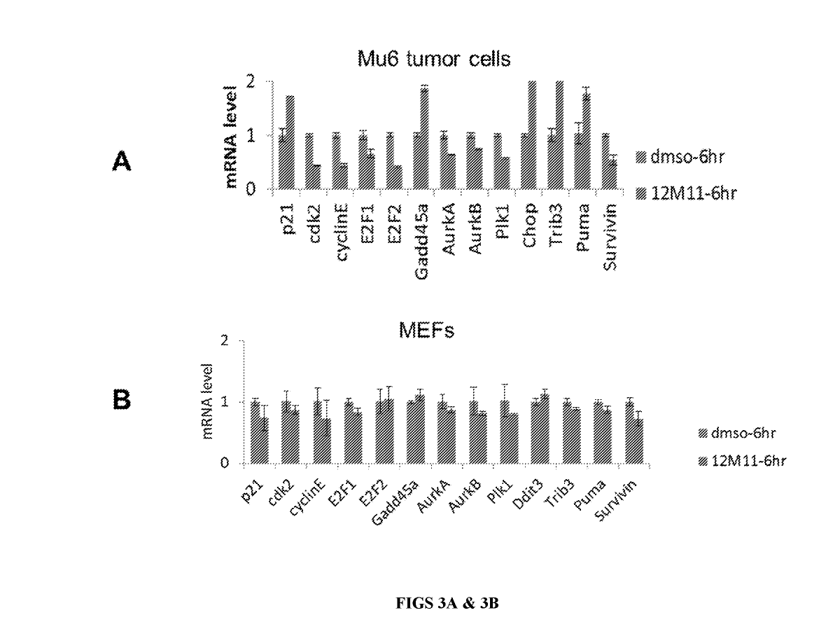

C. qRT-PCR (FIGS. 3 & 4)

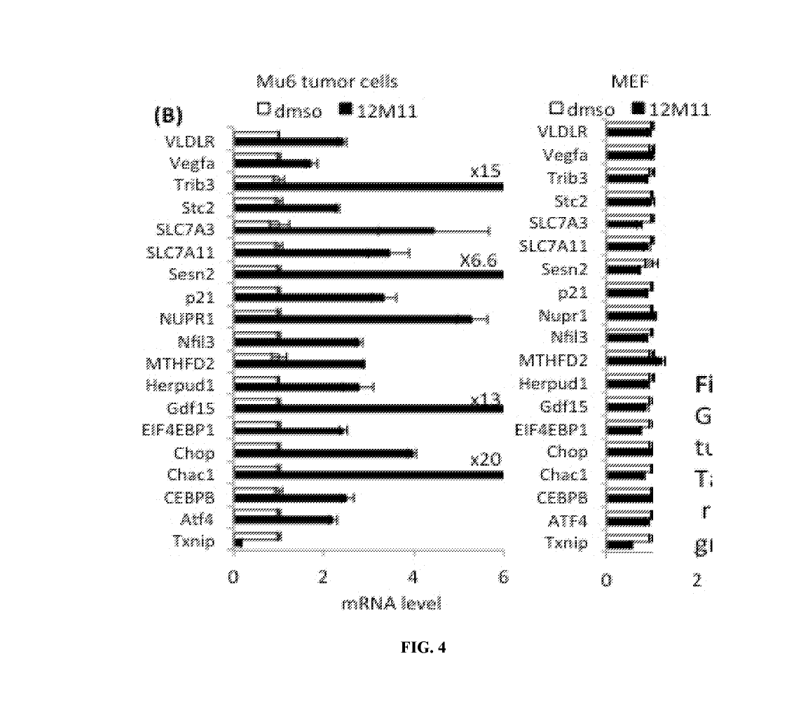

The genes shown in FIGS. 3 and 4 were identified from the gene expression microarray with Mut6 tumor cells (6 hr, 12 hr, and 24 hr time points). In the analysis of cell cycle and apoptosis gene, 13 genes were identified showing differential expression at 6 hr time point. These genes are confirmed by qRT-PCR in the FIG. 3. In search of the genes with highest fold increase/decrease at 6 hr time point, 19 genes were identified, which were confirmed in the FIG. 4 by qRT-PCR.

Mut6 tumor cells or MEFs were treated by DMSO or 1 uM 12M11 for 6 hours, and then RNA samples were isolated, following the protocol of the RNeasy Plus Mini Kit (Qiagen). 1 μg of RNA was used to set up cDNA synthesis with the iScript cDNA Synthesis kit (BioRad). Quantitative real-time PCR was performed, using Power Syber green kit (Life Technologies) and the Applied Biosystems 7500 Real-Time PCR machine. Primers for qPCR are shown below.

| | | |
|---|---|---|
| p21 | CCTGGTGATGTCCGACCTG | CCATGAGCGCATCGCAATC |
| | (SEQ ID NO: 1) | (SEQ ID NO: 2) |
| cdk2 | CCTGCTTATCAATGCAGAGGG | TGCGGGTCACCATTTCAGC |
| | (SEQ ID NO: 3) | (SEQ ID NO: 4) |
| cyclinE | GCGAGGATGAGAGCAGTTC | AAGTCCTGTGCCAAGTA |
| | (SEQ ID NO: 5) | (SEQ ID NO: 6) |
| E2F1 | CTCGACTCCTCGCAGATCG | GATCCAGCCTCCGTTTCACC |
| | (SEQ ID NO: 7) | (SEQ ID NO: 8) |
| E2F2 | ACGGCGCAACCTACAAAGAG | GTCTGCGTGTAAAGCGAAGT |
| | (SEQ ID NO: 9) | (SEQ ID NO: 10) |
| Gadd45a | CCGAAAGGATGGACACGGTG | TTATCGGGGTCTACGTTGAGC |
| | (SEQ ID NO: 11) | (SEQ ID NO: 12) |
| AurkA | CTGGATGCTGCAAACGGATA | CGAAGGGAACAGTGGTCTTAAC |
| | G | A |
| | (SEQ ID NO: 13) | (SEQ ID NO: 14) |
| AurkB | CAGAAGGAGAACGCCTACCC | GAGAGCAAGCGCAGATGTC |
| | (SEQ ID NO: 15) | (SEQ ID NO: 16) |
| Plk1 | CCCGCTGGCGAAAGAAATTC | CATTTGGCGAAGCCTCCTTTA |
| | (SEQ ID NO: 17) | (SEQ ID NO: 18) |
| Ddit3/CHOP | CTGGAAGCCTGGTATGAGGAT | CAGGGTCAAGAGTAGTGAAGGT |
| | (SEQ ID NO: 19) | (SEQ ID NO: 20) |
| Trib3 | TGCAGGAAGAAACCGTTGGA | CTCGTTTTAGGACTGGACACTTG |
| | G | (SEQ ID NO: 22) |
| | (SEQ ID NO: 21) | |
| Puma | AGCAGCACTTAGAGTCGCC | CCTGGGTAAGGGGAGGAGT |
| | (SEQ ID NO: 23) | (SEQ ID NO: 24) |
| Survivin | GAGGCTGGCTTCATCCACTG | CTTTTTGCTTGTTGTTGGTCTCC |
| | (SEQ ID NO: 25) | (SEQ ID NO: 26) |
| Txnip | TCTTTTGAGGTGGTCTTCAAC | GCTTTGACTCGGGTAACTTCACA |
| | G | (SEQ ID NO: 28) |
| | (SEQ ID NO: 27) | |
| ATF4 | CCTTCGACCAGTCGGGTTTG | CTGTCCCGGAAAAGGCATCC |
| | (SEQ ID NO: 29) | (SEQ ID NO: 30) |
| CEBPB | GGGGTTGTTGATGTTTTTGG | CGAAACGGAAAAGGTTCTCA |
| | (SEQ ID NO: 31) | (SEQ ID NO: 32) |
| Chac1 | CTGTGGATTTTCGGGTACGG | CCCCTATGGAAGGTGTCTCC |
| | (SEQ ID NO: 33) | (SEQ ID NO: 34) |
| EIF4EBP1 | GGGGACTACAGCACCACTC | GTTCCGACACTCCATCAGAAAT |
| | (SEQ ID NO: 35) | (SEQ ID NO: 36) |
| Gdf15 | ACTGTGCAGGCAACTCTTGAA | GTTCGCGGAGCGATACAGG |
| | (SEQ ID NO: 37) | (SEQ ID NO: 38) |
| Herpud1 | GCAGTTGGAGTGTGAGTCG | TCTGTGGATTCAGCACCCTTT |
| | (SEQ ID NO: 39) | (SEQ ID NO: 40) |
| MTHFD2 | AGTGCGAAATGAAGCCGTTG | GACTGGCGGGATTGTCACC |
| | (SEQ ID NO: 41) | (SEQ ID NO: 42) |
| NfiL3 | GAACTCTGCCTTAGCTGAGGT | ATTCCCGTTTTCTCCGACACG |
| | (SEQ ID NO: 43) | (SEQ ID NO: 44) |
| Nupr1 | CCCTTCCCAGCAACCTCTAAA | TCTTGGTCCGACCTTTCCGA |
| | (SEQ ID NO: 45) | (SEQ ID NO: 46) |
| Sesn2 | TCCGAGTGCCATTCCGAGAT | TCCGGGTGTAGACCCATCAC |
| | (SEQ ID NO: 47) | (SEQ ID NO: 48) |
| SLC7A3 | GGAGCTGGGTATGGGTGAGA | TGGATGGTCCTGCTTTATCTTTG |
| | (SEQ ID NO: 49) | (SEQ ID NO: 50) |
| SLC7A11 | GGCACCGTCATCGGATCAG | CTCCACAGGCAGACCAGAAAA |
| | (SEQ ID NO: 51) | (SEQ ID NO: 52) |
| Stc2 | CTGGGCCAGTTTGTGACCC | ACGTCATGCAAATCCCATGTAAA |
| | (SEQ ID NO: 53) | (SEQ ID NO: 54) |
| Vegfa | CTTGTTCAGAGCGGAGAAAG | ACATCTGCAAGTACGTTCGTT |
| | C | (SEQ ID NO: 56) |
| | (SEQ ID NO: 55) | |
| Vldlr | GGCAGCAGGCAATGCAATG | GGGCTCGTCACTCCAGTCT |
| | (SEQ ID NO: 57) | (SEQ ID NO: 58) |

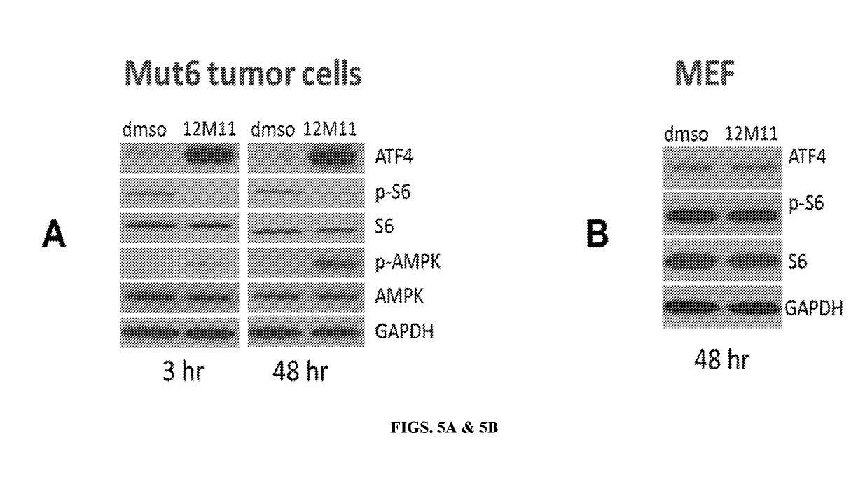

D. ATF4; p-S6 Westerns (FIG. 5)

Mut6 tumor cells and MEFs were treated by DMSO or 1 μM 12M11 for the indicated times. Cell lysate was prepared by RIPA buffer with phosphatase inhibitor (Thermo) and protease inhibitor (Roche). Antibodies are purchased from Santa Cruz (ATF4), Cell Signaling (pS6, S6, AMPK, and pAMPK), and Millipore (Gapdh).

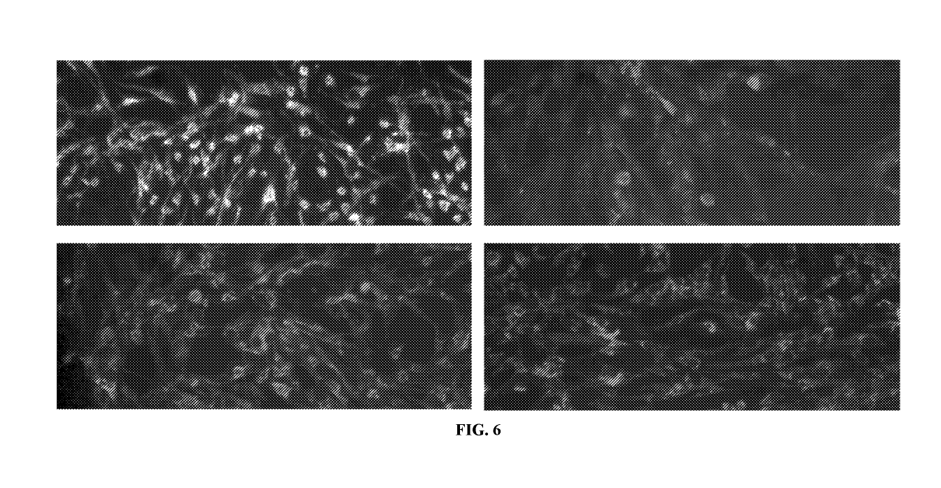

E. TMRE Staining (FIG. 6)

TMRE mitochondrial membrane potential assay kit was purchased from Abcam (113852) and the assay was performed according to the manufacturer's protocol. Briefly, cells were pre-incubated with 1 μM 12M11 (10 min or 18 hrs) or 100 nM FCCP (10 min) and incubated for additional 20 min with 200 nM TMRE before pictures were taken.

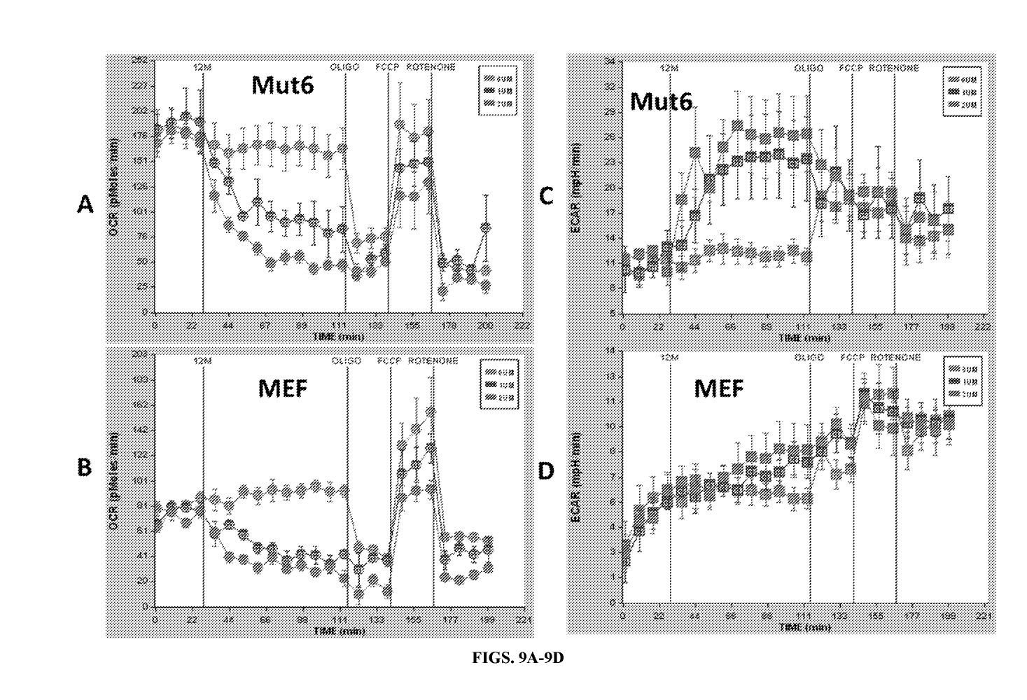

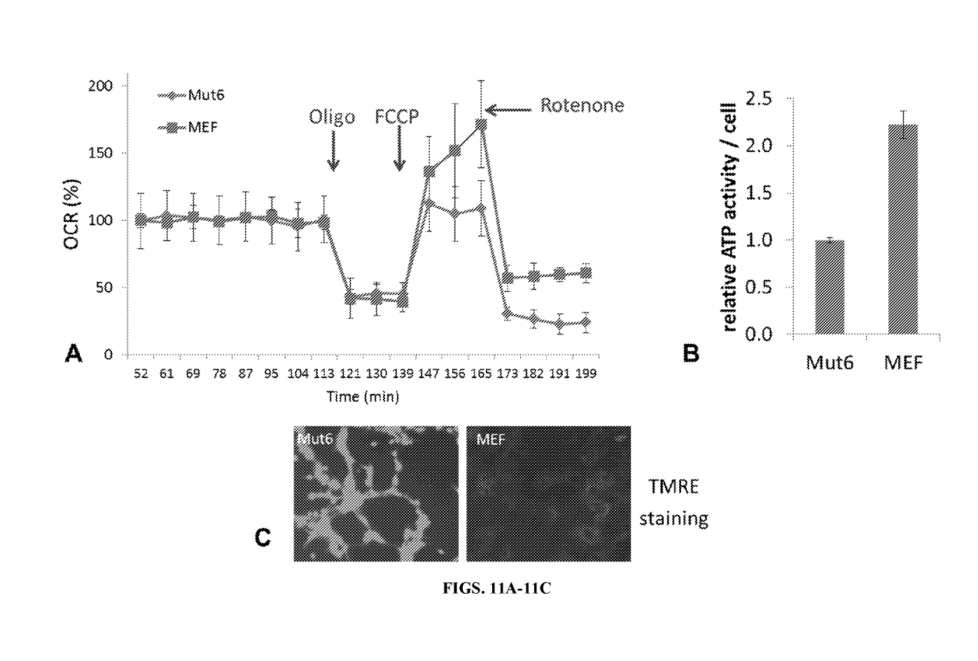

F. OCR and ECAR Measurements Using Seahorse (FIGS. 9 and 11A)

Oxygen consumption rate (OCR) and extracellular acidification rate (ECAR) were measured by Seahorse Bioscience instrument (XF24) with 80-90% confluent cells, according to the manufacturer's protocol. Briefly, cells were equilibrated for 1 hr at 37° C. incubator lacking CO₂. Oxygen concentration and pH in media were measured during sequential treatment with 12M11, Oligomycin (1 μM), FCCP (1 μM), and Rotenone (200 nM). A minimum of three wells were utilized per condition to calculate OCR and ECAR.

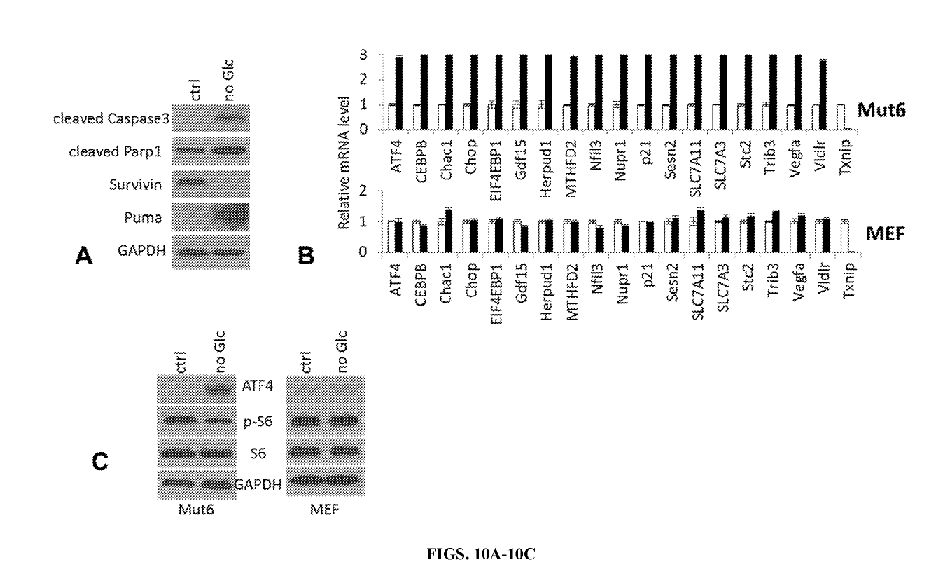

G. Glucose Deprivation (FIG. 10)

Mut6 tumor cells were cultured for 4 days in DMEM-noGlucose-media (ThermoFisher 11966-025) supplemented by 0 mM (no Glc) or 21 mM (ctrl) glucose and all the other ingredients (see above). Cell lysates were subjected to the western blotting to monitor cleaved-caspase3 (Cell Signaling), cleaved-Parpl (Novus), Puma (Cell Signaling), and Survivin (Novus). While anti-apoptotic protein Survivin was decreased by glucose deprivation, pro-apoptoic protein Puma was decreased. Cells underwent apoptosis as indicated by cleavage of Caspase3 and Parp1.

19 stress responsive genes identified from 12M11 response were monitored by qRT-PCR. Mut6 tumor cells and MEFs were deprived for glucose for 15 hours and then RNA samples were collected for qRT-PCR analysis. Primers were listed above.

Mut6 tumor cells and MEFs were starved for glucose for 15 hours and then cell lysates were collected for western blotting analysis. The effect of glucose deprivation on ATF4 and pS6 levels was examined.

H. mRNA Expression Levels (FIG. 11B)

2,000 Mut6 tumor cells or MEFs were plated into 96-well plates and 5 hour later ATP levels were measured by Cell-Titer Glo (Promega). In addition to this result, ATP levels of these cells cultured for three days were also compared and then normalizing them by cell numbers. Similar results were observed. Experiments were performed in five replicates, and results are presented with mean average −/+ standard deviation.

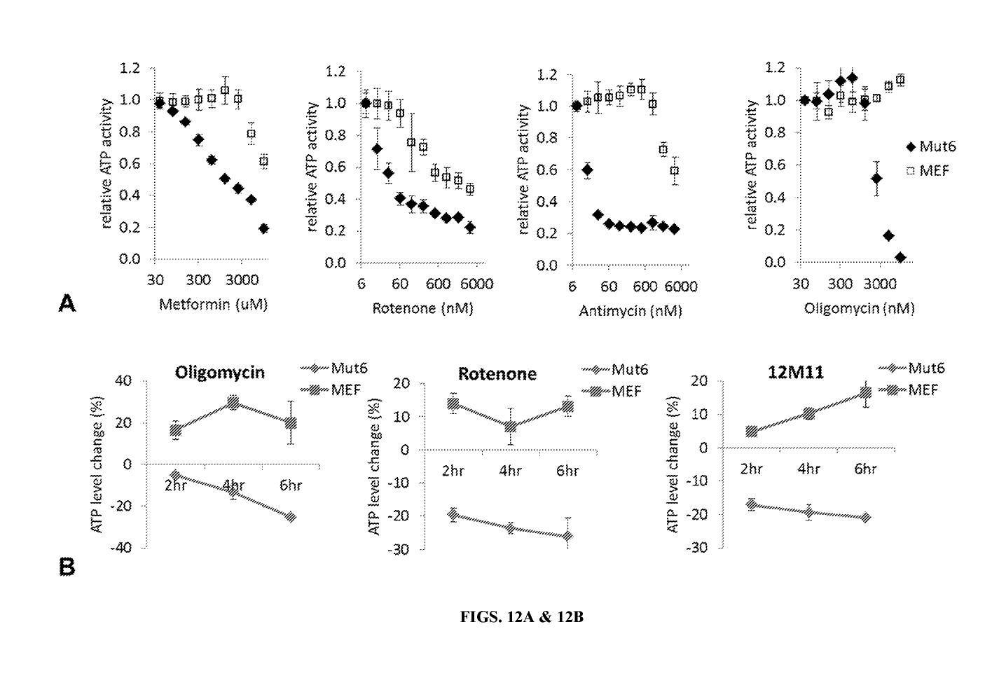

I. Change in ATP Levels (FIG. 12B)

Mut6 tumor cells or MEFs were plated into 96-well plates. One day later, Oligomyin (2 μM), Rotenone (200 nM), or 12M11 (1 μM) were added into each well and then ATP levels were measured by Cell-Titer Glo (Promega) after 2 hr, 4 hr, or 6 hr incubation. Each data is mean of six replicates with ± standard deviation.

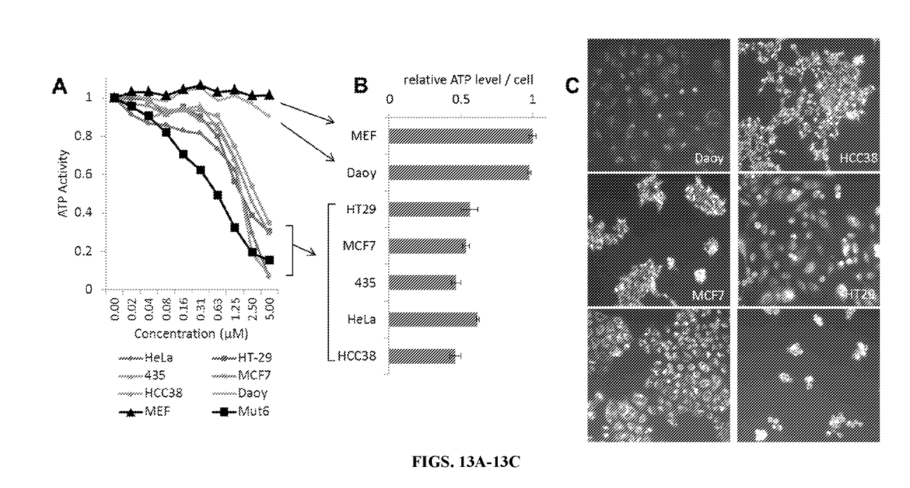

J. Cellular ATP Levels (FIG. 13B)

8,000 cells were plated into 96-well plates and 2 hour later ATP levels were measured by Cell-Titer Glo (Promega). Experiments were performed in triplicate, and results are presented with mean average ± standard deviation.

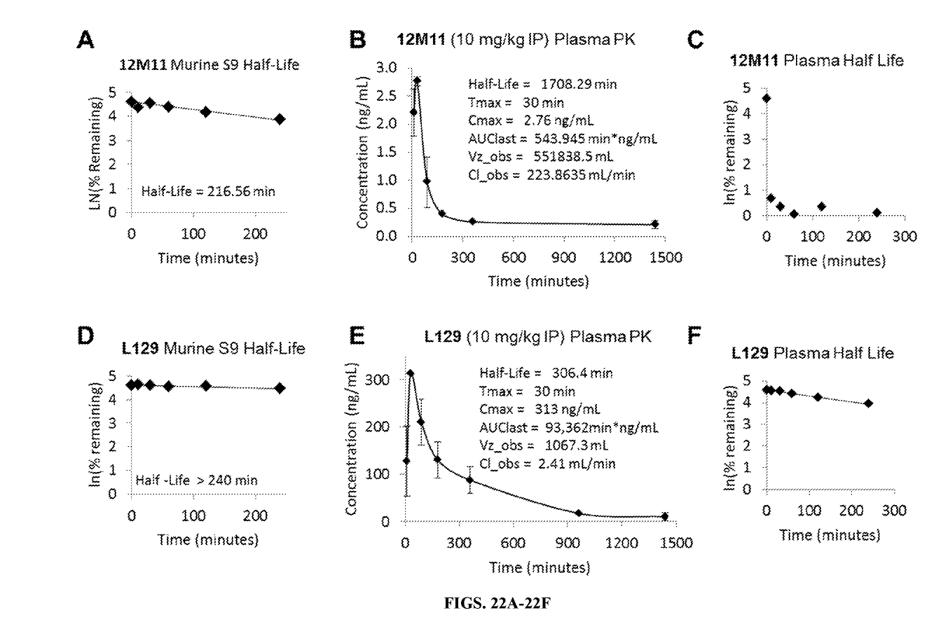

K. Pharmacokinetic Analysis of 12M11 (FIGS. 22A-22F)

12M11 (2 μM final concentration) was incubated with murine plasma and saline for 0-1440 minutes. Reactions were quenched with 200 μl (1:2) of methanol containing 0.1% formic acid and 200 ng/ml IS (IS final conc.=100 ng/mL). Samples were vortexed for 15 seconds, incubated at RT for 10 minutes and spun for 5 minutes at 13.2K rpm. Supernatant (1 mL) was then transferred to an eppendorf tube and spun in a table top, chilled centrifuge for 5 minutes at 13.2K rpm. Supernatant (800 μL) was transferred to an HPLC vial (w/out insert). Analyzed by Qtrap 3200 mass spectrometer. These methods were used data obtained in the FIGS. 22C and 22F.

22D. L129 (2 mM in DMSO) was incubated with Murine S9 (Lot KWB) fraction and Phase I (NADPH Regenerating System) cofactors for 0-240 minutes. Reactions were quenched with 1 mL (1:1) of methanol containing 0.2% formic acid and 100 ng/ml IS (IS final conc.=50 ng/mL). Samples were vortexed for 15 seconds, incubated at RT for 10 minutes and spun for 5 minutes at 2400 rpm. Supernatant (1 mL) was then transferred to an eppendorf tube and spun in a table top, chilled centrifuge for 5 minutes at 13.2K rpm. Supernatant (800 uL) was transferred to an HPLC vial (w/out insert). Analyzed by Qtrap 3200 mass spectrometer. These methods were used data obtained in the FIGS. 22A and 22D.

22E. 21 female CD-1 mice (6 wks) were administered 10 mg/kg L129 IP, 0.2 ml/mouse formulated as 5% DMSO, 10% Cremophor EL, and 85% D₅W. Plasma was processed from whole blood by centrifugation of the ACD treated blood for 10′ at 10,000 rpm in a standard centrifuge. Brains were weighed and snap frozen in liquid nitrogen. Brain homogenates were prepared by mincing the brain tissue and homogenizing in a 3-fold volume of PBS (total volume of homogenate in mL=4× weight in g.) 100 microliters plasma or brain homogenate was mixed with 200 microliters of acetonitrile containing formic acid and an internal standard (final concentration of formic acid=0.1%, IS=25 ng/mL). The samples were vortexed 15 sec, incubated at room temp for 10′ and spun 2×13,200 rpm in a standard microcentrifuge. The supernatant was then analyzed by LC/MS/MS. Buffer A: Water+0.1% formic acid; Buffer B: MeOH+0.1% formic acid; flow rate 1.5 ml/min; column Agilent C18 XDB column, 5 micron packing 50×4.6 mm size; 0-1.5 min 3% B, 1.5-2.5 min gradient to 100% B; 2.5-3.5 min 100% B, 3.5-3.6 min gradient to 3% B; 3.6-4.5 min 3% B; IS N-benzylbenzamide (transition 212.1 to 91.1); Compound transition 474.2 to 336.3. These methods were used data obtained in the FIGS. 22B and 22E.

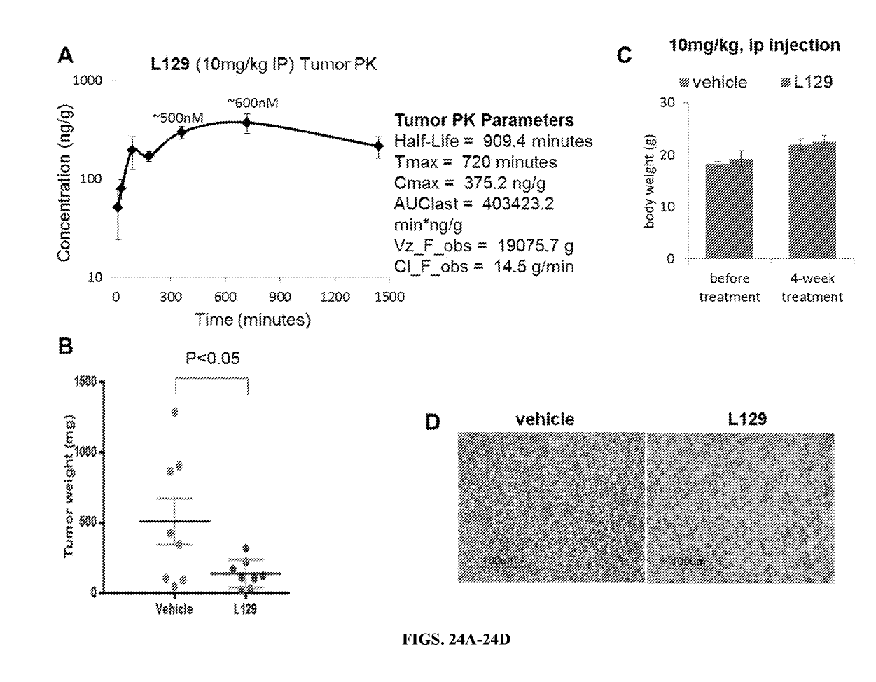

L. Animal Allograft Studies with L129 (FIGS. 24A-D)

24 female nude mice (8 wks) were implanted with 200,000 Mut6 tumor cells by subcutaneous injection on both the left and right shoulders. 24 days post-implantation, the mice were administered 10 mg/kg L129 by intraperitoneal injection (0.2 mL/mouse formulated as 5% DMSO, 10% Cremophor EL, and 85% D₅W). Tumors were weighed and snap frozen in liquid nitrogen. Tumor homogenates were prepared by mincing the tumor tissue and homogenizing in a 3-fold volume of PBS (total volume of homogenate in mL=4× weight in g.) 100 microliters tumor homogenate was mixed with 200 microliters of acetonitrile containing formic acid and an internal standard (final concentration of formic acid=0.1%, IS=25 ng/mL). The samples were vortexed 15 sec, incubated at room temp for 10′ and spun 2×13,200 rpm in a standard microcentrifuge. The supernatant was then analyzed by LC/MS/MS. This data is shown in FIG. 24A.

8 female nude mice (8 wks) were implanted with 200,000 Mut6 tumor cells by subcutaneous injection on both the left and right shoulders. 3 days later, vehicle or L129 were injected daily (10 mg/kg, i.p.) for 4 weeks. Each tumor was weighed, and each group was compared by unpaired t-test (two tailed). This data is shown in FIG. 24B.

Body weights of each group from (B) were compared before and after 4-week injection of vehicle or L129. This data is shown in FIG. 24C.

Tumors from each group were stained for H&E (Hematoxylin and Eosin). This data is shown in FIG. 24D.

2. Biological Activity Results

The compounds described herein were tested using a cell-based cytotoxicity assay against neuronal stem cells in Mut6, which is a mouse glioblastoma model cell line.

TABLE 1
Biological Activity of the Compounds

| | | | Activity in | | | |
|---|---|---|---|---|---|---|
| | | | Mut6 [EC₅₀: | | | |
| | | | <100 nM | | | |
| | | | +++100- | | | |
| | | | 1000 nM | Plasma T_{1/2}/ | | |
| | | | ++1-5 μM + | Buffer | | |
| Structure | CLogP | MW | >5 μM N/A | Stability | S9 T_{1/2} | |
| 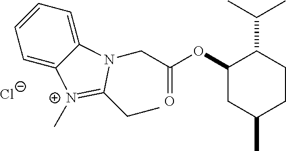 [SMILES: [Cl-] ; CCc1n(CC(=O)O[C@@H]2C[C@H](C)CC[C@H]2C(C)C)c2ccccc2[n+]1C] | 1.1038 | 393 | ++ | <4 min/100% | 217 | min |
| 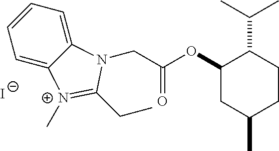 [SMILES: [I-] ; CCc1n(CC(=O)O[C@@H]2C[C@H](C)CC[C@H]2C(C)C)c2ccccc2[n+]1C] | 1.1038 | 484.4 | ++ | | | |
| 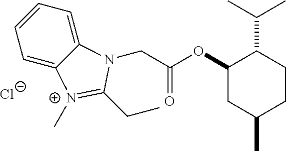 [SMILES: [Cl-] ; CCc1n(CC(=O)O[C@@H]2C[C@H](C)CC[C@H]2C(C)C)c2ccccc2[n+]1C] | 1.1038 | 393 | ++ | | | |
| 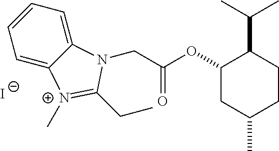 [SMILES: [I-] ; CCc1n(CC(=O)O[C@H]2C[C@@H](C)CC[C@@H]2C(C)C)c2ccccc2[n+]1C] | 1.1038 | 484.4 | ++ | | | |
| 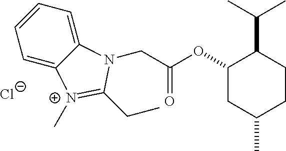 [SMILES: [Cl-] ; CCc1n(CC(=O)O[C@H]2C[C@@H](C)CC[C@@H]2C(C)C)c2ccccc2[n+]1C] | 1.1038 | 393 | ++ | | | |
| 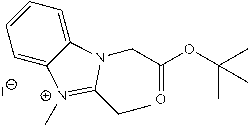 [SMILES: [I-] ; CCc1n(CC(=O)OC(C)(C)C)c2ccccc2[n+]1C] | −1.6562 | 402.3 | N/A | | | |
| 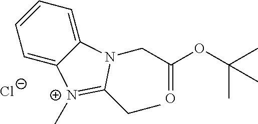 [SMILES: [Cl-] ; CCc1n(CC(=O)OC(C)(C)C)c2ccccc2[n+]1C] | −1.6562 | 310.8 | N/A | | | |
| 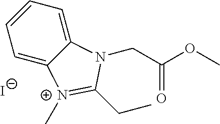 [SMILES: [I-] ; CCc1n(CC(=O)OC)c2ccccc2[n+]1C] | −2.8932 | 360.2 | N/A | | | |
| 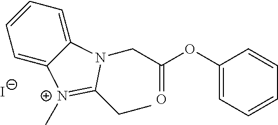 [SMILES: [I-] ; CCc1n(CC(=O)Oc2ccccc2)c2ccccc2[n+]1C] | −1.6706 | 422.3 | N/A | | | |
| 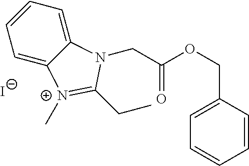 [SMILES: [I-] ; CCc1n(CC(=O)OCc2ccccc2)c2ccccc2[n+]1C] | −1.1824 | 436.3 | N/A | | | |
| 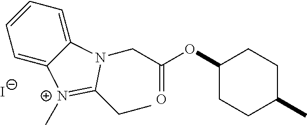 [SMILES: [I-] ; CCc1n(CC(=O)O[C@H]2CC[C@@H](C)CC2)c2ccccc2[n+]1C] | −0.3432 | 442.3 | N/A | | | |
| 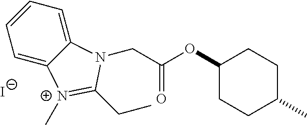 [SMILES: [I-] ; CCc1n(CC(=O)O[C@H]2CC[C@H](C)CC2)c2ccccc2[n+]1C] | −0.3432 | 442.3 | N/A | | | |
| 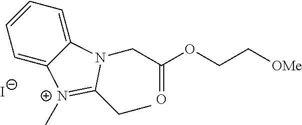 [SMILES: [I-] ; CCc1n(CC(=O)OCCOC)c2ccccc2[n+]1C] | −2.785 | 404.2 | N/A | | | |
| 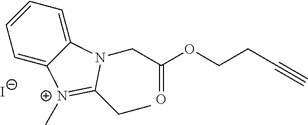 [SMILES: [I-] ; C#CCCOC(=O)Cn1c(CC)[n+](C)c2ccccc21] | −2.3142 | 398.2 | N/A | | | |
| 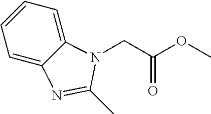 | 2.1508 | 218.3 | N/A | | | |
| 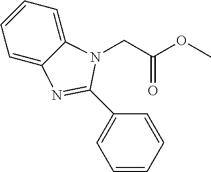 [SMILES: COC(=O)Cn1c(-c2ccccc2)nc2ccccc21] | 3.4508 | 266.3 | N/A | | | |
| 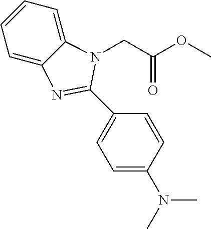 [SMILES: COC(=O)Cn1c(-c2ccc(N(C)C)cc2)nc2ccccc21] | 3.72944 | 309.4 | N/A | | | |
| 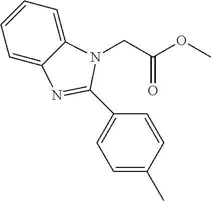 [SMILES: COC(=O)Cn1c(-c2ccc(C)cc2)nc2ccccc21] | 3.9498 | 280.3 | N/A | | | |
| 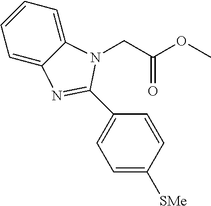 [SMILES: COC(=O)Cn1c(-c2ccc(SC)cc2)nc2ccccc21] | 4.04706 | 312.4 | N/A | | | |
| 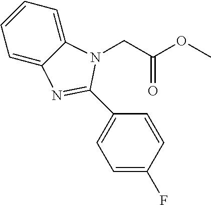 [SMILES: COC(=O)Cn1c(-c2ccc(F)cc2)nc2ccccc21] | 3.60086 | 284.3 | N/A | | | |
| 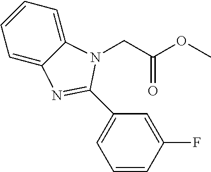 [SMILES: COC(=O)Cn1c(-c2cccc(F)c2)nc2ccccc21] | 3.60086 | 284.3 | N/A | | | |
| 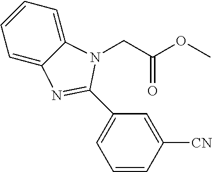 [SMILES: COC(=O)Cn1c(-c2cccc(C#N)c2)nc2ccccc21] | 2.90018 | 291.3 | N/A | | | |
| 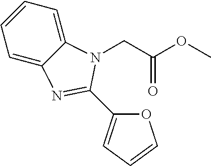 [SMILES: COC(=O)Cn1c(-c2ccco2)nc2ccccc21] | 2.8368 | 256.3 | N/A | | | |
| 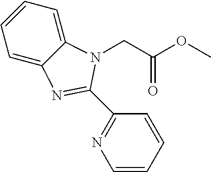 [SMILES: COC(=O)Cn1c(-c2ccccn2)nc2ccccc21] | 2.25119 | 267.3 | N/A | | | |
| 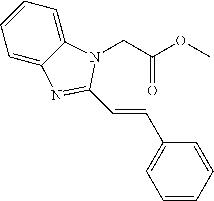 [SMILES: COC(=O)Cn1c(/C=C/c2ccccc2)nc2ccccc21] | 4.0448 | 292.3 | N/A | | | |
| 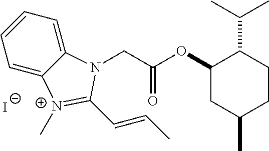 | 2.7588 | 496.4 | + | | | |
| 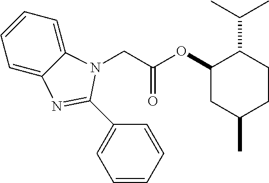 | 7.4478 | 390.5 | N/A | | | |
| 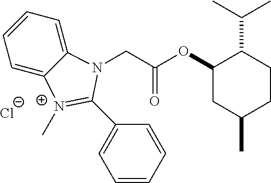 | 2.7038 | 441 | | | | |
| 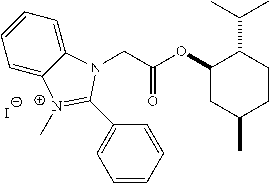 | 2.7038 | 532.5 | ++ | 10.6 min/97% | | |
| 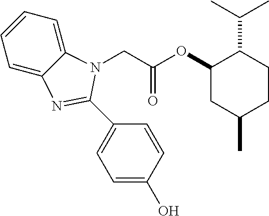 | 6.97828 | 406.5 | N/A | | | |
| 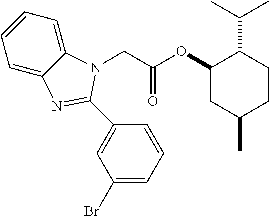 | 8.31786 | 469.4 | N/A | | | |
| 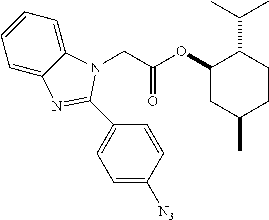 | 8.03984 | 431.5 | N/A | | | |
| 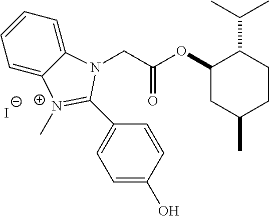 | 2.15266 | 548.5 | + | 9.8 min/114% | 133.3 | min |
| 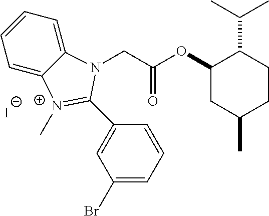 | 3.57072 | 611.4 | ++ | 29 min/94% | | |
| 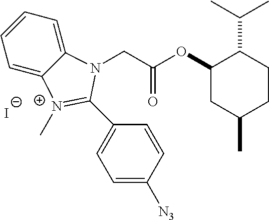 | 3.226 | 573.5 | ++ | 26 min/96% | 26 | min |
| 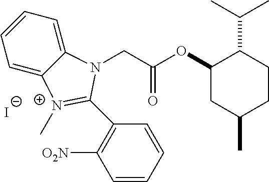 | 2.4552 | 577.5 | ++ | 47 min/100% | >240 | min |
| 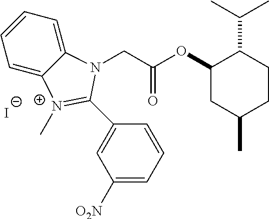 | 2.4552 | 577.5 | + | 330 min/133% | >240 | min |
| 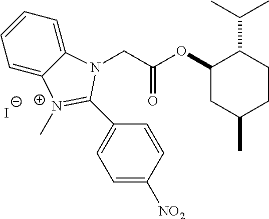 | 2.4552 | 577.5 | + | 90 min/100% | 139 | min |
| 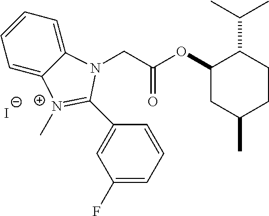 | 2.85072 | 550.5 | ++ | 11 min/100% | | |
| 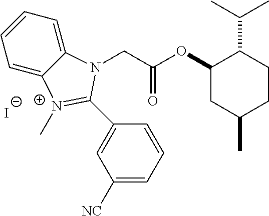 | 2.1459 | 557.5 | N/A | | | |
| 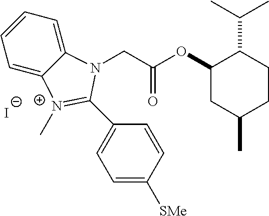 | 3.2826 | 578.6 | ++ | 10 min/100% | | |
| 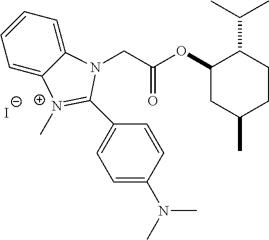 | 2.92919 | 575.5 | +++ | 8 min/9% | | |
| 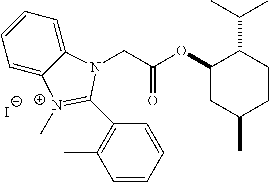 | 2.9028 | 546.5 | ++ | 73 min/100% | | |
| 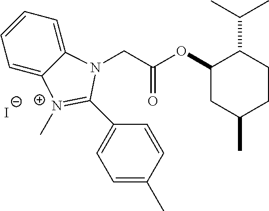 | 3.2028 | 546.5 | ++ | 24 min/95% | | |
| 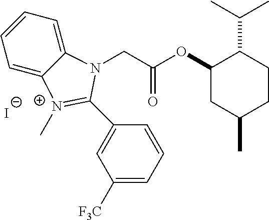 | 3.59366 | 600.5 | ++ | 4 hrs/100% | >240 | min |
| 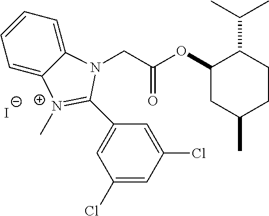 | 4.13509 | 601.4 | ++ | 39 min/91% | | |
| 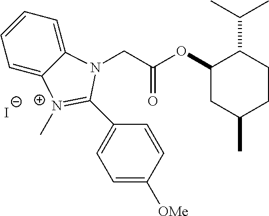 | 2.67468 | 562.5 | ++ | 15 min/100% | | |
| 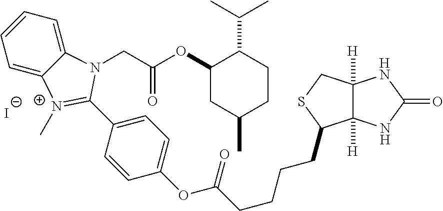 | 0.9668 | 774.8 | + | | | |
| 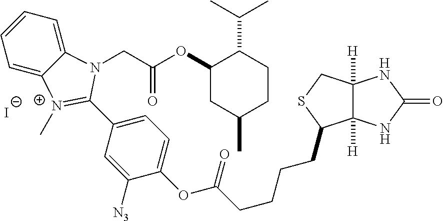 | 1.48936 | 815.8 | NA | | | |
| 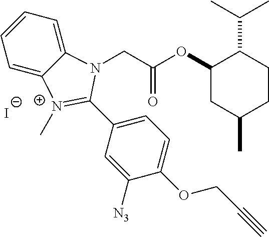 | 3.79724 | 500.6 | ++ | | | |
| 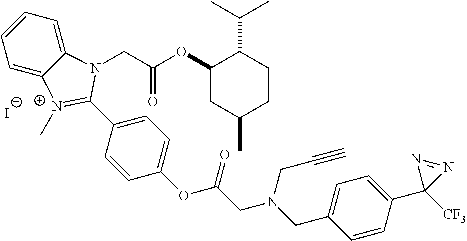 | 4.87288 | 714.8 | + | | | |
| 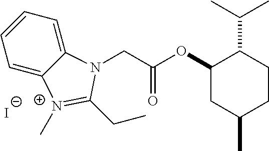 | 1.1038 | 484.4 | +++ | | | |
| 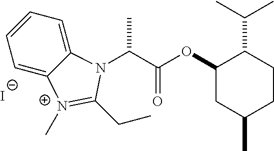 | 1.4128 | 498.4 | +++ | | | |
| 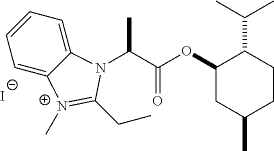 | 1.4128 | 498.4 | +++ | | | |
| 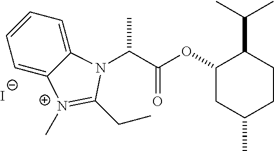 | 1.4128 | 498.4 | +++ | | | |
| 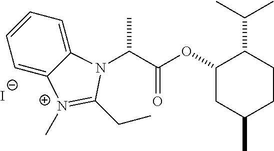 | 1.4128 | 498.4 | ++ | | | |
| 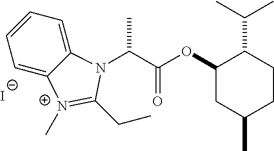 | 2.3408 | 526.5 | ++ | | | |
| 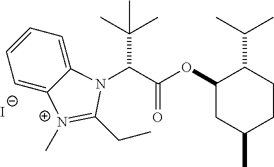 | 2.7398 | 540.5 | ++ | | | |
| 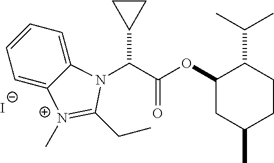 | 1.8568 | 524.5 | +++ | | | |
| 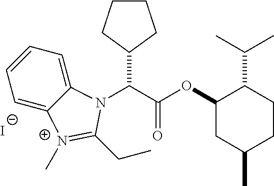 | 2.9748 | 552.5 | +++ | | | |
| 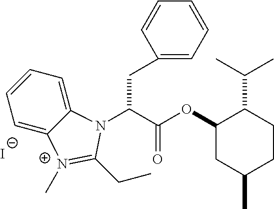 | 2.8308 | 574.5 | ++ | | | |
| 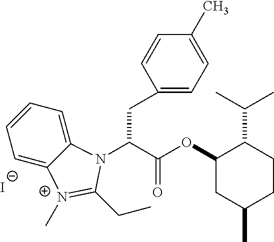 | 3.3298 | 588.6 | +++ | | | |
| 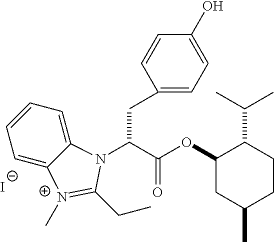 | 2.1638 | 590.5 | + | | | |
| 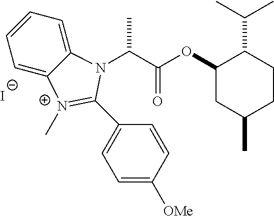 | 2.98368 | 576.5 | +++ | | | |
| 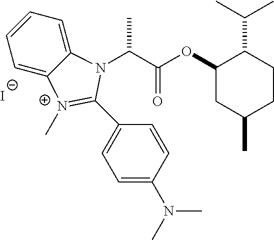 | 3.23819 | 589.6 | +++ | | | |
| 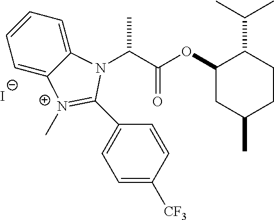 | 3.90266 | 614.5 | ++ | | | |
| 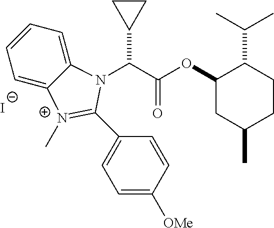 | 3.42768 | 602.6 | +++ | | | |
| 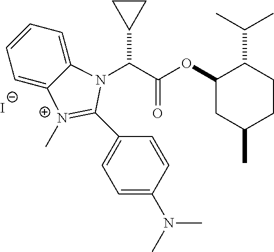 | 3.68219 | 615.6 | +++ | | | |
| 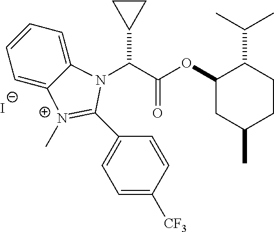 | 4.34666 | 640.5 | ++ | | | |
| 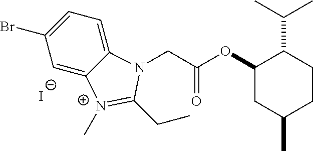 | 1.9815 | 563.3 | ++ | | | |
| 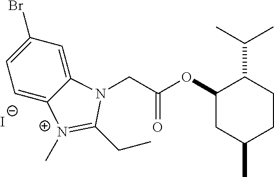 | 1.9815 | 563.3 | ++ | | | |
| 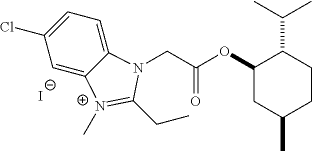 | 1.8315 | 518.8 | ++ | | | |
| 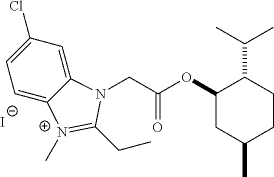 | 1.8315 | 518.8 | ++ | | | |
| 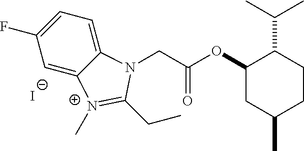 | 1.2615 | 502.4 | ++ | | | |
| 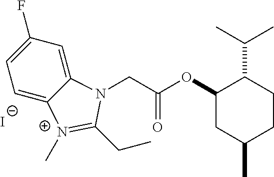 | 1.2615 | 502.4 | +++ | | | |
| 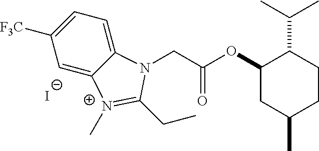 | 2.01252 | 552.4 | + | | | |
| 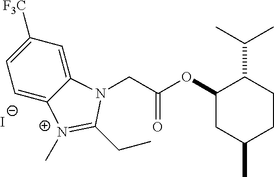 | 2.01252 | 552.4 | + | | | |
| 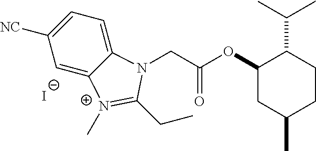 | 0.57092 | 509.4 | + | | | |
| 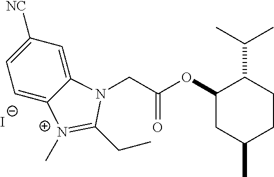 | 0.57092 | 509.4 | + | | | |
| 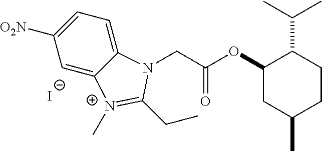 | 0.8783 | 529.4 | + | | | |
| 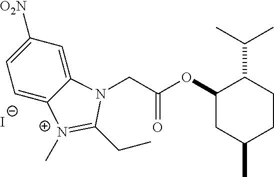 | 0.8783 | 529.4 | + | | | |
| 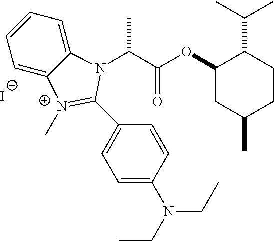 | 4.29619 | 617.616 | +++ | | | |
| 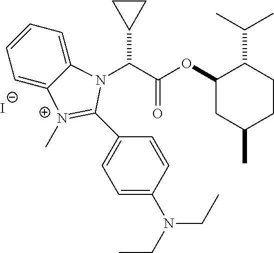 | 4.74019 | 643.654 | +++ | | | |
| 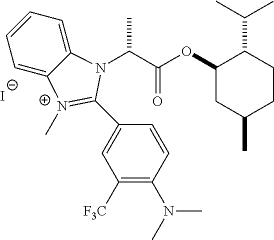 | 4.38289 | 657.561 | +++ | | | |
| 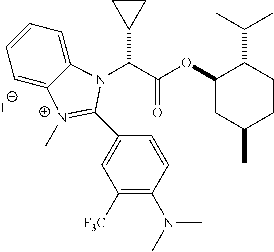 | 4.82689 | 683.599 | ++ | | | |
| 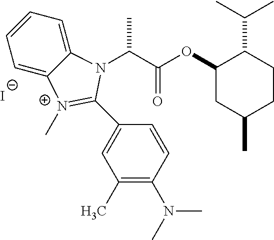 | 3.73719 | 603.589 | +++ | | | |
| 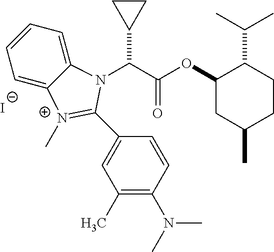 | 4.18119 | 629.627 | +++ | | | |
| 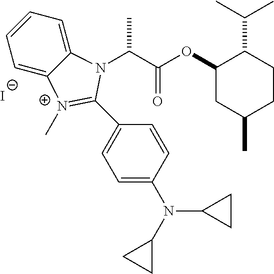 | 4.40619 | 641.638 | +++ | | | |
| 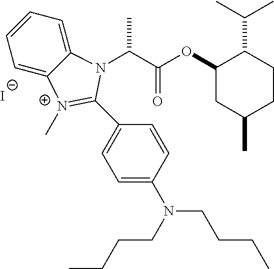 | 6.41219 | 673.724 | +++ | | | |
| 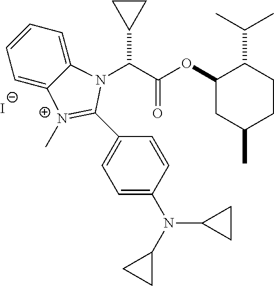 | 4.85019 | 667.676 | +++ | | | |
| 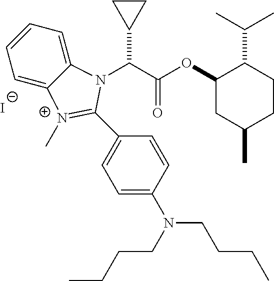 | 6.85619 | 699.762 | +++ | | | |
| 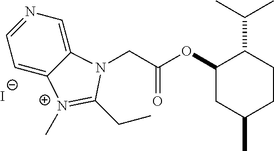 | −0.21183 | 485.41 | NA | | | |
| 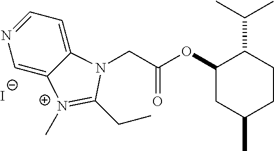 | −0.2218 | 485.41 | ++ | | | |
| 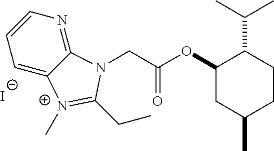 | −0.2218 | 485.41 | ++ | | | |
| 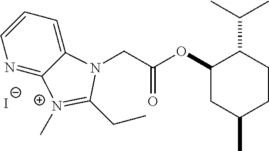 | −0.2218 | 485.41 | ++ | | | |
| 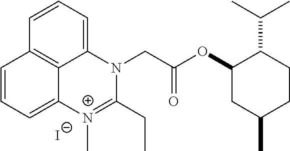 | 8.5864 | 533.47 | NA | | | |
| 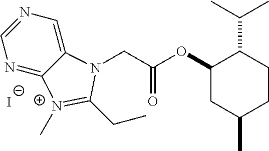 | −1.48301 | 486.4 | NA | | | |
| 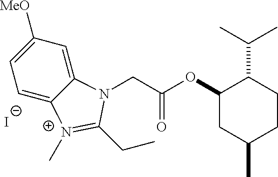 | 1.3766 | 514.45 | +++ | | | |
| 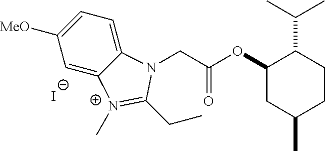 | 1.3766 | 514.45 | +++ | | | |
| 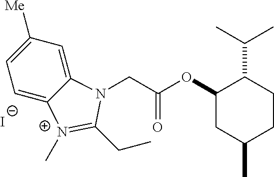 | 1.6028 | 498.45 | +++ | | | |
| 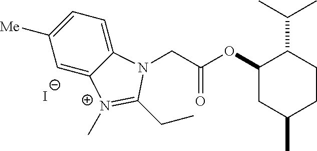 | | | | | | |
| 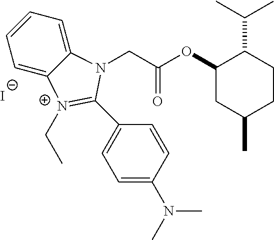 | 3.45819 | 589.62 | +++ | | | |
| 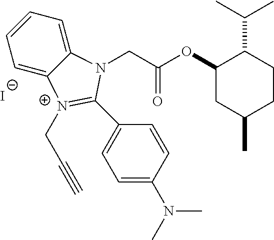 | 3.02919 | 599.557 | +++ | | | |
| 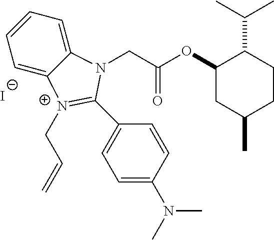 | 3.70319 | 601.573 | +++ | | | |
| 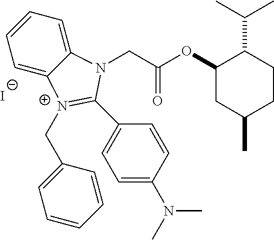 | 3.68719 | 651.633 | +++ | | | |
| 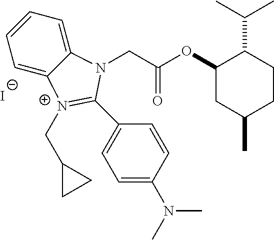 | 3.90219 | 615.6 | +++ | | | |
| 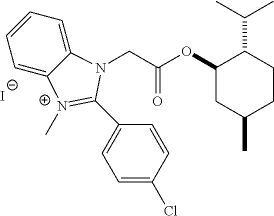 | 3.42072 | 566.908 | ++ | | | |
| 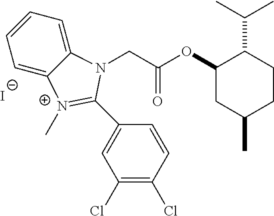 | 4.01509 | 601.35 | ++ | | | |
| 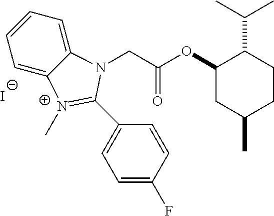 | 2.85072 | 550.457 | ++ | | | |
| 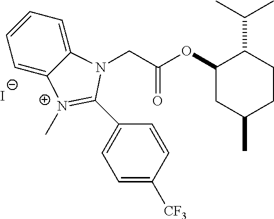 | 3.59366 | 600.465 | ++ | | | |
| 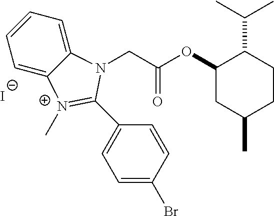 | 3.57072 | 611.362 | ++ | | | |
| 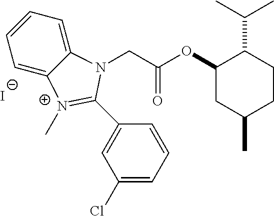 | 3.42072 | 566.908 | ++ | | | |
| 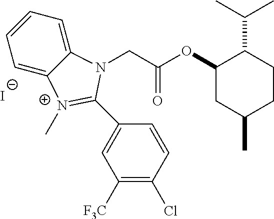 | 4.10803 | 634.907 | ++ | | | |
| 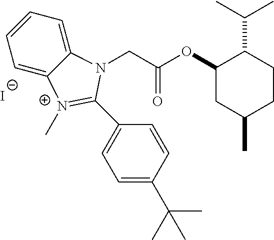 | 4.5298 | 588.574 | +++ | | | |
| 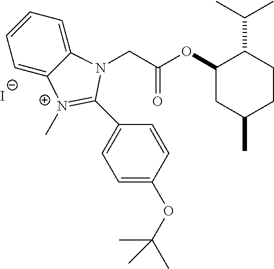 | 3.91168 | 604.573 | +++ | | | |
| 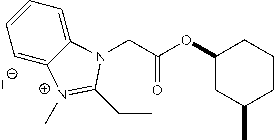 | −0.343201 | 442.3 | NA | | | |
| 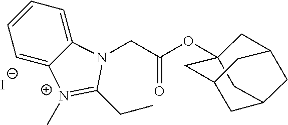 [SMILES: [I-] ; CCc1n(CC(=O)OC23CC4CC(CC(C4)C2)C3)c2ccccc2[n+]1C] | −0.2342 | 480.3 | + | | | |
| 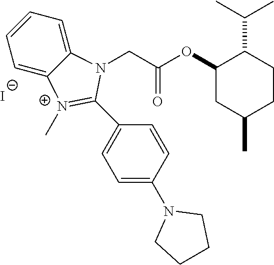 | 3.04319 | 601.5 | +++ | | | |
| 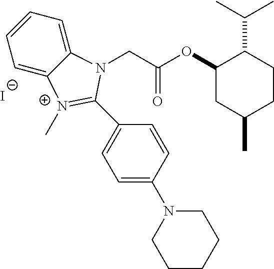 | 3.60219 | 615.6 | +++ | | | |

Glioblastoma multiforme (GBM) is one of the most aggressive and lethal forms of cancer. Most patients diagnosed with GBM die within 2 years of diagnosis and the prognosis has not significantly improved over decades (Chen, et al., 2012a). Standard treatment consists of surgical removal, followed by chemotherapy and radiotherapy. Temozolomide (TMZ), a DNA alkylating agent, is the most common used chemotherapy. However, TMZ treatment only increases patient survival by about two months (Chen, et al., 2012a). A 100%-penetrant, tumor-suppressor-based somatic GBM mouse models caused by tumor suppressor gene mutations frequently found in human GBM (p53, NF1, and Pten) has been generated (Cancer Genome Atlas Research, 2008). The mouse tumors replicate the human disease at the histopathologic and molecular levels (Llaguno, et al., 2009; Chen, et al., 2012b; Kwon, et al., 2008; Zhu, et al., 2005). Primary tissue culture conditions were further adapted that permit efficient culturing of primary tumor derived cells with stem-like properties (TD-Mut6 cells) from these models. Without wishing to be bound by any theory, this GBM models and GSCs is believed to have high relevance to human GBM and that our mouse tools afford a powerful means to probe many questions about GBM.

Under the rationale that these mouse models of GBM replicate the human tumors with fidelity, low passage primary cultures of TD-Mut6 cells were used, pooled from multiple tumors, to perform a high throughput small molecule screen. This has led to the identification of multiple interesting compounds with specific activity on GBM cells, including benzimidazolium compound 12M11. Initially, two hundred thousand compounds were tested in a carefully planned and validated ATP luminescence assay over a 96-hour exposure. Cherry-picked hits (˜5,000) were replicated in triplicate, and then counter screened against toxicity to primary mouse embryo fibroblasts (MEFs) and astrocytes. The use of healthy dividing primary cell cultures was designed to reduce the number of anti-mitotic or DNA repair compounds which would lack specificity for the malignant phenotype, and rather attack the replicative machinery in some non-specific manner. From the resulting compounds that showed specificity for the primary glioma tumor cells (Mut6) at nanomolar EC₅₀'s, a benzimidazolium-based compound (Cpd 12M11; FIG. 1) was selected for further evaluation. 12M11 showed specific toxicity to TD-Mut6 cells, but has no activity against normal astrocytes or MEFs (FIG. 1). Compound 12M11 is also active against certain, but not all, cancer cell lines. FIG. 3C shows data against a panel of primary human glioblastoma cell lines, whereas FIGS. 3A and 3B show results of compound 12M11 treatment against a panel of primary prostate cancer cells (FIG. 3B) and a selection of human cancer cell lines of varying tissue origin (FIG. 3A), demonstrating selectivity depending on the particular cell line.

12M11 causes irrevocable cell death on Mut6 cells after six hours of exposure. Microarray profiling at 6 hrs revealed a distinctive gene expression profile for Mut6 cells exposed to 12M11 (FIG. 3A) compared to MEFs (FIG. 3B) or astrocytes whose expression profiles were not affected, including several cell cycle arrest, pro-apoptotic and stress response genes. Glioma cell specifically induced genes included genes that are associated with glucose metabolism, ER and mitochondrial stress, including the targets of transcription factor ATF4 and genes involved in ROS (reactive oxygen species) regulation. Subsequent quantitative reverse transcription polymerase chain reaction (qRT-PCR) confirmation demonstrated deregulated mRNA levels in Mut6 cells but not in MEFs (FIG. 4). These include induction of ATF4 transcription factor mRNA, a gene whose transcription is induced under various forms of ER and mitochondrial stress and in turn activates a series of known target genes (Armstrong, et al., 2010; Blais, et al., 2004; Lange, et al., 2008; Milani, et al. 2009; Ye, et al., 2010); and repression of txnip, a known indicator of glucose metabolism (FIG. 4). FIG. 5 demonstrates activation of ATF4 protein in response to 12M11, which is accompanied by down regulation of Phospho-S6 only in Mut 6 cells (FIG. 5A) but not in MEFs (FIG. 5B) at 3 and 48 hr time points.

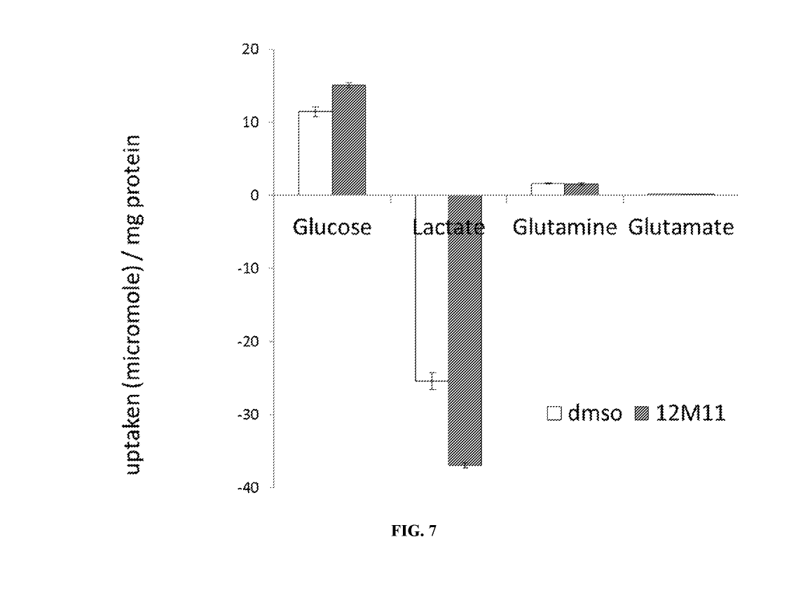

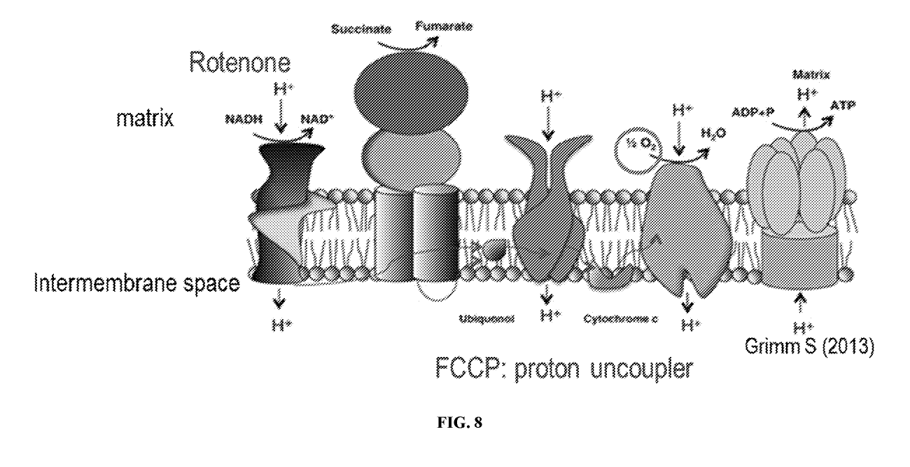

The preceding experiments indicating that 12M11 results in deregulated ROS, coupled with ATF4 induction and Phospho-S6 reduction (FIGS. 4 and 5), prompted investigation of the glucose metabolism in treated cells, which was deregulated as described below, and mitochondrial activity, which was significantly impaired (FIG. 6). Mut6 cells were incubated with 12M11 for 7 hrs, and the collected culture media was measured for glucose, lactate, glutamine, and glutamate (Birsoy, et al., 2014; Lunt and Vander Heiden, 2011). These results indicate increased uptake of glucose and increased lactate secretion in these cells (FIG. 7). Oxygen consumption and extracellular acidification using a XF24 Seahorse analyzer was used to measure cellular oxygen consumption rate (OCR) and extracellular acidification rate (ECAR) (FIG. 8). These data indicate that 12M11 has rapid effects on oxygen consumption on both MEFs and Mut6 cells (FIGS. 9A & 9B), however, the Mut6 cells have dramatically increased extracellular acidification levels indicative of Lactate secretion whereas the MEFs do not increase acidification significantly (FIGS. 9C & 9D). These data indicate a differential effect on glucose metabolism and oxidative phosphorylation by 12M11 on TD-Mut6 cells.

Glucose deprivation of TD-Mut6 cells was found to have many parallel effects to those caused by 12M11. Glucose deprivation induces cell cycle arrest and apoptotic cell death (FIG. 10A) in Mut6 cells with similar kinetics as described for 12M11. Many of the same ATF4 response genes are also activated (FIG. 10B) concomitant with elevation of ATF4 protein and suppression of Phospho-S6 (FIG. 10C). These data provide additional support for the idea that the primary GBM cells (Mut6) have unique sensitivities to perturbations in metabolism and energy production and consumption. Without wishing to be bound by any theory, it is believed that the benzimidazolium compound 12M11 targets oxidative phosphorylation in a manner that TD-Mut6 cells cannot overcome, leading to activation of cellular stress response mechanisms and eventually to apoptotic cell death. These latter events may be mediated directly by ATF4 and the mTOR pathways.

To get some insight as to why Mut6 cells are potentially more sensitive to Oxydative Phosphorylation inhibitors, OCR of Mut6 and MEF were measured (see FIG. 11A, copied from FIGS. 8A & 8B dark gray line), basal ATP levels (FIG. 11B; CellTiter-Glo® assay from Promega), and cellular OxyPhos activity as measured by TMRE-staining (FIG. 11C). As shown in FIG. 11A, Mut6 and MEF cells have identical basal oxygen consumption rates (see blue and red curves before olgomycin addition). However, whereas Mut6 cells return to the same basal OCR after FCCP (proton uncoupler) treatment, MEF cells have additional OCR capacity (see squares curve after FCCP treatment. Interestingly, the basal ATP levels of Mut6 cells are lower than for MEF cells, despite having similar/identical basal OCR) (FIG. 11B). Therefore, it appears that Mut 6 cells use their full capacity of mitochondrial oxidative phosphorylation, whereas MEF cells have additional capacity. The higher mitochondrial membrane potential of Mut6 versus MEF cells as measured by TMRE staining supports the notion that Mut 6 have higher oxyphos activity than MEF (FIG. 11C). Without wishing to be bound by any theory, it is believed that the increased OCR-capacity (from basal) of MEF cells is responsible for their resistance to OxyPhos inhibitors. FIG. 12A demonstrates that Mut6 cells are more sensitive than MEF cells to OxyPhos inhibitors (CellTiter-Glo® cell viability assay from Promega), whereas FIG. 12B shows that the ATP levels (% change from basal) of Mut6 cells decrease significantly in a time-dependent manner upon treatment with the OxyPhos inhibitors oligomycin, rotenone, or 12M11, whereas corresponding ATP levels increase for MEF cells upon similar OxyPhos-inhibitor treatment.

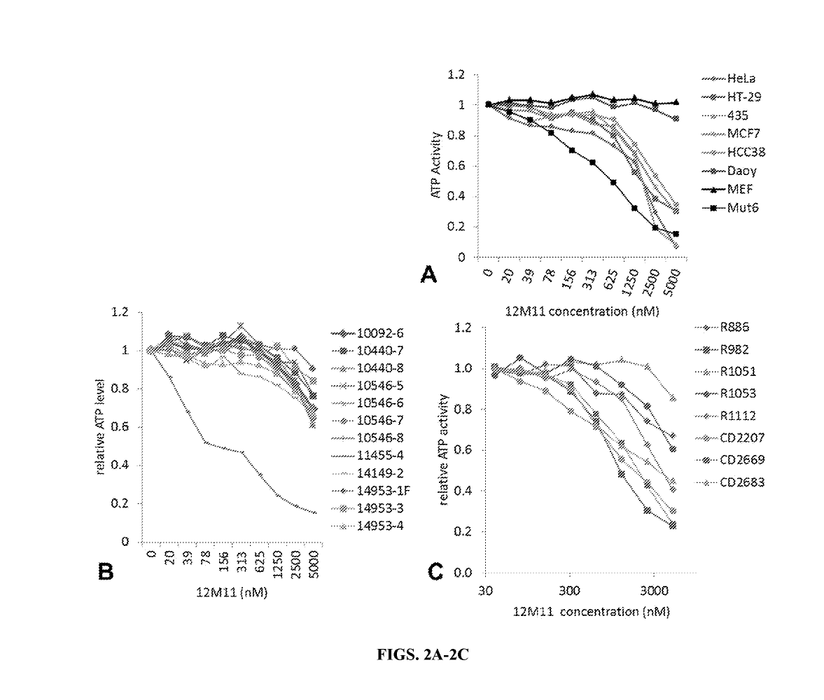

The activity of compound 12M11 was tested against a variety of cancer cell lines (see FIG. 2A), including those are routinely used in the literature such as HeLa, DAOY, MCF7, 435 and HCC38 cells. As indicated in FIG. 13A, 12M11 retains cytotoxic activity on a significant proportion of these cells tested—excluding DAOY (medulloblastoma) and MEF cells—with EC₅₀ in the mid nanomolar to low micromolar range (CellTiter-Glo® cell viability assay from Promega). Just as for MEF cells, without wishing to be bound by any theory, it is believed that the resistance of DAOY cells to the OxyPhos inhibitor 12M11 can be attributed to their higher ATP levels per cell (FIG. 13B) combined with lower intrinsic OxyPhos activity as assessed by TMRE staining (FIG. 13C).

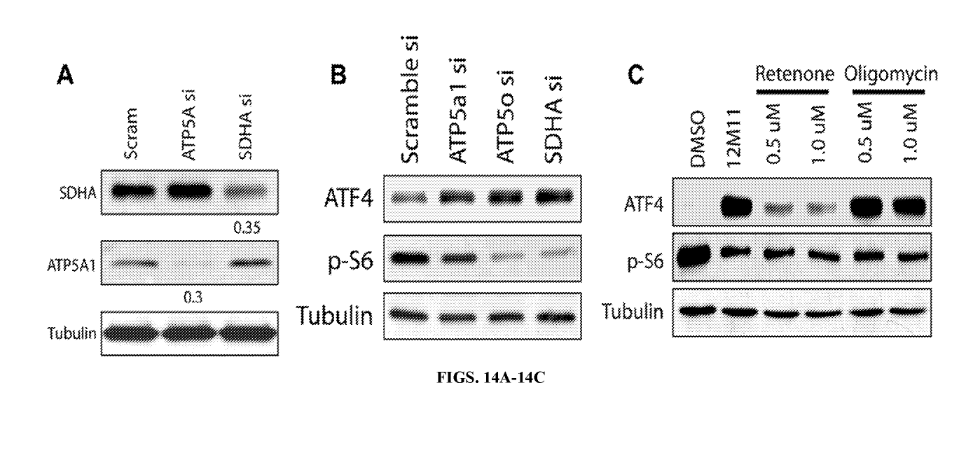

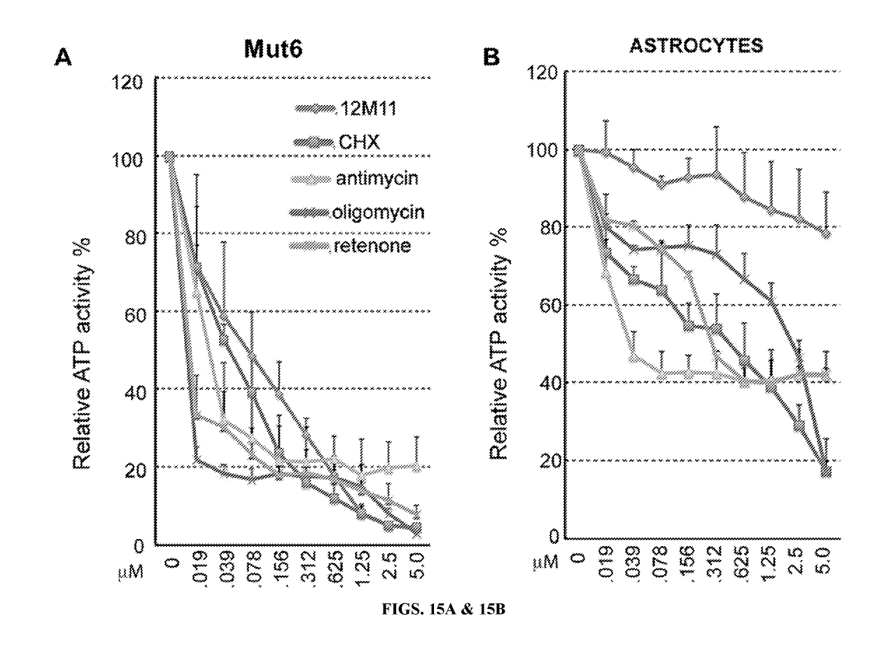

Many of the lines of experimentation described above point toward energy consumption, glucose metabolism as rate limiting steps of cell growth and survival in the presence of compound 12M11. In particular, mitochondrial activity (FIG. 2) and the Oxidative Phosphorylation chain (FIG. 9) appear to be affected and suppressed. To more directly examine the status of oxidative phosphorylation in TD-Mut6 cells, siRNAs against proteins (ATP5a1, ATP5o or SDHA) that are part of the OxPhos complex were validated (FIG. 14A) and demonstrated that knockdown of such proteins in TD-Mut6 glioma cells results in activation of ATF4 and suppression of Phospho-S6 (FIG. 14B). Well-established general inhibitors of the OxPhos pathway (Rotenone and Oligomycin) were tested and found that exposure of Mut6 cells to rotenone and oligomycin also results in induction of ATF4 and suppression of Phospho-S6 (FIG. 14C). The cell growth and metabolism activity of 12M11, cycloheximide (a protein synthesis inhibitor), and OxPhos inhibitors (antimycin, oligomycin and rotenone) on Mut6 were compared to primary astrocytes. FIG. 15 shows that whereas both Mut 6 cells and astrocytes are sensitive to a general protein synthesis inhibitor (cycloheximide) and known OxyPhos inhibitors (rotenone, antimycin, oligomycin), astrocytes are uniquely resistant to OxyPhos inhibitor 12M11 and remain sensitive to protein synthesis inhibition (cycloheximide) and general OxPhos inhibitors (rotenone, antimycin, oligomycin). Therefore, 12M11 appears to behave differently than other known OxyPhos inhibitors (rotenone, oligomycin, antimycin) inasmuch that it has unique selectivity towards Mut6 cell growth inhibition versus MEF and astrocytes (see also FIG. 1). Taken together, these data point to a convergence on Oxidative Phosphorylation, upstream of ATF4 and associated cellular stress responses as the targets for 12M11, but in a manner distinct from other known OxyPhos inhibitors such as rotenone, oligomycin, or antimycin.

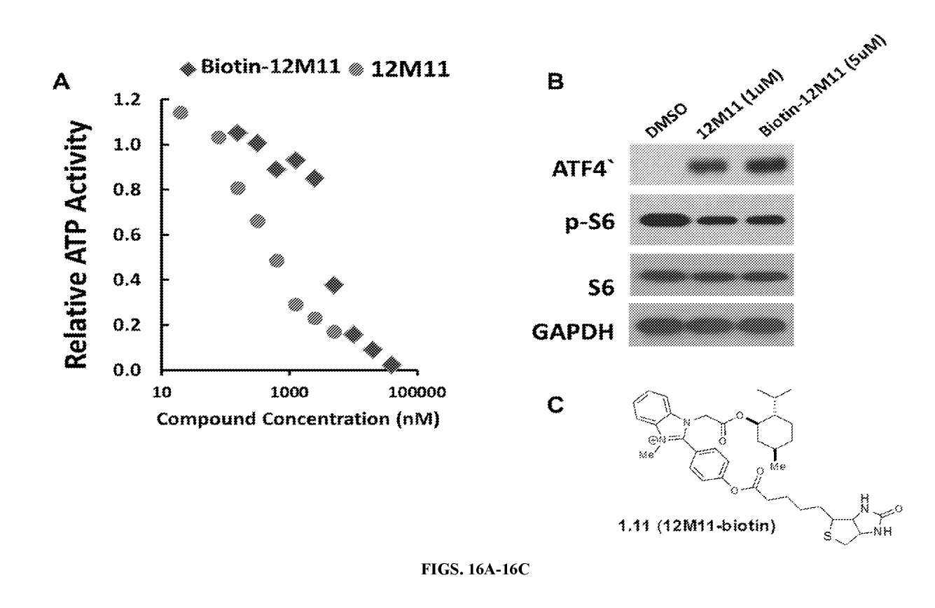

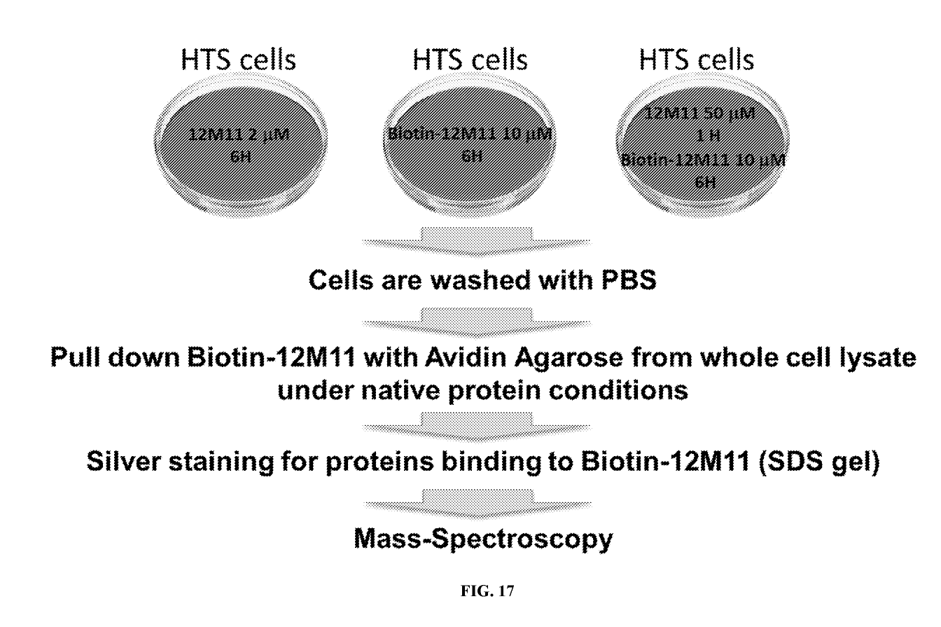

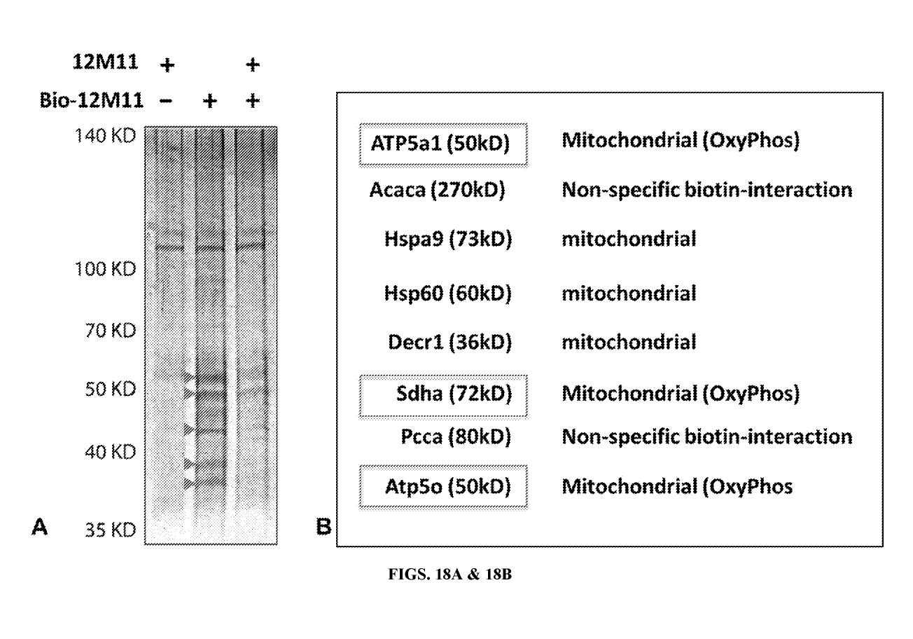

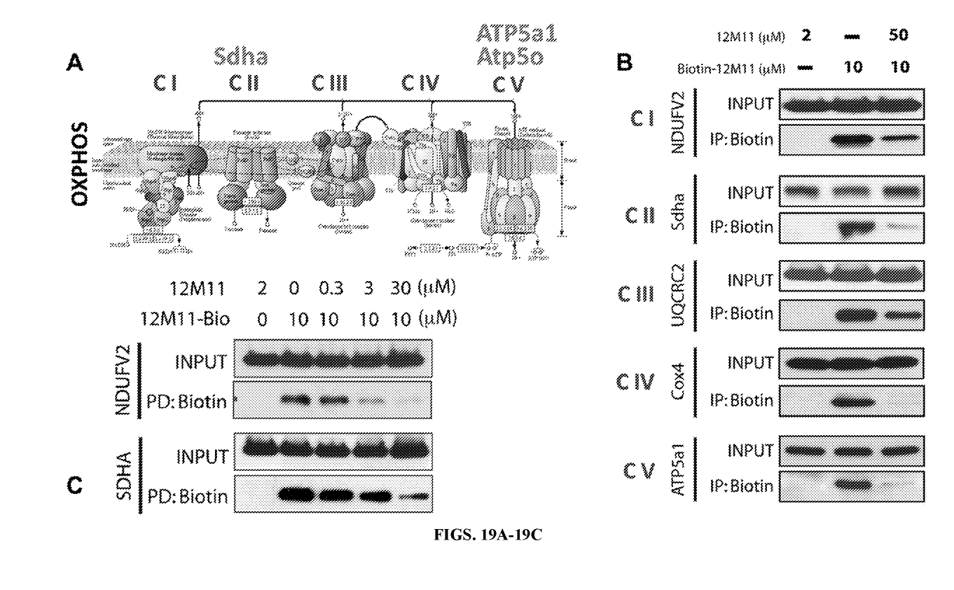

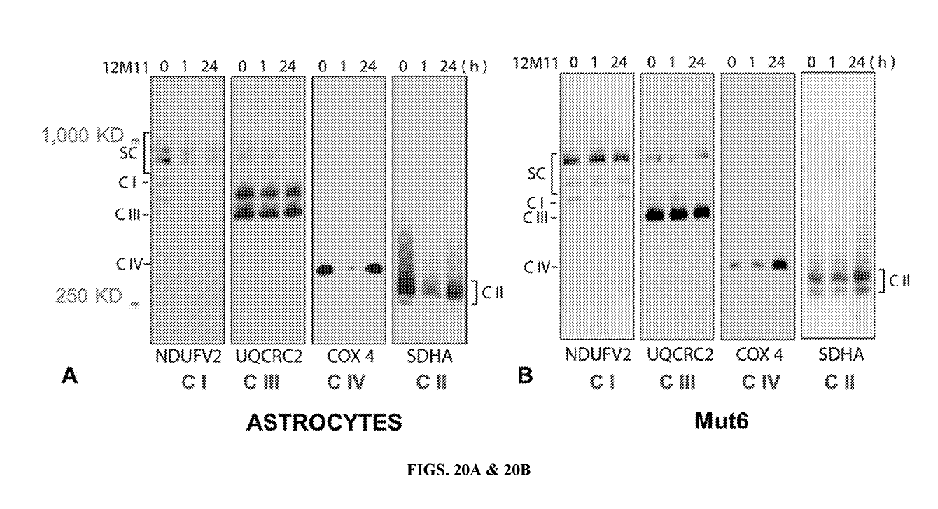

To understand the unique selectivity of 12M11 to affect the viability of Mut6 cells, the precise molecular target(s) of 12M11 were determined. A 12M11 biotinylated variant (see Cpd 1.11, FIG. 16C) was generated to attempt to directly pull down interacting proteins. Compound 1.11 (hereafter called Biot-12M11) is biologically active in arresting Mut6 cell growth (FIG. 16A), inducing ATF4, and suppressing Phospho-S6 (FIG. 16B). Like 12M11, the biotinylated variant (Biot-12M11) has no growth suppression activity on primary MEFs or astrocytes. Mut6 cells were treated with 12M11, Biot-12M11, or pre-incubated with 12M11 followed by addition of Biot-12M11 (see FIG. 17 for a general schematic protocol). As indicated in the silver stain of a denaturing gel (FIG. 18A) with the pull down reactions, several bands that appear in the Biot-12M11 tract (Lane 2) are substantially diluted or absent in Lane 3 where the cells were pre-incubated with excess 12M11. Based on these results, samples of the pull down reactions for each of the conditions were submitted for Mass Spectroscopy sequencing. The results from Mass-Spec analysis were as follows: 1) Lysate from Mut6 cells incubated with 12M11 alone yielded no proteins; 2) Lysate from Mut6 cells incubated with Biot-12M11 yielded 8 proteins (Atp5a1, Acaca, Hspa9, Hsp60, Decr1, Sdha, Pcca, and Atp5o; FIG. 19B). Two of the proteins, Acac and Pcca, were discounted as known to nonspecifically bind Biotin (Tong, 2005; Diacovich, et al., 2004; Kalousek, et al., 1980). The remaining 6 proteins all localize to the mitochondrion and 3 of the pulled down proteins are components of the oxidative phosphorylation complexes. Taking into consideration the reduced mitochondrial activity (FIG. 6) and the altered glucose metabolism and requirements (FIGS. 7-11) of TD-Mut6 cells, and that several mitochondrial proteins were specifically pulled down with Biot-12M11 (FIGS. 17 and 18), with wishing to be bound by any theory, it is believed that compound 12M11 may directly or indirectly interact with some component of the electron transport oxidative phosphorylation machinery (FIG. 20A). To test this pull down experiments were performed with lysates from Mut6 cells that had been preincubated with either 12M11, Biot-12M11, or with excess 12M11 (for 1 hr) followed by Biot-12M11 (see FIG. 17 for protocol) and run on an SDS gel (FIG. 19B). The gel was subjected to Western blot analysis and probed with well-characterized antibodies to proteins contained in each one of the oxidative phosphorylation complexes I-V (see FIG. 19A for a diagram of OxyPhos complexes I-V). The results in FIG. 19B indicate that Biot-12M11 specifically binds to, and results in pull down of, the entire oxidative phosphorylation machinery as evidenced by the western blot presence of proteins from each of the complexes I through V. These data however, do not distinguish between direct and indirect binding of 12M11 to component(s) of the OxPhos complexes. FIG. 19C further demonstrates that incubation of Mut6 cells with 12M11 and Biot-12M11 at the same time results in a progressive dose-response dilution of the pull down of the Complex I and Complex II component proteins, NDUFV2 and SDHA, respectively. Collectively, the above data corroborate the hypothesis that compound 12M11 may exert its toxic effects on Mut6 cells by binding to a critical component of the oxidative phosphorylation machinery.

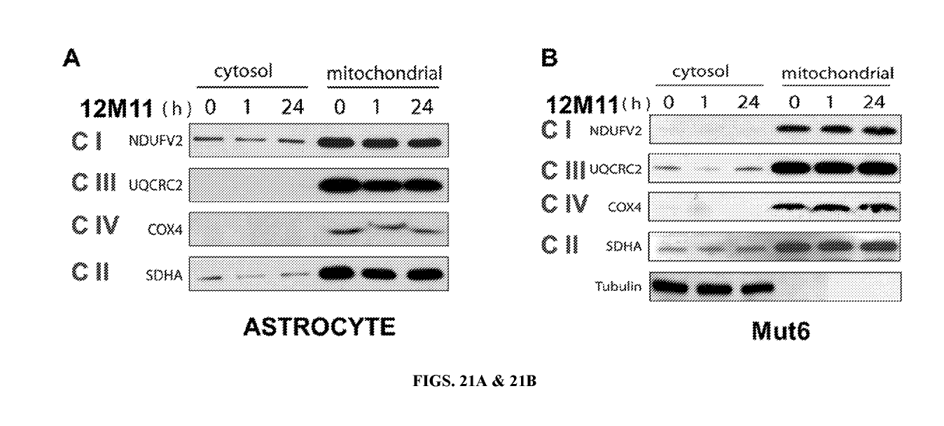

The intact OxPhos complexes was examined by running native gels of mitochondrial extracts from Mut6 cells and primary astrocytes that had been pre-incubated for one hour or 24 hours with 12M11 and compared to untreated controls (FIG. 20A & 20B). As a general observation, it appears that astrocytes and Mut6 cells incorporate complex I protein NDUFV2 and complex III protein UQCRC2 in different amounts and in complexes of different size. Notably, astrocytes form two complexes incorporating more or less equal amounts of complex III protein UQCRC2 (FIG. 20A, two major bands, second set of three lanes), whereas Mut6 cells only form one of the two complexes (FIG. 20B, one major band, second set of three lanes). Astrocytes treated with 12M11 showed a transient and substantial reduction of complexes containing Complex I, II and IV proteins (but not complex III) at the one hour time point but complexes II and IV regained normal levels by 24 hours (FIG. 20A). In contrast, the amount and nature of complexes in 12M11 treated Mut6 cells did not change significantly at the 1-hour time-period, but apparently accumulated complexes containing Complex II and IV proteins over 24 hours (FIG. 21B). As shown in FIG. 21A & 21B, when total levels of complex I-V proteins were examined from cells treated with 12M11 (as assessed by running denaturing SDS gels), neither cell type appeared to have changes in overall protein levels over the 24 hour period, or from the untreated controls (time 0 hour). Thus wild type 12M11-treated astrocytes (resistant to 12M11), apparently disassemble components of the OxPhos complex transiently (1 hour time-period) and then reassemble them into functional components (24 hour time-period) as evidenced by their continued ability of generate ATP and survive. In contrast, the sensitive Mut6 glioma cells maintain the OxPhos structures intact in the face of 12M11 and even accumulate them overtime despite their relative nonfunctional state.

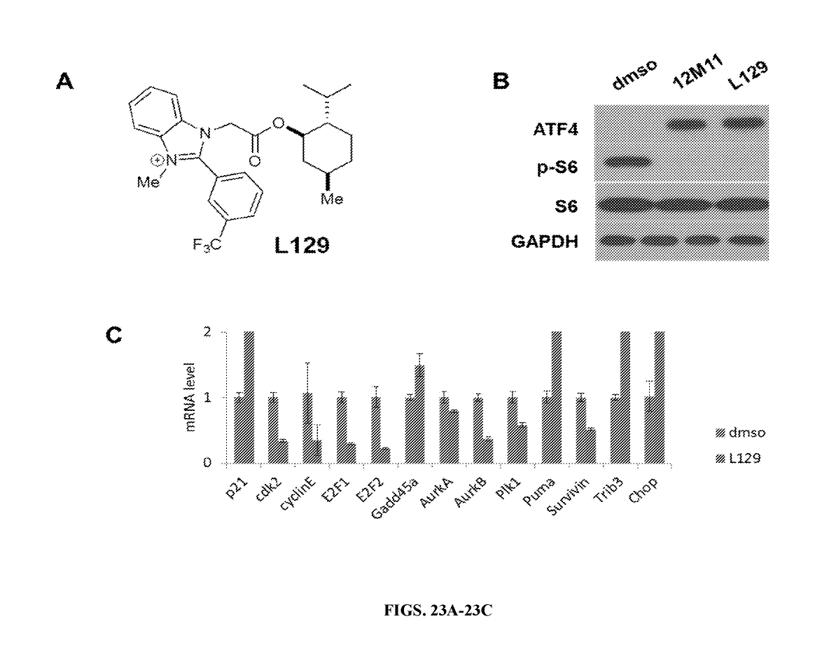

Although 12M11 possessed acceptable potency (˜100 nM, see FIG. 1), excellent in vitro metabolic stability (S9 T_{1/2}=217 min, FIG. 22A; hepatocyte T_{1/2}=257 min), it had a short in vitro plasma half-life (4 min, FIG. 22C) and low in vivo Cₘₐₓ (2.7 ng/mL, FIG. 22B). An equipotent analog termed L129 (FIG. 23A) retained excellent in vitro S9 metabolic stability (T½>240 min, FIG. 23D), but has much improved in vitro plasma stability (T_{1/2}=306 min, FIG. 22F), and better plasma PK (FIG. 22E) and tumor PK (T_{1/2}=909 min; Cₘₐₓ 375 ng/g) when dosed at 10 mg/kg IP. The data indicate that Mut6 cell-derived allograft tumors in the flanks of nude mice treated daily for 3 weeks 12M11-analog, L129 (FIG. 23A), exhibit tumor growth retardation compared to DMSO treated mice. These results confirm the stability of the compound and its relative safety in mice as they exhibited no overt adverse effects and histopathological examination of major organs following 3 week treatment did not reveal evidence of necrosis, cell death, fibrosis, or other signs of organ toxicity.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Armstrong, et al. , J. Biol. Chem., 285(9):6091-6100.
Blais, et al., Mol. Cell. Biol., 24(17):7469-7482, 2004.
Bisroy, et al., Nature, 508(7494):108-112, 2014.
Cancer Genome Atlas Research, Nature, 455(7216):1061-1068.
Chen, et al., Cell, 149(1):36-47, 2012a.
Chen, et al., Nature, 488(7412):522-526, 2012b.
Diacovich, et al., Biochemistry, 43(44):14027-14036, 2004.
Handbook of Pharmaceutical Salts: Properties, and Use, Stahl and Wermuth Eds.), Verlag Helvetica Chimica Acta, 2002.
Kalousek, et al., J. Biol. Chem., 255(1):60-65, 1980.
Kwon, et al., Cancer Res., 68(9):3286-3294, 2008.
Lange, et al., J. Exp. Med., 205(5):1227-1242, 2008.
Llanguno, et al., Cancer Cell, 15(1):45-56, 2009.
Lust and Vander Heiden, Annual Rev. of Cell & Developmental Biology, 27:441-464, 2011.
March's Advanced Organic Chemistry: Reactions, Mechanisms, and Structure, 2007.
Milani, et al., Cancer Res., 69(10):4415-4423, 2009.
Tong, CMLS, 62(16):1784-1803, 2005.
Ye, et al., The EMBO Journal, 29(12):2082-2096, 2010.
Zhu, et al., Cancer Cell, 8(2):119-130, 2005.

1. A compound of the formula: 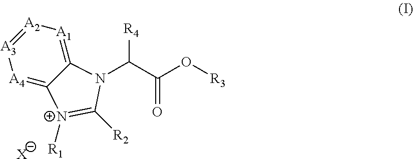
wherein:
R₁ is alkyl_{(C≤12)}, cycloalkyl_{(C≤12)}, alkenyl_{(C≤12)}, alkynyl_{(C≤12)}, aralkyl_{(C≤12)}, heteroaralkyl_{(C≤12)}, -alkanediyl_{(C≤6)}-cycloalkyl_{(C≤12)}, or a substituted version of any of these groups;
R₂ is alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, aralkenyl_{(C≤12)}, heteroaryl_{(C≤12)}, heteroaralkyl_{(C≤12)}, heteroaralkenyl_{(C≤12)}, heterocycloalkyl_{(C≤12)}, or a substituted version of any of these groups; or a group of the formula: 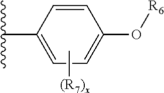
wherein:
R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula: 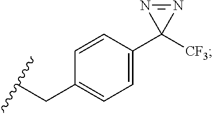 [SMILES: *Cc1ccc(C2(C(F)(F)F)N=N2)cc1]
R₇ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl _{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
x is 0, 1, 2, or 3; or
a group of the formula: 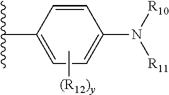
wherein:
R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
R₁₂ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl _{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
y is 0, 1, 2, or 3;
R₃ is alkyl_{(C≤12)}, cycloalkyl_{(C≤12)}, bicycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, or a substituted version of any of these groups;
R₄ is hydrogen or alkyl_{(C≤12)}, cycloalkyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, -alkanediyl_{(C≤6)}-heterocycloalkyl_{(C≤12)} or a substituted version of any of these groups;
A₁, A₂, A₃, and A₄ are independently selected from the group CH, N, or CR₅, wherein:
R₅ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)}; and
X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, nitrate, or another pharmaceutically acceptable salt;
provided that when R₁ is methyl, R₂ is ethyl, A₁, A₂, A₃, and A₄ are CH, and R₄ is hydrogen then R₃ is not menthol or methyl;
or a stereoisomer thereof.

2. The compound of claim 1, wherein the compound is further defined as: 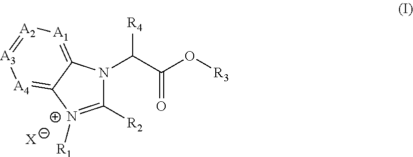
wherein:
R₁ is alkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, -alkanediyl_{(C≤4)}-cycloalkyl_{(C≤6)}, or a substituted version of any of these groups;
R₂ is alkyl_{(C≤12)}, cycloalkyl_{(C≤12)}, alkynyl_{(C≤12)}, aryl_{(C≤12)}, aralkyl_{(C≤12)}, heteroaryl_{(C≤12)}, heteroararyl_{(C≤12)}, heterocycloalkyl_{(C≤12)}, or a substituted version of any of these groups; or a group of the formula: 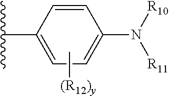
wherein:
R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
R₁₂ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤8)}, alkenylthio_{(C≤8)}, alkynylthio_{(C≤8)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)}; and
y is 0, 1, 2, or 3;
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aryl_{(C≤12)}, aralkyl_{(C≤12)}, aryl_{(C≤12)}, aralkyl_{(C≤12)}, heteroaryl_{(C≤12)}, heterocycloalkyl_{(C≤12)}, -alkanediyl_{(C≤6)}-heterocycloalkyl_{(C≤12)}, or a substituted version of any of these groups;
A₁, A₂, A₃, and A₄ are independently selected from the group CH, N, or CR₅,
wherein:
R₅ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)}; and
X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, or nitrate;
provided that when R₁ is methyl, R₂ is ethyl, A₁, A₂, A₃, and A₄ are CH, and R₄ is hydrogen then R₃ is not menthol or methyl; or
a stereoisomer thereof.

3. The compound of claim 1, wherein the compound is further defined as: 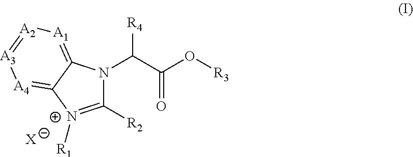
wherein:
R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
R₂ is alkyl_{(C≤12)}, cycloalkyl_{(C≤12)}, alkynyl_{(C≤12)}, aryl_{(C≤12)}, aralkyl_{(C≤12)}, heteroaryl_{(C≤12)}, heteroararyl_{(C≤12)}, heterocycloalkyl_{(C≤12)}, or a substituted version of any of these groups; or a group of the formula: 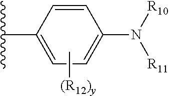
wherein:
R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
R₁₂ is azido, carboxy, cyano, halo, nitro, or
alkyl_{(C≤8)}, acyl_{(C≤8)}, alkoxy_{(C≤8)}, alkylthio_{(C≤12)}, or a substituted version of any of these groups; or
y is 0, 1, 2, or 3;
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aralkyl_{(C≤12)}, heteroaralkyl_{(C≤12)}, or a substituted version of any of these groups;
A₁, A₂, A₃, and A₄ are independently selected from the group CH, N, or CR₅,
wherein:
R₅ is azido, cyano, halo, nitro, or
alkyl_{(C≤8)}, alkoxy_{(C≤8)}, alkylthio_{(C≤12)}, or a substituted version of any of these groups;
X is halide, hydroxide, bicarbonate, biphosphate, acetate, formate, citrate, tosylate, mesylate, camphorsulfonate, benzenesulfonate, picrate, nitrate, or a pharmaceutically acceptable salt;
or a stereoisomer thereof.

4. The compound according to claim 1, wherein the compound is further defined as: 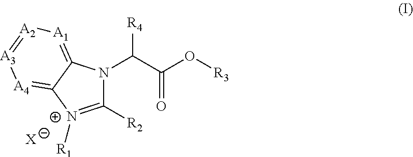
wherein:
R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
R₂ is aryl_{(C≤12)}, heteroaryl_{(C≤12)}, or a substituted version of either of these groups wherein the substitution is:
amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aralkyl_{(C≤12)}, heteroaralkyl_{(C≤12)}, or a substituted version of any of these groups;
A₁, A₂, A₃, and A₄ are independently selected from the group CH, N, or CR₅,
wherein:
R₅ is azido, cyano, halo, nitro, or
alkyl_{(C≤8)}, alkoxy_{(C≤8)}, alkylthio_{(C≤12)}, or a substituted version of any of these groups;
X is halide, hydroxide, bicarbonate, biphosphate, acetate, formate, citrate, tosylate, mesylate, camphorsulfonate, benzenesulfonate, picrate, nitrate, or a pharmaceutically acceptable salt; or
a stereoisomer thereof.

5. The compound of claim 1, wherein the compound is further defined as: 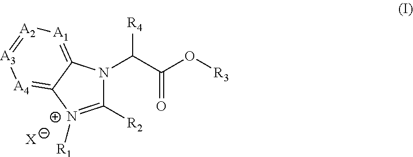
wherein:
R₁ is alkyl_{(C≤6)}, haloalkyl_{(C≤6)}, cycloalkyl_{(C≤6)}, alkenyl_{(C≤6)}, alkynyl_{(C≤6)}, aralkyl_{(C≤8)}, heteroaralkyl_{(C≤8)}, or -alkanediyl_{(C≤4)}-cycloalkyl_{(C≤6)};
R₂ is aryl_{(C≤12)}, heteroaryl_{(C≤12)}, or a substituted version of either of these groups wherein the substitution is azido, cyano, or halo; or alkyl_{(C≤8)}, cycloalkyl_{(C≤8)}, heterocycloalkyl_{(C≤8)}, alkoxy_{(C≤8)}, alkenyloxy_{(C≤8)}, alkynyloxy_{(C≤8)}, alkylthio_{(C≤8)}, alkenylthio_{(C≤8)}, alkynylthio_{(C≤8)}, alkylamino_{(C≤8)}, dialkylamino_{(C≤8)}, cycloalkylamino_{(C≤8)}, dicycloalkylamino_{(C≤8)}, or a substituted version of any of these groups;
R₃ is cycloalkyl_{(C≤12)}, fused cycloalkyl_{(C≤12)}, or a substituted version of any of either of these groups;
R₄ is hydrogen, alkyl_{(C≤12)}, cycloalkyl_{(C≤12)}, aralkyl_{(C≤12)}, heteroaralkyl_{(C≤12)}, or a substituted version of any of these groups;
A₁, A₂, A₃, and A₄ are independently selected from the group CH, N, or CR₅,
wherein:
R₅ is azido, cyano, halo, nitro, or
alkyl_{(C≤8)}, alkoxy_{(C≤8)}, alkylthio_{(C≤12)}, or a substituted version of any of these groups;
X is halide, hydroxide, bicarbonate, biphosphate, acetate, formate, citrate, tosylate, mesylate, camphorsulfonate, benzenesulfonate, picrate, nitrate, or a pharmaceutically acceptable salt; or
a stereoisomer thereof.

6. The compound according to claim 1, wherein R₁ is alkyl_{(C≤12)} or substituted alkyl_{(C≤12)}.

7-10. (canceled)

11. The compound according to claim 1, wherein R₁ is -alkanediyl_{(C≤4)}-cycloalkyl_{(C≤6)} or substituted -alkanediyl_{(C≤4)}-cycloalkyl_{(C≤6)}.

12-13. (canceled)

14. The compound according to claim 1, wherein R₁ is alkenyl_{(C≤12)}.

15. (canceled)

16. The compound according to claim 1, wherein R₁ is alkynyl_{(C≤12)}.

17. (canceled)

18. The compound according to claim 1, wherein R₁ is aralkyl_{(C≤12)}.

19. (canceled)

20. The compound according to claim 1, wherein R₂ is alkyl_{(C≤12)}.

21. (canceled)

22. The compound according to claim 1, wherein R₂ is alkenyl_{(C≤12)}.

23. (canceled)

24. The compound according to claim 2, wherein R₂ is aryl_{(C≤12)} or substituted aryl_{(C≤12)}.

25. (canceled)

26. The compound according to claim 1, wherein R₂ is aralkenyl_{(C≤12)}.

27. (canceled)

28. The compound according to claim 2, wherein R₂ is heteroaryl_{(C≤12)}.

29. (canceled)

30. The compound according to claim 1, wherein R₂ is: 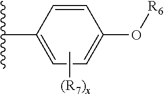
wherein:
R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula: 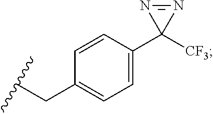 [SMILES: *Cc1ccc(C2(C(F)(F)F)N=N2)cc1]
R₇ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups; or
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
x is 0, 1, 2, or 3.

31-34. (canceled)

35. The compound according to claim 1, wherein R₂ is: 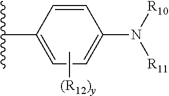
wherein:
R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
R₁₂ is azido, carboxy, cyano, halo, nitro, or
acyl_{(C≤8)}, alkoxy_{(C≤8)}, alkylthio_{(C≤12)}, or a substituted version of any of these groups;
y is 0, 1, 2, or 3.

36-51. (canceled)

52. The compound according to claim 1, wherein R₃ is cycloalkyl_{(C≤12)} or substituted cycloalkyl_{(C≤12)}.

53-61. (canceled)

62. The compound according to claim 1, wherein R₃ is aralkyl_{(C≤12)} or substituted aralkyl_{(C≤12)}.

63. (canceled)

64. The compound according to claim 1, wherein R₄ is hydrogen.

65. The compound according to claim 1, wherein R₄ is alkyl_{(C≤12)} or substituted alkyl_{(C≤12)}.

66. (canceled)

67. The compound according to claim 1, wherein R₄ is cycloalkyl_{(C≤12)} or substituted cycloalkyl_{(C≤12)}.

68. (canceled)

69. The compound according to claim 2, wherein R₄ is aralkyl_{(C≤12)} or substituted aralkyl_{(C≤12)}.

70-80. (canceled)

81. The compound according to claim 1, wherein R₅ is azido, cyano, halo, nitro, or alkyl_{(C≤6)}, alkoxy_{(C≤6)}, alkylthio_{(C≤6)}, or a substituted version of any of these groups.

82-87. (canceled)

88. The compound according to claim 1, wherein X is halide, hydroxide, bicarbonate, biphosphate, carboxylate, alkylsulfonate_{(C≤12)}, cycloalkylsulfonate_{(C≤12)}, arylsulfonate_{(C≤12)}, picrate, nitrate, or another pharmaceutically acceptable counter-ion.

89-91. (canceled)

92. The compound according to claim 1, wherein the compound is further defined as: 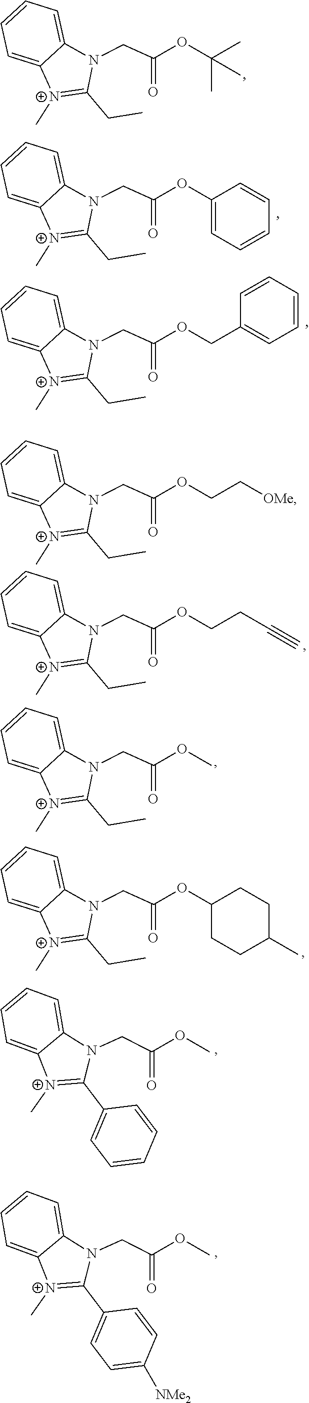 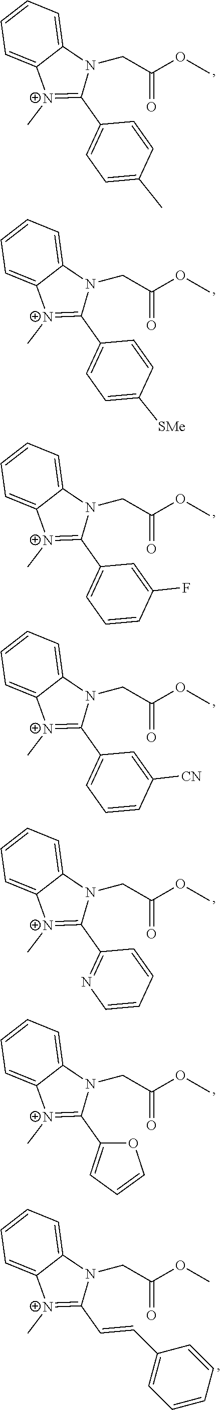 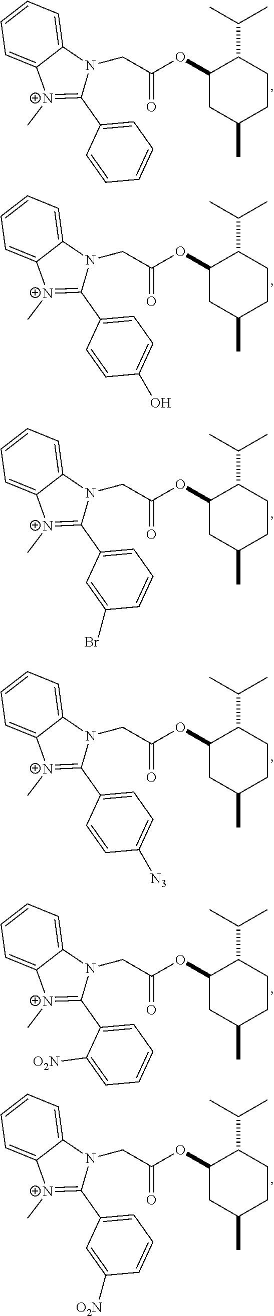 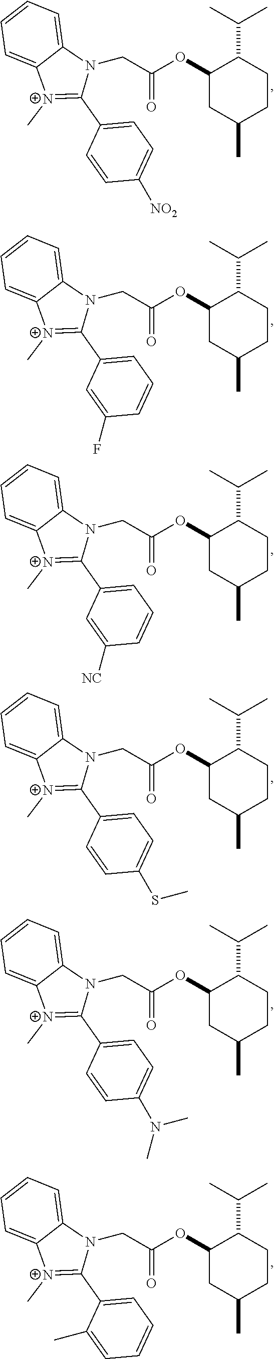 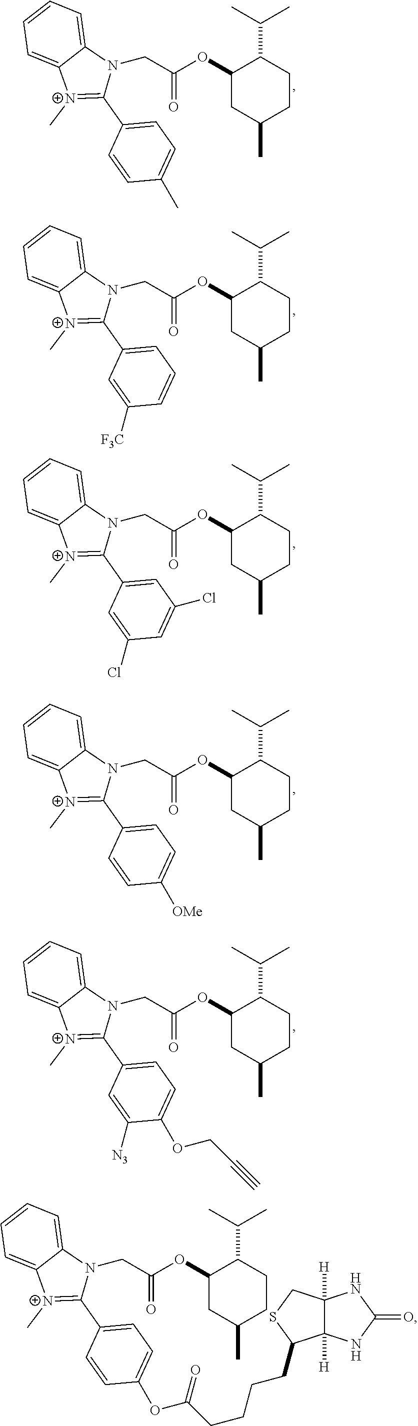 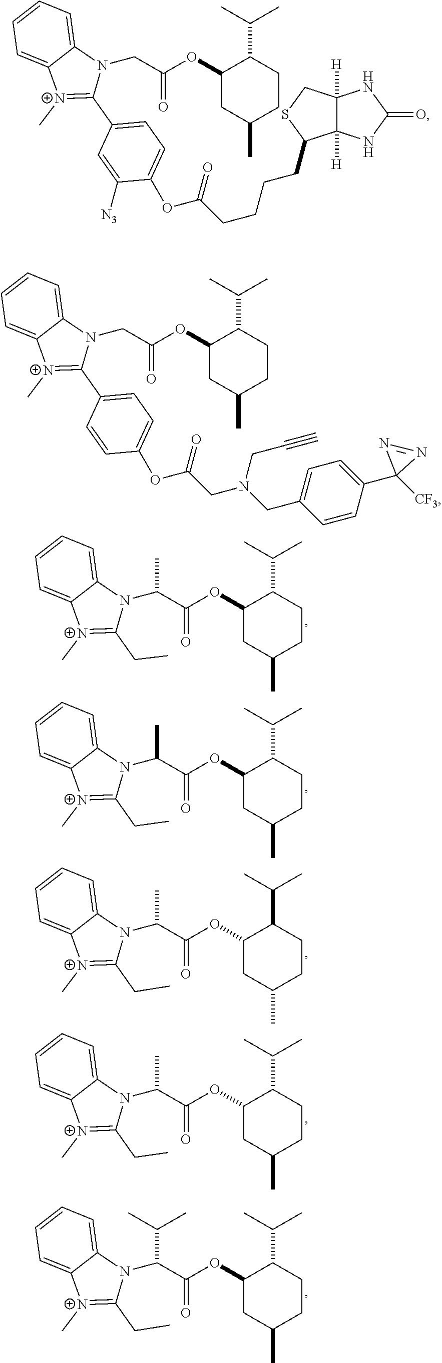 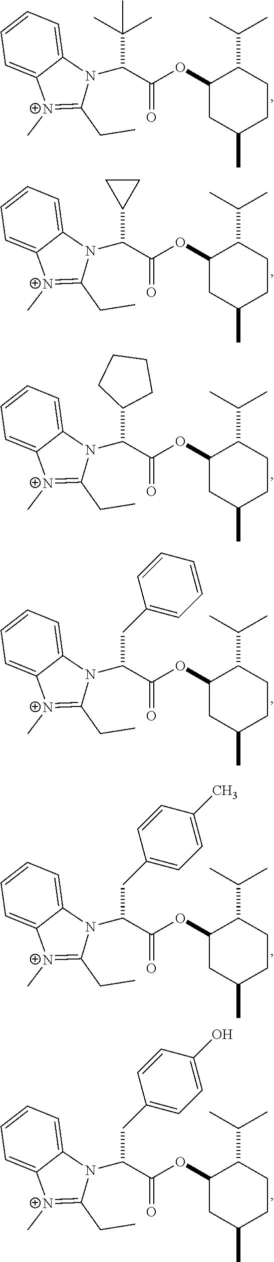 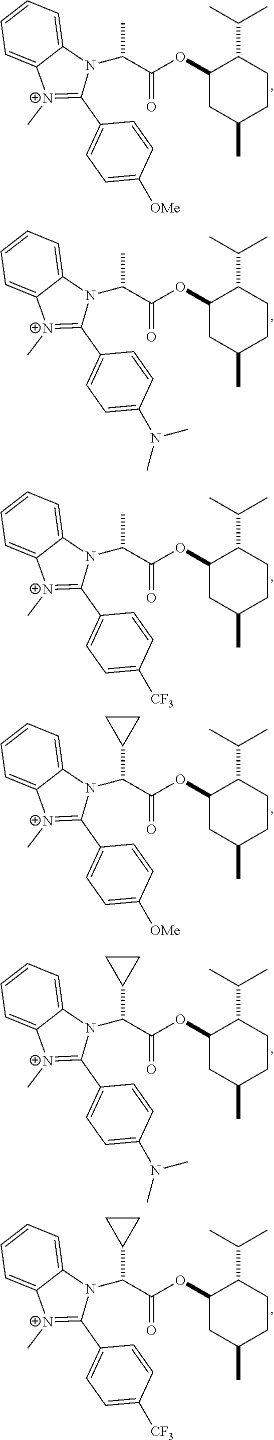 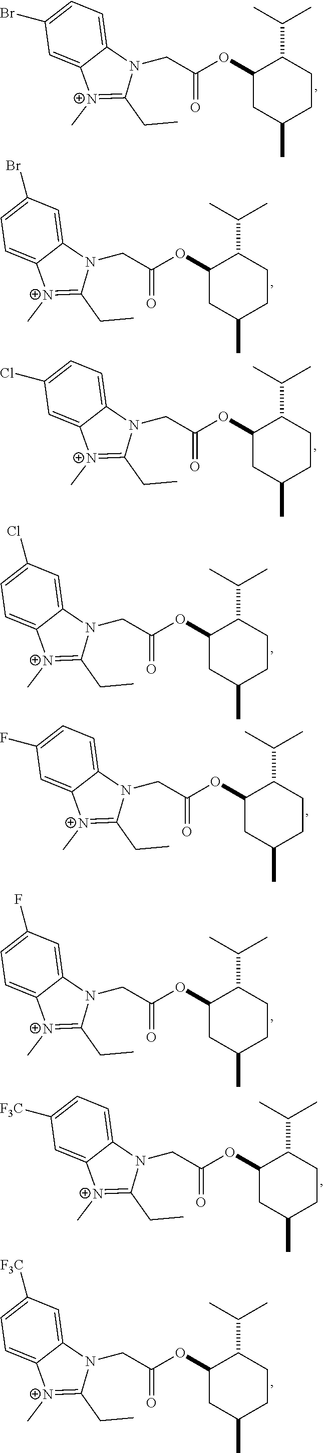 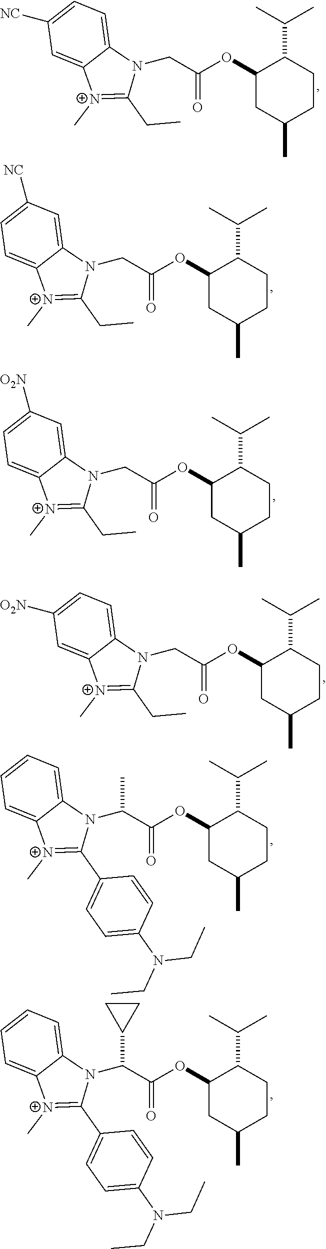 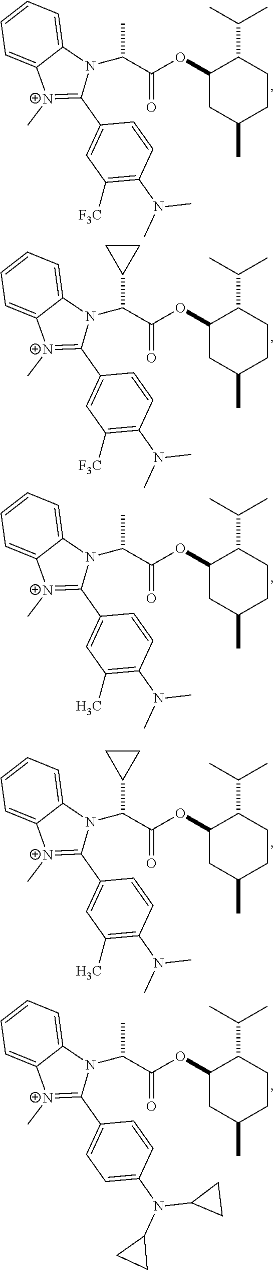 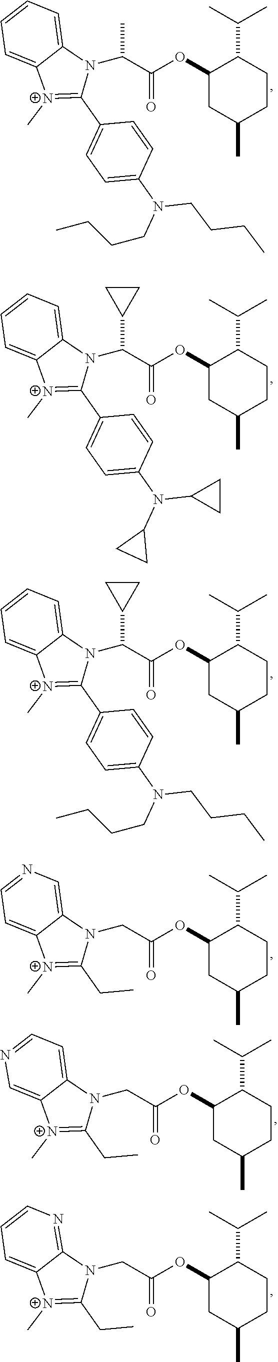 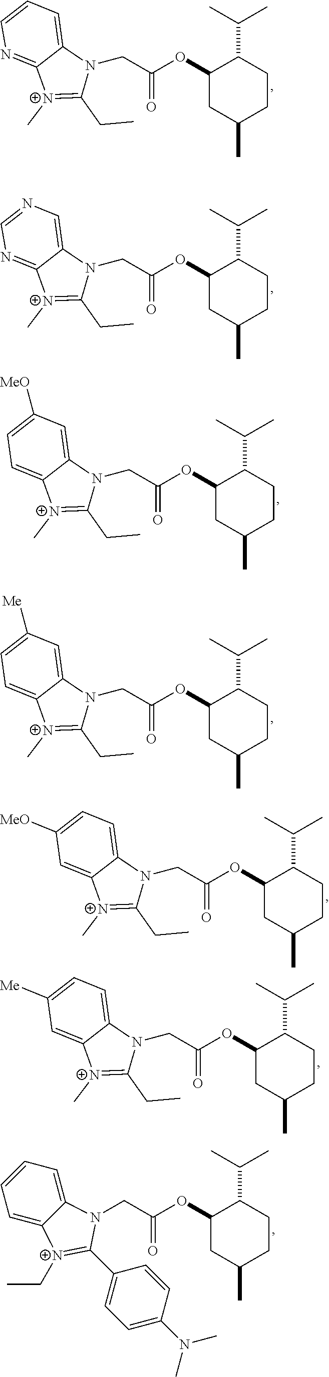 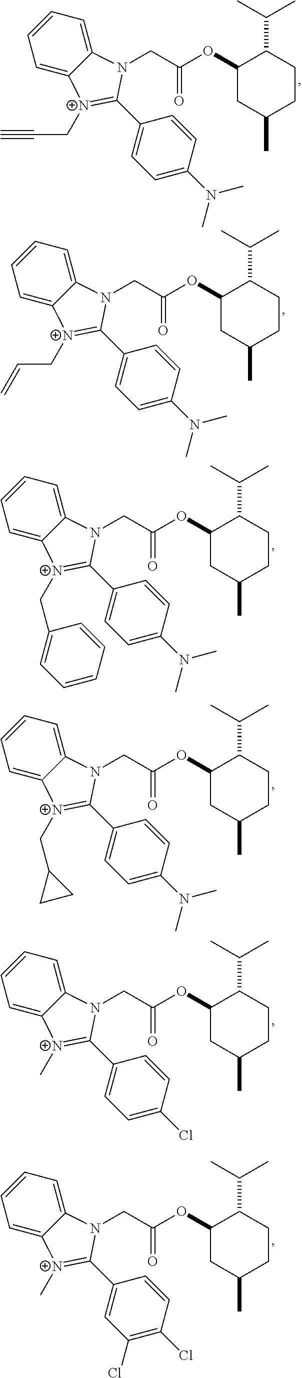 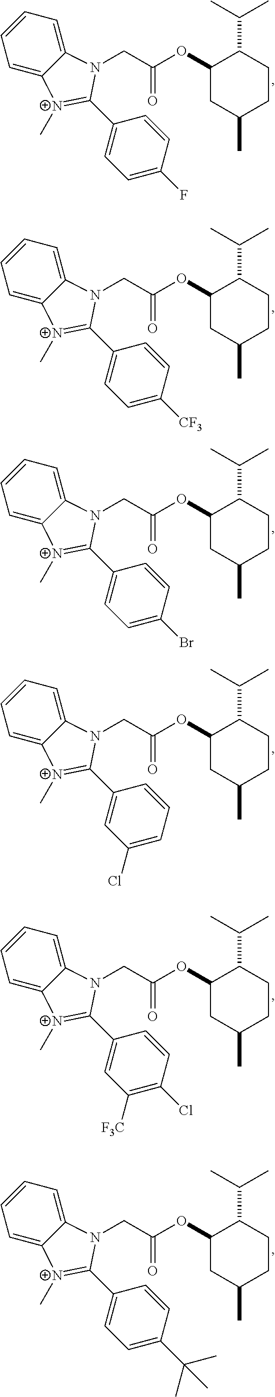 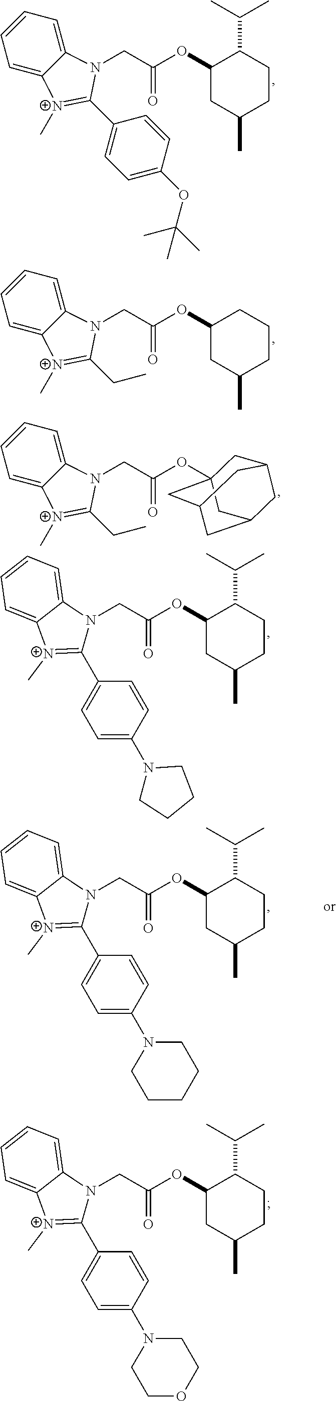
wherein the compound further comprises a pharmaceutically acceptable anion.

93. (canceled)

94. A compound of the formula: 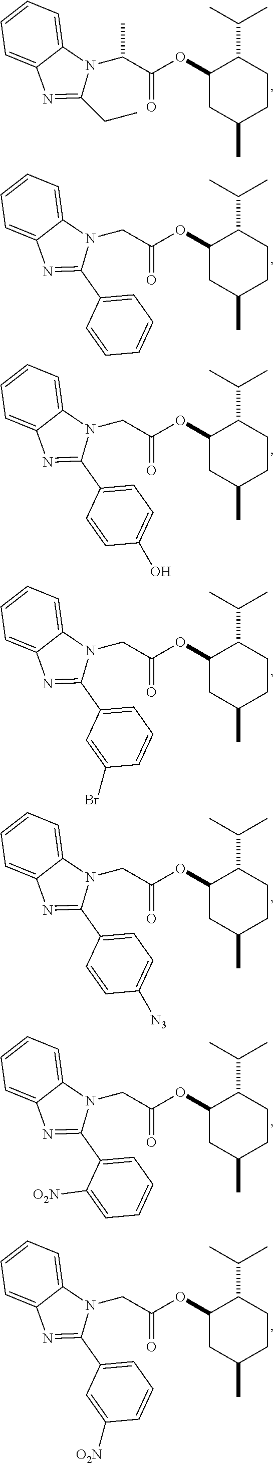 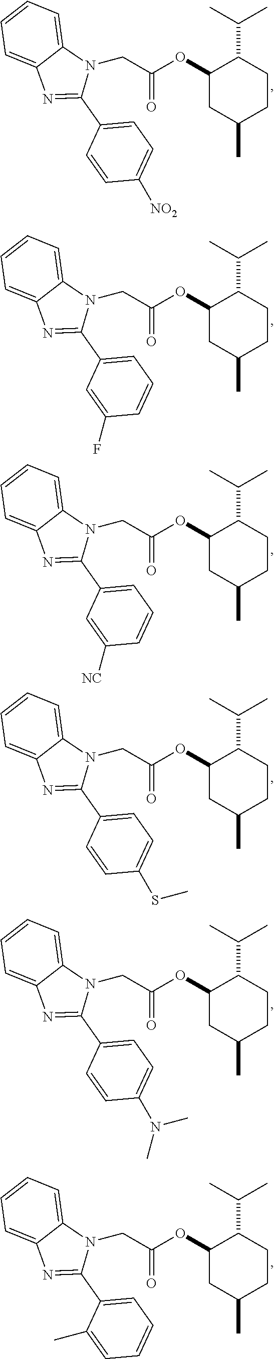 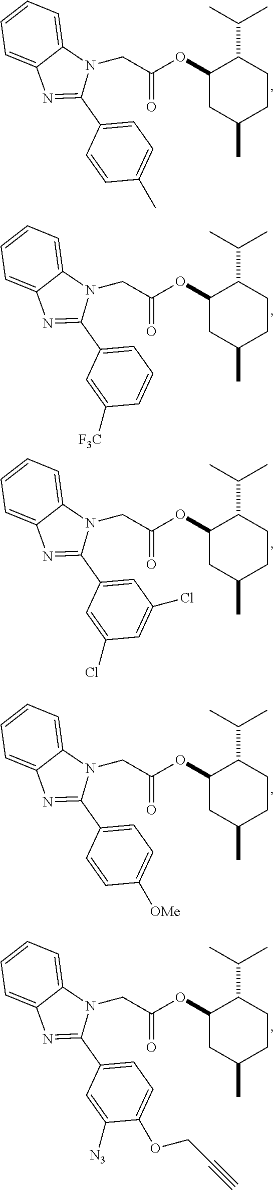 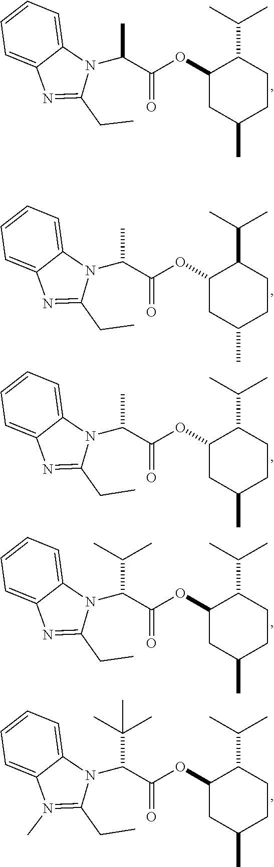 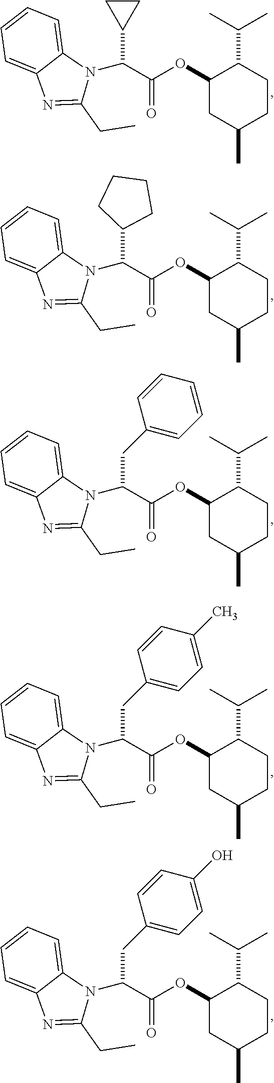 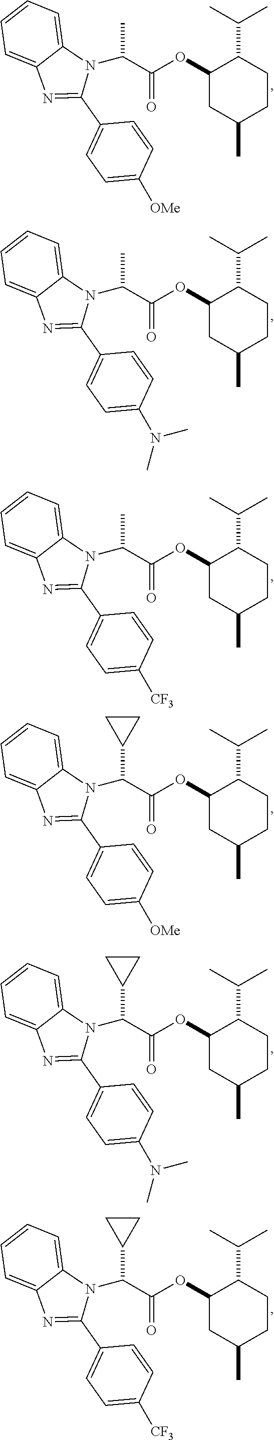 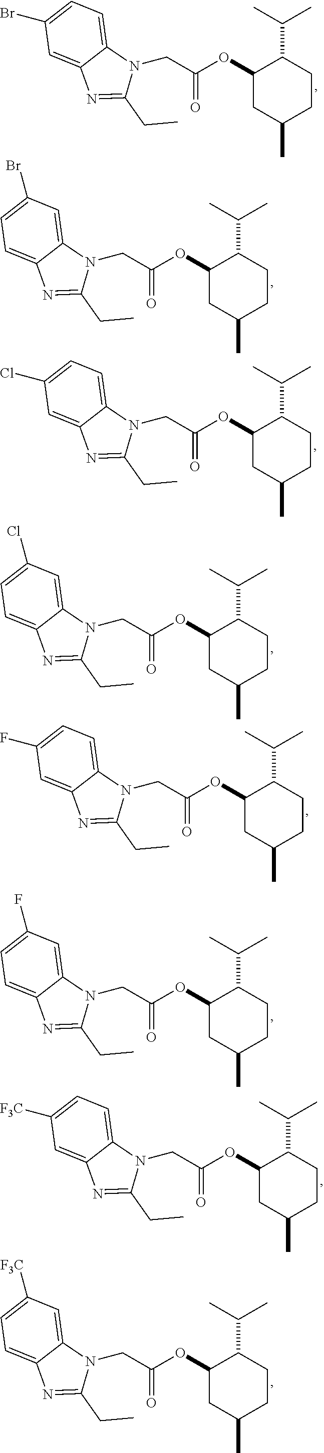 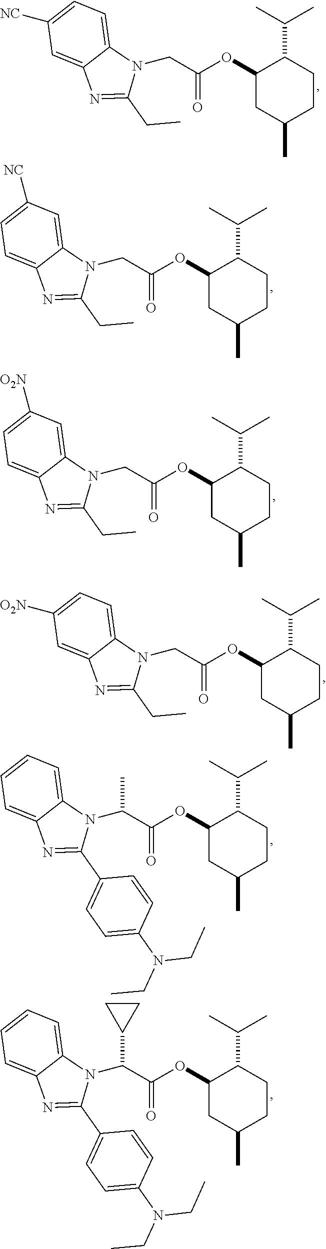 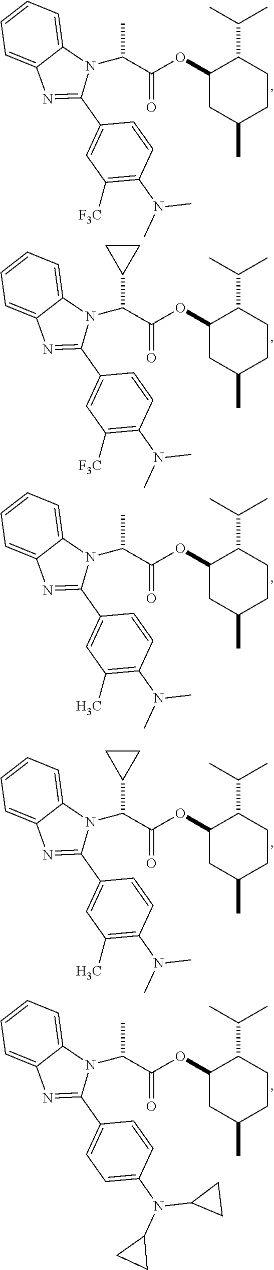 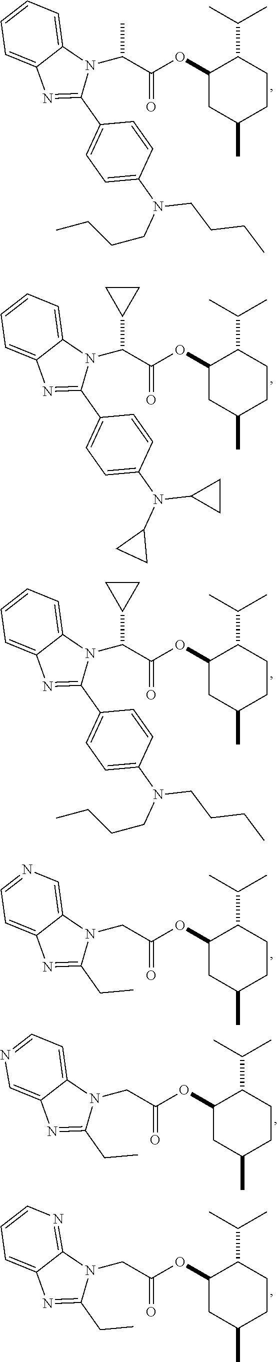 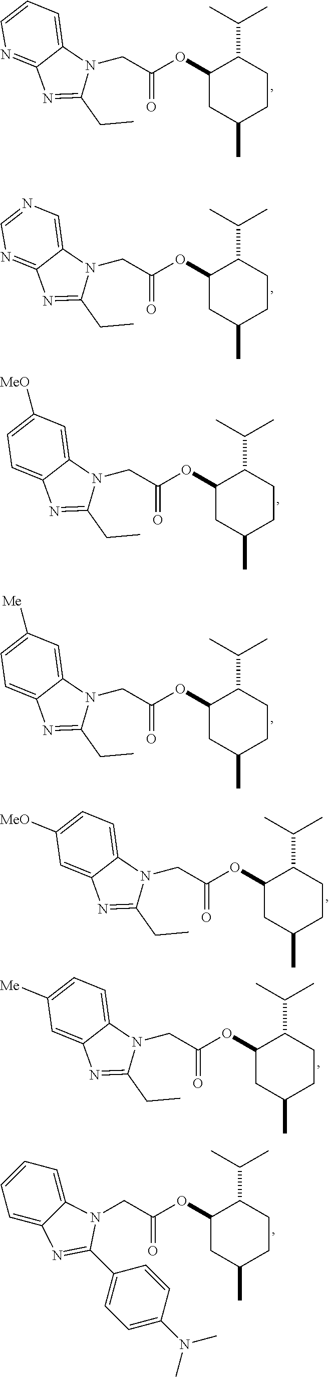 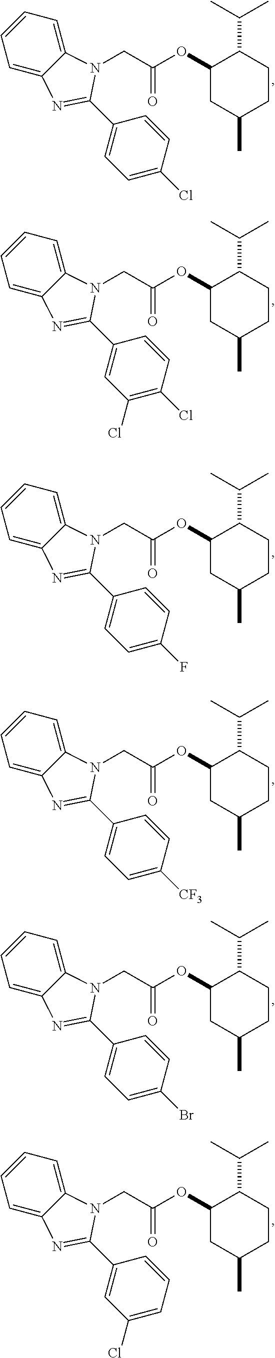 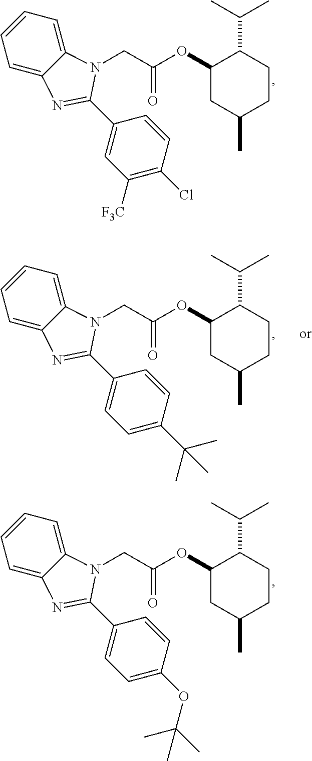
or a pharmaceutically acceptable salt thereof.

95. A pharmaceutical composition comprising:
(a) a compound according to claim 2; and
(b) a pharmaceutically acceptable carrier.

96-97. (canceled)

98. A method of treating a disease or disorder in a patient comprising administering to the patient in need thereof a pharmaceutically effective amount of a compound or composition according to claim 1.

99-113. (canceled)

114. A method of preparing a compound of formula I comprising reacting a compound with a compound of the formula: 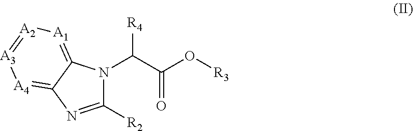
wherein:
R₂ is alkyl_{(C≤12)}, cycloalkyl_{(C≤12)}, alkenyl_{(C≤12)}, cycloalkenyl_{(C≤12)}, alkynyl_{(C≤12)}, aryl_{(C≤12)}, aralkyl_{(C≤12)}, aralkenyl_{(C≤12)}, heteroaryl_{(C≤12)}, heteroaralkyl_{(C≤12)}, heteroaralkenyl_{(C≤12)}, heterocycloalkyl_{(C≤12)}, or a substituted version of any of these groups; or a group of the formula: 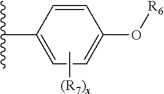
wherein:
R₆ is hydrogen or alkyl_{(C≤8)}, alkenyl_{(C≤8)}, alkynyl_{(C≤8)}, aryl_{(C≤12)}, heteroaryl_{(C≤12)}, aralkyl_{(C≤12)}, heteroaralkyl_{(C≤12)}, acyl_{(C≤8)}, or a substituted version of any of these groups; an ester formed from biotin, or —C(O)CH₂NR₈R₉, wherein:
R₈ and R₉ are each independently alkyl_{(C≤12)}, cycloalkyl_{(C≤12)}, alkenyl_{(C≤12)}, alkynyl_{(C≤12)}, aryl_{(C≤12)}, aralkyl_{(C≤12)}, an amide formed from biotin, or a group of the formula: 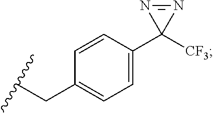 [SMILES: *Cc1ccc(C2(C(F)(F)F)N=N2)cc1]
R₇ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
x is 0, 1, 2, or 3; or
a compound of the formula: 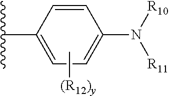
wherein:
R₁₀ and R₁₁ are each independently alkyl_{(C≤8)}, cycloalkyl_{(C≤8)}, or a substituted version of either of these groups; or R₁₀ and R₁₁ are taken together and form a heterocycloalkyl_{(C≤6)} or a substituted version thereof;
R₁₂ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
y is 0, 1, 2, or 3;
R₃ is alkyl_{(C≤12)}, cycloalkyl_{(C≤12)}, bicycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, or a substituted version of any of these groups;
R₄ is hydrogen or alkyl_{(C≤12)}, cycloalkyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, -alkanediyl_{(C≤6)}-heterocycloalkyl_{(C≤12)} or a substituted version of any of these groups; abd
A₁, A₂, A₃, and A₄ are each independently CH, N, or CR₅, wherein:
R₅ is amino, azido, carboxy, cyano, halo, hydroxy, nitro, hydroxysulfonyl, sulfonamide, or
alkyl_{(C≤8)}, cycloalkyl_{(C≤8)}, acyl_{(C≤8)}, alkoxy_{(C≤8)}, —C(O)-alkoxy_{(C≤8)}, acyloxy_{(C≤8)}, aryloxy_{(C≤8)}, heteroaryloxy_{(C≤8)}, heterocycloalkyloxy_{(C≤8)}, alkylthio_{(C≤12)}, aryl_{(C≤8)}, heteroaryl_{(C≤8)}, heterocycloalkyl_{(C≤8)}, alkylsulfonyl_{(C≤8)}, or a substituted version of any of these groups;
—S(O)₂N(Rₐ)R_{b}, —NR_{c}C(O)Rₐ, —C(O)NRₐ(R_{b}), or —NRₐ(R_{b}), or a substituted version of any of these groups;
wherein:
Rₐ and R_{b} are each independently hydrogen, alkyl_{(C≤8)}, cycloalkyl_{(C≤8)}, aryl_{(C≤8)}, heteroaryl_{(C≤8)}, heterocycloalkyl_{(C≤8)}, or a substituted version of any of these groups; and
R_{c} is hydrogen, alkyl_{(C≤8)}, or substituted alkyl_{(C≤8)};
with a compound of the formula:
R₁—X (III)
wherein:
R₁ is alkyl_{(C≤12)}, cycloalkyl_{(C≤12)}, alkenyl_{(C≤12)}, alkynyl_{(C≤12)}, aralkyl_{(C≤12)}, heteroaralkyl_{(C≤12)}, -alkanediyl_{(C≤6)}-cycloalkyl_{(C≤12)}, or a substituted version of any of these groups; and
X is an activating group.

115-122. (canceled)


SEQUENCE LISTING (58 sequences)

SEQ ID NO: 1 - DNA, 19 bp, Artificial sequence
  Synthetic primer
cctggtgatgtccgacctg

SEQ ID NO: 2 - DNA, 19 bp, Artificial sequence
  Synthetic primer
ccatgagcgcatcgcaatc

SEQ ID NO: 3 - DNA, 21 bp, Artificial sequence
  Synthetic primer
cctgcttatcaatgcagaggg

SEQ ID NO: 4 - DNA, 19 bp, Artificial sequence
  Synthetic primer
tgcgggtcaccatttcagc

SEQ ID NO: 5 - DNA, 19 bp, Artificial sequence
  Synthetic primer
gcgaggatgagagcagttc

SEQ ID NO: 6 - DNA, 17 bp, Artificial sequence
  Synthetic primer
aagtcctgtgccaagta

SEQ ID NO: 7 - DNA, 19 bp, Artificial sequence
  Synthetic primer
ctcgactcctcgcagatcg

SEQ ID NO: 8 - DNA, 20 bp, Artificial sequence
  Synthetic primer
gatccagcctccgtttcacc

SEQ ID NO: 9 - DNA, 20 bp, Artificial sequence
  Synthetic primer
acggcgcaacctacaaagag

SEQ ID NO: 10 - DNA, 20 bp, Artificial sequence
  Synthetic primer
gtctgcgtgtaaagcgaagt

SEQ ID NO: 11 - DNA, 20 bp, Artificial sequence
  Synthetic primer
ccgaaaggatggacacggtg

SEQ ID NO: 12 - DNA, 21 bp, Artificial sequence
  Synthetic primer
ttatcggggtctacgttgagc

SEQ ID NO: 13 - DNA, 21 bp, Artificial sequence
  Synthetic primer
ctggatgctgcaaacggatag

SEQ ID NO: 14 - DNA, 23 bp, Artificial sequence
  Synthetic primer
cgaagggaacagtggtcttaaca

SEQ ID NO: 15 - DNA, 20 bp, Artificial sequence
  Synthetic primer
cagaaggagaacgcctaccc

SEQ ID NO: 16 - DNA, 19 bp, Artificial sequence
  Synthetic primer
gagagcaagcgcagatgtc

SEQ ID NO: 17 - DNA, 20 bp, Artificial sequence
  Synthetic primer
cccgctggcgaaagaaattc

SEQ ID NO: 18 - DNA, 21 bp, Artificial sequence
  Synthetic primer
catttggcgaagcctccttta

SEQ ID NO: 19 - DNA, 21 bp, Artificial sequence
  Synthetic primer
ctggaagcctggtatgaggat

SEQ ID NO: 20 - DNA, 22 bp, Artificial sequence
  Synthetic primer
cagggtcaagagtagtgaaggt

SEQ ID NO: 21 - DNA, 21 bp, Artificial sequence
  Synthetic primer
tgcaggaagaaaccgttggag

SEQ ID NO: 22 - DNA, 23 bp, Artificial sequence
  Synthetic primer
ctcgttttaggactggacacttg

SEQ ID NO: 23 - DNA, 19 bp, Artificial sequence
  Synthetic primer
agcagcacttagagtcgcc

SEQ ID NO: 24 - DNA, 19 bp, Artificial sequence
  Synthetic primer
cctgggtaaggggaggagt

SEQ ID NO: 25 - DNA, 20 bp, Artificial sequence
  Synthetic primer
gaggctggcttcatccactg

SEQ ID NO: 26 - DNA, 23 bp, Artificial sequence
  Synthetic primer
ctttttgcttgttgttggtctcc

SEQ ID NO: 27 - DNA, 22 bp, Artificial sequence
  Synthetic primer
tcttttgaggtggtcttcaacg

SEQ ID NO: 28 - DNA, 23 bp, Artificial sequence
  Synthetic primer
gctttgactcgggtaacttcaca

SEQ ID NO: 29 - DNA, 20 bp, Artificial sequence
  Synthetic primer
ccttcgaccagtcgggtttg

SEQ ID NO: 30 - DNA, 20 bp, Artificial sequence
  Synthetic primer
ctgtcccggaaaaggcatcc

SEQ ID NO: 31 - DNA, 20 bp, Artificial sequence
  Synthetic primer
ggggttgttgatgtttttgg

SEQ ID NO: 32 - DNA, 20 bp, Artificial sequence
  Synthetic primer
cgaaacggaaaaggttctca

SEQ ID NO: 33 - DNA, 20 bp, Artificial sequence
  Synthetic primer
ctgtggattttcgggtacgg

SEQ ID NO: 34 - DNA, 20 bp, Artificial sequence
  Synthetic primer
cccctatggaaggtgtctcc

SEQ ID NO: 35 - DNA, 19 bp, Artificial sequence
  Synthetic primer
ggggactacagcaccactc

SEQ ID NO: 36 - DNA, 22 bp, Artificial sequence
  Synthetic primer
gttccgacactccatcagaaat

SEQ ID NO: 37 - DNA, 21 bp, Artificial sequence
  Synthetic primer
actgtgcaggcaactcttgaa

SEQ ID NO: 38 - DNA, 19 bp, Artificial sequence
  Synthetic primer
gttcgcggagcgatacagg

SEQ ID NO: 39 - DNA, 19 bp, Artificial sequence
  Synthetic primer
gcagttggagtgtgagtcg

SEQ ID NO: 40 - DNA, 21 bp, Artificial sequence
  Synthetic primer
tctgtggattcagcacccttt

SEQ ID NO: 41 - DNA, 20 bp, Artificial sequence
  Synthetic primer
agtgcgaaatgaagccgttg

SEQ ID NO: 42 - DNA, 19 bp, Artificial sequence
  Synthetic primer
gactggcgggattgtcacc

SEQ ID NO: 43 - DNA, 21 bp, Artificial sequence
  Synthetic primer
gaactctgccttagctgaggt

SEQ ID NO: 44 - DNA, 21 bp, Artificial sequence
  Synthetic primer
attcccgttttctccgacacg

SEQ ID NO: 45 - DNA, 21 bp, Artificial sequence
  Synthetic primer
cccttcccagcaacctctaaa

SEQ ID NO: 46 - DNA, 20 bp, Artificial sequence
  Synthetic primer
tcttggtccgacctttccga

SEQ ID NO: 47 - DNA, 20 bp, Artificial sequence
  Synthetic primer
tccgagtgccattccgagat

SEQ ID NO: 48 - DNA, 20 bp, Artificial sequence
  Synthetic primer
tccgggtgtagacccatcac

SEQ ID NO: 49 - DNA, 20 bp, Artificial sequence
  Synthetic primer
ggagctgggtatgggtgaga

SEQ ID NO: 50 - DNA, 23 bp, Artificial sequence
  Synthetic primer
tggatggtcctgctttatctttg

SEQ ID NO: 51 - DNA, 19 bp, Artificial sequence
  Synthetic primer
ggcaccgtcatcggatcag

SEQ ID NO: 52 - DNA, 21 bp, Artificial sequence
  Synthetic primer
ctccacaggcagaccagaaaa

SEQ ID NO: 53 - DNA, 19 bp, Artificial sequence
  Synthetic primer
ctgggccagtttgtgaccc

SEQ ID NO: 54 - DNA, 23 bp, Artificial sequence
  Synthetic primer
acgtcatgcaaatcccatgtaaa

SEQ ID NO: 55 - DNA, 21 bp, Artificial sequence
  Synthetic primer
cttgttcagagcggagaaagc

SEQ ID NO: 56 - DNA, 21 bp, Artificial sequence
  Synthetic primer
acatctgcaagtacgttcgtt

SEQ ID NO: 57 - DNA, 19 bp, Artificial sequence
  Synthetic primer
ggcagcaggcaatgcaatg

SEQ ID NO: 58 - DNA, 19 bp, Artificial sequence
  Synthetic primer
gggctcgtcactccagtct